(19) United States
(12) Patent Application Publication
(10) Pub. No.: US 2026/0242395 A1
(43) Pub. Date: Aug. 20, 2026

(54) AMINOHETEROARYL KINASE INHIBITORS

(71) Applicant: ALLORION THERAPEUTICS INC, Natick, MA (US)

(72) Inventors: Dai CHENG, Guangzhou, Guangdong (CN); Yang ZHOU, Guangzhou, Guangdong (CN); Sen ZENG, Guangzhou, Guangdong (CN); Qiming YUE, Guangzhou, Guangdong (CN); Zhixiang HE, Guangzhou, Guangdong (CN); Xiaohang YIN, Guangzhou, Guangdong (CN); Zeqiang XIE, Guangzhou, Guangdong (CN); Wei HAN, Guangzhou, Guangdong (CN); Pengfei LEI, Guangzhou, Guangdong (CN); Xiaobo ZHOU, Guangzhou, Guangdong (CN); Yangyin XIE, Guangzhou, Guangdong (CN); Yuhong FU, Beijing (CN); Jianwei CHE, Boston, MA (US); Guosheng WU, Beijing (CN)

(21) Appl. No.: 19/099,391

(22) PCT Filed: Jul. 28, 2023
(86) PCT No.: PCT/CN2023/109836

§ 371 Date: Jan. 28, 2025

(30) Foreign Application Priority Data
Jul. 29, 2022 (WO) PCT/CN2022/109075
Oct. 31, 2022 (WO) PCT/CN2022/128578
Feb. 28, 2023 (WO) PCT/CN2023/078781

(51) Int. Cl. C07D 498/04; A61K 31/4439; A61K 31/4709; A61K 31/506; A61K 31/5383; C07B 59/00; C07D 401/14; C07D 405/14; C07D 471/04; C07D 471/10; C07D 471/18; C07D 491/052

(52) U.S. Cl. CPC C07D 498/04; A61K 31/4439; A61K 31/4709; A61K 31/506; A61K 31/5383; C07B 59/002; C07D 401/14; C07D 405/14; C07D 471/04; C07D 471/10; C07D 471/18; C07D 491/052; C07B 2200/05

(57) ABSTRACT

Provided herein are novel compounds (e.g., Formula I), pharmaceutical compositions, and methods of using related to cyclin dependent kinases (CDKs). The compounds herein are typically CDK4 inhibitors, which can be used for treating a variety of diseases or disorders, such as cancer.

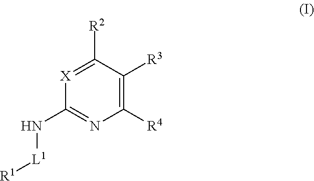

### CROSS-REFERENCE TO RELATED APPLICATIONS

This application claims priority to International Patent Application Nos. PCT/CN2022/109075, filed on Jul. 29, 2022, PCT/CN2022/128578, filed on Oct. 31, 2022, and PCT/CN2023/078781, filed on Feb. 28, 2023, the entirety of each of which is incorporated herein by reference.

### FIELD

In various embodiments, the present disclosure generally relates to novel heteroaryl compounds, compositions comprising the same, methods of preparing and methods of using the same, e.g., for inhibiting cyclin-dependent kinases and/or for treating or preventing various diseases or disorders described herein.

### BACKGROUND

Unrestrained cell growth is the hall mark of cancer and disrupted cell-cycle regulation is a common feature in malignant cells. Cyclin-dependent kinases (CDKs) are a family of serine/threonine protein kinases that regulate mammalian cell division and proliferation. At least 20 CDKs and 29 cyclins have been identified in human cells (Cao et. al., BMC Evol. Biol, 2014; 14:10). In addition to regulate cell cycle progression, these CDKs and cyclins also play important roles in regulating transcription, DNA repair, differentiation and apoptosis (Palmer and Kaldis, Semin Cell Dev Biol, 2020; 107:54-62).

CDK4 and CDK6 are key regulators for G1-S transition. D type cyclins form complexes with CDK4/6 and phosphorylates the retinoblastoma (Rb) protein. This relieves the Rb-mediated inhibition of the transcription factor E2F and leads to S phase entrance. CDK4/6-CyclinD-Rb pathway is frequently perturbed in human cancers. Amplification of CDK4/6 and Cyclin D1, over-expression of D type cyclins, mutation, genetic deletion, or transcriptional silencing of endogenous CDK4/6 inhibitor p16 (CDKN2A) have all been reported as mechanisms which leads to pathway activation (Dickson, Mol Cancer Res. 2014; 20: 3379-3383). In breast cancer, dysregulation of CDK4/6 pathway is associated with resistance to endocrine therapy (Ding et. al., Int J Mol Sci. 2020; 21:1960).

In recent years, targeted inhibition of CDKs has shown considerable therapeutic benefit in a variety of tumor types. The use of CDK4/6 inhibitors together with endocrine therapies were shown to be effective in treating human epidermal growth factor 2 (HER2) negative, estrogen receptor (ER) positive breast cancer. As a result, there different CDK4/6 inhibitors, palbociclib, ribociclib and abemaciclib were approved in combination with endocrine therapies in both first or second line setting (Cogliati, et. al., Life, 2022; 12:378). Most recently abemaciclib is also approved for adjuvant treatment of early breast cancer.

Despite the success of CDK4/6 inhibitors in clinic, treatment related adverse effects, especially hematologic toxicity such as neutropenia, prevented continued dosing of CDK4/6 inhibitors. Currently, both Palbociclib and Ribociclib are administrated on a three week on/one week off schedule. Mouse genetic and other emerging data suggest that the observed hematologic toxicity is likely linked to CDK6-cyclin D3 inhibition (Sicinska et. al., Mol Cell Biol, 2006; 26: 8052-8060; Cooper et. al., Nat Immunol, 2006; 5:489-497). On the other hand, CDK6-cyclin D3 is expressed at very low level in HR+/HER2− breast cancer and CDK4 was identified as the oncogenic driver in this tumor type (Zhang et. al., Cancer Res, 2022; epub).

### BRIEF SUMMARY

Inhibitors specific to a particular CDK such as CDK4 may have the advantage of improved safety profile and may enable improved dosing schedule and higher dosing, and may lead to deeper target inhibition, better efficacy and potentially overcome drug resistance. There remains a need for CDK inhibitors, such as selective CDK4 inhibitors, such as those with improved efficacy and reduced side effects in clinic.

In various embodiments, the present disclosure provides novel compounds, which can inhibit CDKs, such as selectively inhibit CDK4. The compounds and compositions herein are useful for treating various diseases or disorders assocatied with abnormal CDK4 activity, such as Hormonal receptor positive, HER2-negative breast cancer.

Some embodiments of the present disclosure are directed to a compound of Formula I, or a pharmaceutically acceptable salt thereof,

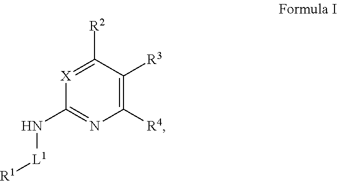

wherein the variables are defined herein. In some embodiments, the compound of Formula I can have a structure according to any sub-formula provided herein, such as I-A, I-B, A, A-S1, A-S2, A-S3, A-S4, A-1, A-2, I-1, I-1-D, I-2, I-2-D, I-3, or I-4, as defined herein. In some embodiments, the present disclosure also provides specific compounds selected from any of Examples 1-111, or any of the specific compounds disclosed in Table 1 herein, or a pharmaceutically acceptable salt thereof.

In some embodiments, the present disclosure provides a pharmaceutical composition comprising one or more compounds of the present disclosure and optionally a pharmaceutically acceptable excipient. The pharmaceutical composition can be typically formulated for oral administration.

In some embodiments, the present disclosure also provides a method of inhibiting CDK activity such as CDK4 activity in a subject or biological sample. In some embodiments, the method comprises contacting the subject or biological sample with an effective amount of one or more compounds of the present disclosure, e.g., a compound of Formula I (or any sub-formula provided herein, e.g., Formula I-A, I-B, A, A-S1, A-S2, A-S3, A-S4, A-1, A-2, I-1, I-1-D, I-2, I-2-D, I-3, or I-4), any of Examples 1-111, or any of the specific compounds disclosed in Table 1 herein, or a pharmaceutically acceptable salt thereof, or a pharmaceutical composition comprising the same.

In some embodiments, the present disclosure provides a method of treating or preventing a CDK-mediated disease or disorder in a subject in need thereof. In some embodiments, the method comprises administering to the subject an effective amount of one or more compounds of the present disclosure or the pharmaceutical composition herein. In some embodiments, the method comprises administering to the subject an effective amount of a compound of Formula I (or any sub-formula provided herein, e.g., Formula I-A, I-B, A, A-S1, A-S2, A-S3, A-S4, A-1, A-2, I-1, I-1-D, I-2, I-2-D, I-3, or I-4), any of Examples 1-111, or any of the specific compounds disclosed in Table 1 herein, or a pharmaceutically acceptable salt thereof, or a pharmaceutical composition comprising the same.

In some embodiments, the present disclosure also provides a method of treating or preventing cancer in a subject in need thereof, which comprises administering to the subject an effective amount of a compound of the present disclosure (e.g., a compound of Formula I (or any sub-formula provided herein, e.g., Formula I-A, I-B, A, A-S1, A-S2, A-S3, A-S4, A-1, A-2, I-1, I-1-D, 1-2, I-2-D, I-3, or 1-4), any of Examples 1-111, or any of the specific compounds disclosed in Table 1 herein, or a pharmaceutically acceptable salt thereof) or an effective amount of a pharmaceutical composition described herein. In some embodiments, the cancer is selected from breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer, thyroid cancer, and combinations thereof. In some embodiments, the cancer is breast cancer selected from ER-positive/HR-positive, HER2-negative breast cancer; ER-positive/HR-positive, HER2-positive breast cancer; triple negative breast cancer (TNBC); and inflammatory breast cancer. In some embodiments, the cancer is breast cancer. In some embodiments, the cancer is breast cancer selected from endocrine resistant breast cancer, trastuzumab resistant breast cancer, or breast cancer demonstrating primary or acquired resistance to CDK4/CDK6 inhibition. In some embodiments, the cancer is advanced or metastatic breast cancer. In some embodiments, the cancer is ovarian cancer.

The administering in the methods herein is not limited to any particular route of administration. For example, in some embodiments, the administering can be orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the administering is orally. In some embodiments, the administering is a parenteral injection, such as an intraveneous injection.

Compounds of the present disclosure can be used as a monotherapy or in a combination therapy. In some embodiments according to the methods described herein, one or more compounds of the present disclosure can be administered as the only active ingredient(s). In some embodiments, the method herein further comprises administering to the subject an additional therapeutic agent, such as additional anticancer agents described herein.

It is to be understood that both the foregoing summary and the following detailed description are exemplary and explanatory only, and are not restrictive of the invention herein.

### DETAILED DESCRIPTION

In various embodiments, the present disclosure provides compounds and compositions that are useful for inhibiting CDKs such as CDK4 and/or treating or preventing various diseases or disorders described herein, e.g., cancer.

### Compounds

The compounds of the present disclosure are generally aminopyridine or aminopyrimidine derivatives having a Formula I described herein. The compounds herein can typically inhibit CDKs, such as CDK4. In some embodiments, the compounds herein can selectively inhibit CDK4 over other CDKs.

### Formula I

In some embodiments, the present disclosure provides a compound of Formula I, or a pharmaceutically acceptable salt thereof:

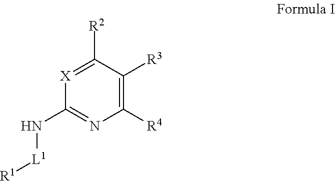

wherein:
L¹ is an optionally substituted phenylene, optionally substituted 5- or 6-membered heteroarylene, optionally substituted 4-8-membered heterocyclylene, or optionally substituted C₃₋₈ carbocyclylene;
R¹ is hydrogen, OH, NH₂, NHCH₃, or N(CH₃)₂;
X is N or CR¹⁰;
R² is an optionally substituted ring system selected from a 10 membered heteroaryl, 9-12 membered bicyclic heterocyclic ring, or 12-16 membered heteroaryl or heterocyclic ring structure having three or more rings, wherein the ring system includes at least one phenyl or heteroaryl ring, and wherein R² connects to the remainder of Formula I through a ring atom of a phenyl or heteroaryl portion of the ring system;
R³ is hydrogen, deuterium, halogen (e.g., F, Cl), CN, OR¹¹, NR¹²R¹³, C(O)NR¹²R¹³, COOR^{A}, COR^{B}, optionally substituted C₁₋₆ alkyl, optionally substituted C₂₋₄ alkenyl, optionally substituted C₂₋₄ alkynyl, optionally substituted C₁₋₄ heteroalkyl, optionally substituted C₃₋₈ carbocyclyl, optionally substituted 4-10 membered heterocyclyl, or optionally substituted 5-10 membered heteroaryl;
R⁴ is hydrogen, deuterium, halogen (e.g., F), optionally substituted C₁₋₆ alkyl, or NR¹²R¹³; wherein:
R¹⁰ is hydrogen, halogen (e.g., F), CN, —OH, an optionally substituted C₁₋₄ alkyl, optionally substituted C₁₋₄ heteroalkyl, optionally substituted C₃₋₈ carbocyclyl, or optionally substituted 4-10 membered heterocyclyl;
R¹¹ is hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or an oxygen protecting group;
each of R¹² and R¹³, at each occurrence, is independently hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or a nitrogen protecting group; or R¹² and R¹³ can be joined to form an optionally substituted 4-10 membered heterocyclyl or 5- or 6-membered heteroaryl;
R^{A} is hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or an oxygen protecting group; and
R^{B} is hydrogen, an optionally substituted C₁₋₆ alkyl, optionally substituted C₃₋₈ carbocyclyl, optionally substituted phenyl, optionally substituted 4-10 membered heterocyclyl, or optionally substituted heteroaryl (e.g., 5- or 6-membered heteroaryl).

In one embodiment, said phenyl or heteroaryl portion of R² (that connects to the remainder of Formula I) is not fused to a 5-membered heteroaryl.

In some embodiments, the compound of Formula I (including any of the applicable sub-formulae as described herein) can comprise one or more asymmetric centers and/or axial chirality, and thus can exist in various stereoisomeric forms, e.g., enantiomers and/or diastereomers. In some embodiments, the compound of Formula I can exist in the form of an individual enantiomer and/or diastereomer, as applicable, or a mixture of stereoisomers, including racemic mixtures and mixtures enriched in one or more stereoisomers. In some embodiments, when applicable, the compound of Formula I (including any of the applicable sub-formulae as described herein) can have an enantiomeric excess (“ee”) of greater than 60%, such as having greater than 80% ee, greater than 90% ee, greater than 95% ee, greater than 98% ee, greater than 99% ee, or with a non-detectable amount of the other enantiomer. In some embodiments, when applicable, the compound of Formula I (including any of the applicable sub-formulae as described herein) can also exist as a mixture of stereoisomers in any ratio, such as a racemic mixture.

In some embodiments, the compound of Formula I (including any of the applicable sub-formulae as described herein) can exist as an isotopically labeled compound, particularly, a deuterated analog, wherein one or more of the hydrogen atoms of the compound of Formula I is/are substituted with a deuterium atom with an abundance above its natural abundance, e.g., a CD₃ analog when the compound has a CH₃ group. Without wishing to be bound by theories, it is believed that deuterium substitution at certain positions (e.g., those explicitly specified herein) can lead to compounds with a better pharmacokinetic profile when compared with their hydrogen counterparts (i.e., at natural abundance) and thus can have a better pharmacodynamic outcome in vivo. Compounds with specific deuterium substitutions can be prepared by methods using commercially available deuterium enriched reagents, such as deuterium gas, deuterium oxide (D₂O), deuterated formic acid (DCOOD), deuterated methyl iodide (CD₃I), deuterated methanol (CD₃OD), sodium deuteroxide (NaOD), sodium borodeuteride (NaBD₄), lithium aluminum deuteride (LiAlD₄), etc.

It should be apparent to those skilled in the art that in certain cases, the compound of Formula I may exist as a mixture of tautomers. The present disclosure is not limited to any specific tautomer. Rather, the present disclosure encompasses any and all of such tautomers whether or not explicitly drawn or referred to.

Typically, X in Formula I is N, and the compound of Formula I can be characterized as having Formula I-A:

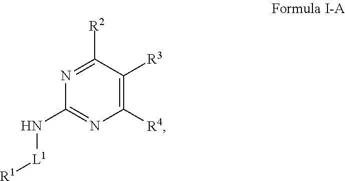

wherein L¹, R¹, R², R³, and R⁴ include any of those described herein in any combination.

In some embodiments, X in Formula I can be CR¹⁰, wherein R¹⁰ is defined herein. For example, in some embodiments, R¹⁰ can be hydrogen, and the compound of Formula I can be characterized as having Formula I-B:

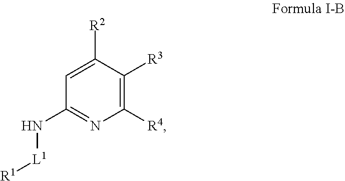

wherein L¹, R¹, R², R³, and R⁴ include any of those described herein in any combination.

Various groups are suitable as L¹ in Formula I. For example, in some embodiments, L¹ in Formula I can be an optionally substituted phenylene. In some embodiments, L¹ in Formula I can be an optionally substituted 5- or 6-membered heteroarylene, e.g., those having 1-3 ring heteroatoms independently selected from N, O, and S, such as pyridylene, e.g.,

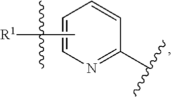

which is optionally substituted. For example, in some embodiments, L¹ in Formula I can be

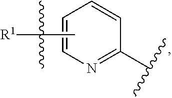

wherein the pyridylene is optionally substituted with a 5-membered heteroaryl, such as a pyrazole or imidazole. In some embodiments, L¹ in Formula I can be selected from:

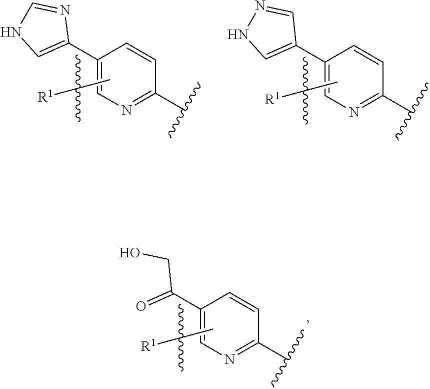

typically, in such embodiments, R¹ can be hydrogen. In some embodiments, L¹ in Formula I can be an optionally substituted 4-8-membered heterocyclylene, e.g., a monocyclic or bicyclic (e.g., fused, bridged, or spiro bicyclic) 4-8 membered heterocyclylene having 1-2 ring heteroatoms independently selected from N, O, and S. In some embodiments, L¹ in Formula I can be an optionally substituted C₃-8 carbocyclylene, e.g., a monocyclic or bicyclic (e.g., fused, bridged, or spiro bicyclic) carbocyclylene.

In some specific embodiments, the compound of Formula I herein can be characterized as having a structure according to Formula A:

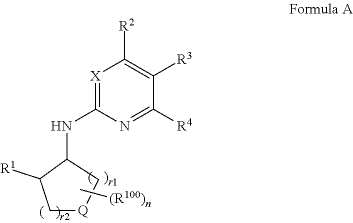

wherein:
   Q is
   (1) O;
   (2) NR¹⁴, wherein R¹⁴ is hydrogen, G^{A}, SO₂G^{A}, SO₂NG^{B}G^{C}, S(O)(NH)G^{A}, COG^{A}, COOG^{A}, or C(O)NG^{B}G^{C};
   (3) CR¹⁵R¹⁶, wherein R¹⁵ and R¹⁶ are joined with the carbon atom they are attached to form an optionally substituted 4-6 membered heterocyclic ring having 1 or 2 ring heteroatoms independently selected from 0 and N; or
   (4) absent;
   r1 is 1, 2, or 3; and
   r2 is 0, 1, or 2;
   n is 0, 1, 2, 3, or 4, as valency permits; and
   (i) R¹⁰⁰ at each occurrence is independently selected from halogen (e.g., F or Cl), CN, OH, COOH, G^{A}, OG^{A}, NG^{B}G^{C}, NG^{B}G^{C}SO₂G^{A}, NG^{B}G^{C}SO₂NG^{B}G^{C}, NG^{B}G^{C}S(O)(NH)G^{A}, NG^{B}G^{C}COG^{A}, NG^{B}G^{C}COOG^{A}, NG^{B}G^{C}C(O)NG^{B}G^{C}, SO₂G^{A}, SO₂NG^{B}G^{C}, S(O)(NH)G^{A}, COG^{A}, COOG^{A}, or C(O)NG^{B}G^{C}; or
   (ii) two instances of R¹⁰⁰ are joined together with the intervening atom(s) to form an optionally substituted ring, such as an optionally substituted 3-6 membered ring, and any remaining R¹⁰⁰ at each occurrence is as defined in (i);
   wherein:
   G^{A} at each occurrence is independently an optionally substituted C₁₋₆ alkyl, optionally substituted C₃₋₈ carbocyclyl, optionally substituted phenyl, optionally substituted heteroaryl (e.g., 5- or 6-membered heteroaryl), or optionally substituted 4-10 membered heterocyclyl; and
   each of G^{B} and G^{C}, at each occurrence, is independently hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or a nitrogen protecting group; or G^{B} and G^{C} can be joined to form an optionally substituted 4-10 membered heterocyclyl or 5- or 6-membered heteroaryl,
   wherein R¹, R², R³, and R⁴ include any of those described herein in any combination.

In some embodiments, the compound of Formula A can be a compound having a stereochemistry as shown in Formula A-S1 or A-S2:

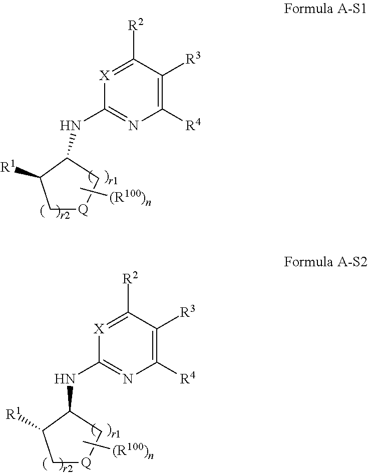

In some preferred embodiments, the compound of Formula A can exist as the stereoisomer of Formula A-S1, such as having an ee of greater than 60%, preferably, with greater than 80%, greater than 90%, greater than 95%, greater than 98%, greater than 99% ee, or with a non-detectable amount of the enantiomer according to Formula A-S2.

In some embodiments, the compound of Formula A can be a compound having a stereochemistry as shown in Formula A-S3 or A-S4:

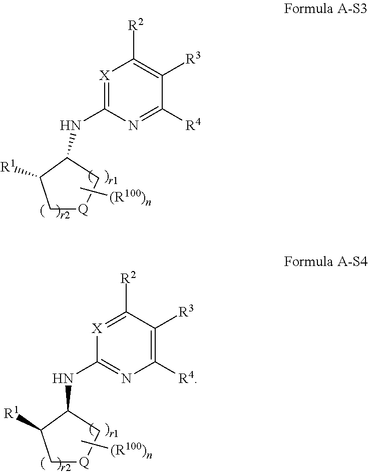

In some embodiments according to Formula A, n is 0. In some embodiments according to Formula A, n is 1 or 2, and R¹⁰⁰ at each occurrence is independently selected from F, Cl, CN, OH, C₁₋₄ alkyl optionally substituted with F, C₁₋₄ alkoxy optionally substituted with F, and C₁₋₄ heteroalkyl optionally substituted with F.

In some embodiments according to Formula A, n is 1, and R¹⁰⁰ is F, Cl, CN, OH, methyl, fluorine-substituted methyl such as CF₃, methoxy, or fluorine-substituted methoxy.

In some embodiments, in Formula A, r1 is 1 and r2 is 1. In some embodiments, r1 is 2 and r2 is 1. In some embodiments, r1 is 1 and r2 is 2. In some embodiments, r1 is 2 and r2 is 2.

In some embodiments, Q in Formula A is absent (i.e., Q is a bond connecting the two carbon atoms adjacent to Q in Formula A).

In some embodiments, Q in Formula A is O.

In some embodiments, in Formula A, r1 is 1, r2 is 1, and Q is O. For example, in some embodiments, the

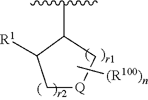

moiety in Formula A can be

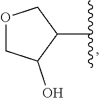 [SMILES: *C1COCC1O]

preferably, with a stereochemistry of

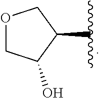 [SMILES: *[C@@H]1COC[C@H]1O]

In one embodiment, the moiety is

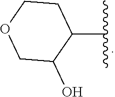 [SMILES: *C1CCOCC1O]

In one embodiment, the moiety is

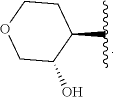 [SMILES: *[C@@H]1CCOC[C@H]1O]

In some embodiments, the compound of Formula A can be characterized as having a structure according to Formula A-1 or A-2:

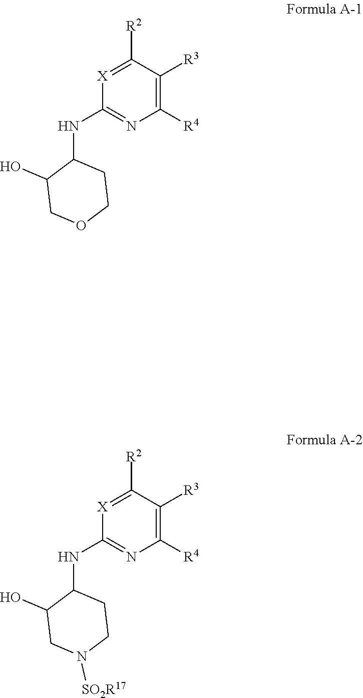

wherein:
R¹⁷ is an optionally substituted C₁₋₄ alkyl, optionally substituted C₃₋₆ cycloalkyl, optionally substituted phenyl, optionally substituted 4-8 membered heterocyclic ring, or optionally substituted 5 or 6 membered heteroaryl.

For example, in some embodiments, R¹⁷ is
(1) C₁₋₄ alkyl optionally substituted with 1-3 substituents independently selected from deuterium, F, and OH, such as CHF₂, CF₃, etc.;
(2) phenyl, pyridyl, or pyrimidyl, each of which is optionally substituted with 1-3 substituents independently selected from deuterium, halogen, CN, OH, C₁₋₃ alkyl optionally substituted with F, or C₁₋₃ alkoxy optionally substituted with F; or
(3) 5-membered heteroaryl optionally substituted with 1-3 substituents independently selected from deuterium, halogen, CN, OH, C₁₋₃ alkyl optionally substituted with F, or C₁₋₃ alkoxy optionally substituted with F.

In some embodiments, R¹⁷ can be C₁₋₄ alkyl, (C₁₋₄ alkylene)j-C₃₋₆ cycloalkyl, (C₁₋₄ alkylene)ⱼ-(4-8 membered monocyclic heterocyclyl having one or two ring heteroatoms independently selected from N, O, and S), or (C₁₋₄ alkylene)ⱼ-(5 or 6 membered heteroaryl having 1-3 ring heteroatoms independently selected from N, O, and S),
wherein j is 0 or 1, and the C₁₋₄ alkylene is straight or branched alkylene chain optionally substituted with F; and
wherein each of the C₁₋₄ alkyl, C₃₋₆ cycloalkyl, 5 or 6 membered heteroaryl and 4-8 membered monocyclic heterocyclyl is optionally substituted with one or more (e.g., 1, 2, or 3) substituents independently selected from oxo (as valency permits), halo (e.g., F), G¹, OH, O-G¹, NH₂, NH(G¹), and N(G¹)(G¹),
wherein G¹ at each occurrence is independently (1) a C₁₋₄ alkyl optionally substituted with 1-3 substituents independently selected from F, CN, OH, and C₁₋₄ heteroalkyl, or (2) a C₃₋₆ cycloalkyl optionally substituted with 1-3 substituents independently selected from F, CN, OH, and C₁₋₄ heteroalkyl;
   when substituted, the C₁₋₄ alkyl, C₃₋₆ cycloalkyl, 5 or 6 membered heteroaryl, or 4-8 membered monocyclic heterocyclyl is preferably substituted with 1, 2, or 3 substituents each independently F, Cl, CN, OH, C₁₋₄ alkyl optionally substituted with F, or C₁₋₄ alkoxy optionally substituted with F, more preferably, with 1, 2, or 3 substituents each independently F, OH, methyl, fluorine-substituted methyl such as CF₃, methoxy, or fluorine-substituted methoxy. In some embodiments, j is 0. In some embodiments, j is 1, and the C₁₋₄ alkylene is straight or branched alkylene chain, such as CH₂.

In some embodiments, R¹⁷ can be C₁₋₄ alkyl, such as methyl, ethyl, isopropyl, etc. In some embodiments, R¹⁷ can be C₁₋₄ alkyl optionally substituted with F.

In some embodiments, R¹⁷ can be C₃₋₆ cycloalkyl, which is optionally substituted, e.g., with a substituent described herein.

In some embodiments, R¹⁷ can be 4-8 membered monocyclic heterocyclyl having one or two ring heteroatoms independently selected from N, O, and S, which is optionally substituted, e.g., with a substituent described herein.

In some embodiments, R¹⁷ can be a 5-membered heteroaryl, which is optionally substituted, such as with a C₁₋₄ alkyl, for example, R¹⁷ can be selected from:

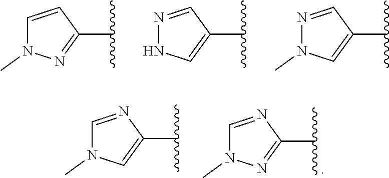

In some embodiments, R¹⁷ can be a phenyl or 6-membered heteroaryl having 1-3 ring nitrogen atoms, wherein the phenyl or 6-membered heteroaryl is optionally substituted with one or more (e.g., 1, 2, or 3) substituents independently selected from halo (e.g., F), G¹, OH, O-G¹, NH₂, NH(G¹), and N(G¹)(G¹),
wherein G¹ at each occurrence is independently (1) a C₁₋₄ alkyl optionally substituted with 1-3 substituents independently selected from F, CN, OH, and C₁₋₄ heteroalkyl, or (2) a C₃₋₆ cycloalkyl optionally substituted with 1-3 substituents independently selected from F, CN, OH, and C₁₋₄ heteroalkyl,
   when substituted, the phenyl or 6-membered heteroaryl is preferably substituted with 1, 2, or 3 substituents each independently F, Cl, CN, OH, C₁₋₄ alkyl optionally substituted with F, C₁₋₄ alkoxy optionally substituted with F, more preferably, with 1 or 2 substituents each independently F or methyl. For example, in some embodiments, R¹⁷ can be,

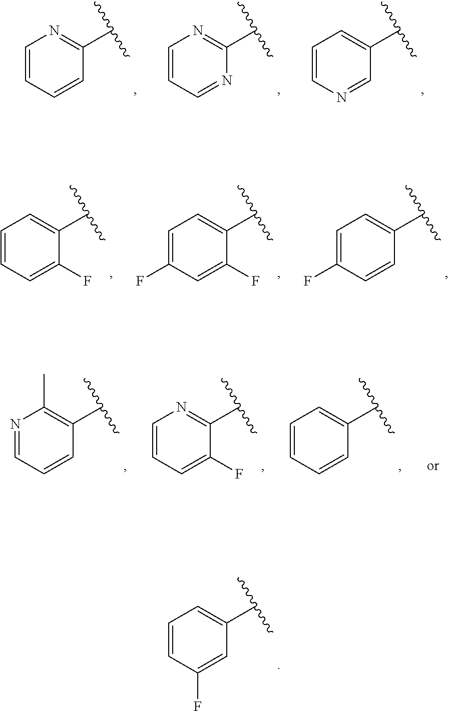

In any of the embodiments herein, unless specified or otherwise contrary from context, R¹ can be OH.

In any of the embodiments herein, unless specified or otherwise contrary from context, X can be N.

In any of the embodiments herein, unless specified or otherwise contrary from context, L¹-R¹ in Formula I (or any of the applicable sub-formulae as described herein, e.g., Formula A, I-A, or I-B) can be

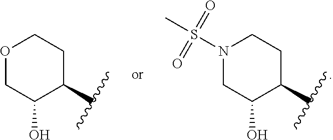 [SMILES: *[C@@H]1CCOC[C@H]1O ; *[C@@H]1CCN(S(C)(=O)=O)C[C@H]1O]

In any of the embodiments herein, unless specified or otherwise contrary from context, L¹-R¹ in Formula I (or any of the applicable sub-formulae as described herein, e.g., Formula A, I-A, or I-B) can be

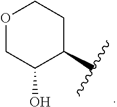 [SMILES: *[C@@H]1CCOC[C@H]1O]

In some embodiments, L¹-R¹ in Formula I (or any of the applicable sub-formulae as described herein, e.g., Formula A, I-A, or I-B) is

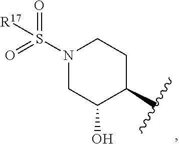

wherein R⁷ is
(1) C₁₋₄ alkyl optionally substituted with 1-3 substituents independently selected from deuterium, F, and OH, such as CHF₂, CF₃, etc.;
(2) phenyl, pyridyl, or pyrimidyl, each of which is optionally substituted with 1-3 substituents independently selected from deuterium, halogen, CN, OH, C₁₋₃ alkyl optionally substituted with F, or C₁₋₃ alkoxy optionally substituted with F; or
(3) 5-membered heteroaryl optionally substituted with 1-3 substituents independently selected from deuterium, halogen, CN, OH, C₁₋₃ alkyl optionally substituted with F, or C₁₋₃ alkoxy optionally substituted with F.
   For example, in some embodiments, R¹⁷ can be C₁₋₄ alkyl optionally substituted with 1-3 F. In some embodiments, R¹⁷ can be phenyl, pyridyl, or pyrimidyl, each of which is unsubstituted or substituted with 1 or 2 substituents each independently F or methyl. In some embodiments, R¹⁷ can be a pyrazole, imidazole, or triazol, each of which is unsubstituted or substituted with a C₁₋₃ alkyl optionally substituted with F. In some specific embodiments, R¹⁷ can be

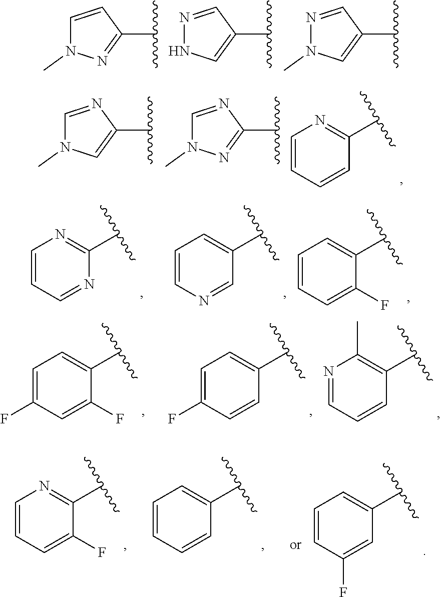

In some embodiments, L¹-R¹ in Formula I (e.g., Formula A, I-A, or I-B) can be selected from:

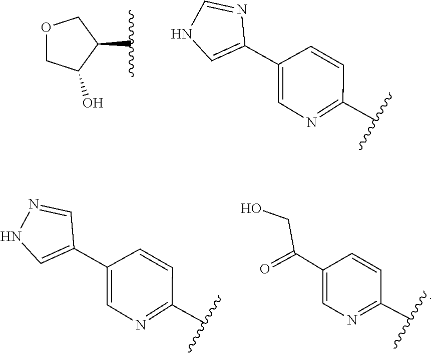 [SMILES: *[C@@H]1COC[C@H]1O ; *c1ccc(-c2c[nH]cn2)cn1 ; *c1ccc(-c2cn[nH]c2)cn1 ; *c1ccc(C(=O)CO)cn1]

Various groups are suitable for use as R² in Formula I. For example, in some embodiments, R² can be an optionally substituted 10 membered heteroaryl. In some embodiments, R² can be an optionally substituted 9-12 membered bicyclic heterocyclic ring, in which one of the two rings is a phenyl or heteroaryl ring, and R² connects to the remainder of Formula I through a ring atom of the phenyl or heteroaryl portion of the bicyclic ring system. In some embodiments, R² can be an optionally substituted 12-16 membered heteroaryl or heterocyclic ring structure having three or more rings, in which at least one of the rings is a phenyl or heteroaryl ring, and R² connects to the remainder of Formula I through a ring atom of a phenyl or heteroaryl portion of the ring system. In one embodiment, said phenyl or heteroaryl portion of R² (that connects to the remainder of Formula I) is not fused to a 5-membered heteroaryl.

In one embodiment, R² is an optionally substituted 10 membered heteroaryl, wherein R² connects to the remainder of Formula I through a phenyl, and the phenyl is fused to a 6-membered heteroaryl.

In one embodiment, R² is an optionally substituted 9-12 membered bicyclic heterocyclic ring, wherein R² connects to the remainder of Formula I through a phenyl, and the phenyl is fused to a 5-8 membered heterocyclic ring.

In one embodiment, R² is an optionally substituted tricyclic heteroaryl, wherein R² connects to the remainder of Formula I through a phenyl (the first ring), the phenyl is fused to a 6-membered heteroaryl (the second ring), and the 6-membered heteroaryl is further fused to a 5- or 6-membered heteroaryl (the third ring). In one embodiment, the third ring is a 5-membered heteroaryl. In one embodiment, the third ring is a 6-membered heteroaryl.

In one embodiment, R² is an optionally substituted tricyclic heterocyclic ring, wherein R² connects to the remainder of Formula I through a phenyl (the first ring), the phenyl is fused to a 6-membered heteroaryl (the second ring), and the 6-membered heteroaryl is further fused to a 5- or 6-membered heteroaryl or a 5- or 6-membered carbocyclic ring (the third ring). In one embodiment, the third ring is a 5-membered heterocyclic ring. In one embodiment, the third ring is a 6-membered heterocyclic ring. In one embodiment, the third ring is a 5-membered carbocyclic ring. In one embodiment, the third ring is a 6-membered carbocyclic ring.

In one embodiment, R² is an optionally substituted tricyclic heterocyclic ring, wherein R² connects to the remainder of Formula I through a phenyl (the first ring), the phenyl is fused to a 5-7 membered heterocyclic ring (the second ring), and the 5-7 membered heterocyclic ring is further fused to a 5- or 6-membered heteroaryl or a 5- or 6-membered heterocyclic ring (the third ring). In one embodiment, the second ring is a 6-membered heterocyclic ring, and the third ring is a 6-membered heterocyclic ring. In one embodiment, the second ring is a 7-membered heterocyclic ring, and the third ring is a 5-membered heteroaryl.

Typically, R² includes at least two heteroatoms that are nitrogen or oxygen. For example, in some embodiments, the total number of nitrogen and oxygen atoms in R² can be 2, 3, 4, 5, or 6. Typically, R² includes at least one hydrogen bond donor (e.g., an OH, NH₂, or NHR group) and at least one hydrogen bond acceptor (e.g., a C(═O) group, O, N (such as in a pyridine ring), etc.).

In some embodiments, R² is a 10-membered bicyclic heteroaryl having 1-4 ring nitrogen atoms, such as quinolinyl (preferably,

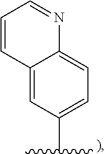 [SMILES: *c1ccc2ncccc2c1]

naphthyridinyl, etc., which is optionally substituted with one or more (e.g., 1, 2, or 3) substituents independently selected from deuterium, halo (e.g., F, Cl), CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²),
wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl;
wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), halo (e.g., F), CN, G¹, OH, O-G¹, NH₂, NH(G¹), and N(G¹)(G¹); and
wherein two optional substituents of the 10-membered bicyclic heteroaryl group, together with the intervening atom(s), can optionally be joined to form a fused ring structure.

In some embodiments, R² is a 9-12 membered bicyclic heterocyclic ring, in which one of the two rings is a phenyl or heteroaryl ring, and R² connects to the remainder of Formula I through a ring atom of the phenyl or heteroaryl portion of the bicyclic ring system, wherein the 9-12 membered bicyclic heterocyclic ring is optionally substituted with one or more (e.g., 1, 2, or 3) substituents independently selected from halo (e.g., F), CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²),
wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl;
wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), halo (e.g., F), CN, G¹, OH, O-G¹, NH₂, NH(G¹), and N(G¹)(G¹); and
wherein two optional substituents of the 9-12 membered bicyclic heterocyclic ring, together with the intervening atom(s), can optionally be joined to form a ring structure.

In some embodiments, R² is a 12-16 membered heteroaryl or heterocyclic ring structure having three or more rings, in which at least one of the rings is a phenyl or heteroaryl ring, and R² connects to the remainder of Formula I through a ring atom of a phenyl or heteroaryl portion of the ring system, such as

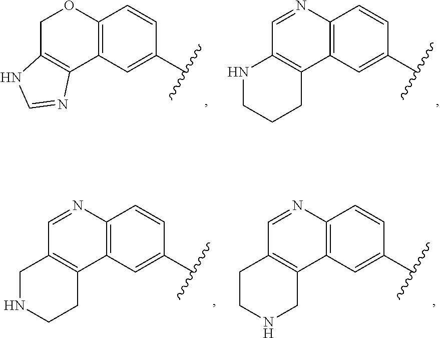

etc., wherein the 12-16 membered heteroaryl or heterocyclic ring is optionally substituted with one or more (e.g., 1, 2, or 3) substituents independently selected from deuterium, halo (e.g., F, Cl), CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²),
wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl;
wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), halo (e.g., F), CN, G¹, OH, O-G¹, NH₂, NH(G¹), and N(G¹)(G¹); and
wherein two optional substituents of the 12-16 membered heteroaryl or heterocyclic ring, together with the intervening atom(s), can optionally be joined to form a ring structure.

In some preferred embodiments, R² in Formula I can be a structure according to M-1:

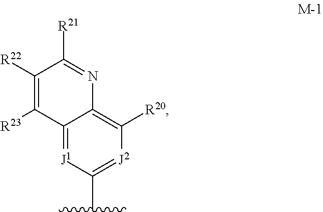

wherein:
J¹ and J² are independently N or CR³⁰, wherein R³⁰ at each occurrence is independently hydrogen, halogen, C₁₋₄ alkyl optionally substituted with F, or C₁₋₄ heteroalkyl optionally substituted with F;
R²⁰ is hydrogen, halogen (e.g., F or Cl), CN, C₁₋₄ alkyl optionally substituted with F, C₁₋₄ heteroalkyl optionally substituted with F, or an optionally substituted 3-6 membered ring structure (e.g., cyclopropyl, cyclobutyl, 4-6 membered heterocyclic ring, or 5 membered heteroaryl);
R²¹, R²², and R²³ are each independently: (1) hydrogen or deuterium; (2) halogen (e.g., F or Cl), (3) CN, (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) a 3-6 membered ring structure (e.g., cyclopropyl, cyclobutyl, 4-6 membered heterocyclic ring, or 5 membered heteroaryl) optionally substituted 1-3 substituents each independently oxo, halo (e.g., F), OH, NH₂, C₁₋₄ alkyl optionally substituted with F, or C₁₋₄ heteroalkyl optionally substituted with F, or R²¹ and R²², together with the intervening atoms, are joined to form a 5-8 membered ring (e.g., 5-7 membered carbocyclic or heterocyclic ring), which is optionally substituted 1-3 substituents each independently oxo, deuterium, halo (e.g., F), OH, NH₂, C₁₋₄ alkyl optionally substituted with F, or C₁₋₄ heteroalkyl optionally substituted with F, and R²³ is defined above;
or R²² and R²³, together with the intervening atoms, are joined to form a 5-8 membered ring (e.g., 5-7 membered carbocyclic, aryl, heteroaryl, or heterocyclic ring), which is optionally substituted 1-3 substituents each independently oxo, deuterium, halo (e.g., F), OH, NH₂, C₁₋₄ alkyl optionally substituted with F, or C₁₋₄ heteroalkyl optionally substituted with F, and R²¹ is defined above.

J¹ in M-1 is typically CR³⁰, wherein R³⁰ is defined herein, preferably, R³⁰ is hydrogen. However, in some embodiments, J¹ can be N.

J² in M-1 is typically also CR³⁰, wherein R³⁰ is defined herein, preferably, R³⁰ is hydrogen. However, in some embodiments, J² can also be N. Preferably, J¹ and J² are not both N.

In some preferred embodiments, both J¹ and J² in M-1 are CH.

In one embodiment, R² has a structure according to M-1-1:

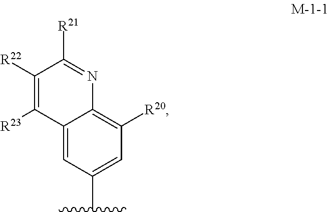

wherein:
R²¹, R²², and R²³ are each independently: (1) hydrogen or deuterium; (2) halogen (e.g., F or Cl), (3) CN, (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) a 3-6 membered ring structure (e.g., cyclopropyl, cyclobutyl, 4-6 membered heterocyclic ring, or 5 membered heteroaryl) optionally substituted 1-3 substituents each independently oxo, halo (e.g., F), OH, NH₂, C₁₋₄ alkyl optionally substituted with F, or C₁₋₄ heteroalkyl optionally substituted with F.

In some embodiments, the compound of Formula I can have a structure according

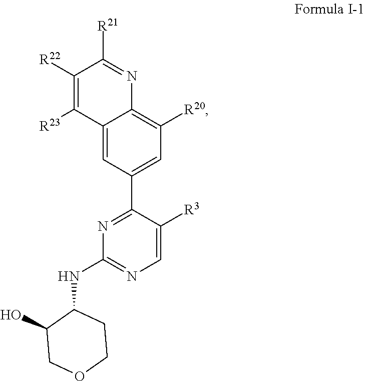

wherein R³, R²⁰, R²¹, R²², and R²³ are defined herein. In some embodiments, the compound of Formula I can be a deuterated analog of Formula I-1, e.g., having a structure according to Formula I-1-D:

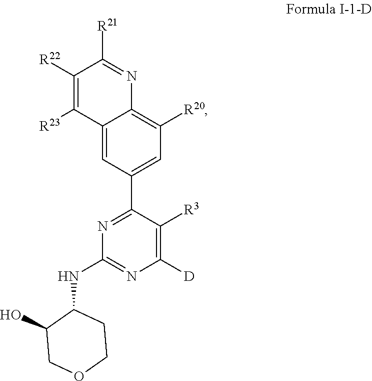

wherein R³, R²⁰, R²¹, R²², and R²³ are defined herein.

In one embodiment, R² has a structure according to M-6-1:

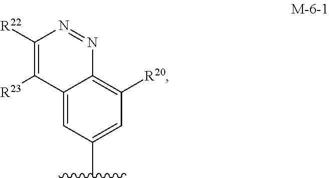

wherein:
R²⁰ is hydrogen, halogen (e.g., F or Cl), CN, C₁₋₄ alkyl optionally substituted with F, C₁₋₄ heteroalkyl optionally substituted with F, or an optionally substituted 3-6 membered ring structure (e.g., cyclopropyl, cyclobutyl, 4-6 membered heterocyclic ring, or 5 membered heteroaryl); R²² and R²³ are each independently: (1) hydrogen or deuterium; (2) halogen (e.g., F or Cl), (3) CN, (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) a 3-6 membered ring structure (e.g., cyclopropyl, cyclobutyl, 4-6 membered heterocyclic ring, or 5 membered heteroaryl) optionally substituted 1-3 substituents each independently oxo, halo (e.g., F), OH, NH₂, C₁₋₄ alkyl optionally substituted with F, or C₁₋₄ heteroalkyl optionally substituted with F.

In some embodiments, R²⁰ is hydrogen. In some embodiments, R²⁰ is halogen (e.g., F or Cl) or CN, such as F. In some embodiments, R²⁰ is C₁₋₄ alkyl optionally substituted with F. In some embodiments, R²⁰ is C₁₋₄ heteroalkyl optionally substituted with F. Typically, the C₁₋₄ heteroalkyl has 1 or 2 heteroatoms independently N or O. In some embodiments, R²⁰ can also be an optionally substituted 3-6 membered ring structure (e.g., cyclopropyl, cyclobutyl, 4-6 membered heterocyclic ring, or 5 membered heteroaryl).

R²⁰ is typically hydrogen, halogen, or C₁₋₄ alkyl. For example, in some preferred embodiments, R²⁰ is hydrogen, F, Cl, methyl, or ethyl, more preferably, F.

Preferably, at least one of R²¹, R²², and R²³ contains a hydrogen bond donor, such as an OH, NH₂, or NHR group, more preferably, only one of R²¹, R²², and R²³ contains a hydrogen bond donor, or the ring structure formed by R²¹ and R²², or R²² and R²³, contains a hydrogen bond donor.

When not containing a hydrogen bond donor, R²¹, R²², and R²³ can typically be hydrogen, deuterium, halogen, or C₁₋₄ alkyl which may be optionally substituted with 1-3 suitable substituents as defined herein.

In some embodiments, one or more of R²¹, R²², and R²³ can also be C₁₋₄ heteroalkyl optionally substituted with F, in which the heteroalkyl contains 1 or 2 heteroatoms independently oxygen or nitrogen.

For example, in some embodiments, R²¹ can be a C₁₋₄ alkoxy. In some embodiments, R²¹ can be NH(C₁₋₄ alkyl), N(C₁₋₃ alkyl)(C₁₋₃ alkyl), wherein each C₁₋₃ alkyl is independently selected, provided the total number of carbons are equal or less than 4.

In some embodiments, R²² can be a C₁₋₄ alkoxy. In some embodiments, R²² can be NH(C₁₋₄ alkyl), N(C₁₋₃ alkyl)(C₁₋₃ alkyl), wherein each C₁₋₃ alkyl is independently selected, provided the total number of carbons are equal or less than 4.

In some embodiments, R²³ can be a C₁₋₄ alkoxy. In some embodiments, R²³ can be NH(C₁₋₄ alkyl), N(C₁₋₃ alkyl)(C₁₋₃ alkyl), wherein each C₁₋₃ alkyl is independently selected, provided the total number of carbons are equal or less than 4.

In some embodiments, one or more of R²¹, R²², and R²³ can also be a 3-6 membered ring structure (e.g., cyclopropyl, cyclobutyl, 4-6 membered heterocyclic ring, or 5 membered heteroaryl) which can be optionally substituted with 1-3 suitable substituents as defined herein. For example, in some embodiments, the 3-6 membered ring structure can be unsubstituted. In some embodiments, the 3-6 membered ring structure can be substituted with 1-3 substituents each independently oxo, OH, halo (e.g., F), or C₁₋₄ alkyl. For example, in some embodiments, one of R²¹, R²², and R²³ can be

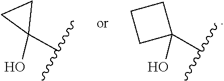 [SMILES: *C1(O)CC1 ; *C1(O)CCC1]

In some embodiments, R²¹ can be hydrogen, deuterium, C₁₋₄ alkyl (e.g., methyl or ethyl), or C₁₋₄ heteroalkyl having 1 or 2 heteroatoms independently oxygen or nitrogen. In some embodiments, R²¹ can be halogen, such as F or Cl. In some preferred embodiments, R²¹ is hydrogen, deuterium, F, or methyl.

In some embodiments, R^{2′} has a hydrogen bond donor. For example, in some embodiments, R²¹ can be C₁₋₄ alkyl substituted with OH, NH₂, or NH(C₁₋₄ alkyl), preferably,

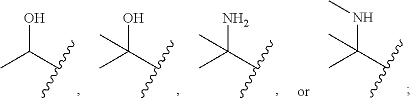

and in some embodiments, R²¹ can be C₃₋₄ cycloalkyl substituted with OH, NH₂, or NH(C₁₋₄ alkyl), such as

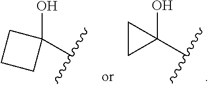 [SMILES: *C1(O)CCC1 ; *C1(O)CC1]

In such embodiments, typically, R²² and R²³ do not also have an OH, NH₂, or NH(C₁₋₄ alkyl) group, for example, in some embodiments, R²² and R²³ are each independently hydrogen, F, Cl, methyl, or ethyl.

In some embodiments, R²¹ can be a 4-6 membered heterocyclic ring having 1 or 2 ring heteroatoms, e.g., azetidinyl, pyrrolidinyl, piperidinyl, piperazinyl, etc., wherein the 4-6 membered heterocyclic ring is optionally substituted with 1-3 substituents each independently oxo, F, OH, NH₂, C₁₋₄ alkyl optionally substituted with F, C₁₋₄ alkoxy optionally substituted with F, or NH(C₁₋₄ alkyl). For example, in some embodiments, R²¹ is azetidinyl or pyrrolidinyl, such as

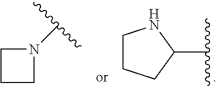 [SMILES: *N1CCC1 ; *C1CCCN1]

Typically, in such embodiments, R²² and R²³ are not a heteroaryl or heterocyclic ring.

In some embodiments, R²¹ can be a 5 membered heteroaryl ring having 2-4 ring heteroatoms, e.g., imidazolyl, pyrazolyl, etc., wherein the 5 membered heteroaryl ring is optionally substituted with 1-3 substituents each independently F, OH, NH₂, C₁₋₄ alkyl optionally substituted with F, C₁₋₄ alkoxy optionally substituted with F, or NH(C₁₋₄ alkyl). For example, in some embodiments, R²¹ is an imidazolyl or pyrazolyl, optionally substituted with one or two C₁₋₄ alkyl such as methyl, such as

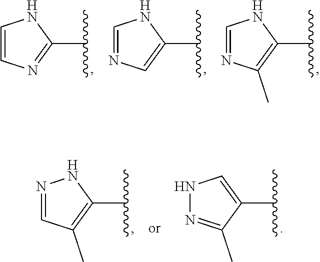

Typically, in such embodiments, R²² and R²³ are not a heteroaryl or heterocyclic ring.

Various groups are also suitable for the R²² position. For example, in some embodiments, R²² can be hydrogen, C₁₋₄ alkyl (e.g., methyl or ethyl), or C₁₋₄ heteroalkyl having 1 or 2 heteroatoms independently oxygen or nitrogen. In some embodiments, R²² can be halogen, such as F or Cl.

In some preferred embodiments, R²² has a hydrogen bond donor. For example, in some embodiments, R²² can be C₁₋₄ alkyl substituted with OH, NH₂, or NH(C₁₋₄ alkyl), preferably,

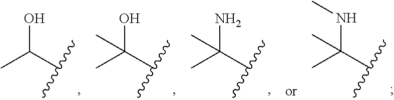

and in some embodiments, R²² can be C₃₋₄ cycloalkyl substituted with OH, NH₂, or NH(C₁₋₄ alkyl), such as

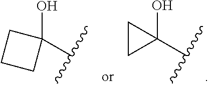 [SMILES: *C1(O)CCC1 ; *C1(O)CC1]

In some embodiments, R²² is

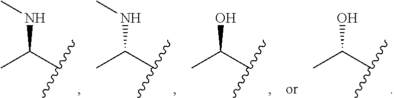

In some embodiments, R²² can be C₁₋₄ alkyl substituted with NH-(3-7 membered ring), wherein the 3-7 membered ring is preferably a 3, 4, or 5 membered ring, such as cyclopropyl. For example, in some embodiments, R²² can be C₁₋₄ alkyl (e.g., ethyl) substituted with NH-cyclopropyl. In some embodiments, R²² is

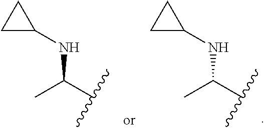

In some embodiments, R²² is or

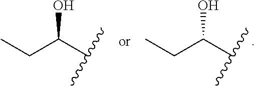

In such embodiments, typically, R²¹ and R²³ do not also have an OH, NH₂, or NH(C₁₋₄ alkyl) group, for example, in some embodiments, R²¹ and R²³ are each independently hydrogen, deuterium, F, Cl, methyl, or ethyl.

In some embodiments, R²² can be a 4-6 membered heterocyclic ring having 1 or 2 ring heteroatoms, e.g., azetidinyl, pyrrolidinyl, piperidinyl, piperazinyl, etc., wherein the 4-6 membered heterocyclic ring is optionally substituted with 1-3 substituents each independently oxo, F, OH, NH₂, C₁₋₄ alkyl optionally substituted with F, C₁₋₄ alkoxy optionally substituted with F, or NH(C₁₋₄ alkyl). For example, in some embodiments, R²² is azetidinyl or pyrrolidinyl, such as

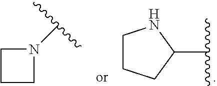 [SMILES: *N1CCC1 ; *C1CCCN1]

In some embodiments, R²² is azetidinyl or pyrrolidinyl, which is optionally substituted with a C₁₋₄ alkyl optionally substituted with F, such as

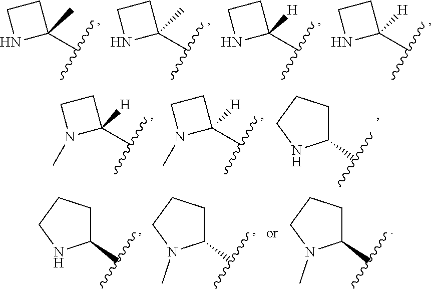

Typically, in such embodiments, R²¹ and R²³ are not a heteroaryl or heterocyclic ring. For example, in some embodiments, R²¹ and R²³ are each independently hydrogen, deuterium, F, Cl, methyl, or ethyl.

In some embodiments, R²² can be a 5 membered heteroaryl ring having 2-4 ring heteroatoms, e.g., imidazolyl, pyrazolyl, etc., wherein the 5 membered heteroaryl ring is optionally substituted with 1-3 substituents each independently F, OH, NH₂, C₁₋₄ alkyl optionally substituted with F, C₁₋₄ alkoxy optionally substituted with F, or NH(C₁₋₄ alkyl). For example, in some embodiments, R²² is an imidazolyl or pyrazolyl, optionally substituted with one or two C₁₋₄ alkyl such as methyl, such as

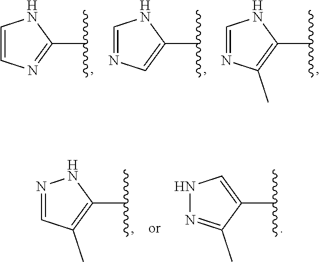

Typically, in such embodiments, R²¹ and R²³ are not a heteroaryl or heterocyclic ring. For example, in some embodiments, R²¹ and R²³ are each independently hydrogen, deuterium, F, Cl, methyl, or ethyl

Various groups are also suitable for the R²³ position. For example, in some embodiments, R²³ can be hydrogen, deuterium, C₁₋₄ alkyl (e.g., methyl or ethyl), or C₁₋₄ heteroalkyl having 1 or 2 heteroatoms independently oxygen or nitrogen, such as N(CH₃)₂. In some embodiments, R²³ can be halogen, such as F or Cl. In some preferred embodiments, R²³ is hydrogen, deuterium, F, Cl, or methyl.

In some embodiments, R²³ has a hydrogen bond donor. For example, in some embodiments, R²³ can be C₁₋₄ alkyl substituted with OH, NH₂, or NH(C₁₋₄ alkyl), preferably,

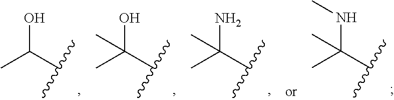

and in some embodiments, R²³ can be C₃₋₄ cycloalkyl substituted with OH, NH₂, or NH(C₁₋₄ alkyl), such as

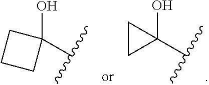 [SMILES: *C1(O)CCC1 ; *C1(O)CC1]

In such embodiments, typically, R²¹ and R²² do not also have an OH, NH₂, or NH(C₁₋₄ alkyl) group, for example, in some embodiments, R²¹ and R²² are each independently hydrogen, F, Cl, methyl, or ethyl.

In some embodiments, R²³ can be a 4-6 membered heterocyclic ring having 1 or 2 ring heteroatoms, e.g., azetidinyl, pyrrolidinyl, piperidinyl, piperazinyl, etc., wherein the 4-6 membered heterocyclic ring is optionally substituted with 1-3 substituents each independently oxo, F, OH, NH₂, C₁₋₄ alkyl optionally substituted with F, C₁₋₄ alkoxy optionally substituted with F, or NH(C₁₋₄ alkyl). For example, in some embodiments, R²³ is azetidinyl or pyrrolidinyl, such as

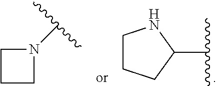 [SMILES: *N1CCC1 ; *C1CCCN1]

Typically, in such embodiments, R²¹ and R²² are not a heteroaryl or heterocyclic ring.

In some embodiments, R²³ can be a 5 membered heteroaryl ring having 2-4 ring heteroatoms, e.g., imidazolyl, pyrazolyl, etc., wherein the 5 membered heteroaryl ring is optionally substituted with 1-3 substituents each independently F, OH, NH₂, C₁₋₄ alkyl optionally substituted with F, C₁₋₄ alkoxy optionally substituted with F, or NH(C₁₋₄ alkyl). For example, in some embodiments, R²³ is an imidazolyl or pyrazolyl, optionally substituted with one or two C₁₋₄ alkyl such as methyl, such as

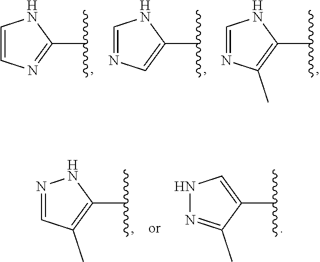

Typically, in such embodiments, R²¹ and R²² are not a heteroaryl or heterocyclic ring.

In some embodiments, R²¹ and R²², together with the intervening atoms, are joined together to form a 5 or 6 membered ring, preferably, a 5 or 6 membered carbocyclic ring, which is substituted with 1-3 substituents each independently F, OH, NH₂, C₁₋₄ alkyl optionally substituted with F, C₁₋₄ alkoxy optionally substituted with F, or NH(C₁₋₄ alkyl), preferably, one of the substitutent(s) comprises a hydrogen bond donor, such as OH, NH₂, or NH(C₁₋₄ alkyl). In such embodiments, R²³ is typically hydrogen, halogen (e.g., F, or Cl), C₁₋₄ alkyl (e.g., methyl or ethyl), or C₁₋₄ heteroalkyl having 1 or 2 heteroatoms independently oxygen or nitrogen.

In some embodiments, R²² and R²³, together with the intervening atoms, are joined together to form a 5 or 6 membered ring, preferably, a 5 or 6 membered carbocyclic ring, which is substituted with 1-3 substituents each independently F, OH, NH₂, C₁₋₄ alkyl optionally substituted with F, C₁₋₄ alkoxy optionally substituted with F, or NH(C₁₋₄ alkyl), preferably, one of the substitutent(s) comprises a hydrogen bond donor, such as OH, NH₂, or NH(C₁₋₄ alkyl). In such embodiments, R²¹ is typically hydrogen, halogen (e.g., F, or Cl), C₁₋₄ alkyl (e.g., methyl or ethyl), or C₁₋₄ heteroalkyl having 1 or 2 heteroatoms independently oxygen or nitrogen.

For example, in some embodiments, R² can be selected from

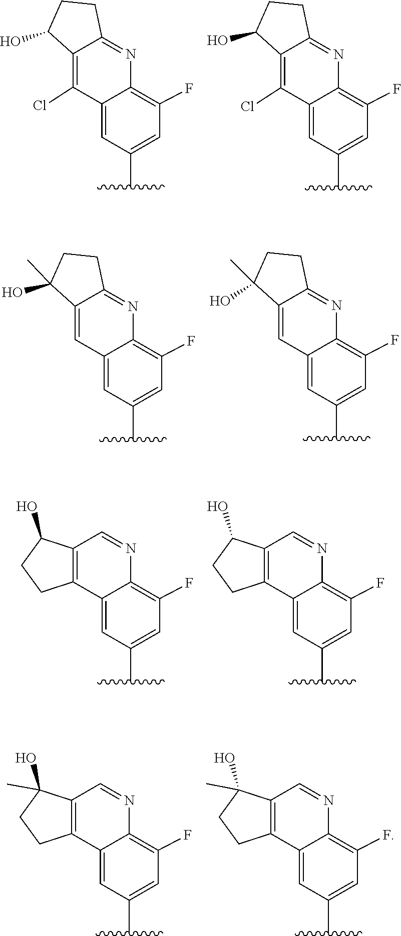

In some preferred embodiments, R²² contains a hydrogen bond donor, such as having an OH, NH₂, or NH group/moiety. For example, in some embodiments, R² in Formula I can be selected from:

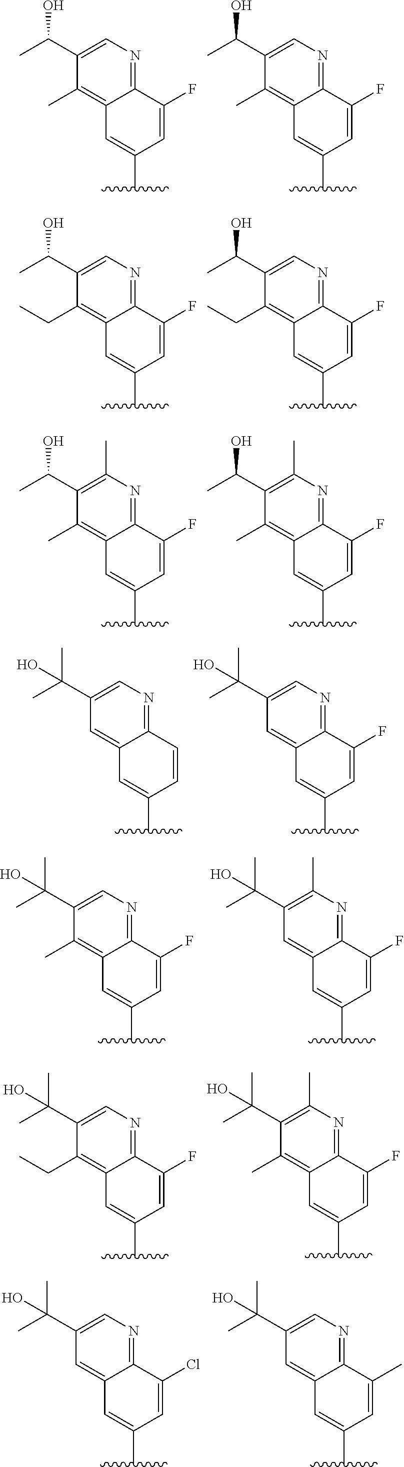

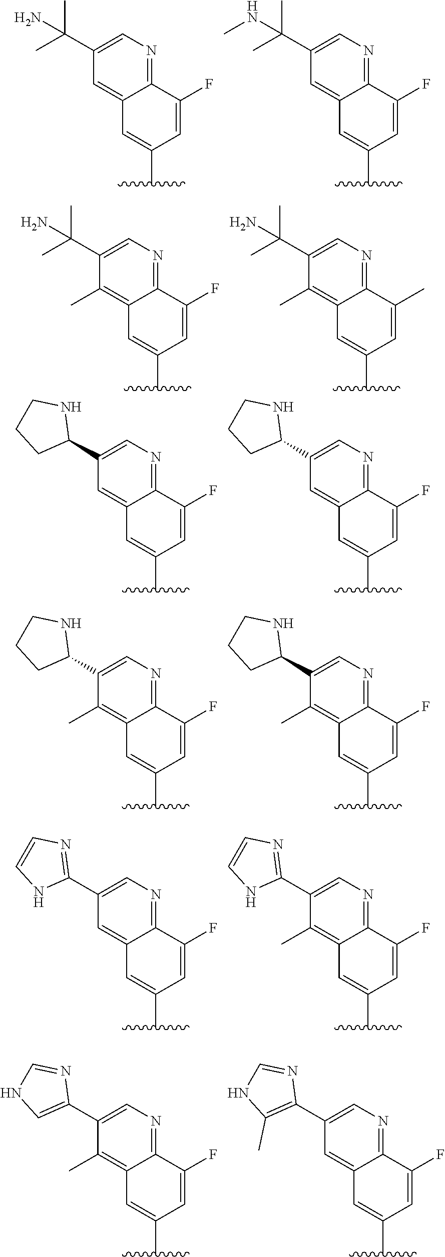

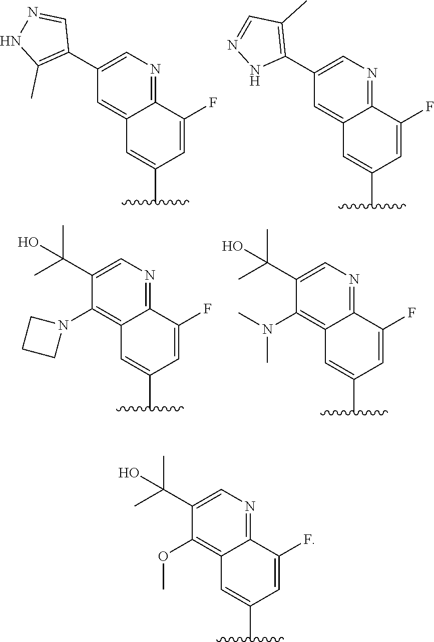

In some preferred embodiments, R² in Formula I can be selected from:

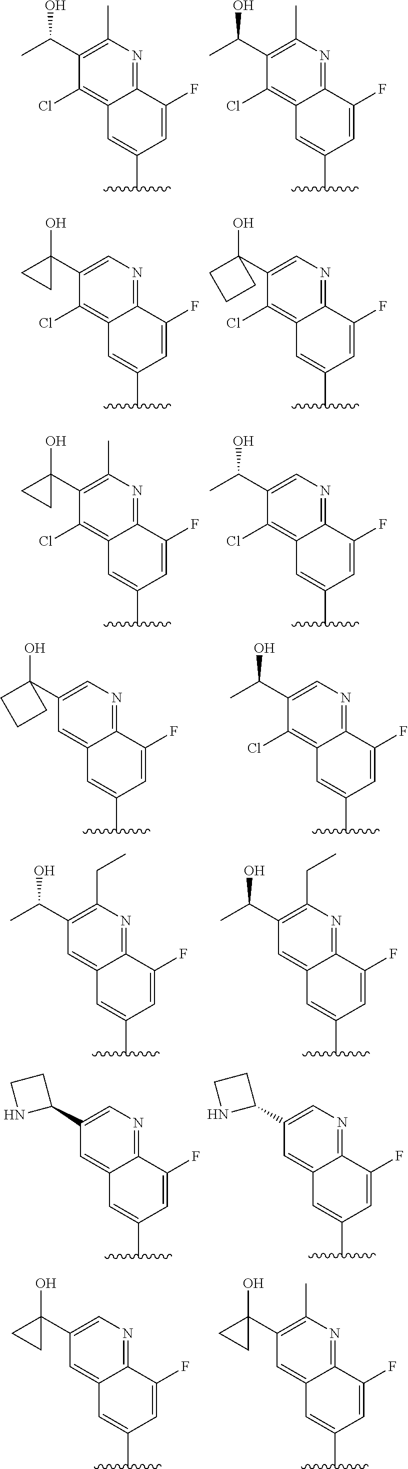

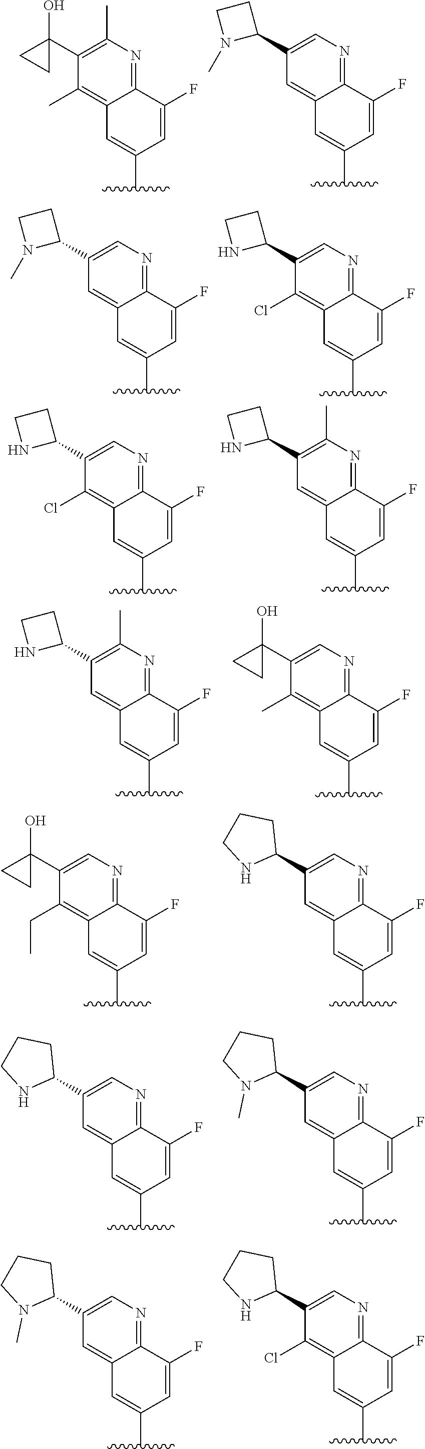

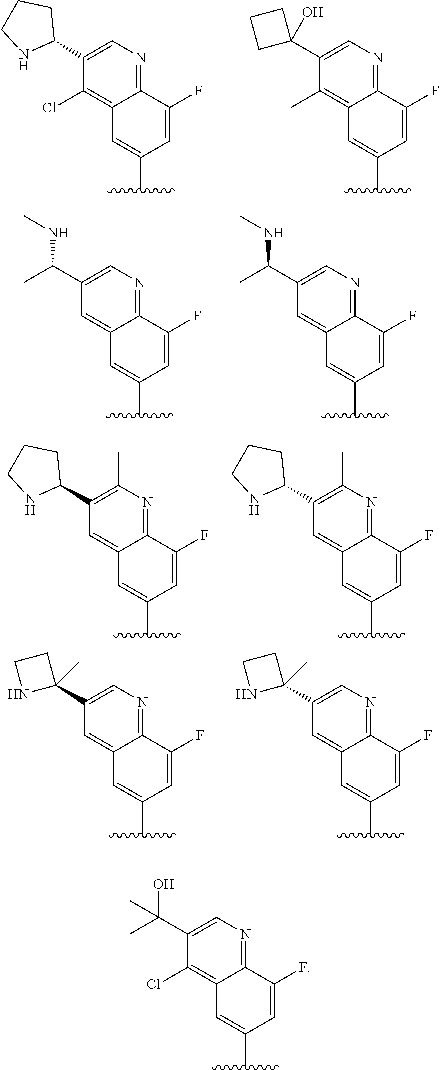

In some preferred embodiments, R² in Formula I can be selected from:

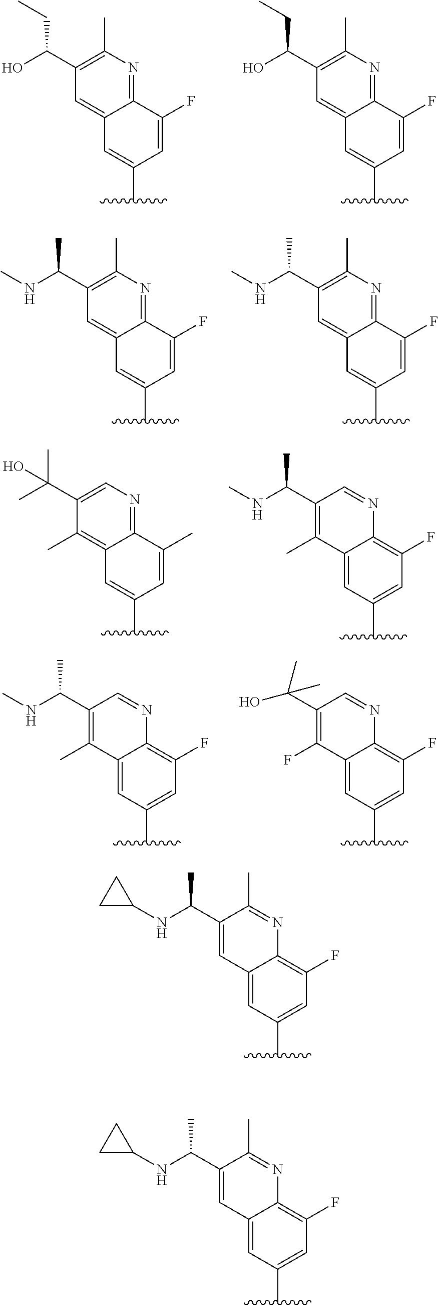

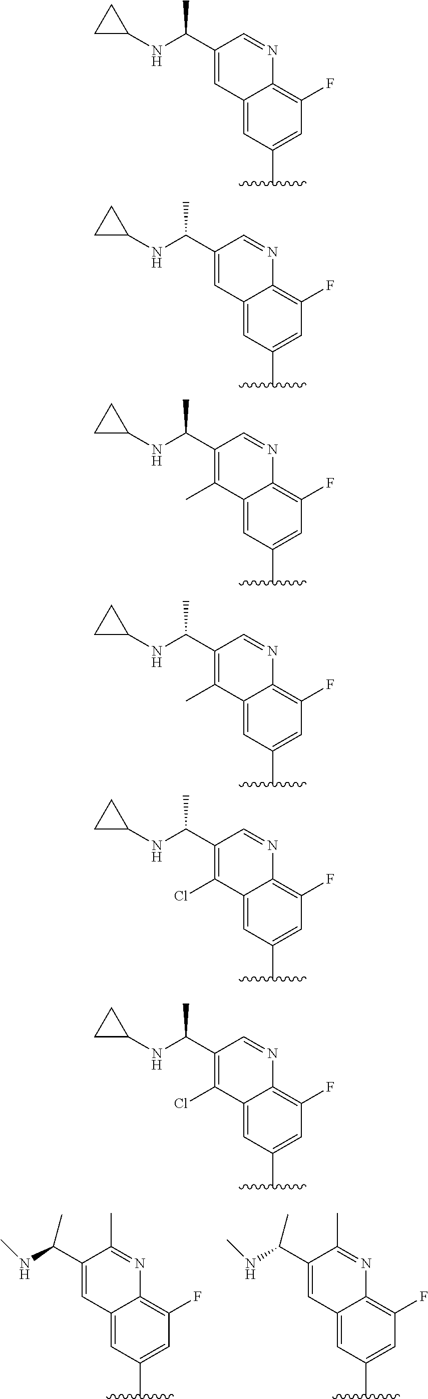

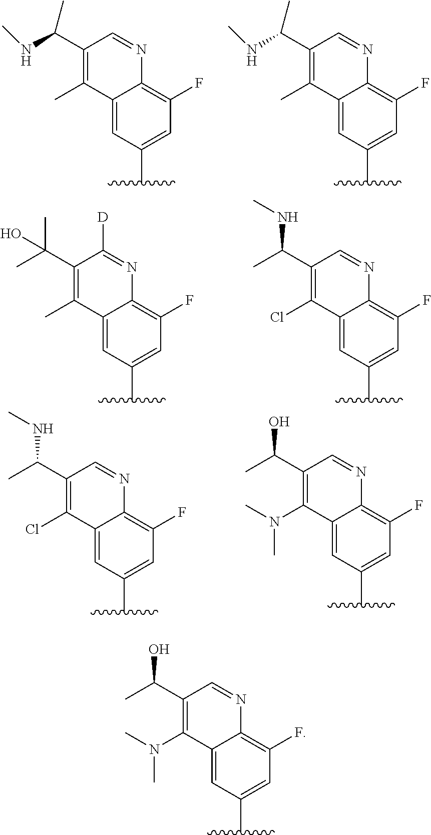

In some embodiments, R²¹ or R²³ contains a hydrogen bond donor, such as having an OH, NH₂, or NH group/moiety. For example, in some embodiments, R² in Formula I can be selected from:

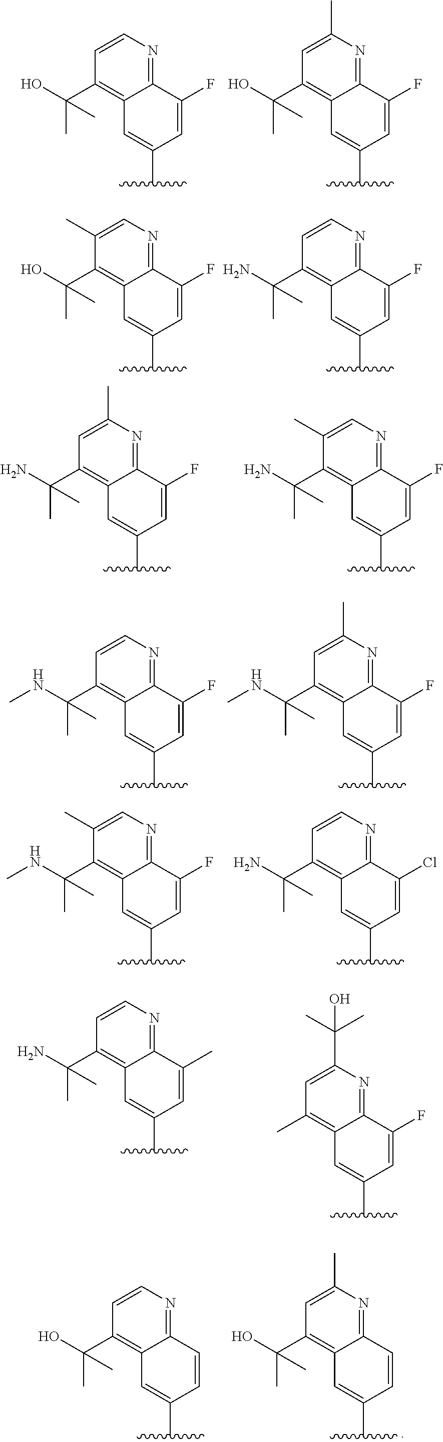

In some embodiments, R² in Formula I can be a structure according to M-2 or M-5:

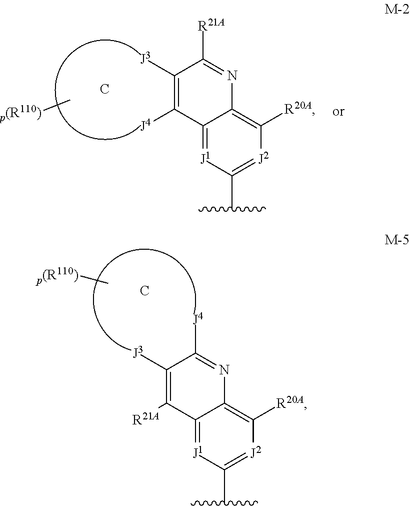

wherein:
J¹ and J² are independently N or CR³⁰, wherein R³⁰ at each occurrence is independently hydrogen, halogen, C₁₋₄ alkyl optionally substituted with F, or C₁₋₄ heteroalkyl optionally substituted with F;
Ring C is a heterocyclic ring or heteroaryl ring, which is optionally substituted with 1-5 instances of R¹¹⁰, i.e., p is 0-5, as valency permits, wherein
   R¹¹⁰ at each occurrence is independently (1) oxo, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) two instances of R¹¹⁰, together with the intervening atom(s), are joined to form a 3-6 membered ring structure (e.g., cyclopropyl, cyclobutyl, 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F,
J³ and J⁴ are independently N, NR³¹, CR³²R³³, or CR³⁴, wherein
   R³¹ at each occurrence is independently hydrogen, C₁₋₄ alkyl optionally substituted with F, or an optionally substituted 3-6 membered ring structure (e.g., cyclopropyl, cyclobutyl, 4-6 membered heterocyclic ring, or 5 membered heteroaryl),
   each of R³² and R³³ at each occurrence is independently (1) hydrogen or deuterium, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) an optionally substituted 3-6 membered ring structure (e.g., cyclopropyl, cyclobutyl, 4-6 membered heterocyclic ring, or 5 membered heteroaryl); or (7) R³² and R³³, together with the carbon atom they are both bond to, are joined to form a carbonyl (CO) or a 3-6 membered ring structure (e.g., cyclopropyl, cyclobutyl, or 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F; or
   two instances of R³², or one instance of R³² and one instance of R¹⁰⁰, together with the intervening atom(s), are joined to form a 3-6 membered ring structure (e.g., cyclopropyl, cyclobutyl, 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F;
   R³⁴ at each occurrence is independently (1) hydrogen, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) an optionally substituted 3-6 membered ring structure (e.g., cyclopropyl, cyclobutyl, 4-6 membered heterocyclic ring, or 5 membered heteroaryl); and
   R^{20A} and R^{21A} are each independently hydrogen, deuterium, halogen (e.g., F or Cl), CN, C₁₋₄ alkyl optionally substituted with F, C₁₋₄ heteroalkyl optionally substituted with F, or an optionally substituted 3-6 membered ring structure (e.g., cyclopropyl, cyclobutyl, 4-6 membered heterocyclic ring, or 5 membered heteroaryl).

Ring C in M-2 or M-5 can typically be a heterocyclic ring, for example, those having 1 or 2 ring heteroatoms, such as a 5 or 6 membered heterocyclic ring having 1 ring nitrogen atom, e.g., a tetrahydropyridine ring or a dihydropyrrole ring. In some embodiments, Ring C in M-2 or M-5 can be a heteroaryl ring, such as a 5-membered heteroaryl, preferably, those having 1 or 2 ring heteroatoms, for example, an imidazole or pyrazole ring. When two instances of R¹⁰, together with the intervening atom(s), are joined to form a 3-6 membered ring structure, Ring C together with the 3-6 membered ring structure can be a spiro, bridged, or fused ring structure.

J¹ in M-2 or M-5 is typically CR³⁰, wherein R³⁰ is defined herein, preferably, R³⁰ is hydrogen. However, in some embodiments, J¹ can be N.

J² in M-2 or M-5 is typically also CR³⁰, wherein R³⁰ is defined herein, preferably, R³⁰ is hydrogen. However, in some embodiments, J² can also be N. Preferably, J^{P} and J² in M-2 or M-5 are not both N.

In some preferred embodiments, both J^{P} and J² in M-2 or M-5 are CH. For example, in some embodiments, the compound of Formula I can have a structure according to Formula I-2:

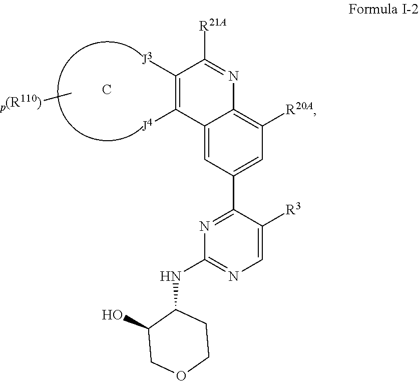

wherein R³, R^{20A}, R^{21A}, J³, J⁴, p, R¹¹⁰ and Ring C are defined herein. In some embodiments, the compound of Formula I can be a deuterated analog of Formula I-2, e.g., having a structure according to Formula I-2-D:

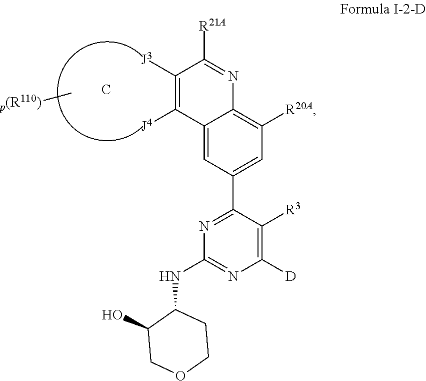

wherein R³, R^{20A}, R^{21A} J³, J⁴, p, R¹¹⁰ and Ring C are defined herein.

In some embodiments, R^{20A} is hydrogen. In some embodiments, R^{20A} is halogen (e.g., F or Cl) or CN, such as F. In some embodiments, R^{20A} is C₁₋₄ alkyl optionally substituted with F. In some embodiments, R^{20A} is C₁₋₄ heteroalkyl optionally substituted with F. Typically, the C₁₋₄ heteroalkyl has 1 or 2 heteroatoms independently N or O. In some embodiments, R^{20A} can also be an optionally substituted 3-6 membered ring structure (e.g., cyclopropyl, cyclobutyl, 4-6 membered heterocyclic ring, or 5 membered heteroaryl).

R^{20A} is typically hydrogen, halogen, or C₁₋₄ alkyl. For example, in some preferred embodiments, R^{20A} is hydrogen, F, Cl, methyl, or ethyl, preferably, R^{20A} is F.

In some embodiments, R^{21A} is hydrogen. In some embodiments, R^{21A} is deuterium. In some embodiments, R^{21A} is halogen (e.g., F or Cl) or CN, such as F. In some embodiments, R^{21A} is C₁₋₄ alkyl optionally substituted with F. In some embodiments, R^{21A} is C₁₋₄ heteroalkyl optionally substituted with F. Typically, the C₁₋₄ heteroalkyl has 1 or 2 heteroatoms independently N or O. In some embodiments, R^{21A} can also be an optionally substituted 3-6 membered ring structure (e.g., cyclopropyl, cyclobutyl, 4-6 membered heterocyclic ring, or 5 membered heteroaryl).

R^{21A} is typically hydrogen, halogen, or C₁₋₄ alkyl. For example, in some preferred embodiments, R^{21A} is hydrogen, F, Cl, methyl, or ethyl. In some preferred embodiments, R^{21A} is hydrogen, deuterium, or methyl.

In some embodiments, the integer p is 0. In some embodiments, the integer p is 1 and R¹¹⁰ is OH, NH₂, NH(C₁₋₄ alkyl), or N(C₁₋₄ alkyl)(C₁₋₄ alkyl), or C₁₋₄ alkyl.

In some embodiments, the integer p is 2. In such embodiments, typically, R¹⁰ at each occurrence is independently C₁₋₄ alkyl (e.g., methyl, ethyl); or two R¹¹⁰ are joined together with the intervening atom(s) to form a 3-6 membered ring, such as a cyclopropyl ring.

In some embodiments, the integer p is 2, 3 or 4, and two instances of R¹¹⁰, together with any intervening atom(s), are joined to form a 5 or 6-membered ring, and any remaining instance(s) of R¹¹⁰ are defined herein. For example, in some embodiments, any remaining non-ring forming instance(s) of R¹¹⁰ at each occurrence is independently C₁₋₄ alkyl (e.g., methyl, ethyl).

J³ and J⁴ are independently N, NR³¹, CR³²R³³, or CR³⁴. For example, in some embodiments, J³ is NR³¹, and R³¹ is hydrogen or C₁₋₄ alkyl (e.g., methyl or ethyl). In some embodiments, J³ is N. In some embodiments, J³ is CR³⁴, and R³⁴ is hydrogen or C₁₋₄ alkyl (e.g., methyl or ethyl). In some embodiments, J³ is CR³²R³³, and R³² is hydrogen, deuterium, F, or C₁₋₄ alkyl (e.g., methyl or ethyl) and R³³ is hydrogen, deuterium, F, or C₁₋₄ alkyl (e.g., methyl or ethyl), e.g., J³ is CH₂, CD₂, CF₂, CH(CH₃), or C(CH₃)₂. In some embodiments, J³ is CR³²R³³, and R³² and R³³ together with the carbon atom they are both bonded to form a 3-4 membered ring, such as a cyclopropyl. In some embodiments, J⁴ is NR³¹, and R³¹ is hydrogen or C₁₋₄ alkyl (e.g., methyl or ethyl). In some embodiments, J⁴ is N. In some embodiments, J⁴ is CR³⁴, and R³⁴ is hydrogen or C₁₋₄ alkyl (e.g., methyl or ethyl). In some embodiments, J⁴ is CR³²R³³, and R³² is hydrogen, deuterium, F, or C₁₋₄ alkyl (e.g., methyl or ethyl) and R³³ is hydrogen, deuterium, F, or C₁₋₄ alkyl (e.g., methyl or ethyl), e.g., J⁴ is CH₂, CD₂, CF₂, CH(CH₃), or C(CH₃)₂. In some embodiments, J⁴ is CR³²R³³, and R³² and R³³ together with the carbon atom they are both bonded to form a 3-4 membered ring, such as a cyclopropyl. In some embodiments, J⁴ can be C(O). Typically, when J³ or J⁴ is N or CR³⁴, it is meant that J³ or J⁴, as applicable, forms a double bond with another ring atom in Ring C, such as those exemplified herein.

In one embodiment, one instance of R³² and one instance of R¹⁰⁰, together with the intervening atom(s), are joined to form a 3-6 membered ring structure (e.g., cyclopropyl, cyclobutyl, 4-6 membered heterocyclic ring) which is optionally substituted as described herein. In one embodiment, J⁴ is CR³²R³³, and R³² and one instance of R¹⁰⁰ (on a ring atom not adjacent to J⁴) form a bridge (e.g., —NH— bridge), and R³³ is hydrogen, deuterium, F, or C₁₋₄ alkyl (e.g., methyl or ethyl).

For example, in some embodiments, R² is characterized as having a structure according to formula F-1, F-2, F-3, F-4, F-5, F-6, F-7, F-8, F-9, F-10, F-11, F-12, F-13, or F-14:

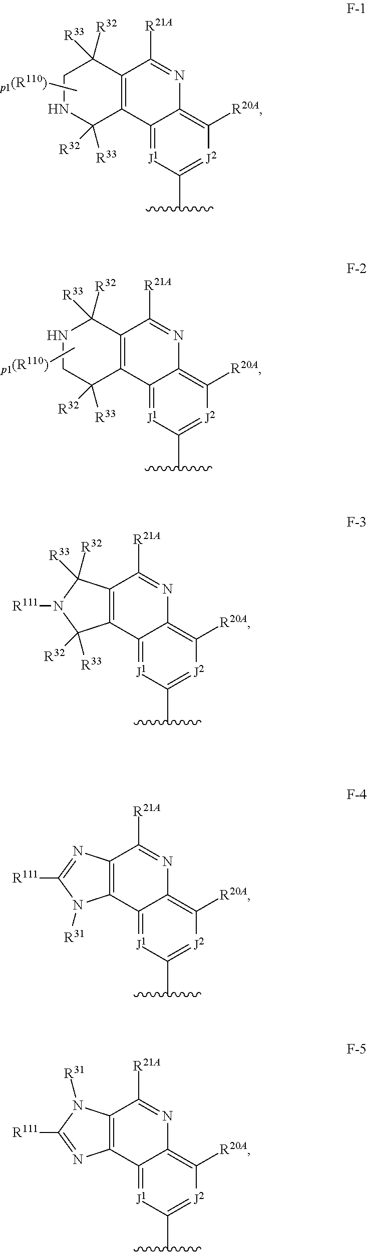

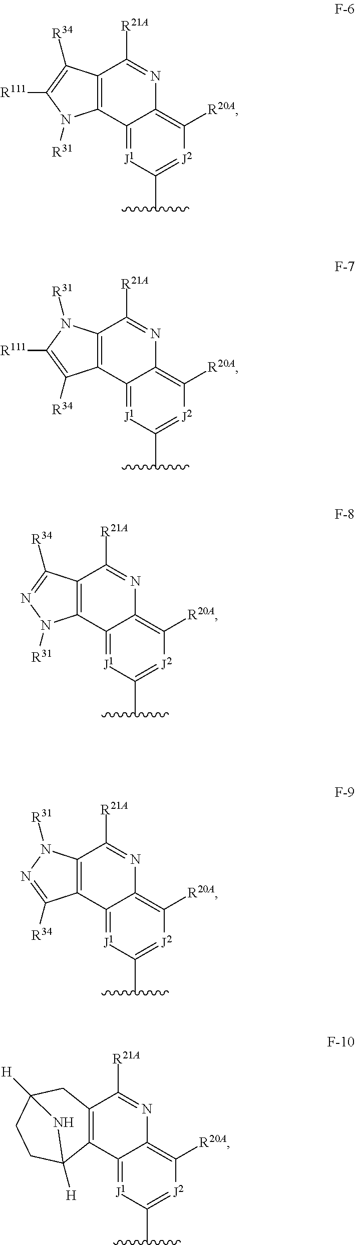

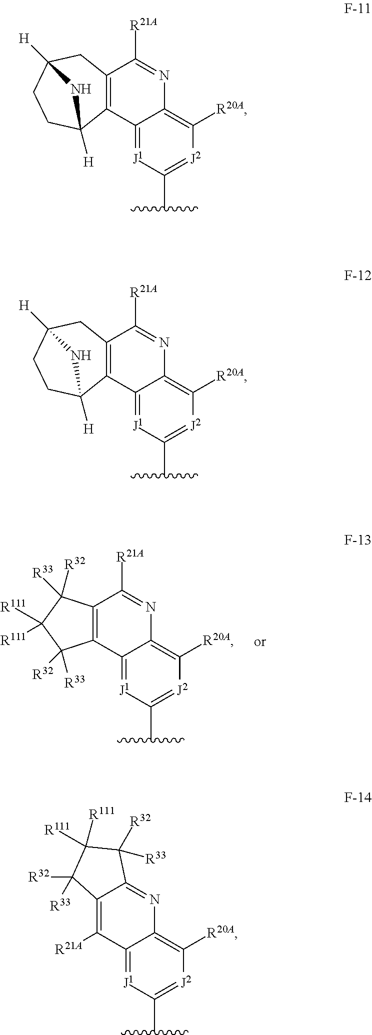

wherein:
p1 is 0, 1, 2, or 3, as valency permits; and
R¹¹¹ is hydrogen or R¹¹⁰, and the variables J¹, J², R^{20A}, R^{21A}, R³¹, R³², R³³, R³⁴, and R¹¹⁰ are as defined herein. For example, in some embodiments, both J¹ and J² in F-1 to F-12 are CH. In some embodiments, in F-1 to F-12, R^{20A} and R^{21A} are each independently hydrogen, halogen, or C₁₋₄ alkyl, preferably, R^{20A} and R^{21A} are each independently hydrogen, F, Cl, methyl, or ethyl, for example, in some preferred embodiments, R^{20A} can be F. In some embodiments, in F-1 to F-9, R³¹, R³², R³³, and R³⁴ are each independently hydrogen, halogen, or C₁₋₄ alkyl, preferably, hydrogen, methyl, or ethyl, or one or both pairs of R³² and R³³, together with the carbon atom they are both bond to, are joined to form a cyclopropyl ring. In some preferred embodiments, R² is characterized as having a structure according to formula F-1.

In one embodiment, R² is characterized as having a structure according to formula F-1-1, F-2-1, F-3-1, F-4-1, F-5-1, F-6-1, F-7-1, F-8-1, F-9-1, F-10-1, F-1-1, F-12-1, F-13-1, or F-14-1:

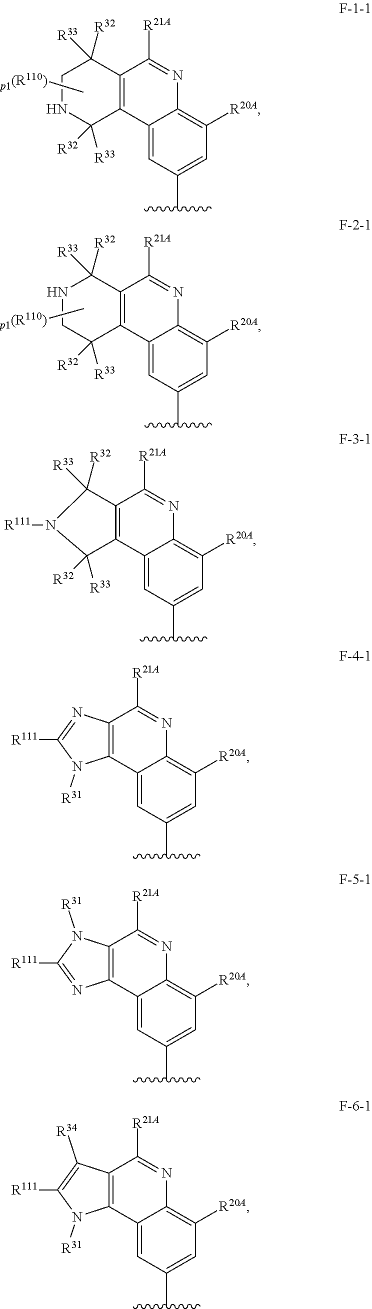

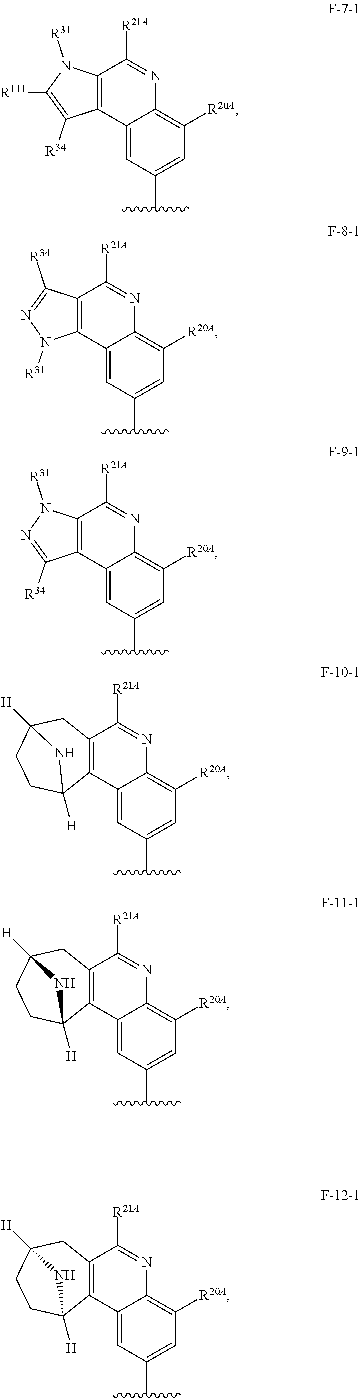

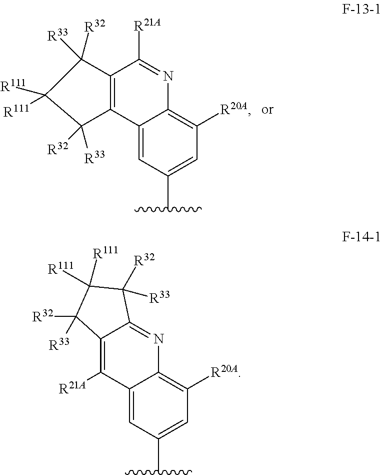

In some embodiments, in F-1 or F-2 (or sub-formula thereof), p1 is 0, or p1 is 1 and R¹¹⁰ is C₁₋₄ alkyl. In some embodiments, in F-1 or F-2 (or sub-formula thereof), p is 2, wherein R¹¹⁰ at each occurrence is independently C₁₋₄ alkyl (e.g., methyl, ethyl); or two R¹¹⁰ are joined together with the intervening atom(s) to form a 3-6 membered ring, such as a cyclopropyl ring.

In some embodiments, in F-3, F-4, F-5, F-6, or F-7 (or sub-formula thereof), R¹¹¹ is hydrogen or C₁₋₄ alkyl.

In some embodiments, in F-4, F-5, F-6, or F-7 (or sub-formula thereof), R¹¹¹ is NH₂ or NH(C₁₋₄ alkyl).

For example, in some specific embodiments, Ring C in M-2 is a heterocyclic ring, and R² in Formula I can be selected from:

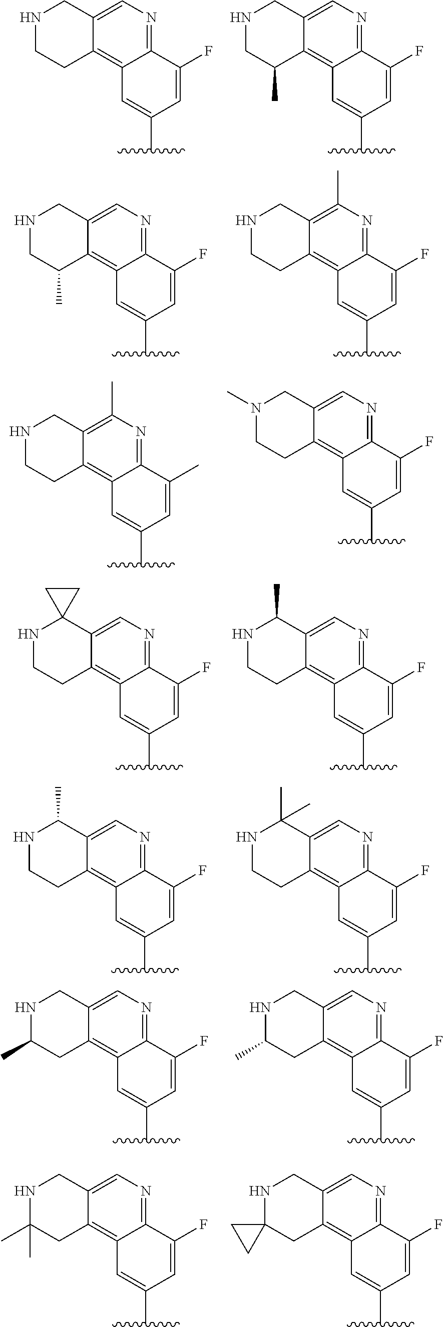

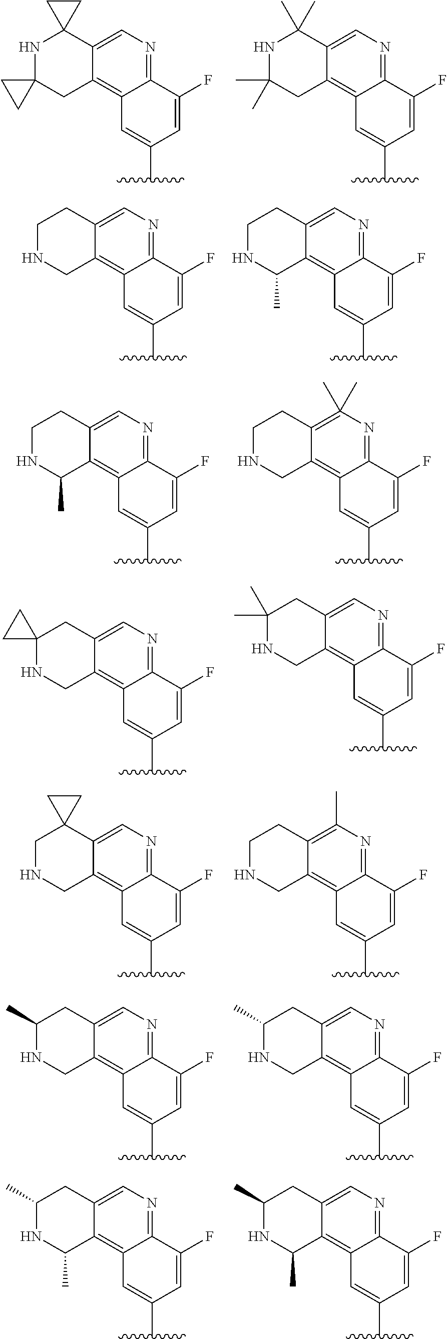

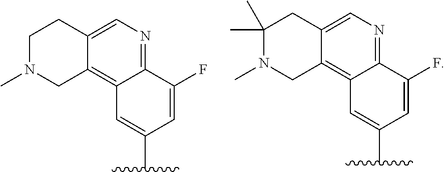

In some specific embodiments, Ring C in M-2 is a heterocyclic ring, and R² in Formula I can be selected from:

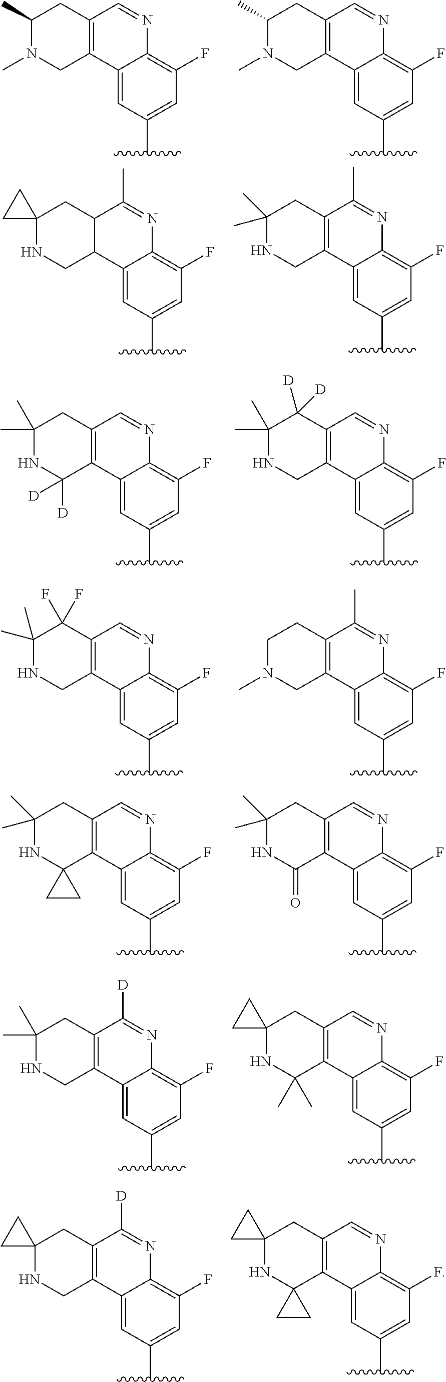

In some specific embodiments, Ring C in M-2 is a 5-membered heterocyclic or heteroaryl ring, and R² in Formula I can be selected from:

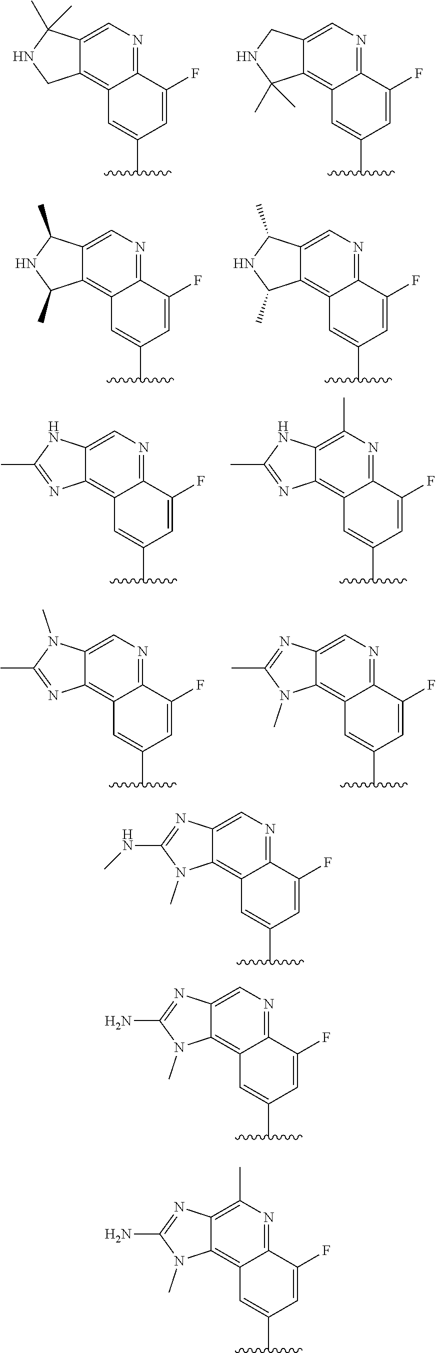

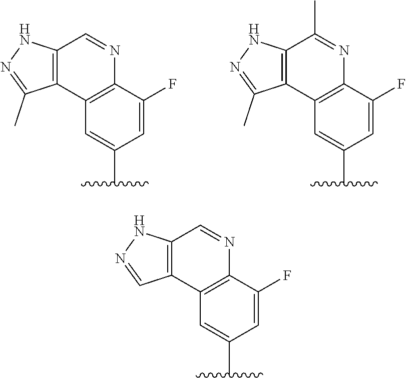 [SMILES: *c1cc(F)c2ncc3[nH]nc(C)c3c2c1 ; *c1cc(F)c2nc(C)c3[nH]nc(C)c3c2c1 ; *c1cc(F)c2ncc3[nH]ncc3c2c1]

In some specific embodiments, Ring C in M-2 together with two instances of R¹¹⁰ are joined to form a bridged heterocyclic ring, for example, R² in Formula I can be

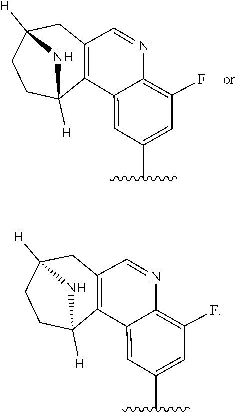 [SMILES: *c1cc(F)c2ncc3c(c2c1)[C@@H]1CC[C@H](C3)N1 ; *c1cc(F)c2ncc3c(c2c1)[C@H]1CC[C@@H](C3)N1]

In some embodiments, R² in Formula I can be a structure according to M-3 or M-4:

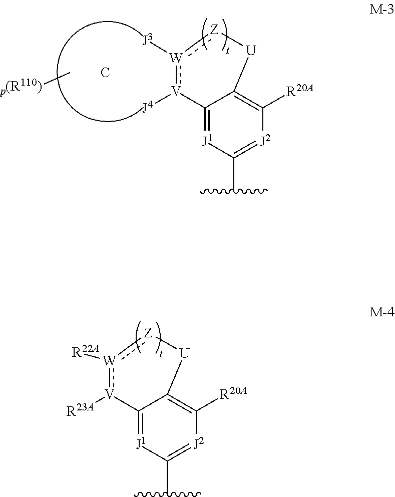

wherein:
J¹ and J² are independently N or CR³⁰, wherein R³⁰ at each occurrence is independently hydrogen, halogen, C₁₋₄ alkyl optionally substituted with F, or C₁₋₄ heteroalkyl optionally substituted with F;
Ring C is a heterocyclic ring or heteroaryl ring, which is optionally substituted with 1-5 instances of R¹¹⁰, i.e., p is 0-5, as valency permits, wherein
   R¹¹⁰ at each occurrence is independently (1) oxo, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) two instances of R¹⁰, together with the intervening atom(s), are joined to form a 3-6 membered ring structure (e.g., cyclopropyl, cyclobutyl, 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F,
U is O or C(═O);
W and V are independently C, N, or CR³⁴;
t is 0, 1, 2, or 3, each Z is independently O, N, NR³¹, CR³²R³³, or CR³⁴;
wherein the bond between W and V, W and Z, or two consecutive Z, is a single bond or a double bond, as valency permits,
J³ and J⁴ are independently N, NR³¹, CR³²R³³, or CR³⁴, wherein
   R³¹ at each occurrence is independently hydrogen, C₁₋₄ alkyl optionally substituted with F, or an optionally substituted 3-6 membered ring structure (e.g., cyclopropyl, cyclobutyl, 4-6 membered heterocyclic ring, or 5 membered heteroaryl),
   each of R³² and R³³ at each occurrence is independently (1) hydrogen, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) an optionally substituted 3-6 membered ring structure (e.g., cyclopropyl, cyclobutyl, 4-6 membered heterocyclic ring, or 5 membered heteroaryl); or (7) R³² and R³³, together with the carbon atom they are both bond to, are joined to form a carbonyl (CO) or a 3-6 membered ring structure (e.g., cyclopropyl, cyclobutyl, or 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F;
   R³⁴ at each occurrence is independently (1) hydrogen, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) an optionally substituted 3-6 membered ring structure (e.g., cyclopropyl, cyclobutyl, 4-6 membered heterocyclic ring, or 5 membered heteroaryl);
   or J³ and one instance of R¹¹⁰ or J⁴ and one instance of R¹⁰ are joined together with the intervening atom(s) to form a ring structure;
R^{20A} is hydrogen, halogen (e.g., F or Cl), CN, C₁₋₄ alkyl optionally substituted with F, C₁₋₄ heteroalkyl optionally substituted with F, or an optionally substituted 3-6 membered ring structure (e.g., cyclopropyl, cyclobutyl, 4-6 membered heterocyclic ring, or 5 membered heteroaryl); and
R^{22A} and R^{23A} are each independently: (1) hydrogen; (2) halogen (e.g., F or Cl), (3) CN, (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) a 3-6 membered ring structure (e.g., cyclopropyl, cyclobutyl, 4-6 membered heterocyclic ring, or 5 membered heteroaryl) optionally substituted 1-3 substituents each independently oxo, halo (e.g., F), OH, NH₂, C₁₋₄ alkyl optionally substituted with F, C₁₋₄ heteroalkyl optionally substituted with F.

Ring C in M-3 can typically be a heterocyclic ring, for example, those having 1 or 2 ring heteroatoms, such as a 5 or 6 membered heterocyclic ring having 1 ring nitrogen atom, e.g., a tetrahydropyridine ring or a dihydropyrrole ring. In some embodiments, Ring C in M-3 can be a heteroaryl ring, such as a 5-membered heteroaryl, preferably, those having 1 or 2 ring heteroatoms, for example, an imidazole or pyrazole ring.

The bond between W and V may be a single bond or a double bond, as valency permits. Similarly, the bond between W and Z may be a single bond or a double bond, as valency permits. And two consecutive Z may be connected through a single bond or a double bond, as valency permits.

J¹ in M-3 or M-4 is typically CR³⁰, wherein R³⁰ is defined herein, preferably, R³⁰ is hydrogen. However, in some embodiments, J^{P} can be N.

J² in M-3 or M-4 is typically also CR³⁰, wherein R³⁰ is defined herein, preferably, R³⁰ is hydrogen. However, in some embodiments, J² can also be N. Preferably, J¹ and J² in M-3 or M-4 are not both N.

In some preferred embodiments, both J^{P} and J² in M-3 or M-4 are CH. For example, in some embodiments, the compound of Formula I can have a structure according to Formula I-3 or I-4:

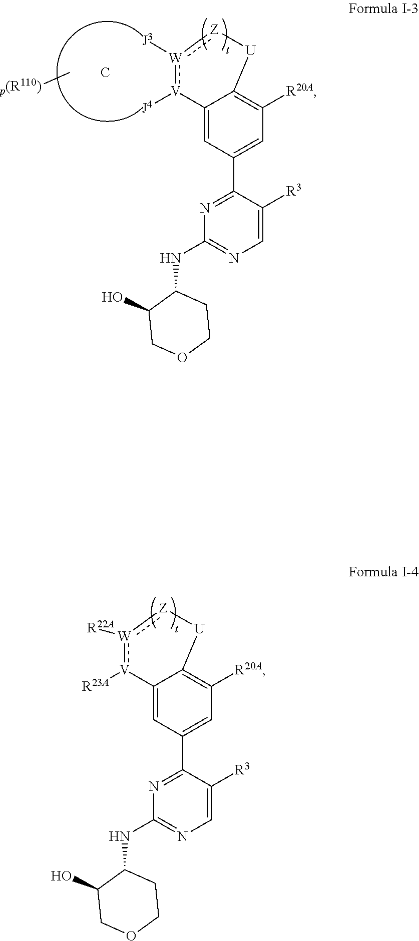

wherein R³, R^{20A}, R^{22A}, R^{23A}, J³, J⁴, p, R¹¹⁰, t, U, V, W, Z, and Ring C are defined herein.

In some embodiments, R^{20A} is hydrogen. In some embodiments, R^{20A} is halogen (e.g., F or Cl) or CN, such as F. In some embodiments, R^{20A} is C₁₋₄ alkyl optionally substituted with F. In some embodiments, R^{20A} is C₁₋₄ heteroalkyl optionally substituted with F. Typically, the C₁₋₄ heteroalkyl has 1 or 2 heteroatoms independently N or O. In some embodiments, R^{20A} can also be an optionally substituted 3-6 membered ring structure (e.g., cyclopropyl, cyclobutyl, 4-6 membered heterocyclic ring, or 5 membered heteroaryl).

R^{20A} is typically hydrogen, halogen, or C₁₋₄ alkyl. For example, in some preferred embodiments, R^{20A} is hydrogen, F, Cl, methyl, or ethyl.

In some embodiments, the integer p is 0. In some embodiments, the integer p is 1 and R¹¹⁰ is OH, NH₂, NH(C₁₋₄ alkyl), or N(C₁₋₄ alkyl)(C₁₋₄ alkyl), or C₁₋₄ alkyl.

In some embodiments, the integer p is 2. In such embodiments, typically, R¹¹⁰ at each occurrence is independently C₁₋₄ alkyl (e.g., methyl, ethyl); or two R¹¹⁰ are joined together with the intervening atom(s) to form a 3-6 membered ring, such as a cyclopropyl ring.

J³ and J⁴ are independently N, NR³¹, CR³²R³³, or CR³⁴. For example, in some embodiments, J³ is NR³¹, and R³¹ is hydrogen or C₁₋₄ alkyl (e.g., methyl or ethyl). In some embodiments, J³ is N. In some embodiments, J³ is CR³⁴, and R³⁴ is hydrogen or C₁₋₄ alkyl (e.g., methyl or ethyl). In some embodiments, J³ is CR³²R³³, and R³² is hydrogen or C₁₋₄ alkyl (e.g., methyl or ethyl) and R³³ is hydrogen or C₁₋₄ alkyl (e.g., methyl or ethyl), e.g., J³ is CH₂, CH(CH₃), or C(CH₃)₂. In some embodiments, J³ is CR³²R³³, and R³² and R³³ together with the carbon atom they are both bonded to form a 3-4 membered ring, such as a cyclopropyl. In some embodiments, J⁴ is NR³¹, and R³¹ is hydrogen or C₁₋₄ alkyl (e.g., methyl or ethyl). In some embodiments, J⁴ is N. In some embodiments, J⁴ is CR³⁴, and R³⁴ is hydrogen or C₁₋₄ alkyl (e.g., methyl or ethyl). In some embodiments, J⁴ is CR³²R³³, and R³² is hydrogen or C₁₋₄ alkyl (e.g., methyl or ethyl) and R³³ is hydrogen or C₁₋₄ alkyl (e.g., methyl or ethyl), e.g., J⁴ is CH₂, CH(CH₃), or C(CH₃)₂. In some embodiments, J⁴ is CR³²R³³, and R³² and R³³ together with the carbon atom they are both bonded to form a 3-4 membered ring, such as a cyclopropyl. Typically, when J³ or J⁴ is N or CR³⁴, it is meant that J³ or J⁴, respectively, forms a double bond with another ring atom in Ring C, such as those exemplified herein.

U in M-3 or M-4 is typically O. In some embodiments, U can also be C(═O).

The integer t is typically 1 or 2.

Typically, Z at each occurrence is independently CR³²R³³, and R³² is hydrogen or C₁₋₄ alkyl (e.g., methyl or ethyl) and R³³ is hydrogen or C₁₋₄ alkyl (e.g., methyl or ethyl), e.g., Z is CH₂, CH(CH₃), or C(CH₃)₂. In some embodiments, one instance of Z is CR³²R³³, wherein R³² and R³³ together with the carbon atom they are both bonded to form a 3-4 membered ring, such as a cyclopropyl; and any remaining instances of Z is independently CR³²R³³, wherein R³² is hydrogen or C₁₋₄ alkyl (e.g., methyl or ethyl) and R³³ is hydrogen or C₁₋₄ alkyl (e.g., methyl or ethyl), e.g., the remaining instances of Z is independently CH₂, CH(CH₃), or C(CH₃)₂.

In some embodiments, W is C, and the Z directly connecting to W is CR³⁴, wherein R³⁴ is hydrogen or C₁₋₄ alkyl (e.g., methyl or ethyl), and the bond between Z and W is a double bond.

In some embodiments, W is C. In some embodiments, W is CH.

In some embodiments, V is C. In some embodiments, V is CH. In some embodiments, V is N.

For example, in some embodiments, M-3 or M-4 can have a structure of:

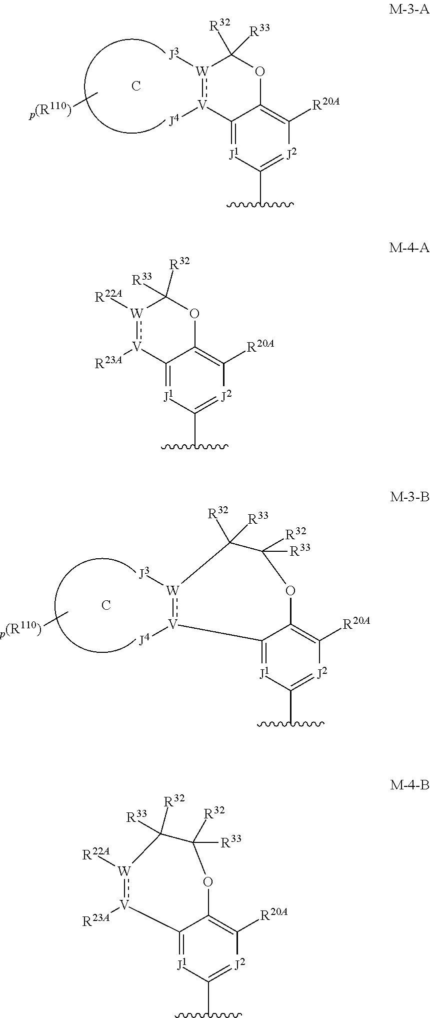

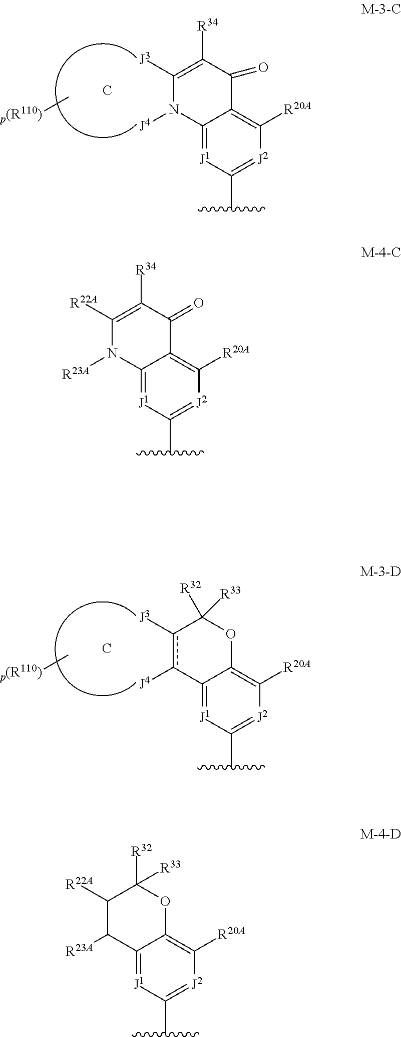

wherein the variables are defined herein.

In one embodiment, R² has a structure according to M-3-A-1, M-3-A-2, M-3-A-3, M-4-A-1, M-3-1B-1, M-3-1B-2, or M-3-C-1:

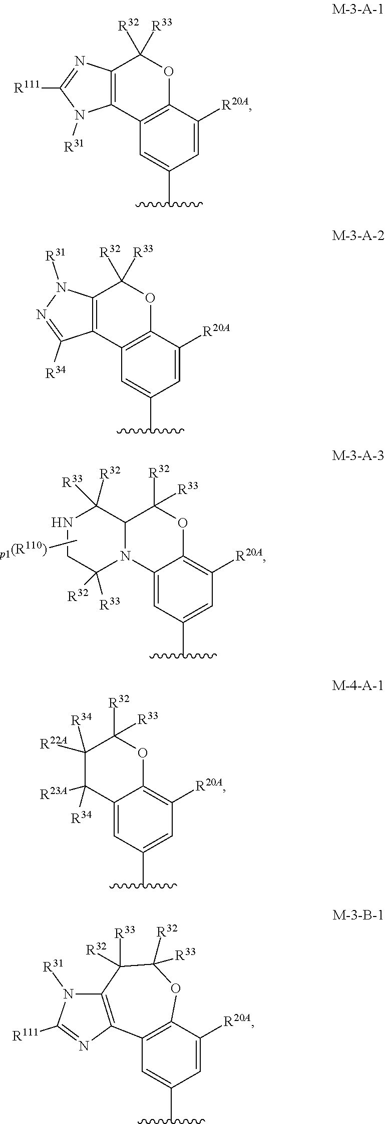

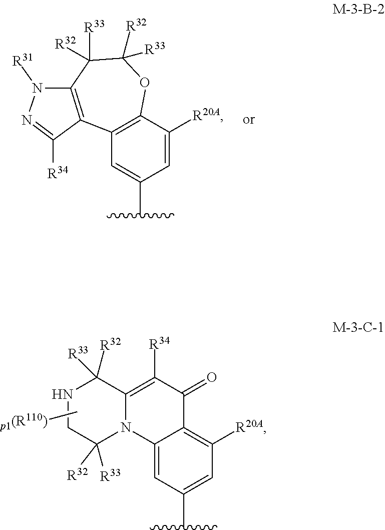

wherein:
R¹¹¹ is hydrogen or R¹¹⁰ (wherein R¹¹⁰ is defined herein or elsewhere).

R^{22A} in M-4 can typically be hydrogen, C₁₋₄ alkyl (e.g., methyl or ethyl), or C₁₋₄ heteroalkyl having 1 or 2 heteroatoms independently oxygen or nitrogen. For example, in some embodiments, R^{22A} can be hydrogen or methyl.

In some embodiments, R^{22A} in M-4 (e.g., M-4-D) can be C₁₋₄ alkyl substituted with OH, NH₂, or NH(C₁₋₄ alkyl), for example,

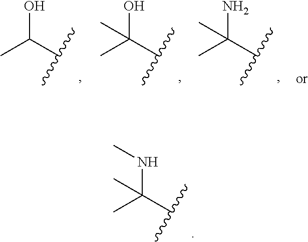

R^{23A} in M-4 can typically be hydrogen, C₁₋₄ alkyl (e.g., methyl or ethyl), or C₁₋₄ heteroalkyl having 1 or 2 heteroatoms independently oxygen or nitrogen. For example, in some embodiments, R^{23A} can be hydrogen or methyl.

In some embodiments, R^{23A} in M-4 can be C₁₋₄ alkyl substituted with OH, NH₂, or NH(C₁₋₄ alkyl).

In some embodiments, one of R^{22A} and R^{23A} is a group containing a hydrogen bond donor.

In some specific embodiments, ring C in M-3 is a 5-membered heteroaryl, and R² can be selected from:

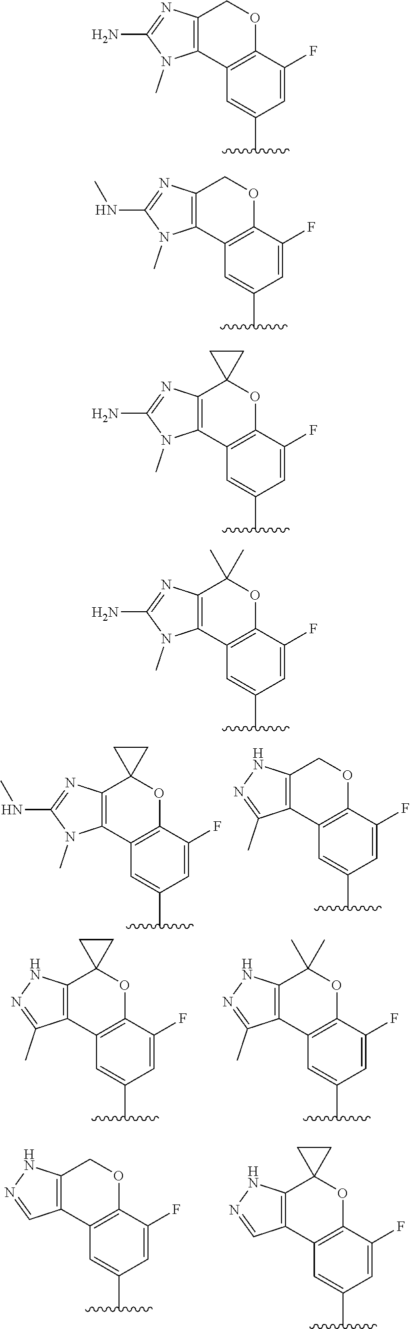

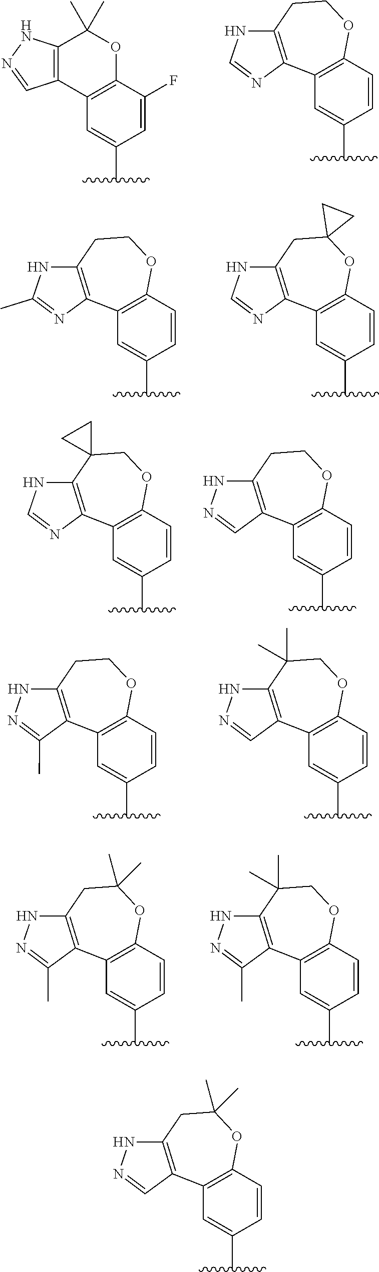

In some specific embodiments, Ring C in M-3 is a heterocyclic structure, and R² can be selected from:

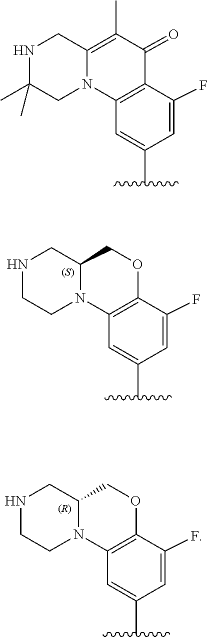

In some preferred embodiments, R² can be selected from

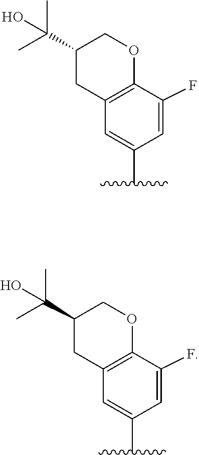 [SMILES: *c1cc(F)c2c(c1)C[C@H](C(C)(C)O)CO2 ; *c1cc(F)c2c(c1)C[C@@H](C(C)(C)O)CO2]

Various groups are suitable for R³ in Formula I. For example, in some embodiments, R³ is hydrogen. In some embodiments, R³ is halogen (e.g., F). In some embodiments, R³ is CN. In some embodiments, R³ is C(O)NR¹¹R¹², wherein R¹¹ and R¹² are defined herein, for example, both R¹¹ and R¹² can be hydrogen. In some embodiments, R³ is an optionally substituted C₃₋₈ carbocyclyl. In some embodiments, R³ is an optionally substituted 4-10 membered heterocyclyl having 1 or 2 ring heteroatoms independently selected from N, O, and S. In some embodiments, R³ is an optionally substituted 5-10 membered heteroaryl having 1-4 ring heteroatoms independently selected from N, O, and S.

In any of the embodiments herein, unless specified or otherwise contrary from context, R³ in Formula I (or any sub-formula provided herein, e.g., Formula I-A, I-B, A, A-S1, A-S2, A-S3, A-S4, A-1, A-2, I-1, I-1-D, I-2, I-2-D, 1-3, or I-4) can be hydrogen, F, Cl, Br, C₁₋₄ alkyl optionally substituted with F, or CN.

In some embodiments, R³ in Formula I (or any of the applicable sub-formulae as described herein, e.g., Formula I-A, I-B, A, A-S1, A-S2, A-S3, A-S4, A-1, A-2, I-1, I-1-D, I-2, I-2-D, 1-3, or I-4) can be an optionally substituted C₁₋₄ alkyl. In some embodiments, R³ can be C₁₋₄ alkyl optionally substituted with one or more, such as 1-3 substituents independently selected from deuterium, F, CN, or OR^{C}, wherein R^{C} at each occurrence is independently hydrogen, C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl. For example, in some embodiments, R³ can be methyl, CD₃, CH₂—OMe, CH₂—OCD₃, ethyl, CHF₂, CF₂CH₃, CH₂CH₂F, CH₂CF₂H, or CF₃. In some embodiments, R³ can be CF₂CF₃. In some preferred embodiments, R³ can be F or Cl.

In some embodiments, R³ in Formula I (or any of the applicable sub-formulae as described herein, e.g., Formula I-A, I-B, A, A-S1, A-S2, A-S3, A-S4, A-1, A-2, I-1, I-1-D, I-2, I-2-D, 1-3, or 1-4) can be an optionally substituted C₂₋₄ alkenyl, such as

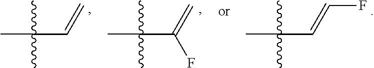

In some embodiments, R³ in Formula I (or any of the applicable sub-formulae as described herein, e.g., Formula I-A, I-B, A, A-S1, A-S2, A-S3, A-S4, A-1, A-2, 1-1, I-1-D, I-2, I-2-D, 1-3, or 1-4) can be an optionally substituted C₂₋₄ alkynyl, such as

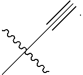 [SMILES: *C#C]

In some embodiments, R³ in Formula I (or any of the applicable sub-formulae as described herein, e.g., Formula I-A, I-B, A, A-S1, A-S2, A-S3, A-S4, A-1, A-2, I-1, I-1-D, I-2, I-2-D, 1-3, or I-4) can be OR^{A}. For example, in some embodiments, R³ is OR^{A}, and R^{A} is hydrogen, C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl.

In some embodiments, R³ in Formula I (or any of the applicable sub-formulae as described herein, e.g., Formula I-A, I-B, A, A-S1, A-S2, A-S3, A-S4, A-1, A-2, 1-1, I-1-D, I-2, I-2-D, I-3, or 1-4) can be C(O)R^{B}. For example, in some embodiments, R³ is C(O)R^{B} and R^{B} is hydrogen, C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl.

In some embodiments, R³ in Formula I (or any of the applicable sub-formulae as described herein, e.g., Formula I-A, I-B, A, A-S1, A-S2, A-S3, A-S4, A-1, A-2, I-1, I-1-D, I-2, I-2-D, I-3, or 1-4) can also be a C₃₋₆ cycloalkyl (e.g., cyclopropyl, cyclobutyl, cyclopentyl, etc.), 4-6 membered heterocyclyl having 1-2 ring heteroatoms independently selected from N, O, and S, such as oxetanyl, tetrahydrofuranyl, or 5-6 membered heteroaryl having 1-4 ring heteroatoms independently selected from N, O, and S, such as thiazolyl, each of which is optionally substituted with 1-3 substituents independently selected from oxo (as applicable), deuterium, F, CN, G¹, OH, O-G¹, NH₂, NH(G¹), N(G¹)(G¹), C(O)—NH₂, C(O)—NH(G¹), and C(O)—N(G¹)(G¹), wherein G¹ at each occurrence is independently a C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl.

In any of the embodiments herein, unless specified or otherwise contrary from context, R³ in Formula I (or any of the applicable sub-formulae as described herein, e.g., Formula I-A, I-B, A, A-S1, A-S2, A-S3, A-S4, A-1, A-2, I-1, I-1-D, 1-2, I-2-D, 1-3, or 1-4) can be selected from:

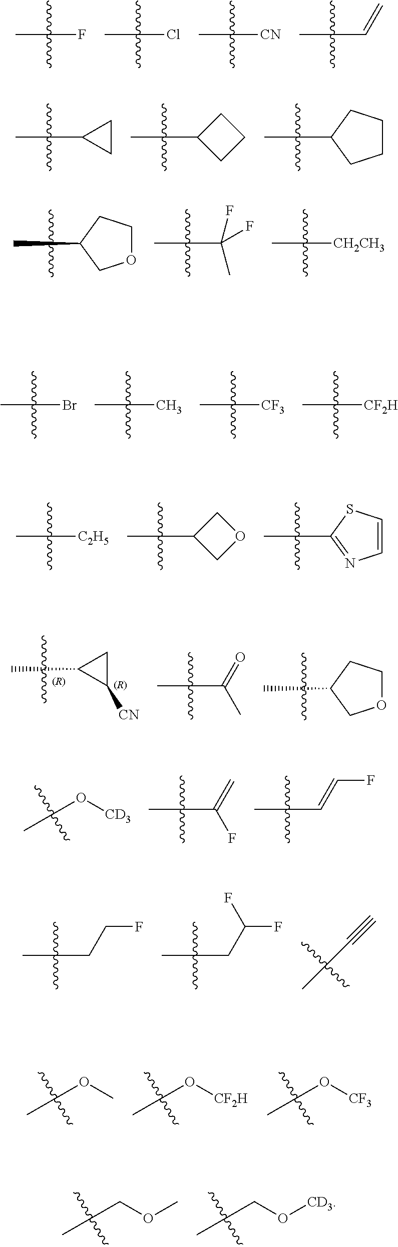

R⁴ in Formula I (or any of the applicable sub-formulae as described herein, e.g., Formula I-A, I-B, A, A-S1, A-S2, A-S3, A-S4, A-1, A-2, I-1, 1-2, I-3, or 1-4) is typically hydrogen. In some embodiments, R⁴ in Formula I can be deuterium. In some embodiments, R⁴ in Formula I can also be a halogen (e.g., F), optionally substituted C₁₋₆ alkyl, or NR¹¹R¹². For example, in some embodiments, R⁴ in Formula I is NH₂.

In some embodiments, in Formula I (or any of the applicable sub-formulae as described herein, e.g., Formula I-A, I-B, A, A-S1, A-S2, A-S3, A-S4, A-1, or A-2), R³ and R⁴, together with the intervening atoms, can also be joined to form an optionally substituted 4-8 membered ring structure, such as 4-8 membered heterocyclic structure or 5 or 6 membered heteroaryl structure. For example, in any of the embodiments herein, unless specified or otherwise contrary from context, in Formula I (or any of the applicable sub-formulae as described herein, e.g., Formula I-A, I-B, A, A-S1, A-S2, A-S3, A-S4, A-1, or A-2), R³ and R⁴, together with the intervening atoms, can be joined to form one of the following:

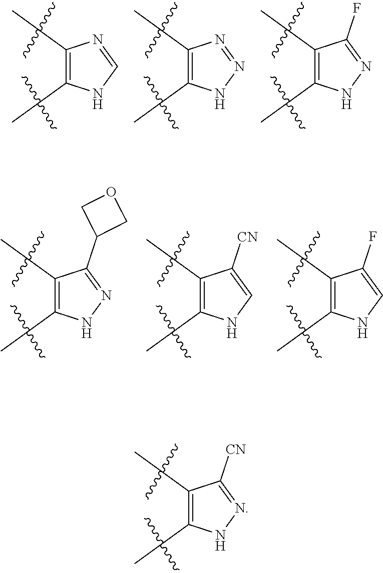

In some embodiments, each C₁₋₄ alkyl of R²¹, R²², R²³, R¹¹⁰, R³², R³³, R³⁴, R^{22A}, or R^{23A} is independently optionally substituted with 1-3 substituents each independently F, OH, NH₂, NH(C₁₋₄ alkyl), or N(C₁₋₄ alkyl)(C₁₋₄ alkyl).

In some embodiments, the present disclosure also provide a compound selected from Table 1 below, a deuterated analog thereof, a stereoisomer thereof, or a pharmaceutically acceptable salt thereof:

TABLE 1
List of Compounds

| | | |
|---|---|---|
| 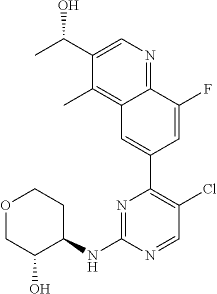 [SMILES: Cc1c([C@H](C)O)cnc2c(F)cc(-c3nc(N[C@@H]4CCOC[C@H]4O)ncc3Cl)cc12] | 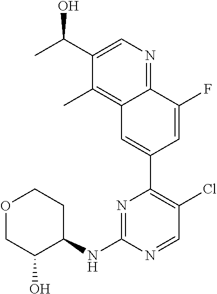 [SMILES: Cc1c([C@@H](C)O)cnc2c(F)cc(-c3nc(N[C@@H]4CCOC[C@H]4O)ncc3Cl)cc12] | 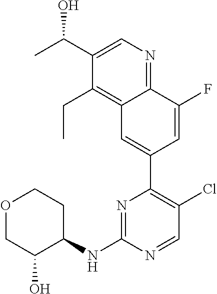 [SMILES: CCc1c([C@H](C)O)cnc2c(F)cc(-c3nc(N[C@@H]4CCOC[C@H]4O)ncc3Cl)cc12] |
| 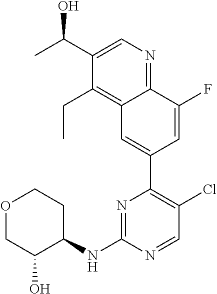 [SMILES: CCc1c([C@@H](C)O)cnc2c(F)cc(-c3nc(N[C@@H]4CCOC[C@H]4O)ncc3Cl)cc12] | 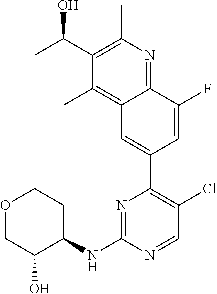 [SMILES: Cc1nc2c(F)cc(-c3nc(N[C@@H]4CCOC[C@H]4O)ncc3Cl)cc2c(C)c1[C@@H](C)O] | 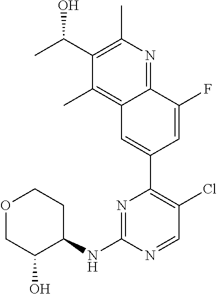 [SMILES: Cc1nc2c(F)cc(-c3nc(N[C@@H]4CCOC[C@H]4O)ncc3Cl)cc2c(C)c1[C@H](C)O] |
| 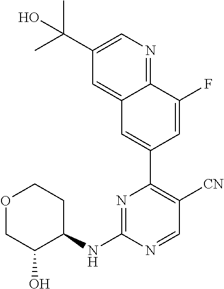 [SMILES: CC(C)(O)c1cnc2c(F)cc(-c3nc(N[C@@H]4CCOC[C@H]4O)ncc3C#N)cc2c1] | 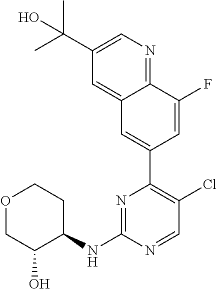 [SMILES: CC(C)(O)c1cnc2c(F)cc(-c3nc(N[C@@H]4CCOC[C@H]4O)ncc3Cl)cc2c1] | 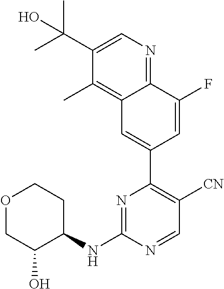 [SMILES: Cc1c(C(C)(C)O)cnc2c(F)cc(-c3nc(N[C@@H]4CCOC[C@H]4O)ncc3C#N)cc12] |
| 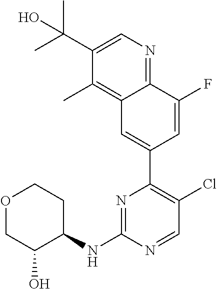 [SMILES: Cc1c(C(C)(C)O)cnc2c(F)cc(-c3nc(N[C@@H]4CCOC[C@H]4O)ncc3Cl)cc12] | 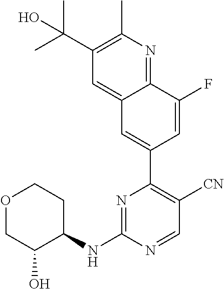 [SMILES: Cc1nc2c(F)cc(-c3nc(N[C@@H]4CCOC[C@H]4O)ncc3C#N)cc2cc1C(C)(C)O] | 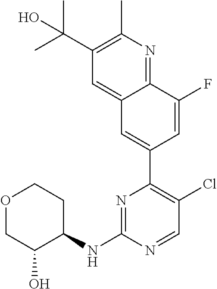 [SMILES: Cc1nc2c(F)cc(-c3nc(N[C@@H]4CCOC[C@H]4O)ncc3Cl)cc2cc1C(C)(C)O] |
| 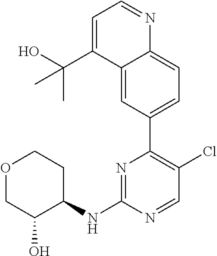 [SMILES: CC(C)(O)c1ccnc2ccc(-c3nc(N[C@@H]4CCOC[C@H]4O)ncc3Cl)cc12] | 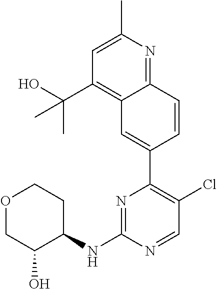 [SMILES: Cc1cc(C(C)(C)O)c2cc(-c3nc(N[C@@H]4CCOC[C@H]4O)ncc3Cl)ccc2n1] | 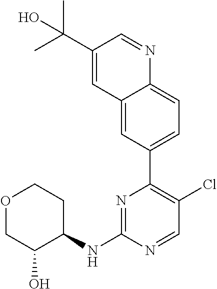 [SMILES: CC(C)(O)c1cnc2ccc(-c3nc(N[C@@H]4CCOC[C@H]4O)ncc3Cl)cc2c1] |
| 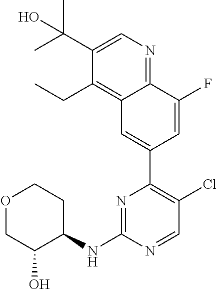 [SMILES: CCc1c(C(C)(C)O)cnc2c(F)cc(-c3nc(N[C@@H]4CCOC[C@H]4O)ncc3Cl)cc12] | 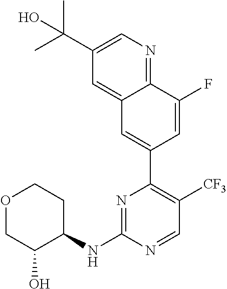 [SMILES: CC(C)(O)c1cnc2c(F)cc(-c3nc(N[C@@H]4CCOC[C@H]4O)ncc3C(F)(F)F)cc2c1] | 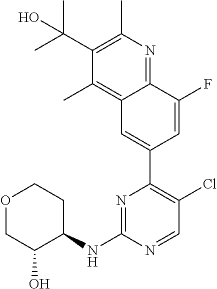 [SMILES: Cc1nc2c(F)cc(-c3nc(N[C@@H]4CCOC[C@H]4O)ncc3Cl)cc2c(C)c1C(C)(C)O] |
| 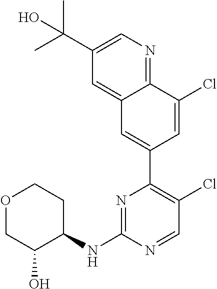 [SMILES: CC(C)(O)c1cnc2c(Cl)cc(-c3nc(N[C@@H]4CCOC[C@H]4O)ncc3Cl)cc2c1] | 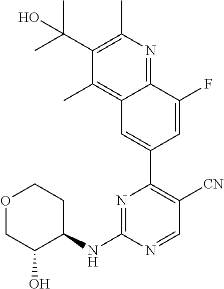 [SMILES: Cc1nc2c(F)cc(-c3nc(N[C@@H]4CCOC[C@H]4O)ncc3C#N)cc2c(C)c1C(C)(C)O] | 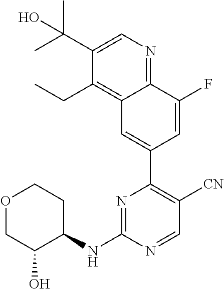 [SMILES: CCc1c(C(C)(C)O)cnc2c(F)cc(-c3nc(N[C@@H]4CCOC[C@H]4O)ncc3C#N)cc12] |
| 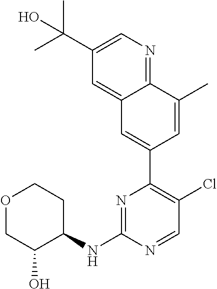 [SMILES: Cc1cc(-c2nc(N[C@@H]3CCOC[C@H]3O)ncc2Cl)cc2cc(C(C)(C)O)cnc12] | 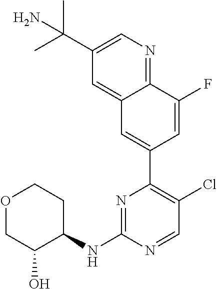 [SMILES: CC(C)(N)c1cnc2c(F)cc(-c3nc(N[C@@H]4CCOC[C@H]4O)ncc3Cl)cc2c1] | 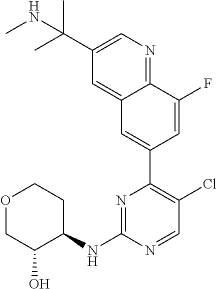 [SMILES: CNC(C)(C)c1cnc2c(F)cc(-c3nc(N[C@@H]4CCOC[C@H]4O)ncc3Cl)cc2c1] |
| 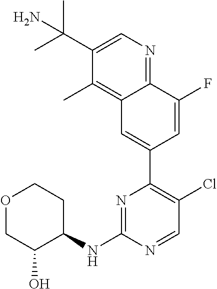 [SMILES: Cc1c(C(C)(C)N)cnc2c(F)cc(-c3nc(N[C@@H]4CCOC[C@H]4O)ncc3Cl)cc12] | 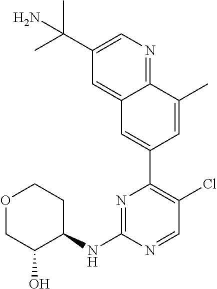 [SMILES: Cc1cc(-c2nc(N[C@@H]3CCOC[C@H]3O)ncc2Cl)cc2cc(C(C)(C)N)cnc12] | 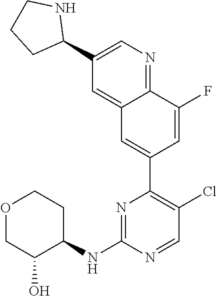 [SMILES: O[C@@H]1COCC[C@H]1Nc1ncc(Cl)c(-c2cc(F)c3ncc([C@H]4CCCN4)cc3c2)n1] |
| 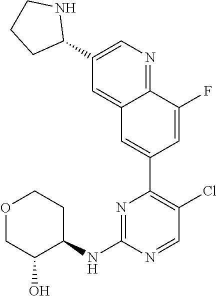 [SMILES: O[C@@H]1COCC[C@H]1Nc1ncc(Cl)c(-c2cc(F)c3ncc([C@@H]4CCCN4)cc3c2)n1] | 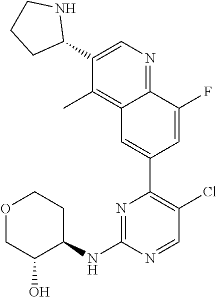 [SMILES: Cc1c([C@@H]2CCCN2)cnc2c(F)cc(-c3nc(N[C@@H]4CCOC[C@H]4O)ncc3Cl)cc12] | 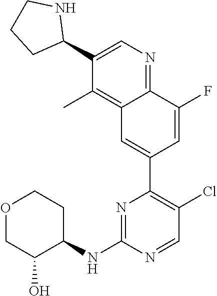 [SMILES: Cc1c([C@H]2CCCN2)cnc2c(F)cc(-c3nc(N[C@@H]4CCOC[C@H]4O)ncc3Cl)cc12] |
| 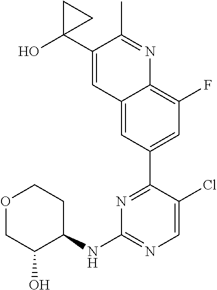 [SMILES: Cc1nc2c(F)cc(-c3nc(N[C@@H]4CCOC[C@H]4O)ncc3Cl)cc2cc1C1(O)CC1] | 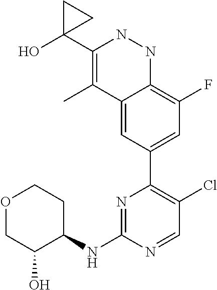 [SMILES: CC1=C(C2(O)CC2)NNc2c(F)cc(-c3nc(N[C@@H]4CCOC[C@H]4O)ncc3Cl)cc21] | 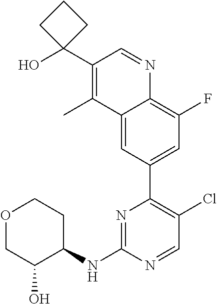 [SMILES: Cc1c(C2(O)CCC2)cnc2c(F)cc(-c3nc(N[C@@H]4CCOC[C@H]4O)ncc3Cl)cc12] |
| 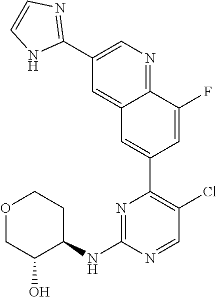 [SMILES: O[C@@H]1COCC[C@H]1Nc1ncc(Cl)c(-c2cc(F)c3ncc(-c4ncc[nH]4)cc3c2)n1] | 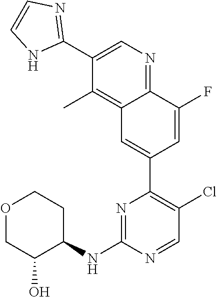 [SMILES: Cc1c(-c2ncc[nH]2)cnc2c(F)cc(-c3nc(N[C@@H]4CCOC[C@H]4O)ncc3Cl)cc12] | 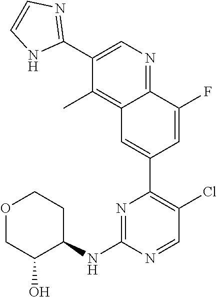 [SMILES: Cc1c(-c2ncc[nH]2)cnc2c(F)cc(-c3nc(N[C@@H]4CCOC[C@H]4O)ncc3Cl)cc12] |
| 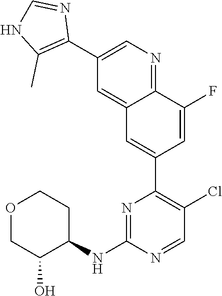 [SMILES: Cc1[nH]cnc1-c1cnc2c(F)cc(-c3nc(N[C@@H]4CCOC[C@H]4O)ncc3Cl)cc2c1] | 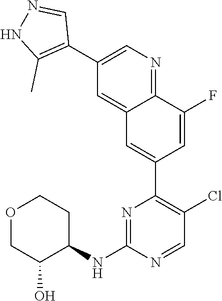 [SMILES: Cc1[nH]ncc1-c1cnc2c(F)cc(-c3nc(N[C@@H]4CCOC[C@H]4O)ncc3Cl)cc2c1] | 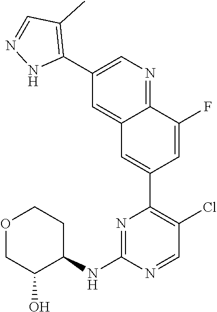 [SMILES: Cc1cn[nH]c1-c1cnc2c(F)cc(-c3nc(N[C@@H]4CCOC[C@H]4O)ncc3Cl)cc2c1] |
| 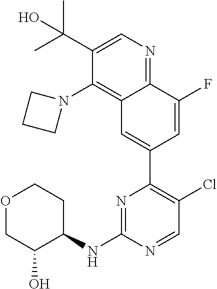 [SMILES: CC(C)(O)c1cnc2c(F)cc(-c3nc(N[C@@H]4CCOC[C@H]4O)ncc3Cl)cc2c1N1CCC1] | 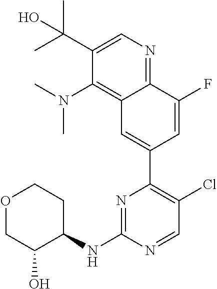 [SMILES: CN(C)c1c(C(C)(C)O)cnc2c(F)cc(-c3nc(N[C@@H]4CCOC[C@H]4O)ncc3Cl)cc12] | 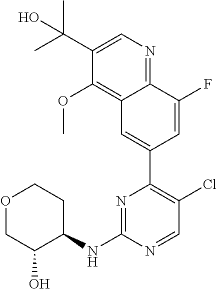 [SMILES: COc1c(C(C)(C)O)cnc2c(F)cc(-c3nc(N[C@@H]4CCOC[C@H]4O)ncc3Cl)cc12] |
| 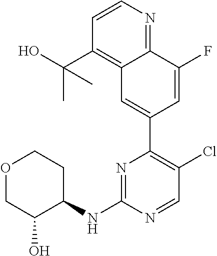 [SMILES: CC(C)(O)c1ccnc2c(F)cc(-c3nc(N[C@@H]4CCOC[C@H]4O)ncc3Cl)cc12] | 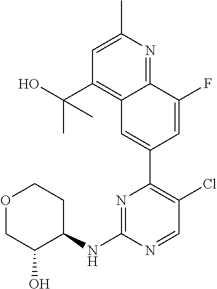 [SMILES: Cc1cc(C(C)(C)O)c2cc(-c3nc(N[C@@H]4CCOC[C@H]4O)ncc3Cl)cc(F)c2n1] | 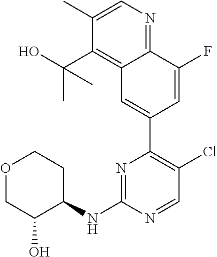 [SMILES: Cc1cnc2c(F)cc(-c3nc(N[C@@H]4CCOC[C@H]4O)ncc3Cl)cc2c1C(C)(C)O] |
| 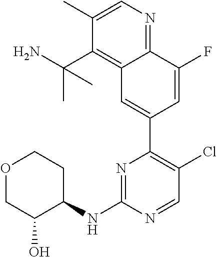 [SMILES: Cc1cnc2c(F)cc(-c3nc(N[C@@H]4CCOC[C@H]4O)ncc3Cl)cc2c1C(C)(C)N] | 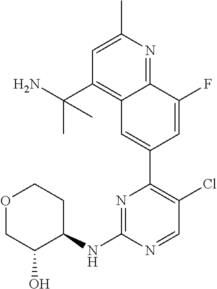 [SMILES: Cc1cc(C(C)(C)N)c2cc(-c3nc(N[C@@H]4CCOC[C@H]4O)ncc3Cl)cc(F)c2n1] | 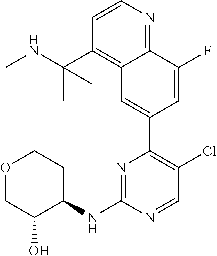 [SMILES: CNC(C)(C)c1ccnc2c(F)cc(-c3nc(N[C@@H]4CCOC[C@H]4O)ncc3Cl)cc12] |
| 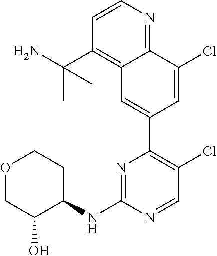 [SMILES: CC(C)(N)c1ccnc2c(Cl)cc(-c3nc(N[C@@H]4CCOC[C@H]4O)ncc3Cl)cc12] | 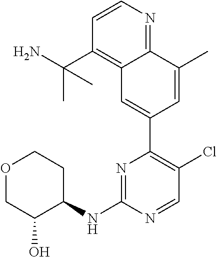 [SMILES: Cc1cc(-c2nc(N[C@@H]3CCOC[C@H]3O)ncc2Cl)cc2c(C(C)(C)N)ccnc12] | 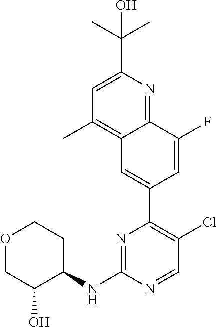 [SMILES: Cc1cc(C(C)(C)O)nc2c(F)cc(-c3nc(N[C@@H]4CCOC[C@H]4O)ncc3Cl)cc12] |
| 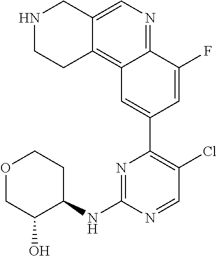 [SMILES: O[C@@H]1COCC[C@H]1Nc1ncc(Cl)c(-c2cc(F)c3ncc4c(c3c2)CCNC4)n1] | 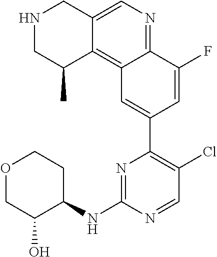 [SMILES: C[C@H]1CNCc2cnc3c(F)cc(-c4nc(N[C@@H]5CCOC[C@H]5O)ncc4Cl)cc3c21] | 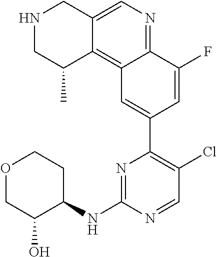 [SMILES: C[C@@H]1CNCc2cnc3c(F)cc(-c4nc(N[C@@H]5CCOC[C@H]5O)ncc4Cl)cc3c21] |
| 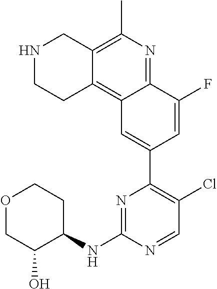 [SMILES: Cc1nc2c(F)cc(-c3nc(N[C@@H]4CCOC[C@H]4O)ncc3Cl)cc2c2c1CNCC2] | 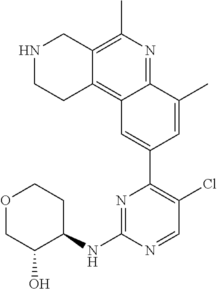 [SMILES: Cc1nc2c(C)cc(-c3nc(N[C@@H]4CCOC[C@H]4O)ncc3Cl)cc2c2c1CNCC2] | 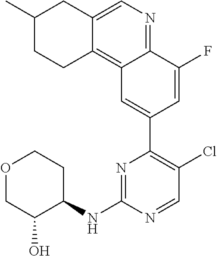 [SMILES: CC1CCc2c(cnc3c(F)cc(-c4nc(N[C@@H]5CCOC[C@H]5O)ncc4Cl)cc23)C1] |
| 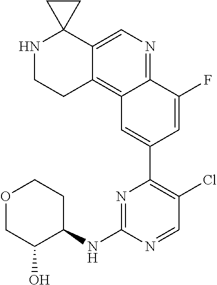 [SMILES: O[C@@H]1COCC[C@H]1Nc1ncc(Cl)c(-c2cc(F)c3ncc4c(c3c2)CCNC42CC2)n1] | 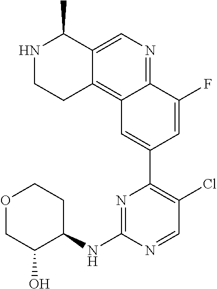 [SMILES: C[C@@H]1NCCc2c1cnc1c(F)cc(-c3nc(N[C@@H]4CCOC[C@H]4O)ncc3Cl)cc21] | 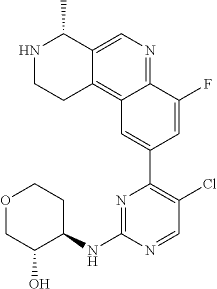 [SMILES: C[C@H]1NCCc2c1cnc1c(F)cc(-c3nc(N[C@@H]4CCOC[C@H]4O)ncc3Cl)cc21] |
| 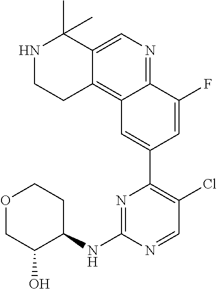 [SMILES: CC1(C)NCCc2c1cnc1c(F)cc(-c3nc(N[C@@H]4CCOC[C@H]4O)ncc3Cl)cc21] | 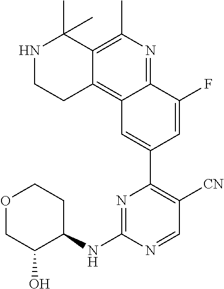 [SMILES: Cc1nc2c(F)cc(-c3nc(N[C@@H]4CCOC[C@H]4O)ncc3C#N)cc2c2c1C(C)(C)NCC2] | 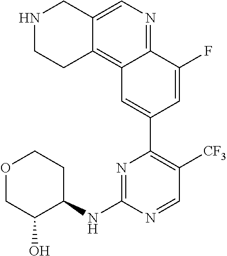 [SMILES: O[C@@H]1COCC[C@H]1Nc1ncc(C(F)(F)F)c(-c2cc(F)c3ncc4c(c3c2)CCNC4)n1] |
| 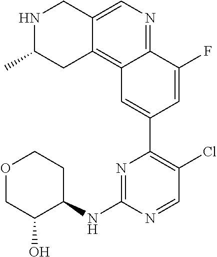 [SMILES: C[C@H]1Cc2c(cnc3c(F)cc(-c4nc(N[C@@H]5CCOC[C@H]5O)ncc4Cl)cc23)CN1] | 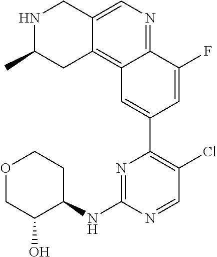 [SMILES: C[C@@H]1Cc2c(cnc3c(F)cc(-c4nc(N[C@@H]5CCOC[C@H]5O)ncc4Cl)cc23)CN1] | 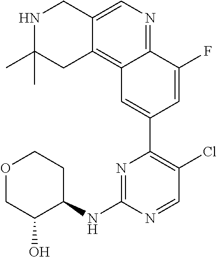 [SMILES: CC1(C)Cc2c(cnc3c(F)cc(-c4nc(N[C@@H]5CCOC[C@H]5O)ncc4Cl)cc23)CN1] |
| 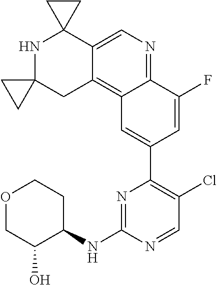 [SMILES: O[C@@H]1COCC[C@H]1Nc1ncc(Cl)c(-c2cc(F)c3ncc4c(c3c2)CC2(CC2)NC42CC2)n1] | 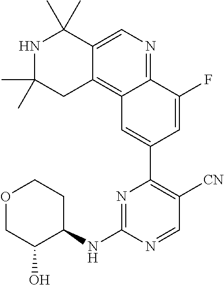 [SMILES: CC1(C)Cc2c(cnc3c(F)cc(-c4nc(N[C@@H]5CCOC[C@H]5O)ncc4C#N)cc23)C(C)(C)N1] | 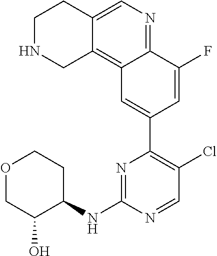 [SMILES: O[C@@H]1COCC[C@H]1Nc1ncc(Cl)c(-c2cc(F)c3ncc4c(c3c2)CNCC4)n1] |
| 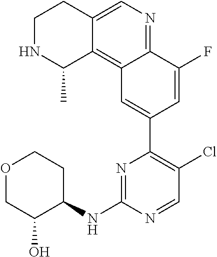 [SMILES: C[C@@H]1NCCc2cnc3c(F)cc(-c4nc(N[C@@H]5CCOC[C@H]5O)ncc4Cl)cc3c21] | 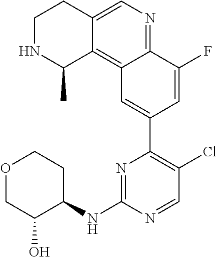 [SMILES: C[C@H]1NCCc2cnc3c(F)cc(-c4nc(N[C@@H]5CCOC[C@H]5O)ncc4Cl)cc3c21] | 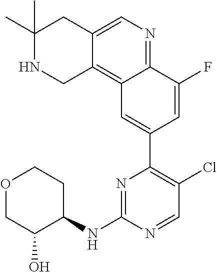 [SMILES: CC1(C)Cc2cnc3c(F)cc(-c4nc(N[C@@H]5CCOC[C@H]5O)ncc4Cl)cc3c2CN1] |
| 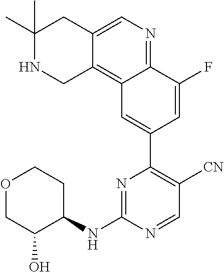 [SMILES: CC1(C)Cc2cnc3c(F)cc(-c4nc(N[C@@H]5CCOC[C@H]5O)ncc4C#N)cc3c2CN1] | 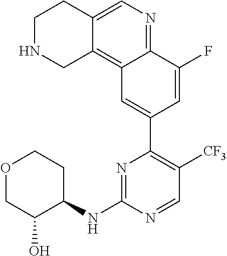 [SMILES: O[C@@H]1COCC[C@H]1Nc1ncc(C(F)(F)F)c(-c2cc(F)c3ncc4c(c3c2)CNCC4)n1] | 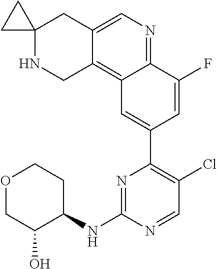 [SMILES: O[C@@H]1COCC[C@H]1Nc1ncc(Cl)c(-c2cc(F)c3ncc4c(c3c2)CNC2(CC2)C4)n1] |
| 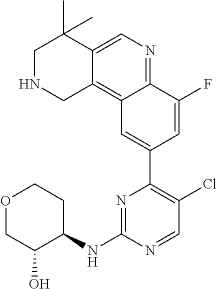 [SMILES: CC1(C)CNCc2c1cnc1c(F)cc(-c3nc(N[C@@H]4CCOC[C@H]4O)ncc3Cl)cc21] | 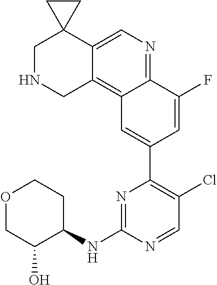 [SMILES: O[C@@H]1COCC[C@H]1Nc1ncc(Cl)c(-c2cc(F)c3ncc4c(c3c2)CNCC42CC2)n1] | 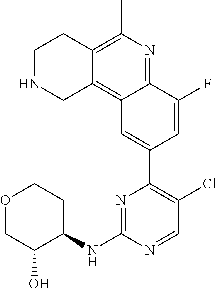 [SMILES: Cc1nc2c(F)cc(-c3nc(N[C@@H]4CCOC[C@H]4O)ncc3Cl)cc2c2c1CCNC2] |
| 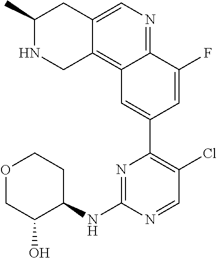 [SMILES: C[C@H]1Cc2cnc3c(F)cc(-c4nc(N[C@@H]5CCOC[C@H]5O)ncc4Cl)cc3c2CN1] | 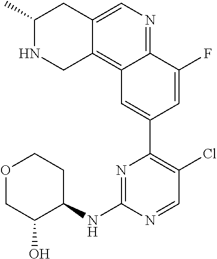 [SMILES: C[C@@H]1Cc2cnc3c(F)cc(-c4nc(N[C@@H]5CCOC[C@H]5O)ncc4Cl)cc3c2CN1] | 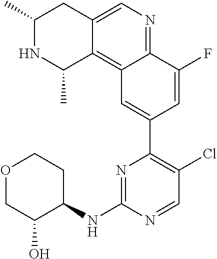 [SMILES: C[C@@H]1Cc2cnc3c(F)cc(-c4nc(N[C@@H]5CCOC[C@H]5O)ncc4Cl)cc3c2[C@H](C)N1] |
| 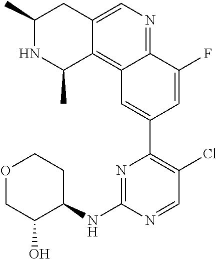 [SMILES: C[C@H]1Cc2cnc3c(F)cc(-c4nc(N[C@@H]5CCOC[C@H]5O)ncc4Cl)cc3c2[C@@H](C)N1] | 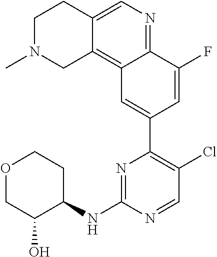 [SMILES: CN1CCc2cnc3c(F)cc(-c4nc(N[C@@H]5CCOC[C@H]5O)ncc4Cl)cc3c2C1] | 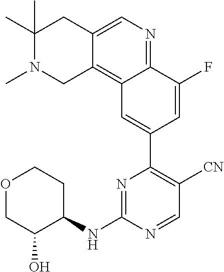 [SMILES: CN1Cc2c(cnc3c(F)cc(-c4nc(N[C@@H]5CCOC[C@H]5O)ncc4C#N)cc23)CC1(C)C] |
| 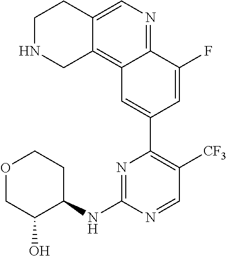 [SMILES: O[C@@H]1COCC[C@H]1Nc1ncc(C(F)(F)F)c(-c2cc(F)c3ncc4c(c3c2)CNCC4)n1] | 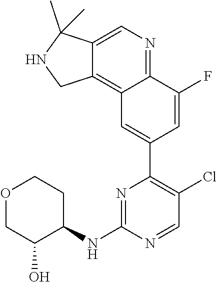 [SMILES: CC1(C)NCc2c1cnc1c(F)cc(-c3nc(N[C@@H]4CCOC[C@H]4O)ncc3Cl)cc21] | 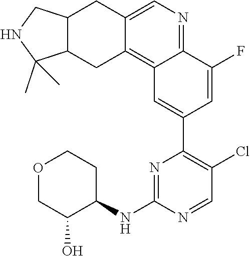 [SMILES: CC1(C)NCC2Cc3cnc4c(F)cc(-c5nc(N[C@@H]6CCOC[C@H]6O)ncc5Cl)cc4c3CC21] |
| 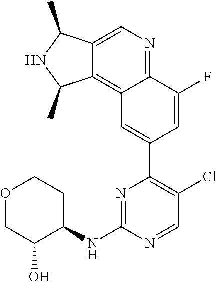 [SMILES: C[C@@H]1N[C@H](C)c2c1cnc1c(F)cc(-c3nc(N[C@@H]4CCOC[C@H]4O)ncc3Cl)cc21] | 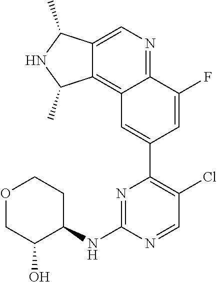 [SMILES: C[C@@H]1N[C@H](C)c2cnc3c(F)cc(-c4nc(N[C@@H]5CCOC[C@H]5O)ncc4Cl)cc3c21] | 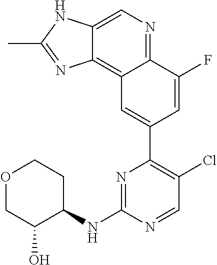 [SMILES: Cc1nc2c(cnc3c(F)cc(-c4nc(N[C@@H]5CCOC[C@H]5O)ncc4Cl)cc32)[nH]1] |
| 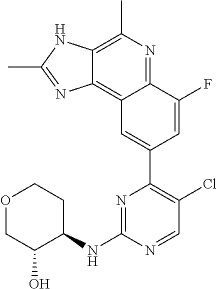 [SMILES: Cc1nc2c([nH]1)c(C)nc1c(F)cc(-c3nc(N[C@@H]4CCOC[C@H]4O)ncc3Cl)cc12] | 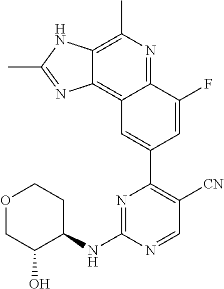 [SMILES: Cc1nc2c([nH]1)c(C)nc1c(F)cc(-c3nc(N[C@@H]4CCOC[C@H]4O)ncc3C#N)cc12] | 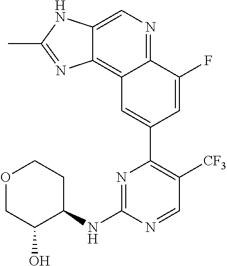 [SMILES: Cc1nc2c(cnc3c(F)cc(-c4nc(N[C@@H]5CCOC[C@H]5O)ncc4C(F)(F)F)cc32)[nH]1] |
| 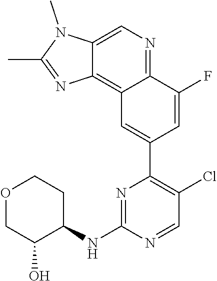 [SMILES: Cc1nc2c3cc(-c4nc(N[C@@H]5CCOC[C@H]5O)ncc4Cl)cc(F)c3ncc2n1C] | 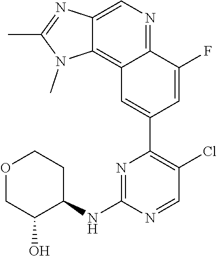 [SMILES: Cc1nc2cnc3c(F)cc(-c4nc(N[C@@H]5CCOC[C@H]5O)ncc4Cl)cc3c2n1C] | 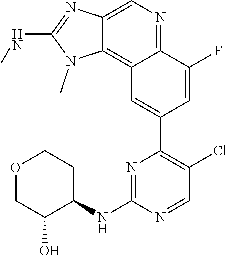 [SMILES: CNc1nc2cnc3c(F)cc(-c4nc(N[C@@H]5CCOC[C@H]5O)ncc4Cl)cc3c2n1C] |
| 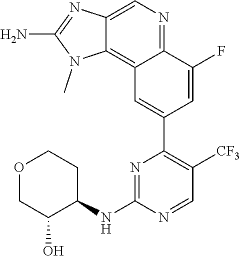 [SMILES: Cn1c(N)nc2cnc3c(F)cc(-c4nc(N[C@@H]5CCOC[C@H]5O)ncc4C(F)(F)F)cc3c21] | 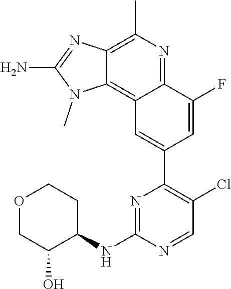 [SMILES: Cc1nc2c(F)cc(-c3nc(N[C@@H]4CCOC[C@H]4O)ncc3Cl)cc2c2c1nc(N)n2C] | 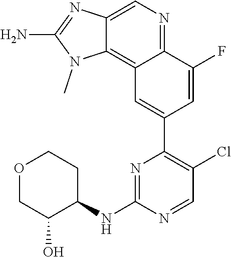 [SMILES: Cn1c(N)nc2cnc3c(F)cc(-c4nc(N[C@@H]5CCOC[C@H]5O)ncc4Cl)cc3c21] |
| 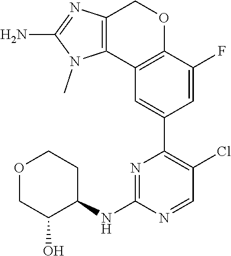 [SMILES: Cn1c(N)nc2c1-c1cc(-c3nc(N[C@@H]4CCOC[C@H]4O)ncc3Cl)cc(F)c1OC2] | 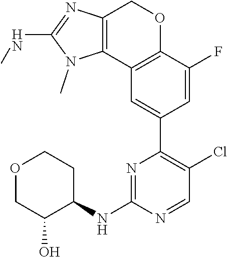 [SMILES: CNc1nc2c(n1C)-c1cc(-c3nc(N[C@@H]4CCOC[C@H]4O)ncc3Cl)cc(F)c1OC2] | 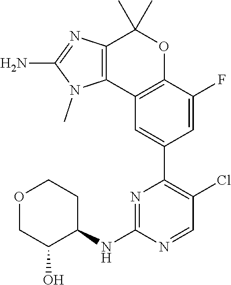 [SMILES: Cn1c(N)nc2c1-c1cc(-c3nc(N[C@@H]4CCOC[C@H]4O)ncc3Cl)cc(F)c1OC2(C)C] |
| 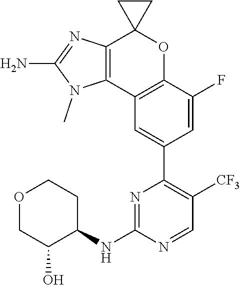 [SMILES: Cn1c(N)nc2c1-c1cc(-c3nc(N[C@@H]4CCOC[C@H]4O)ncc3C(F)(F)F)cc(F)c1OC21CC1] | 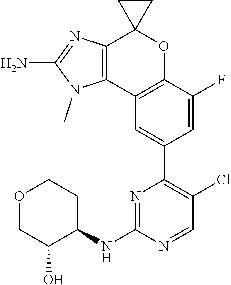 [SMILES: Cn1c(N)nc2c1-c1cc(-c3nc(N[C@@H]4CCOC[C@H]4O)ncc3Cl)cc(F)c1OC21CC1] | 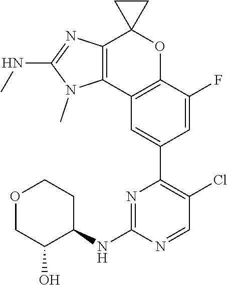 [SMILES: CNc1nc2c(n1C)-c1cc(-c3nc(N[C@@H]4CCOC[C@H]4O)ncc3Cl)cc(F)c1OC21CC1] |
| 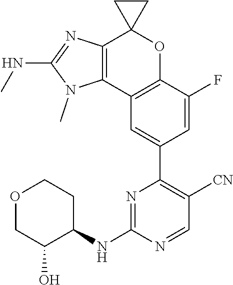 [SMILES: CNc1nc2c(n1C)-c1cc(-c3nc(N[C@@H]4CCOC[C@H]4O)ncc3C#N)cc(F)c1OC21CC1] | 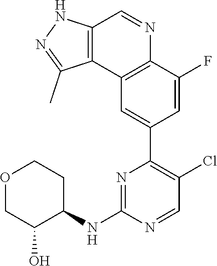 [SMILES: Cc1n[nH]c2cnc3c(F)cc(-c4nc(N[C@@H]5CCOC[C@H]5O)ncc4Cl)cc3c12] | 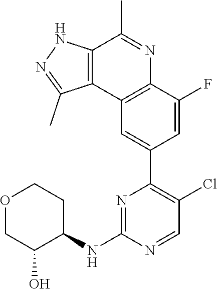 [SMILES: Cc1nc2c(F)cc(-c3nc(N[C@@H]4CCOC[C@H]4O)ncc3Cl)cc2c2c(C)n[nH]c12] |
| 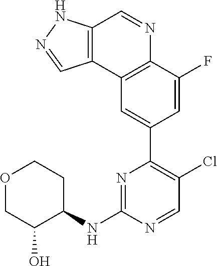 [SMILES: O[C@@H]1COCC[C@H]1Nc1ncc(Cl)c(-c2cc(F)c3ncc4[nH]ncc4c3c2)n1] | 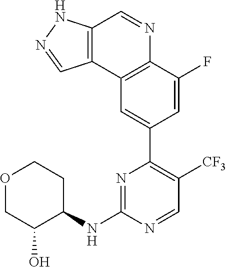 [SMILES: O[C@@H]1COCC[C@H]1Nc1ncc(C(F)(F)F)c(-c2cc(F)c3ncc4[nH]ncc4c3c2)n1] | 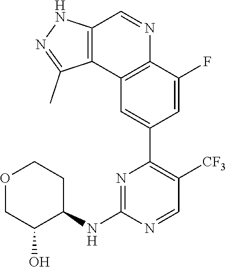 [SMILES: Cc1n[nH]c2cnc3c(F)cc(-c4nc(N[C@@H]5CCOC[C@H]5O)ncc4C(F)(F)F)cc3c12] |
| 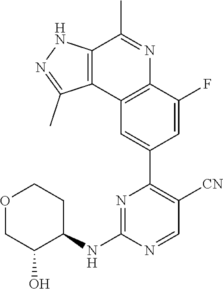 [SMILES: Cc1nc2c(F)cc(-c3nc(N[C@@H]4CCOC[C@H]4O)ncc3C#N)cc2c2c(C)n[nH]c12] | 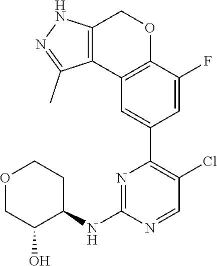 [SMILES: Cc1n[nH]c2c1-c1cc(-c3nc(N[C@@H]4CCOC[C@H]4O)ncc3Cl)cc(F)c1OC2] | 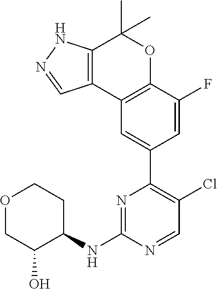 [SMILES: CC1(C)Oc2c(F)cc(-c3nc(N[C@@H]4CCOC[C@H]4O)ncc3Cl)cc2-c2cn[nH]c21] |
| 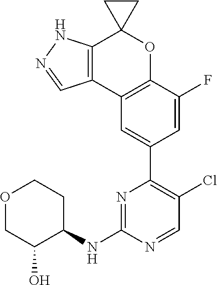 [SMILES: O[C@@H]1COCC[C@H]1Nc1ncc(Cl)c(-c2cc(F)c3c(c2)-c2cn[nH]c2C2(CC2)O3)n1] | 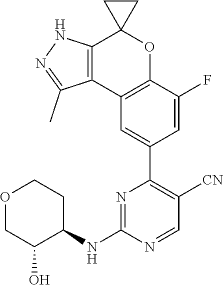 [SMILES: Cc1n[nH]c2c1-c1cc(-c3nc(N[C@@H]4CCOC[C@H]4O)ncc3C#N)cc(F)c1OC21CC1] | 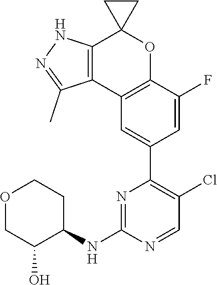 [SMILES: Cc1n[nH]c2c1-c1cc(-c3nc(N[C@@H]4CCOC[C@H]4O)ncc3Cl)cc(F)c1OC21CC1] |
| 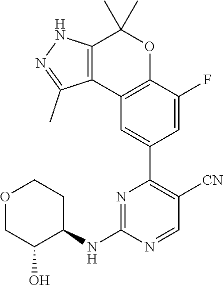 [SMILES: Cc1n[nH]c2c1-c1cc(-c3nc(N[C@@H]4CCOC[C@H]4O)ncc3C#N)cc(F)c1OC2(C)C] | 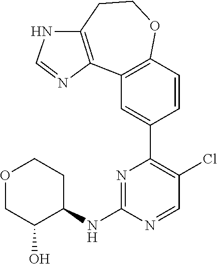 [SMILES: O[C@@H]1COCC[C@H]1Nc1ncc(Cl)c(-c2ccc3c(c2)-c2nc[nH]c2CCO3)n1] | 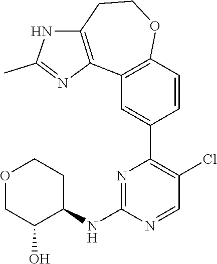 [SMILES: Cc1nc2c([nH]1)CCOc1ccc(-c3nc(N[C@@H]4CCOC[C@H]4O)ncc3Cl)cc1-2] |
| 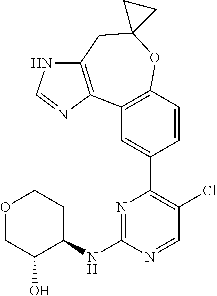 [SMILES: O[C@@H]1COCC[C@H]1Nc1ncc(Cl)c(-c2ccc3c(c2)-c2nc[nH]c2CC2(CC2)O3)n1] | 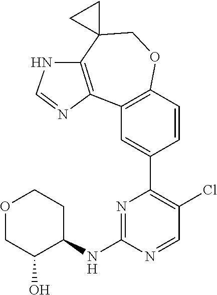 [SMILES: O[C@@H]1COCC[C@H]1Nc1ncc(Cl)c(-c2ccc3c(c2)-c2nc[nH]c2C2(CC2)CO3)n1] | 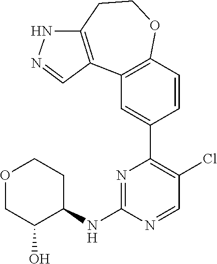 [SMILES: O[C@@H]1COCC[C@H]1Nc1ncc(Cl)c(-c2ccc3c(c2)-c2cn[nH]c2CCO3)n1] |
| 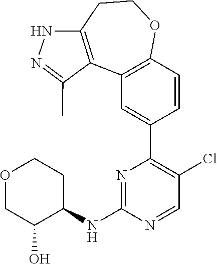 [SMILES: Cc1n[nH]c2c1-c1cc(-c3nc(N[C@@H]4CCOC[C@H]4O)ncc3Cl)ccc1OCC2] | 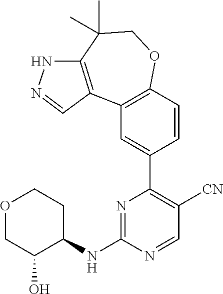 [SMILES: CC1(C)COc2ccc(-c3nc(N[C@@H]4CCOC[C@H]4O)ncc3C#N)cc2-c2cn[nH]c21] | 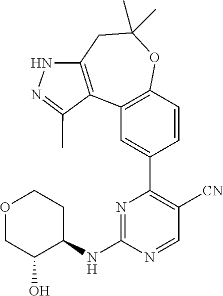 [SMILES: Cc1n[nH]c2c1-c1cc(-c3nc(N[C@@H]4CCOC[C@H]4O)ncc3C#N)ccc1OC(C)(C)C2] |
| 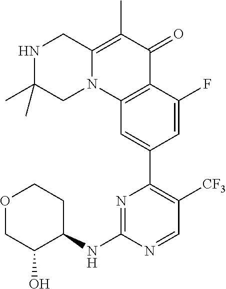 [SMILES: Cc1c2n(c3cc(-c4nc(N[C@@H]5CCOC[C@H]5O)ncc4C(F)(F)F)cc(F)c3c1=O)CC(C)(C)NC2] | 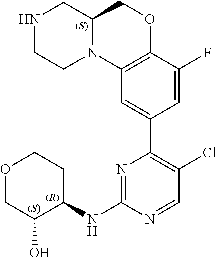 | 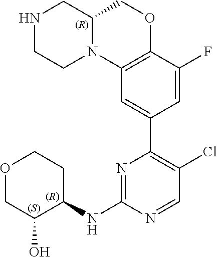 |
| 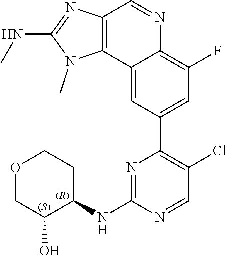 | 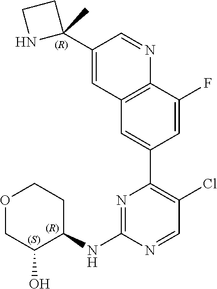 | 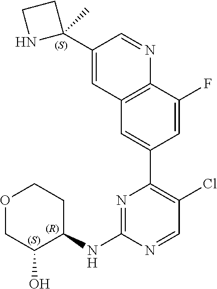 |
| 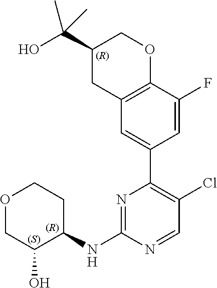 | 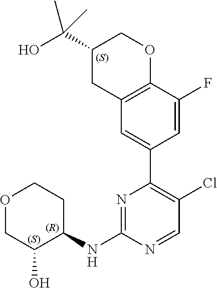 | 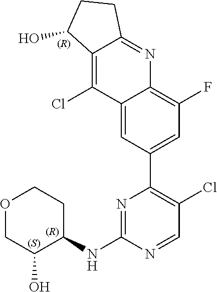 |
| 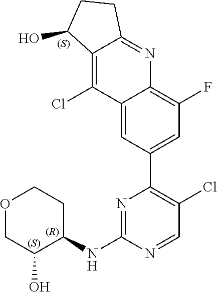 | 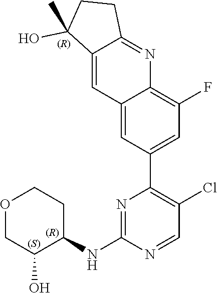 | 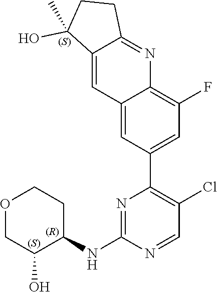 |
| 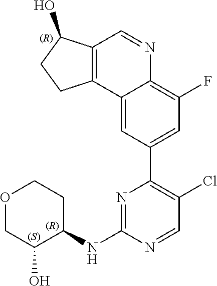 | 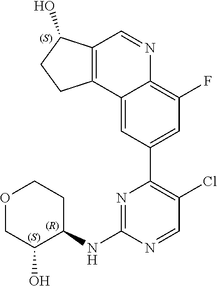 | 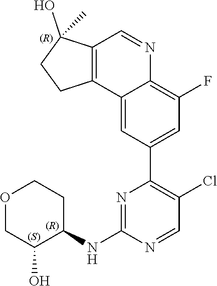 |
| 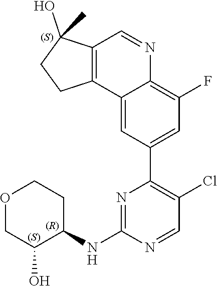 | 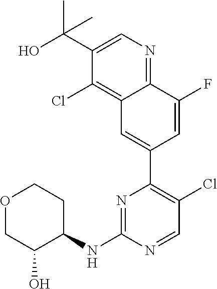 [SMILES: CC(C)(O)c1cnc2c(F)cc(-c3nc(N[C@@H]4CCOC[C@H]4O)ncc3Cl)cc2c1Cl] | 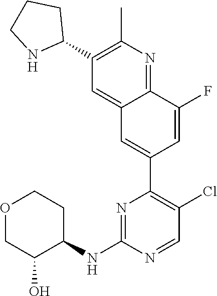 [SMILES: Cc1nc2c(F)cc(-c3nc(N[C@@H]4CCOC[C@H]4O)ncc3Cl)cc2cc1[C@H]1CCCN1] |
| 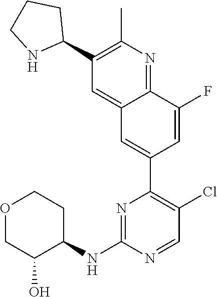 [SMILES: Cc1nc2c(F)cc(-c3nc(N[C@@H]4CCOC[C@H]4O)ncc3Cl)cc2cc1[C@@H]1CCCN1] | 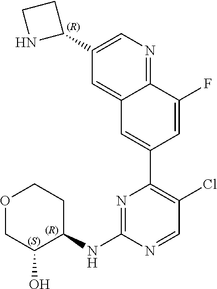 | 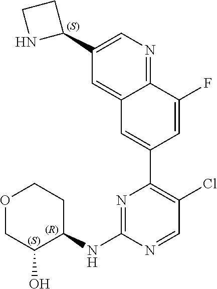 |
| 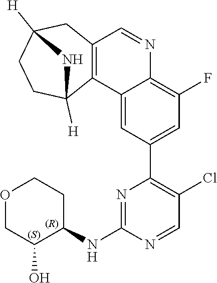 | 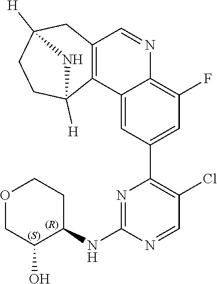 | 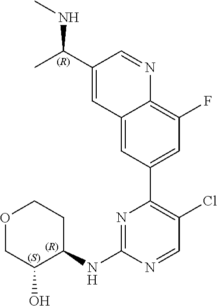 |
| 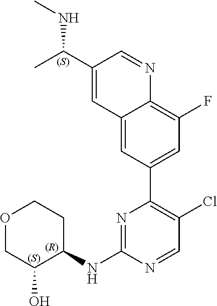 | 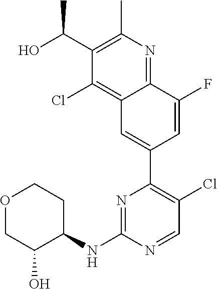 [SMILES: Cc1nc2c(F)cc(-c3nc(N[C@@H]4CCOC[C@H]4O)ncc3Cl)cc2c(Cl)c1[C@H](C)O] | 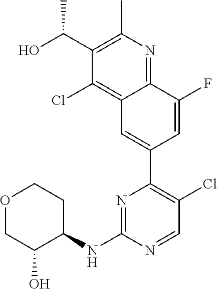 [SMILES: Cc1nc2c(F)cc(-c3nc(N[C@@H]4CCOC[C@H]4O)ncc3Cl)cc2c(Cl)c1[C@@H](C)O] |
| 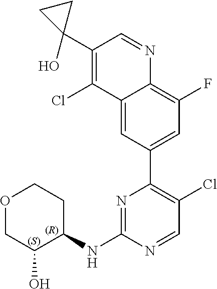 | 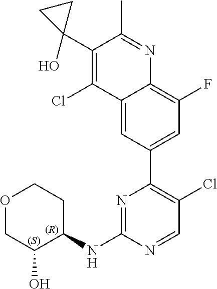 | 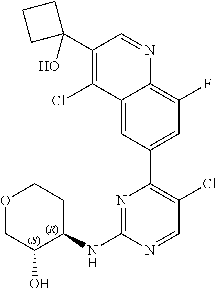 |
| 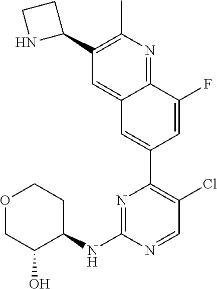 [SMILES: Cc1nc2c(F)cc(-c3nc(N[C@@H]4CCOC[C@H]4O)ncc3Cl)cc2cc1[C@@H]1CCN1] | 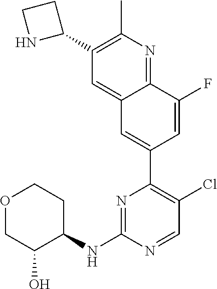 [SMILES: Cc1nc2c(F)cc(-c3nc(N[C@@H]4CCOC[C@H]4O)ncc3Cl)cc2cc1[C@H]1CCN1] | 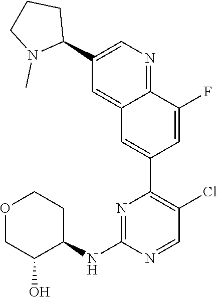 [SMILES: CN1CCC[C@H]1c1cnc2c(F)cc(-c3nc(N[C@@H]4CCOC[C@H]4O)ncc3Cl)cc2c1] |
| 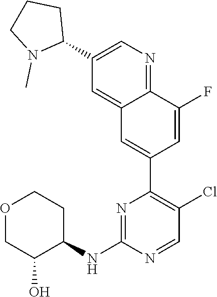 [SMILES: CN1CCC[C@@H]1c1cnc2c(F)cc(-c3nc(N[C@@H]4CCOC[C@H]4O)ncc3Cl)cc2c1] | 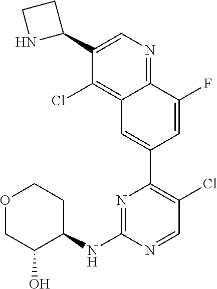 [SMILES: O[C@@H]1COCC[C@H]1Nc1ncc(Cl)c(-c2cc(F)c3ncc([C@@H]4CCN4)c(Cl)c3c2)n1] | 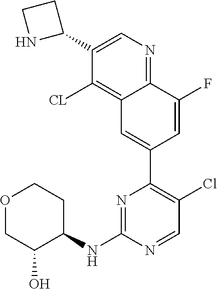 |
| 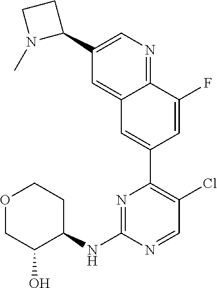 [SMILES: CN1CC[C@H]1c1cnc2c(F)cc(-c3nc(N[C@@H]4CCOC[C@H]4O)ncc3Cl)cc2c1] | 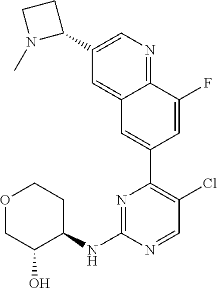 [SMILES: CN1CC[C@@H]1c1cnc2c(F)cc(-c3nc(N[C@@H]4CCOC[C@H]4O)ncc3Cl)cc2c1] | 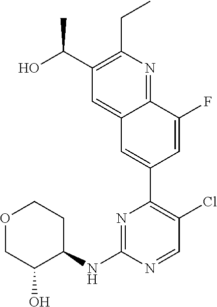 [SMILES: CCc1nc2c(F)cc(-c3nc(N[C@@H]4CCOC[C@H]4O)ncc3Cl)cc2cc1[C@H](C)O] |
| 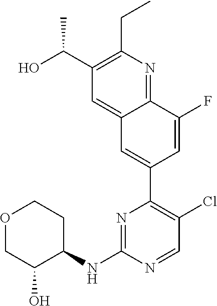 [SMILES: CCc1nc2c(F)cc(-c3nc(N[C@@H]4CCOC[C@H]4O)ncc3Cl)cc2cc1[C@@H](C)O] | 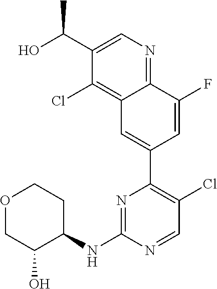 [SMILES: C[C@H](O)c1cnc2c(F)cc(-c3nc(N[C@@H]4CCOC[C@H]4O)ncc3Cl)cc2c1Cl] | 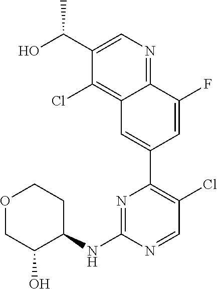 [SMILES: C[C@@H](O)c1cnc2c(F)cc(-c3nc(N[C@@H]4CCOC[C@H]4O)ncc3Cl)cc2c1Cl] |
| 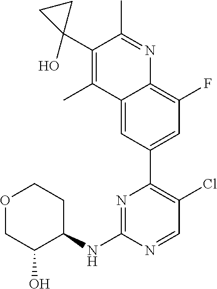 [SMILES: Cc1nc2c(F)cc(-c3nc(N[C@@H]4CCOC[C@H]4O)ncc3Cl)cc2c(C)c1C1(O)CC1] | 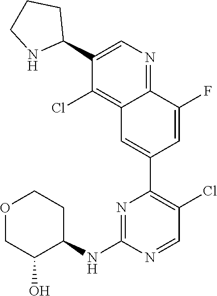 [SMILES: O[C@@H]1COCC[C@H]1Nc1ncc(Cl)c(-c2cc(F)c3ncc([C@@H]4CCCN4)c(Cl)c3c2)n1] | 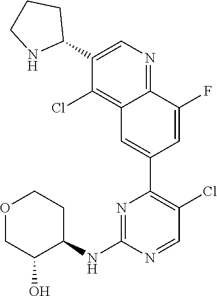 [SMILES: O[C@@H]1COCC[C@H]1Nc1ncc(Cl)c(-c2cc(F)c3ncc([C@H]4CCCN4)c(Cl)c3c2)n1] |
| 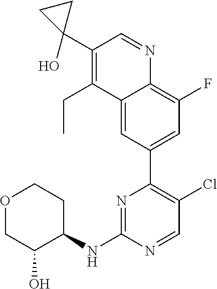 [SMILES: CCc1c(C2(O)CC2)cnc2c(F)cc(-c3nc(N[C@@H]4CCOC[C@H]4O)ncc3Cl)cc12] | 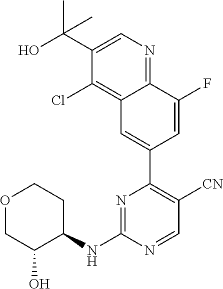 [SMILES: CC(C)(O)c1cnc2c(F)cc(-c3nc(N[C@@H]4CCOC[C@H]4O)ncc3C#N)cc2c1Cl] | 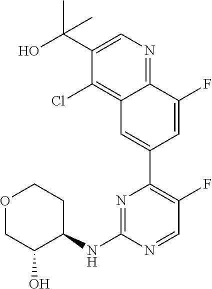 [SMILES: CC(C)(O)c1cnc2c(F)cc(-c3nc(N[C@@H]4CCOC[C@H]4O)ncc3F)cc2c1Cl] |
| 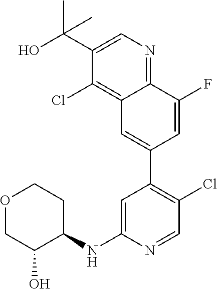 [SMILES: CC(C)(O)c1cnc2c(F)cc(-c3cc(N[C@@H]4CCOC[C@H]4O)ncc3Cl)cc2c1Cl] | 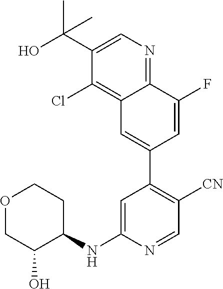 [SMILES: CC(C)(O)c1cnc2c(F)cc(-c3cc(N[C@@H]4CCOC[C@H]4O)ncc3C#N)cc2c1Cl] | 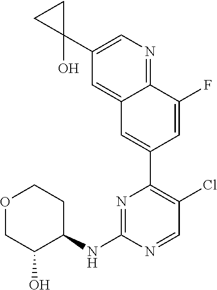 [SMILES: O[C@@H]1COCC[C@H]1Nc1ncc(Cl)c(-c2cc(F)c3ncc(C4(O)CC4)cc3c2)n1] |
| 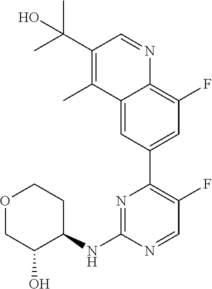 [SMILES: Cc1c(C(C)(C)O)cnc2c(F)cc(-c3nc(N[C@@H]4CCOC[C@H]4O)ncc3F)cc12] | 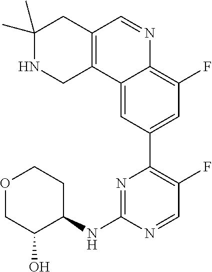 [SMILES: CC1(C)Cc2cnc3c(F)cc(-c4nc(N[C@@H]5CCOC[C@H]5O)ncc4F)cc3c2CN1] | 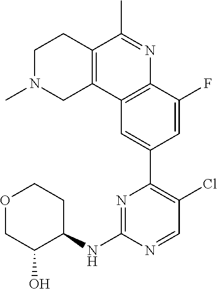 [SMILES: Cc1nc2c(F)cc(-c3nc(N[C@@H]4CCOC[C@H]4O)ncc3Cl)cc2c2c1CCN(C)C2] |
| 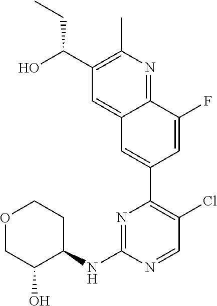 [SMILES: CC[C@@H](O)c1cc2cc(-c3nc(N[C@@H]4CCOC[C@H]4O)ncc3Cl)cc(F)c2nc1C] | 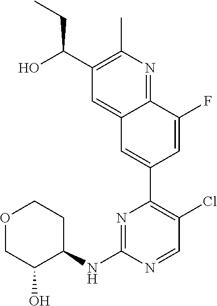 [SMILES: CC[C@H](O)c1cc2cc(-c3nc(N[C@@H]4CCOC[C@H]4O)ncc3Cl)cc(F)c2nc1C] | 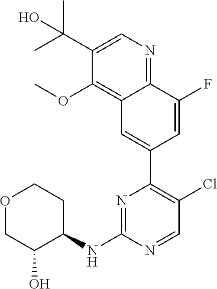 [SMILES: COc1c(C(C)(C)O)cnc2c(F)cc(-c3nc(N[C@@H]4CCOC[C@H]4O)ncc3Cl)cc12] |
| 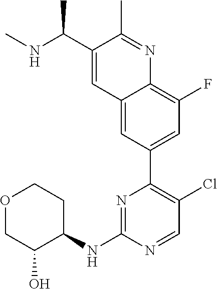 [SMILES: CN[C@@H](C)c1cc2cc(-c3nc(N[C@@H]4CCOC[C@H]4O)ncc3Cl)cc(F)c2nc1C] | 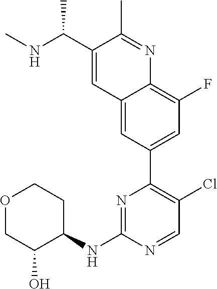 [SMILES: CN[C@H](C)c1cc2cc(-c3nc(N[C@@H]4CCOC[C@H]4O)ncc3Cl)cc(F)c2nc1C] | 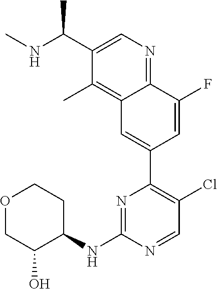 [SMILES: CN[C@@H](C)c1cnc2c(F)cc(-c3nc(N[C@@H]4CCOC[C@H]4O)ncc3Cl)cc2c1C] |
| 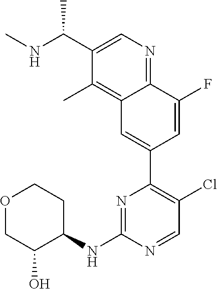 [SMILES: CN[C@H](C)c1cnc2c(F)cc(-c3nc(N[C@@H]4CCOC[C@H]4O)ncc3Cl)cc2c1C] | 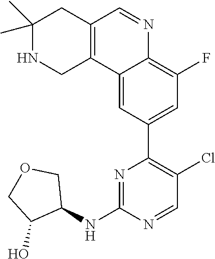 [SMILES: CC1(C)Cc2cnc3c(F)cc(-c4nc(N[C@@H]5COC[C@H]5O)ncc4Cl)cc3c2CN1] | 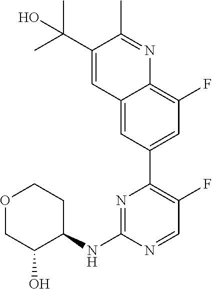 [SMILES: Cc1nc2c(F)cc(-c3nc(N[C@@H]4CCOC[C@H]4O)ncc3F)cc2cc1C(C)(C)O] |
| 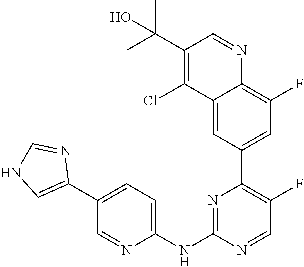 [SMILES: CC(C)(O)c1cnc2c(F)cc(-c3nc(Nc4ccc(-c5c[nH]cn5)cn4)ncc3F)cc2c1Cl] | 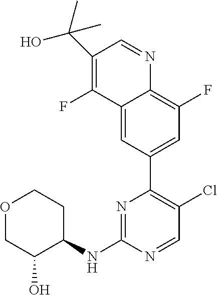 [SMILES: CC(C)(O)c1cnc2c(F)cc(-c3nc(N[C@@H]4CCOC[C@H]4O)ncc3Cl)cc2c1F] | 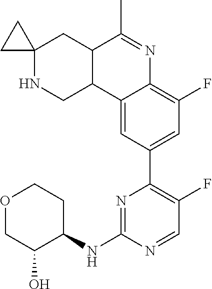 [SMILES: CC1=Nc2c(F)cc(-c3nc(N[C@@H]4CCOC[C@H]4O)ncc3F)cc2C2CNC3(CC3)CC12] |
| 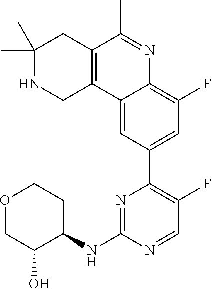 [SMILES: Cc1nc2c(F)cc(-c3nc(N[C@@H]4CCOC[C@H]4O)ncc3F)cc2c2c1CC(C)(C)NC2] | 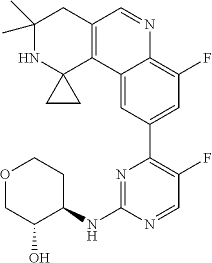 [SMILES: CC1(C)Cc2cnc3c(F)cc(-c4nc(N[C@@H]5CCOC[C@H]5O)ncc4F)cc3c2C2(CC2)N1] | 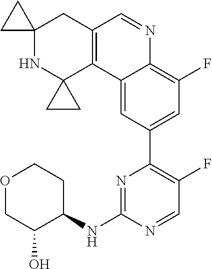 [SMILES: O[C@@H]1COCC[C@H]1Nc1ncc(F)c(-c2cc(F)c3ncc4c(c3c2)C2(CC2)NC2(CC2)C4)n1] |
| 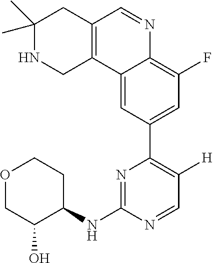 [SMILES: CC1(C)Cc2cnc3c(F)cc(-c4ccnc(N[C@@H]5CCOC[C@H]5O)n4)cc3c2CN1] | 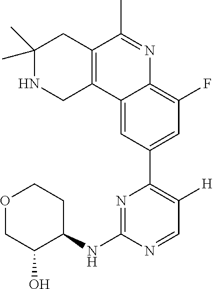 [SMILES: Cc1nc2c(F)cc(-c3ccnc(N[C@@H]4CCOC[C@H]4O)n3)cc2c2c1CC(C)(C)NC2] | 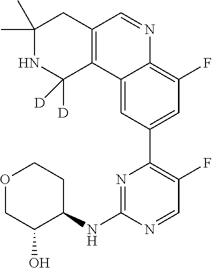 [SMILES: [2H]C1([2H])NC(C)(C)Cc2cnc3c(F)cc(-c4nc(N[C@@H]5CCOC[C@H]5O)ncc4F)cc3c21] |
| 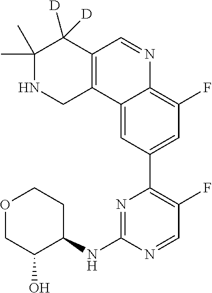 [SMILES: [2H]C1([2H])c2cnc3c(F)cc(-c4nc(N[C@@H]5CCOC[C@H]5O)ncc4F)cc3c2CNC1(C)C] | 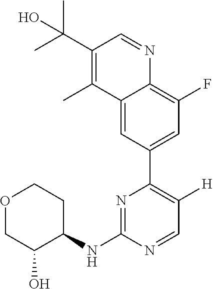 [SMILES: Cc1c(C(C)(C)O)cnc2c(F)cc(-c3ccnc(N[C@@H]4CCOC[C@H]4O)n3)cc12] | 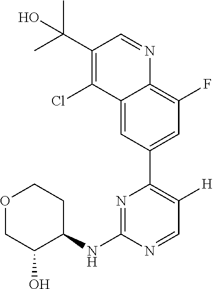 [SMILES: CC(C)(O)c1cnc2c(F)cc(-c3ccnc(N[C@@H]4CCOC[C@H]4O)n3)cc2c1Cl] |
| 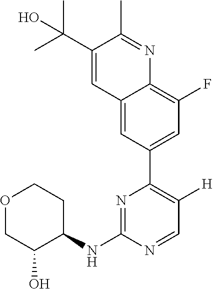 [SMILES: Cc1nc2c(F)cc(-c3ccnc(N[C@@H]4CCOC[C@H]4O)n3)cc2cc1C(C)(C)O] | 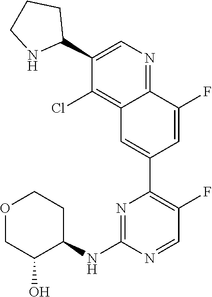 | 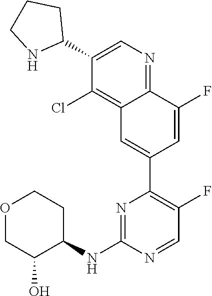 |
| 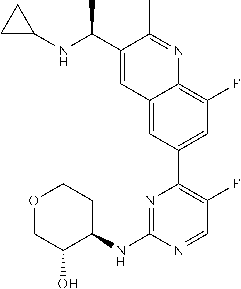 [SMILES: Cc1nc2c(F)cc(-c3nc(N[C@@H]4CCOC[C@H]4O)ncc3F)cc2cc1[C@H](C)NC1CC1] | 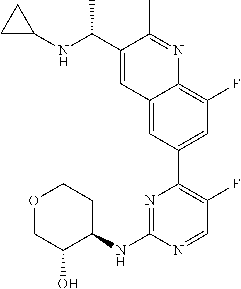 [SMILES: Cc1nc2c(F)cc(-c3nc(N[C@@H]4CCOC[C@H]4O)ncc3F)cc2cc1[C@@H](C)NC1CC1] | 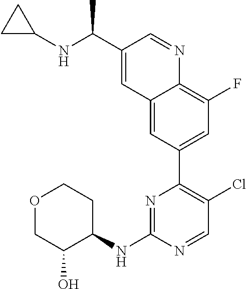 [SMILES: C[C@H](NC1CC1)c1cnc2c(F)cc(-c3nc(N[C@@H]4CCOC[C@H]4O)ncc3Cl)cc2c1] |
| 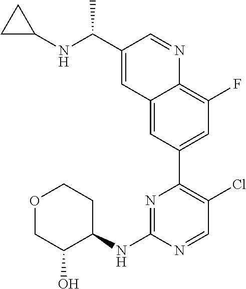 [SMILES: C[C@@H](NC1CC1)c1cnc2c(F)cc(-c3nc(N[C@@H]4CCOC[C@H]4O)ncc3Cl)cc2c1] | 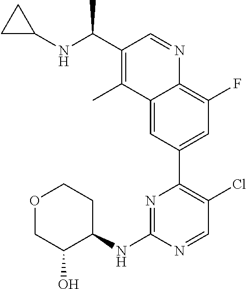 [SMILES: Cc1c([C@H](C)NC2CC2)cnc2c(F)cc(-c3nc(N[C@@H]4CCOC[C@H]4O)ncc3Cl)cc12] | 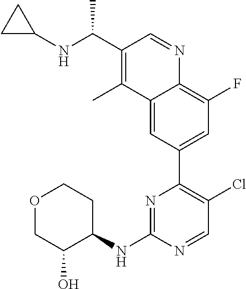 [SMILES: Cc1c([C@@H](C)NC2CC2)cnc2c(F)cc(-c3nc(N[C@@H]4CCOC[C@H]4O)ncc3Cl)cc12] |
| 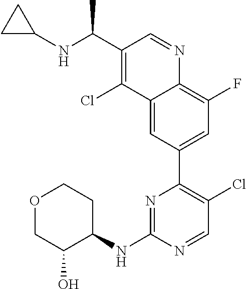 [SMILES: C[C@H](NC1CC1)c1cnc2c(F)cc(-c3nc(N[C@@H]4CCOC[C@H]4O)ncc3Cl)cc2c1Cl] | 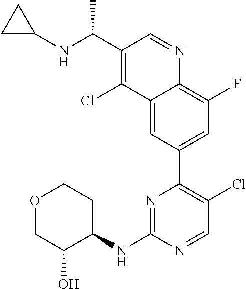 [SMILES: C[C@@H](NC1CC1)c1cnc2c(F)cc(-c3nc(N[C@@H]4CCOC[C@H]4O)ncc3Cl)cc2c1Cl] | 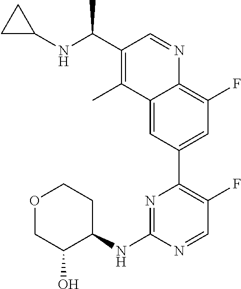 [SMILES: Cc1c([C@H](C)NC2CC2)cnc2c(F)cc(-c3nc(N[C@@H]4CCOC[C@H]4O)ncc3F)cc12] |
| 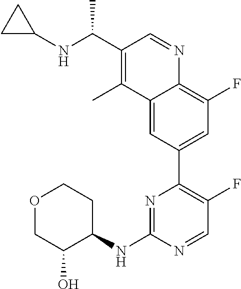 [SMILES: Cc1c([C@@H](C)NC2CC2)cnc2c(F)cc(-c3nc(N[C@@H]4CCOC[C@H]4O)ncc3F)cc12] | 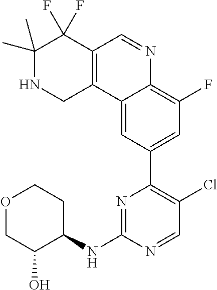 [SMILES: CC1(C)NCc2c(cnc3c(F)cc(-c4nc(N[C@@H]5CCOC[C@H]5O)ncc4Cl)cc23)C1(F)F] | 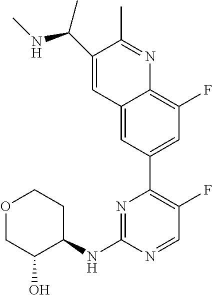 [SMILES: CN[C@@H](C)c1cc2cc(-c3nc(N[C@@H]4CCOC[C@H]4O)ncc3F)cc(F)c2nc1C] |
| 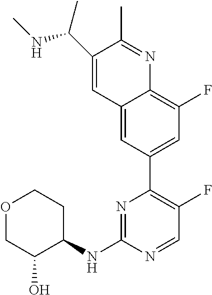 [SMILES: CN[C@H](C)c1cc2cc(-c3nc(N[C@@H]4CCOC[C@H]4O)ncc3F)cc(F)c2nc1C] | 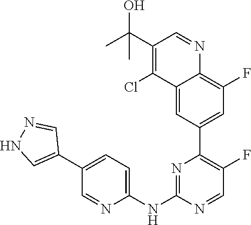 [SMILES: CC(C)(O)c1cnc2c(F)cc(-c3nc(Nc4ccc(-c5cn[nH]c5)cn4)ncc3F)cc2c1Cl] | 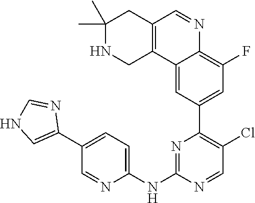 [SMILES: CC1(C)Cc2cnc3c(F)cc(-c4nc(Nc5ccc(-c6c[nH]cn6)cn5)ncc4Cl)cc3c2CN1] |
| 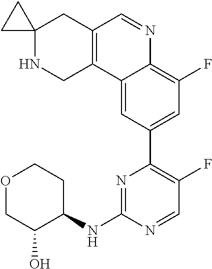 [SMILES: O[C@@H]1COCC[C@H]1Nc1ncc(F)c(-c2cc(F)c3ncc4c(c3c2)CNC2(CC2)C4)n1] | 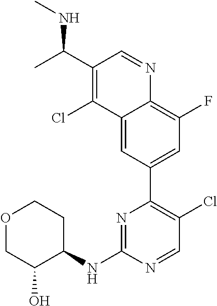 [SMILES: CN[C@H](C)c1cnc2c(F)cc(-c3nc(N[C@@H]4CCOC[C@H]4O)ncc3Cl)cc2c1Cl] | 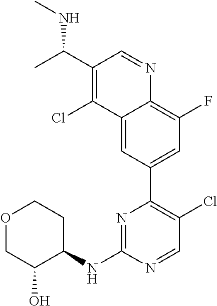 [SMILES: CN[C@@H](C)c1cnc2c(F)cc(-c3nc(N[C@@H]4CCOC[C@H]4O)ncc3Cl)cc2c1Cl] |
| 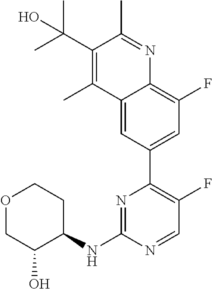 [SMILES: Cc1nc2c(F)cc(-c3nc(N[C@@H]4CCOC[C@H]4O)ncc3F)cc2c(C)c1C(C)(C)O] | 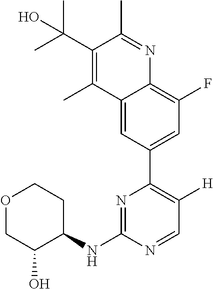 [SMILES: Cc1nc2c(F)cc(-c3ccnc(N[C@@H]4CCOC[C@H]4O)n3)cc2c(C)c1C(C)(C)O] | 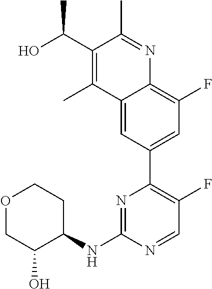 [SMILES: Cc1nc2c(F)cc(-c3nc(N[C@@H]4CCOC[C@H]4O)ncc3F)cc2c(C)c1[C@H](C)O] |
| 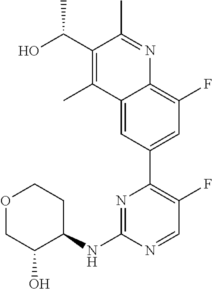 [SMILES: Cc1nc2c(F)cc(-c3nc(N[C@@H]4CCOC[C@H]4O)ncc3F)cc2c(C)c1[C@@H](C)O] | 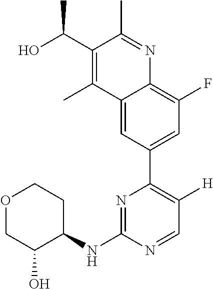 [SMILES: Cc1nc2c(F)cc(-c3ccnc(N[C@@H]4CCOC[C@H]4O)n3)cc2c(C)c1[C@H](C)O] | 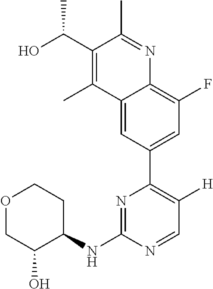 [SMILES: Cc1nc2c(F)cc(-c3ccnc(N[C@@H]4CCOC[C@H]4O)n3)cc2c(C)c1[C@@H](C)O] |
| 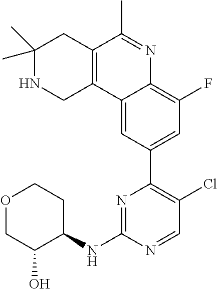 [SMILES: Cc1nc2c(F)cc(-c3nc(N[C@@H]4CCOC[C@H]4O)ncc3Cl)cc2c2c1CC(C)(C)NC2] | 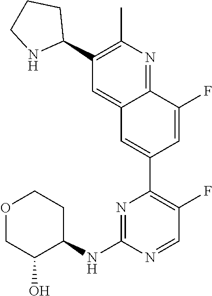 [SMILES: Cc1nc2c(F)cc(-c3nc(N[C@@H]4CCOC[C@H]4O)ncc3F)cc2cc1[C@@H]1CCCN1] | 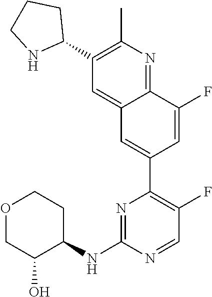 [SMILES: Cc1nc2c(F)cc(-c3nc(N[C@@H]4CCOC[C@H]4O)ncc3F)cc2cc1[C@H]1CCCN1] |
| 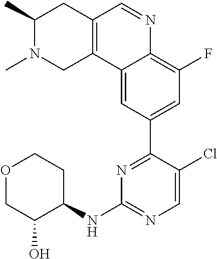 [SMILES: C[C@H]1Cc2cnc3c(F)cc(-c4nc(N[C@@H]5CCOC[C@H]5O)ncc4Cl)cc3c2CN1C] | 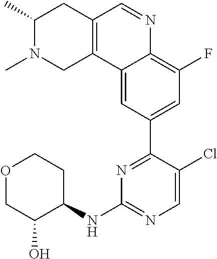 [SMILES: C[C@@H]1Cc2cnc3c(F)cc(-c4nc(N[C@@H]5CCOC[C@H]5O)ncc4Cl)cc3c2CN1C] | 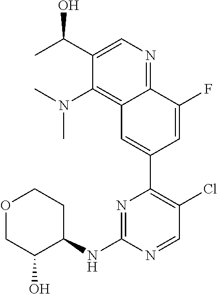 [SMILES: C[C@@H](O)c1cnc2c(F)cc(-c3nc(N[C@@H]4CCOC[C@H]4O)ncc3Cl)cc2c1N(C)C] |
| 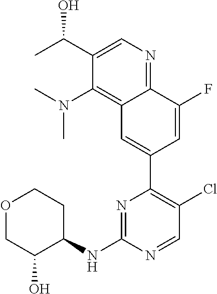 [SMILES: C[C@H](O)c1cnc2c(F)cc(-c3nc(N[C@@H]4CCOC[C@H]4O)ncc3Cl)cc2c1N(C)C] | 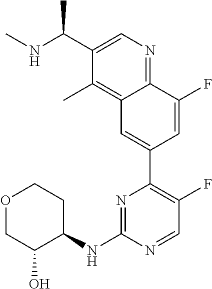 [SMILES: CN[C@@H](C)c1cnc2c(F)cc(-c3nc(N[C@@H]4CCOC[C@H]4O)ncc3F)cc2c1C] | 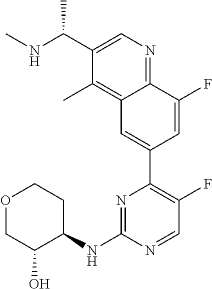 [SMILES: CN[C@H](C)c1cnc2c(F)cc(-c3nc(N[C@@H]4CCOC[C@H]4O)ncc3F)cc2c1C] |
| 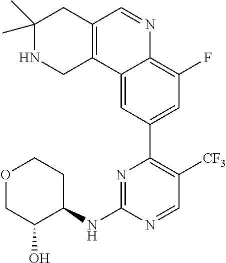 [SMILES: CC1(C)Cc2cnc3c(F)cc(-c4nc(N[C@@H]5CCOC[C@H]5O)ncc4C(F)(F)F)cc3c2CN1] | 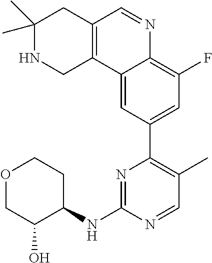 | 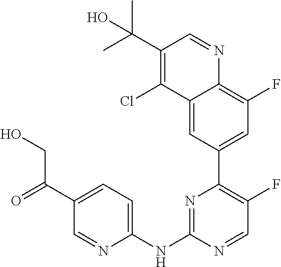 [SMILES: CC(C)(O)c1cnc2c(F)cc(-c3nc(Nc4ccc(C(=O)CO)cn4)ncc3F)cc2c1Cl] |
| 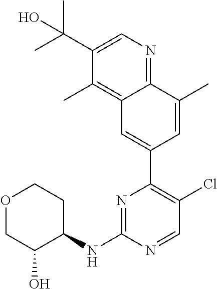 [SMILES: Cc1c(C(C)(C)O)cnc2c(C)cc(-c3nc(N[C@@H]4CCOC[C@H]4O)ncc3Cl)cc12] | 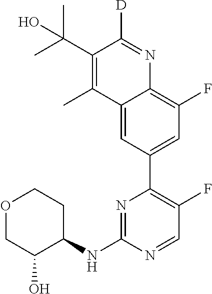 [SMILES: [2H]c1nc2c(F)cc(-c3nc(N[C@@H]4CCOC[C@H]4O)ncc3F)cc2c(C)c1C(C)(C)O] | 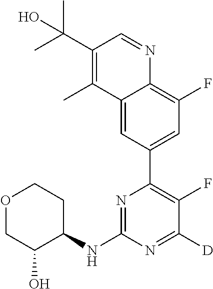 [SMILES: [2H]c1nc(N[C@@H]2CCOC[C@H]2O)nc(-c2cc(F)c3ncc(C(C)(C)O)c(C)c3c2)c1F] |
| 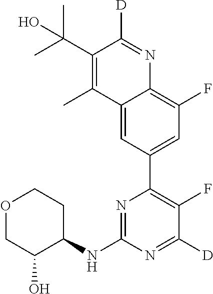 [SMILES: [2H]c1nc(N[C@@H]2CCOC[C@H]2O)nc(-c2cc(F)c3nc([2H])c(C(C)(C)O)c(C)c3c2)c1F] | 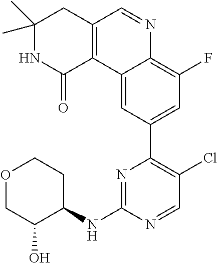 [SMILES: CC1(C)Cc2cnc3c(F)cc(-c4nc(N[C@@H]5CCOC[C@H]5O)ncc4Cl)cc3c2C(=O)N1] | 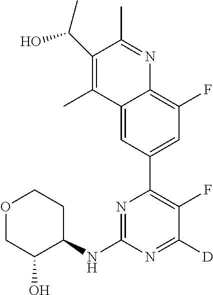 [SMILES: [2H]c1nc(N[C@@H]2CCOC[C@H]2O)nc(-c2cc(F)c3nc(C)c([C@@H](C)O)c(C)c3c2)c1F] |
| 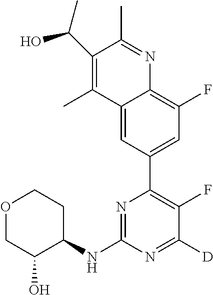 [SMILES: [2H]c1nc(N[C@@H]2CCOC[C@H]2O)nc(-c2cc(F)c3nc(C)c([C@H](C)O)c(C)c3c2)c1F] | 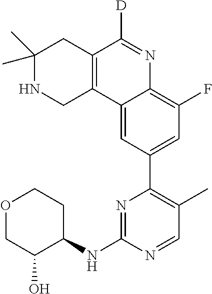 [SMILES: [2H]c1nc2c(F)cc(-c3nc(N[C@@H]4CCOC[C@H]4O)ncc3C)cc2c2c1CC(C)(C)NC2] | 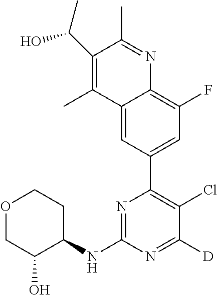 [SMILES: [2H]c1nc(N[C@@H]2CCOC[C@H]2O)nc(-c2cc(F)c3nc(C)c([C@@H](C)O)c(C)c3c2)c1Cl] |
| 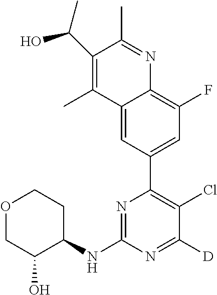 [SMILES: [2H]c1nc(N[C@@H]2CCOC[C@H]2O)nc(-c2cc(F)c3nc(C)c([C@H](C)O)c(C)c3c2)c1Cl] | 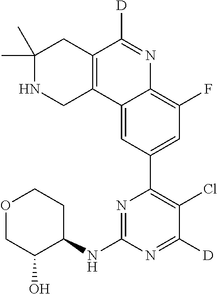 [SMILES: [2H]c1nc(N[C@@H]2CCOC[C@H]2O)nc(-c2cc(F)c3nc([2H])c4c(c3c2)CNC(C)(C)C4)c1Cl] | 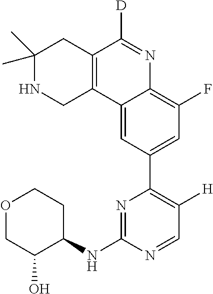 [SMILES: [2H]c1nc2c(F)cc(-c3ccnc(N[C@@H]4CCOC[C@H]4O)n3)cc2c2c1CC(C)(C)NC2] |
| 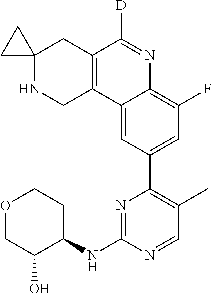 [SMILES: [2H]c1nc2c(F)cc(-c3nc(N[C@@H]4CCOC[C@H]4O)ncc3C)cc2c2c1CC1(CC1)NC2] | 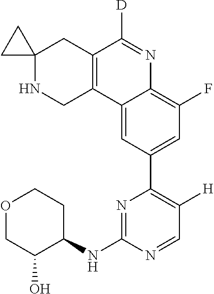 [SMILES: [2H]c1nc2c(F)cc(-c3ccnc(N[C@@H]4CCOC[C@H]4O)n3)cc2c2c1CC1(CC1)NC2] | 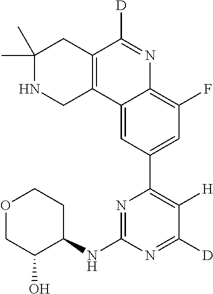 [SMILES: [2H]c1cc(-c2cc(F)c3nc([2H])c4c(c3c2)CNC(C)(C)C4)nc(N[C@@H]2CCOC[C@H]2O)n1] |
| 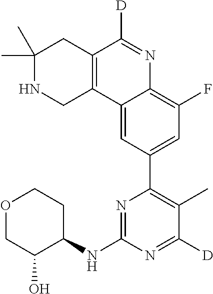 [SMILES: [2H]c1nc(N[C@@H]2CCOC[C@H]2O)nc(-c2cc(F)c3nc([2H])c4c(c3c2)CNC(C)(C)C4)c1C] | 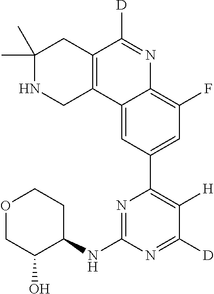 [SMILES: [2H]c1cc(-c2cc(F)c3nc([2H])c4c(c3c2)CNC(C)(C)C4)nc(N[C@@H]2CCOC[C@H]2O)n1] | 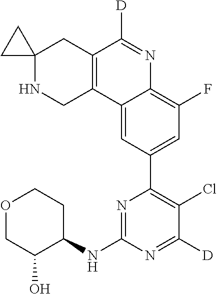 [SMILES: [2H]c1nc(N[C@@H]2CCOC[C@H]2O)nc(-c2cc(F)c3nc([2H])c4c(c3c2)CNC2(CC2)C4)c1Cl] |
| 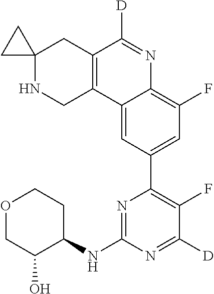 [SMILES: [2H]c1nc(N[C@@H]2CCOC[C@H]2O)nc(-c2cc(F)c3nc([2H])c4c(c3c2)CNC2(CC2)C4)c1F] | 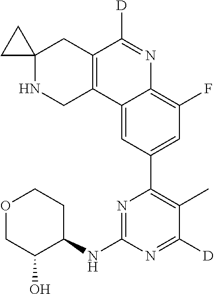 [SMILES: [2H]c1nc(N[C@@H]2CCOC[C@H]2O)nc(-c2cc(F)c3nc([2H])c4c(c3c2)CNC2(CC2)C4)c1C] | 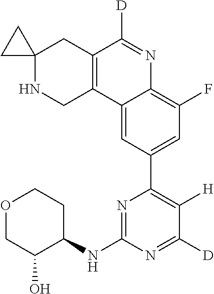 [SMILES: [2H]c1cc(-c2cc(F)c3nc([2H])c4c(c3c2)CNC2(CC2)C4)nc(N[C@@H]2CCOC[C@H]2O)n1] |
| 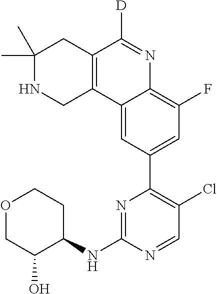 [SMILES: [2H]c1nc2c(F)cc(-c3nc(N[C@@H]4CCOC[C@H]4O)ncc3Cl)cc2c2c1CC(C)(C)NC2] | 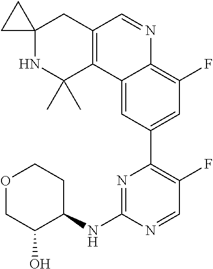 [SMILES: CC1(C)NC2(CC2)Cc2cnc3c(F)cc(-c4nc(N[C@@H]5CCOC[C@H]5O)ncc4F)cc3c21] | 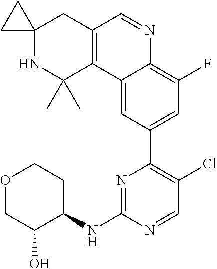 [SMILES: CC1(C)NC2(CC2)Cc2cnc3c(F)cc(-c4nc(N[C@@H]5CCOC[C@H]5O)ncc4Cl)cc3c21] |
| 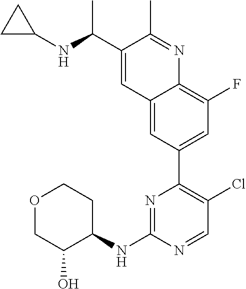 [SMILES: Cc1nc2c(F)cc(-c3nc(N[C@@H]4CCOC[C@H]4O)ncc3Cl)cc2cc1[C@H](C)NC1CC1] | 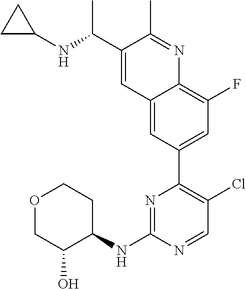 [SMILES: Cc1nc2c(F)cc(-c3nc(N[C@@H]4CCOC[C@H]4O)ncc3Cl)cc2cc1[C@@H](C)NC1CC1] | 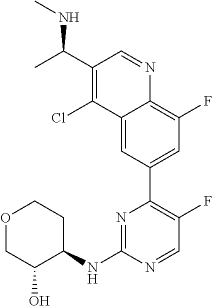 [SMILES: CN[C@H](C)c1cnc2c(F)cc(-c3nc(N[C@@H]4CCOC[C@H]4O)ncc3F)cc2c1Cl] |
| 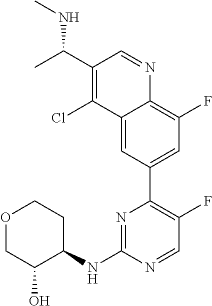 [SMILES: CN[C@@H](C)c1cnc2c(F)cc(-c3nc(N[C@@H]4CCOC[C@H]4O)ncc3F)cc2c1Cl] | 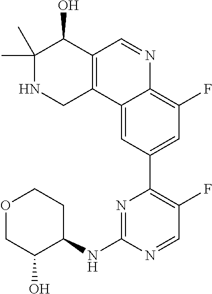 [SMILES: CC1(C)NCc2c(cnc3c(F)cc(-c4nc(N[C@@H]5CCOC[C@H]5O)ncc4F)cc23)[C@@H]1O] | 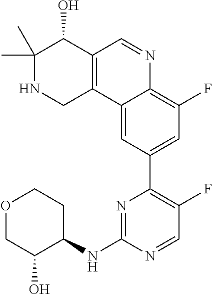 [SMILES: CC1(C)NCc2c(cnc3c(F)cc(-c4nc(N[C@@H]5CCOC[C@H]5O)ncc4F)cc23)[C@H]1O] |
| 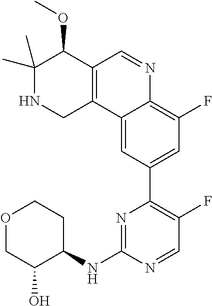 [SMILES: CO[C@H]1c2cnc3c(F)cc(-c4nc(N[C@@H]5CCOC[C@H]5O)ncc4F)cc3c2CNC1(C)C] | 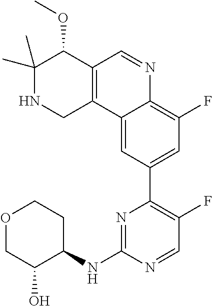 [SMILES: CO[C@@H]1c2cnc3c(F)cc(-c4nc(N[C@@H]5CCOC[C@H]5O)ncc4F)cc3c2CNC1(C)C] | 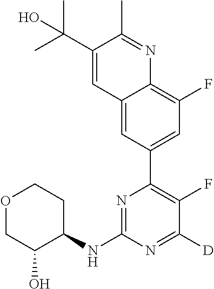 [SMILES: [2H]c1nc(N[C@@H]2CCOC[C@H]2O)nc(-c2cc(F)c3nc(C)c(C(C)(C)O)cc3c2)c1F] |
| 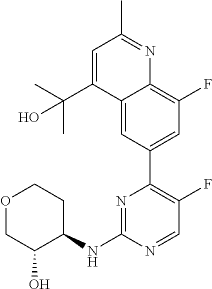 [SMILES: Cc1cc(C(C)(C)O)c2cc(-c3nc(N[C@@H]4CCOC[C@H]4O)ncc3F)cc(F)c2n1] | 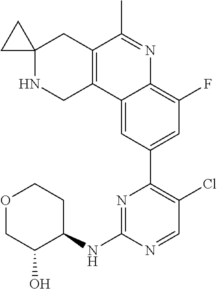 [SMILES: Cc1nc2c(F)cc(-c3nc(N[C@@H]4CCOC[C@H]4O)ncc3Cl)cc2c2c1CC1(CC1)NC2] | 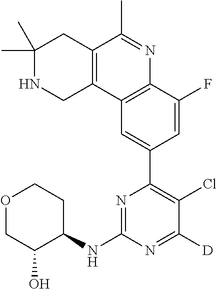 [SMILES: [2H]c1nc(N[C@@H]2CCOC[C@H]2O)nc(-c2cc(F)c3nc(C)c4c(c3c2)CNC(C)(C)C4)c1Cl] |
| 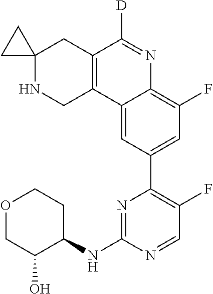 [SMILES: [2H]c1nc2c(F)cc(-c3nc(N[C@@H]4CCOC[C@H]4O)ncc3F)cc2c2c1CC1(CC1)NC2] | 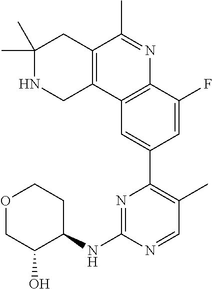 [SMILES: Cc1cnc(N[C@@H]2CCOC[C@H]2O)nc1-c1cc(F)c2nc(C)c3c(c2c1)CNC(C)(C)C3] | 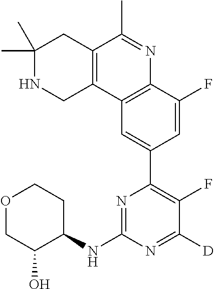 [SMILES: [2H]c1nc(N[C@@H]2CCOC[C@H]2O)nc(-c2cc(F)c3nc(C)c4c(c3c2)CNC(C)(C)C4)c1F] |
| 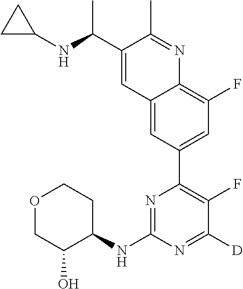 [SMILES: [2H]c1nc(N[C@@H]2CCOC[C@H]2O)nc(-c2cc(F)c3nc(C)c([C@H](C)NC4CC4)cc3c2)c1F] | 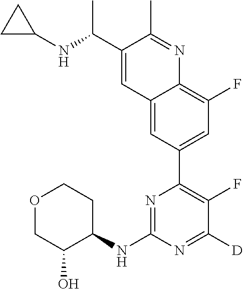 [SMILES: [2H]c1nc(N[C@@H]2CCOC[C@H]2O)nc(-c2cc(F)c3nc(C)c([C@@H](C)NC4CC4)cc3c2)c1F] | 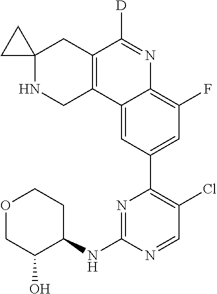 [SMILES: [2H]c1nc2c(F)cc(-c3nc(N[C@@H]4CCOC[C@H]4O)ncc3Cl)cc2c2c1CC1(CC1)NC2] |
| 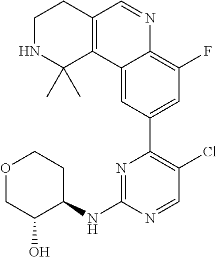 [SMILES: CC1(C)NCCc2cnc3c(F)cc(-c4nc(N[C@@H]5CCOC[C@H]5O)ncc4Cl)cc3c21] | 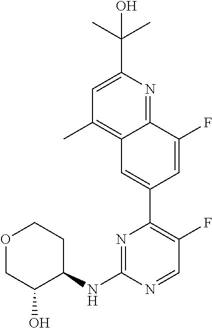 [SMILES: Cc1cc(C(C)(C)O)nc2c(F)cc(-c3nc(N[C@@H]4CCOC[C@H]4O)ncc3F)cc12] | 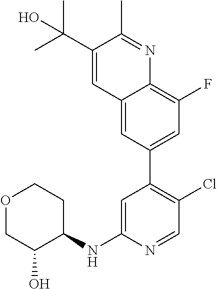 [SMILES: Cc1nc2c(F)cc(-c3cc(N[C@@H]4CCOC[C@H]4O)ncc3Cl)cc2cc1C(C)(C)O] |
| 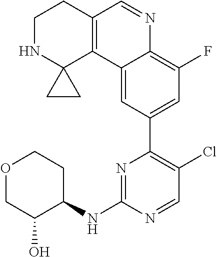 [SMILES: O[C@@H]1COCC[C@H]1Nc1ncc(Cl)c(-c2cc(F)c3ncc4c(c3c2)C2(CC2)NCC4)n1] | 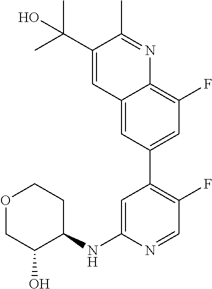 [SMILES: Cc1nc2c(F)cc(-c3cc(N[C@@H]4CCOC[C@H]4O)ncc3F)cc2cc1C(C)(C)O] | 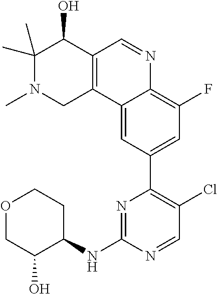 [SMILES: CN1Cc2c(cnc3c(F)cc(-c4nc(N[C@@H]5CCOC[C@H]5O)ncc4Cl)cc23)[C@H](O)C1(C)C] |
| 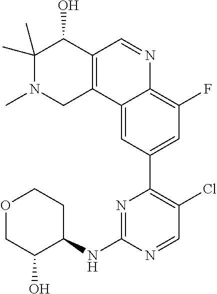 [SMILES: CN1Cc2c(cnc3c(F)cc(-c4nc(N[C@@H]5CCOC[C@H]5O)ncc4Cl)cc23)[C@@H](O)C1(C)C] | 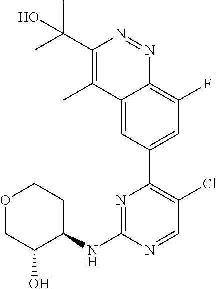 [SMILES: Cc1c(C(C)(C)O)nnc2c(F)cc(-c3nc(N[C@@H]4CCOC[C@H]4O)ncc3Cl)cc12] | 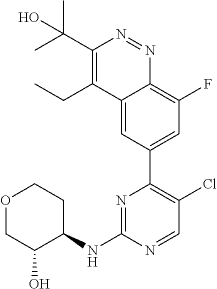 [SMILES: CCc1c(C(C)(C)O)nnc2c(F)cc(-c3nc(N[C@@H]4CCOC[C@H]4O)ncc3Cl)cc12] |
| 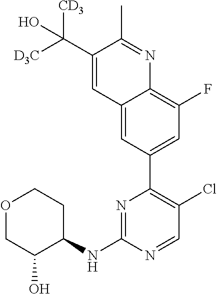 [SMILES: [2H]C([2H])([2H])C(O)(c1cc2cc(-c3nc(N[C@@H]4CCOC[C@H]4O)ncc3Cl)cc(F)c2nc1C)C([2H])([2H])[2H]] | 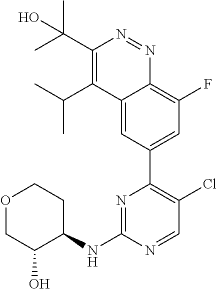 [SMILES: CC(C)c1c(C(C)(C)O)nnc2c(F)cc(-c3nc(N[C@@H]4CCOC[C@H]4O)ncc3Cl)cc12] | 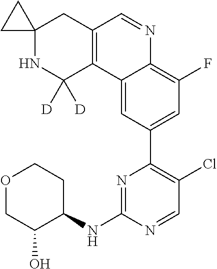 [SMILES: [2H]C1([2H])NC2(CC2)Cc2cnc3c(F)cc(-c4nc(N[C@@H]5CCOC[C@H]5O)ncc4Cl)cc3c21] |
| 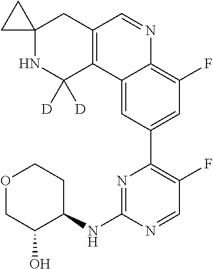 [SMILES: [2H]C1([2H])NC2(CC2)Cc2cnc3c(F)cc(-c4nc(N[C@@H]5CCOC[C@H]5O)ncc4F)cc3c21] | 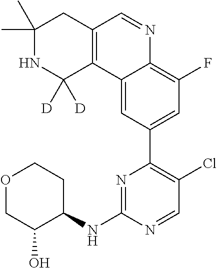 [SMILES: [2H]C1([2H])NC(C)(C)Cc2cnc3c(F)cc(-c4nc(N[C@@H]5CCOC[C@H]5O)ncc4Cl)cc3c21] | 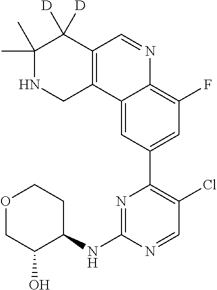 [SMILES: [2H]C1([2H])c2cnc3c(F)cc(-c4nc(N[C@@H]5CCOC[C@H]5O)ncc4Cl)cc3c2CNC1(C)C] |
| 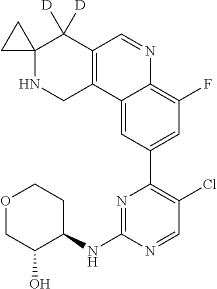 [SMILES: [2H]C1([2H])c2cnc3c(F)cc(-c4nc(N[C@@H]5CCOC[C@H]5O)ncc4Cl)cc3c2CNC12CC2] | 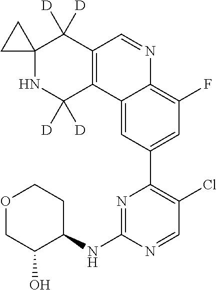 [SMILES: [2H]C1([2H])NC2(CC2)C([2H])([2H])c2cnc3c(F)cc(-c4nc(N[C@@H]5CCOC[C@H]5O)ncc4Cl)cc3c21] | 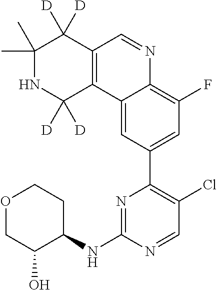 [SMILES: [2H]C1([2H])NC(C)(C)C([2H])([2H])c2cnc3c(F)cc(-c4nc(N[C@@H]5CCOC[C@H]5O)ncc4Cl)cc3c21] |
| 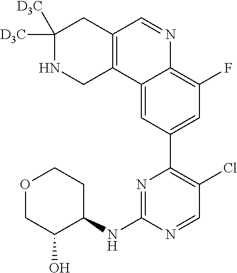 [SMILES: [2H]C([2H])([2H])C1(C([2H])([2H])[2H])Cc2cnc3c(F)cc(-c4nc(N[C@@H]5CCOC[C@H]5O)ncc4Cl)cc3c2CN1] | 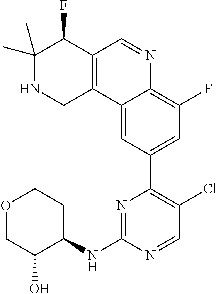 [SMILES: CC1(C)NCc2c(cnc3c(F)cc(-c4nc(N[C@@H]5CCOC[C@H]5O)ncc4Cl)cc23)[C@@H]1F] | 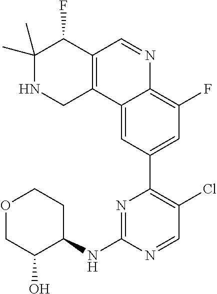 [SMILES: CC1(C)NCc2c(cnc3c(F)cc(-c4nc(N[C@@H]5CCOC[C@H]5O)ncc4Cl)cc23)[C@H]1F] |
| 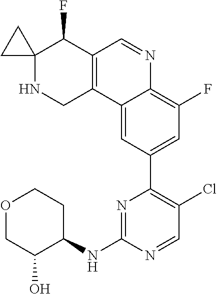 [SMILES: O[C@@H]1COCC[C@H]1Nc1ncc(Cl)c(-c2cc(F)c3ncc4c(c3c2)CNC2(CC2)[C@H]4F)n1] | 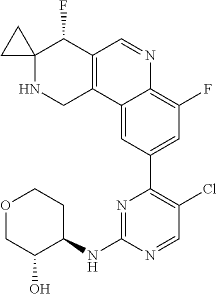 [SMILES: O[C@@H]1COCC[C@H]1Nc1ncc(Cl)c(-c2cc(F)c3ncc4c(c3c2)CNC2(CC2)[C@@H]4F)n1] | 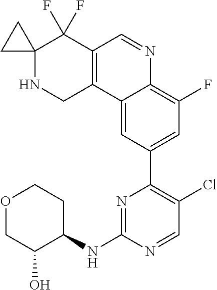 [SMILES: O[C@@H]1COCC[C@H]1Nc1ncc(Cl)c(-c2cc(F)c3ncc4c(c3c2)CNC2(CC2)C4(F)F)n1] |

Compounds of Table 1 can exist in various stereoisomeric forms, such as individual isomer, an individual enantiomer and/or diastereomer, as applicable, or a mixture of stereoisomers, including racemic mixtures and mixtures enriched in one or more stereoisomers. In some embodiments, when applicable, a compound shown Table 1 can have an enantiomeric excess (“ee”) of greater than 60%, such as having greater than 80% ee, greater than 90% ee, greater than 90% ee, greater than 95% ee, greater than 98% ee, greater than 99% ee, or with the other enantiomer in a non-detectable amount. In some embodiments, when applicable, a compound shown Table 1 can also exist as a mixture of stereoisomers in any ratio, such as a racemic mixture.

In some embodiments, to the extent applicable, the genus of compounds described herein also excludes any specifically known single compounds prior to this disclosure. In some embodiments, to the extent applicable, any sub-genus or species of compounds prior to this disclosure that are entirely within a genus of compounds described herein can also be excluded from such genus herein.

The compounds of the present disclosure can be readily synthesized by those skilled in the art in view of the present disclosure. Exemplified synthesis are also shown in the Examples section.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (or any sub-formula provided herein, e.g., Formula I-A, I-B, A, A-S1, A-S2, A-S3, A-S4, A-1, A-2, I-1, I-1-D, I-2, I-2-D, 1-3, or 1-4), any of Examples 1-111, or any of the specific compounds disclosed in Table 1 herein, or a pharmaceutically acceptable salt thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (or any sub-formula provided herein, e.g., Formula I-A, I-B, A, A-S1, A-S2, A-S3, A-S4, A-1, A-2, I-1, I-1-D, 1-2, I-2-D, 1-3, or I-4), any of Examples 1-111, or any of the specific compounds disclosed in Table 1 herein, or a pharmaceutically acceptable salt thereof, e.g., in a therapeutically effective amount. In any of the embodiments described herein, the pharmaceutical composition can comprise a therapeutically effective amount (e.g., for treating breast cancer or ovarian cancer) of a compound selected from any of Examples 1-111, or any of the specific compounds disclosed in Table 1 herein, or a pharmaceutically acceptable salt thereof. In some preferred embodiments, the pharmaceutical composition can comprise a compound selected from the compounds according to Examples 1-111 that have a CDK4/CyclinD1 IC50 level designated as “A” or “B”, preferably, “A” in Table 2 herein.

The pharmaceutical composition herein can be formulated for delivery via any of the known routes of delivery, which include but not limited to administering orally, nasally, transdermally, pulmonary, inhalationally, buccally, sublingually, intraperitoneally, subcutaneously, intramuscularly, intravenously, rectally, intrapleurally, intrathecally or parenterally.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

Compounds of the present disclosure can be used alone, in combination with each other, or in combination with one or more additional therapeutic agents, e.g., in combination with an additional anticancer therapeutic agent, such as mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like. In some embodiments, one or more compounds of the present disclosure can be used in combination with one or more targeted agents, such as inhibitors of PI3 kinase, mTOR, PARP, IDO, TDO, ALK, ROS, MEK, VEGF, FLT3, AXL, ROR2, EGFR, FGFR, Src/Abl, RTK/Ras, Myc, Raf, PDGF, AKT, c-Kit, erbB, CDK2, CDK5, CDK7, CDK9, SMO, CXCR4, HER2, GLS1, EZH2 or Hsp90, or immunomodulatory agents, such as PD-1 or PD-L1 antagonists, OX40 agonists or 4-1BB agonists. In some embodiments, one or more compounds of the present disclosure can be used in combination with a standard of care agent, such as tamoxifen, docetaxel, paclitaxel, cisplatin, capecitabine, gemcitabine, vinorelbine, exemestane, letrozole, fulvestrant, anastrozole or trastuzumab. Suitable additional anticancer therapeutic agent include any of those known in the art, such as those approved for the appropriate cancer by a regulatory agency such as the U.S. Food and Drug Administration. Some examples of suitable additional anticancer therapeutic agents also include those described as suitable for combined use with a CDK inhibitor in WO2019/207463, WO2020/224568, etc., the content of each of which is herein incorporated by reference in its entireties.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, such as breast cancer or ovarian cancer, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of a CDK4-mediated disease or disorder. Accordingly, some embodiments of the present disclosure are also directed to methods of using one or more compounds of the present disclosure or pharmaceutical compositions herein for treating or preventing a CDK4-mediated disease or disorder in a subject in need thereof, such as for treating cancer in a subject in need thereof.

In some embodiments, the present disclosure provides a method of inhibiting abnormal cell growth in a subject in need thereof, comprising administering to the subject a therapeutically effective amount of a compound of the present disclosure or a pharmaceutical composition described herein. In some embodiments, the abnormal cell growth is cancer associated with CDK4.

In some embodiments, the present disclosure also provides a method of inhibiting CDK activity in a subject or biological sample. In some embodiments, the present disclosure provides a method of inhibiting CDK4 activity in a subject or biological sample, which comprises contacting the subject or biological sample with an effective amount of the compound of the present disclosure (e.g., a compound of Formula I (or any sub-formula provided herein, e.g., Formula I-A, I-B, A, A-S1, A-S2, A-S3, A-S4, A-1, A-2, 1-1, I-1-D, I-2, I-2-D, I-3, or 1-4), any of Examples 1-111, or any of the specific compounds disclosed in Table 1 herein, or a pharmaceutically acceptable salt thereof) or a pharmaceutical composition described herein.

In some embodiments, the present disclosure provides a method of treating or preventing a CDK mediated, in particular CDK4-mediated disease or disorder in a subject in need thereof. In some embodiments, the method comprises administering to the subject an effective amount of a compound of the present disclosure (e.g., a compound of Formula I (or any sub-formula provided herein, e.g., Formula I-A, I-B, A, A-S1, A-S2, A-S3, A-S4, A-1, A-2, I-1, I-1-D, I-2, I-2-D, 1-3, or 1-4), any of Examples 1-111, or any of the specific compounds disclosed in Table 1 herein, or a pharmaceutically acceptable salt thereof) or an effective amount of a pharmaceutical composition described herein. In some embodiments, the CDK4-mediated disease or disorder is cancer, e.g., described herein.

In some embodiments, the present disclosure also provides a method of treating or preventing cancer in a subject in need thereof, which comprises administering to the subject an effective amount of a compound of the present disclosure (e.g., a compound of Formula I (or any sub-formula provided herein, e.g., Formula I-A, I-B, A, A-S1, A-S2, A-S3, A-S4, A-1, A-2, I-1, I-1-D, 1-2, I-2-D, I-3, or 1-4), any of Examples 1-111, or any of the specific compounds disclosed in Table 1 herein, or a pharmaceutically acceptable salt thereof) or an effective amount of a pharmaceutical composition described herein. In some embodiments, the cancer is selected from breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer, thyroid cancer, and combinations thereof. In some embodiments of the methods herein, the cancer is breast cancer, ovarian cancer, bladder cancer, uterine cancer, prostate cancer, lung cancer, esophageal cancer, liver cancer, pancreatic cancer and/or stomach cancer.

In some embodiments of the methods herein, the cancer is breast cancer, such as ER-positive/HR-positive, HER2-negative breast cancer; ER-positive/HR-positive, HER2-positive breast cancer; triple negative breast cancer (TNBC); or inflammatory breast cancer. In some embodiments, the breast cancer can be endocrine resistant breast cancer, trastuzumab resistant breast cancer, or breast cancer demonstrating primary or acquired resistance to CDK4/CDK6 inhibition. In some embodiments, the breast cancer can be advanced or metastatic breast cancer.

In some embodiments of the methods herein, the cancer is ovarian cancer.

In some embodiments, the compound of the present disclosure is administered as first line therapy. In other embodiments, the compound of the present disclosure is administered as second (or later) line therapy. In some embodiments, the compound of the present disclosure is administered as second (or later) line therapy following treatment with an endocrine therapeutic agent and/or a CDK4/CDK6 inhibitor. In some embodiments, the compound of the present disclosure is administered as second (or later) line therapy following treatment with an endocrine therapeutic agent, e.g., an aromatase inhibitor, a SERM or a SERD. In some embodiments, the compound of the present disclosure is administered as second (or later) line therapy following treatment with a CDK4/CDK6 inhibitor. In some embodiments, the compound of the present disclosure is administered as second (or later) line therapy following treatment with one or more chemotherapy regimens, e.g., including taxanes or platinum agents. In some embodiments, the compound of the present disclosure is administered as second (or later) line therapy following treatment with HER2 targeted agents, e.g., trastuzumab.

In some embodiments, the present disclosure also provides a method of treating breast cancer in a subject in need thereof, which comprises administering to the subject a therapeutically effective amount of a compound of the present disclosure (e.g., a compound of Formula I (or any sub-formula provided herein, e.g., Formula I-A, I-B, A, A-S1, A-S2, A-S3, A-S4, A-1, A-2, I-1, I-1-D, I-2, I-2-D, I-3, or I-4), any of Examples 1-111, or any of the specific compounds disclosed in Table 1 herein, or a pharmaceutically acceptable salt thereof) or an effective amount of a pharmaceutical composition described herein. In some embodiments, the breast cancer is selected from ER-positive/HR-positive, HER2-negative breast cancer; ER-positive/HR-positive, HER2-positive breast cancer; triple negative breast cancer (TNBC); and inflammatory breast cancer. In some embodiments, the breast cancer is selected from endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer.

In some preferred embodiments, the compound of the present disclosure for the methods herein has a CDK4/CyclinD1 IC50 of less than 100 nM, more preferably, less than 10 nM, measured/calculated according to the Biological Example 1 herein. In some preferred embodiments, the compound of the present disclosure for the methods herein is selected from the compounds according to Examples 1-111 that have a CDK4/CyclinD1 IC50 level designated as “A” or “B”, preferably “A”, in Table 2 herein.

In some preferred embodiments, the compound of the present disclosure for the methods herein are selective for CDK4 over CDK6. In some embodiments, the compound of the present disclosure for the methods herein can also be selective for CDK4 over other CDKs.

In some embodiments, the present disclosure provides a method of inhibiting cancer cell proliferation in a subject, comprising administering to the subject a compound of the present disclosure, or a pharmaceutically acceptable salt thereof, in an amount effective to inhibit cell proliferation.

In some embodiments, the present disclosure provides a method of inhibiting cancer cell invasiveness in a subject, comprising administering to the subject a compound of the present disclosure, or a pharmaceutically acceptable salt thereof, in an amount effective to inhibit cell invasiveness.

In some embodiments, the present disclosure provides a method of inducing apoptosis in cancer cells in a subject, comprising administering to the subject a compound of the present disclosure, or a pharmaceutically acceptable salt thereof, in an amount effective to induce apoptosis.

In some embodiments, the present disclosure provides a method of inhibiting cancer cell metastasis in a subject, comprising administering to the subject a compound of the present disclosure, or a pharmaceutically acceptable salt thereof, in an amount effective to inhibit cell metastasis.

The administering in the methods herein is not limited to any particular route of administration. For example, in some embodiments, the administering can be orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the administering is orally. In some embodiments, the administering is a parenteral injection, such as an intravenous injection.

Compounds of the present disclosure can be used as a monotherapy or in a combination therapy. In some embodiments according to the methods described herein, one or more compounds of the present disclosure can be administered as the only active ingredient(s). In some embodiments according to the methods described herein, one or more compounds of the present disclosure can also be co-administered with an additional therapeutic agent, either concurrently or sequentially in any order, to the subject in need thereof. The additional therapeutic agent can typically be an additional anticancer therapeutic agent, such as mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like. In some embodiments, the additional anticancer agent is an endocrine agent, such as an aromatase inhibitor, a SERD or a SERM. In some embodiments, one or more compounds of the present disclosure can be administered in combination with one or more targeted agents, such as inhibitors of PI3 kinase, mTOR, PARP, IDO, TDO, ALK, ROS, MEK, VEGF, FLT3, AXL, ROR2, EGFR, FGFR, Src/Abl, RTK/Ras, Myc, Raf, PDGF, AKT, c-Kit, erbB, CDK2, CDK5, CDK7, CDK9, SMO, CXCR4, HER2, GLS1, EZH2 or Hsp90, or immunomodulatory agents, such as PD-1 or PD-L1 antagonists, OX40 agonists or 4-1BB agonists. In some embodiments, one or more compounds of the present disclosure can be administered administered in combination with a standard of care agent, such as tamoxifen, docetaxel, paclitaxel, cisplatin, capecitabine, gemcitabine, vinorelbine, exemestane, letrozole, fulvestrant, anastrozole or trastuzumab. Suitable additional anticancer therapeutic agent include any of those known in the art, such as those approved for the appropriate cancer by a regulatory agency such as the U.S. Food and Drug Administration. Some examples of suitable additional anticancer therapeutic agents also include those described as suitable for combined use with a CDK inhibitor in WO2019/207463, WO2020/224568, etc., the contents of each of which is incorporated by reference herein in their entirety.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable groups for the variables in compounds of Formula I, or a subformula thereof, as applicable, are independently selected. Non-limiting useful groups for the variables in compounds of Formula I, or a subformula thereof, as applicable, include any of the respective groups, individually or in any combination, as shown in the Examples or in the specific compounds described in Table 1 herein. Using variable R¹ as an example, in some embodiments, compounds of Formula I can include a R¹ group according to any of the R¹ groups shown in the Examples or in the specific compounds described in Table 1 herein, without regard to the other variables shown in the specific compounds. In some embodiments, compounds of Formula I can include a R¹ group according to any of the R¹ groups shown in the Examples or in the specific compounds described in Table 1 herein in combination at least one other variable (e.g, L¹) according to the Examples or the specific compounds described in Table 1 herein, wherein the R¹ and at least one other variable can derive from the same compound or a different compound. Any of such combinations are contemplated and within the scope of the present disclosure. Unless otherwise specified, a description of a variable in connection with a formula also applies to any other overlapping formulas (e.g., subformulas) where said variable is present.

The described embodiments of the present disclosure can be combined. Such combination is contemplated and within the scope of the present disclosure. For example, it is contemplated that the definition(s) of any one or more of L¹, R¹, R², R³, R⁴, and X of Formula I (or any sub-formula provided herein, e.g., Formula I-A, I-B, A, A-S1, A-S2, A-S3, A-S4, A-1, A-2, I-1, I-1-D, 1-2, I-2-D, 1-3, or I-4) can be combined with the definition of any one or more of the other(s) of L¹, R¹, R², R³, R⁴, and X, as applicable, and the resulted compounds from the combination are within the scope of the present disclosure.

The symbol, [symbol] , displayed perpendicular to or otherwise crossing a bond, e.g.,

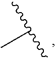

indicates the point at which the displayed moiety is attached to the remainder of the molecule. It should be noted that the immediately connected group or groups maybe shown beyond the symbol, [symbol] , to indicate connectivity, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC), chiral supercritical fluid chromatograph (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC or SFC area, or both, or with a non-detectable amount of the other stereoisomer(s). For example, in some embodiments, the compound can exist predominantly as the as-drawn stereoisomer having an enantiomeric excess (“ee”) of greater than 80%, such as having an ee of 90% or above, 95% or above, 98% or above, 99% or above, or have a non-detectable amount of the other enantiomer. The presence and/or amounts of stereoisomers can be determined by those skilled in the art in view of the present disclosure, including through the use of a chiral HPLC or chiral SFC. As understood by those skilled in the art, when a “*” is shown in the chemical structures herein, unless otherwise contradictory from context, it is to designate that the corresponding chiral center is enantiomerically pure or enriched in either of the configurations or is enantiomerically pure or enriched in the as-dawn configuration, such as with less than 20%, less than 10%, less than 5%, less than 10%, by weight, by HPLC or SFC area, or both, or with a non-detectable amount of the other stereoisomer(s). Also, when no stereochemistry is specifically drawn, and no “*” is used in the chemical structures, unless otherwise contradictory from context, it should be understood that such structures include the corresponding compound in any stereoisomeric forms, including individual isomers substantially free of other isomers and mixtures of various isomers including racemic mixtures.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula I (or any sub-formula provided herein, e.g., Formula I-A, I-B, A, A-S1, A-S2, A-S3, A-S4, A-1, A-2, I-1, I-1-D, 1-2, I-2-D, I-3, or I-4), any of Examples 1-111, or any of the specific compounds disclosed in Table 1 herein, isotopically labeled compound(s) thereof (such as a deuterated analog wherein one or more of the hydrogen atoms is/are substituted with a deuterium atom with an abundance above its natural abundance, e.g., a CD₃ analog when the compound has a CH₃ group), possible regioisomers, possible geometric isomers, possible stereoisomers thereof (including diastereoisomers, enantiomers, and racemic mixtures), tautomers thereof, conformational isomers thereof, pharmaceutically acceptable esters thereof, and/or possible pharmaceutically acceptable salts thereof (e.g., acid addition salt such as HCl salt or base addition salt such as Na salt). To be clear, compounds of Examples 1-111 refer to the compounds in the Examples section, labeled with an integer only, or labeled with an integer followed by “a” “b” etc. to indicate different stereoisomers of the compound labeled with the integer, such as 1, 2, etc. 15a, 15b, up to 111a and 111b. See e.g., Illustrations 1-27 herein. Hydrates and solvates of the compounds of the present disclosure are considered compositions of the present disclosure, wherein the compound(s) is in association with water or solvent, respectively. In some embodiments, compounds of the present disclosure can be any of those according to the claims provided herein.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl can include one to twelve carbon atoms (i.e., C₁₋₁₂ alkyl) or the number of carbon atoms designated. In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like. Unless otherwise specified, an alkyl group is optionally substituted.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl. Unless otherwise specified, an alkenyl group is optionally substituted.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups. Unless otherwise specified, an alkynyl group is optionally substituted.

As used herein, the term “alkoxy” as used by itself or as part of another group refers to a radical of the formula OR^{a1}, wherein R^{a1} is an alkyl as described herein.

As used herein, the term “cycloalkoxy” as used by itself or as part of another group refers to a radical of the formula OR^{a1}, wherein R^{a1} is a cycloalkyl as described herein.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. In any of the embodiments described herein, the C₁₋₄ heteroalkyl preferably has 1 or 2 heteroatoms, such as those having one oxygen, one oxygen and one nitrogen, two oxygen atoms, or two nitrogen atoms. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. Unless otherwise specified, a heteroalkyl group is optionally substituted.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein. Unless otherwise specified, a carbocyclyl group is optionally substituted.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring. As used herein, the term “cycloalkylene” as used by itself or as part of another group refers to a divalent radical derived from a cycloalkyl group, for example,

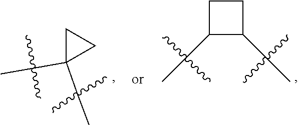 [SMILES: *C1(*)CC1 ; *C1CCC1*]

etc.

“Heterocyclyl” or “heterocyclic” as used by itself or as part of another group refers to a radical of a 3-membered or larger, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings, and the point of attachment can be on any ring. As used herein, the term “heterocyclylene” as used by itself or as part of another group refers to a divalent radical derived from the heterocyclyl group defined herein. The heterocyclyl or heterocylylene can be optionally linked to the rest of the molecule through a carbon or nitrogen atom. Unless otherwise specified, a heterocyclyl group is optionally substituted.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array), e.g., having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). As used herein, the term “arylene” as used by itself or as part of another group refers to a divalent radical derived from the aryl group defined herein. Unless otherwise specified, an aryl group is optionally substituted.

“Aralkyl” as used by itself or as part of another group refers to an alkyl substituted with one or more aryl groups, preferably, substituted with one aryl group. Examples of aralkyl include benzyl, phenethyl, etc. Unless otherwise specified, an aralkyl group is optionally substituted. When an aralkyl is said to be optionally substituted, either the alkyl portion or the aryl portion of the aralkyl can be optionally substituted.

“Heteroaryl” as used by itself or as part of another group refers to a radical of a monocyclic, bicyclic, or tricyclic 4n+2 aromatic ring system (e.g., having 6 or 10 pi electrons shared in a cyclic array) having ring carbon atoms and at least one, preferably, 1-4, ring heteroatoms provided in the aromatic ring system, wherein each heteroatom is independently selected from nitrogen, oxygen and sulfur. In one embodiment, a heteroaryl group has 5-14 membered ring atoms (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. In bicyclic heteroaryl groups wherein one ring does not contain a heteroatom (e.g., indolyl, quinolinyl, and the like), the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein. Unless specified or otherwise contrary from context, when a heteroaryl group is fused to a non-aromatic ring, the resulted fused ring systems is referred to as a heterocyclyl group. Unless otherwise specified, a heteroaryl group is optionally substituted.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. Unless otherwise specified, a heteroaralkyl group is optionally substituted. When a heteroaralkyl is said to be optionally substituted, either the alkyl portion or the heteroaryl portion of the heteroaralkyl can be optionally substituted.

As used herein, and unless otherwise specified, the term “amino” refers to —N(R^{#})(R^{#}), wherein each R^{#} independently can be, but is not limited to, hydrogen, alkyl, heteroalkyl, alkenyl, alkynyl, aryl, cycloalkyl, heteroaryl, heterocyclyl, each of which is defined above. When a —N(R^{#})(R¹⁹⁰) group has two R^{#} other than hydrogen, they can be combined with the nitrogen atom to form a ring. In one embodiment, the ring is a 3-, 4-, 5-, 6-, 7-, or 8-membered ring. In one embodiment, one or more ring atoms are heteroatoms independently selected from nitrogen, oxygen, sulfur, boron, phosphorus, and silicon. The term “amino” also includes N-oxide (—N⁺(R^{#})(R^{#})O⁻). In certain embodiments, each R^{#} or the ring formed by —N(R^{#})(R^{#}) independently may be unsubstituted or substituted with one or more substituents.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable, each of which can be optionally isotopically labeled, such as deuterated. Two of the optional substituents can join to form a ring structure, such as an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N(except S02-N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, OH, hydroxyalkyl, haloalkyl, —O-alkyl, O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), N(Y₁)(Y₂), -alkylene-N(Y₁)(Y₂), C(O)N(Y₁)(Y₂) and S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₅)alkyl groups, (C₂-C₈)alkenyl groups, (C₂-C₈)alkynyl groups, (C₃-C₁₀)cycloalkyl groups, halogen (F, Cl, Br or I), halogenated (C₁-C₅)alkyl groups (for example but not limited to —CF₃), —O—(C₁-C₅)alkyl groups, —OH, —S—(C₁-C₅)alkyl groups, —SH, —NH(C₁-C₅)alkyl groups, —N((C₁-C₈)alkyl)₂ groups, —NH₂, —C(O)NH₂, —C(O)NH(C₁-C₅)alkyl groups, —C(O)N((C₁-C₈)alkyl)₂, —NHC(O)H, NHC(O)(C₁-C₈)alkyl groups, —NHC(O)(C₃-C₈)cycloalkyl groups, —N((C₁-C₈)alkyl)C(O)H, —N((C₁-C₈)alkyl)C(O)(C₁-C₅)alkyl groups, —NHC(O)NH₂, —NHC(O)NH(C₁-C₅)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₅)alkyl)₂ groups, —N((C₁-C₈)alkyl)C(O)NH((C₁-C₅)alkyl), —C(O)H, —C(O)(C₁-C₈)alkyl groups, —CN, —NO₂, —S(O)(C₁-C₅)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₅)alkyl groups, —N((C₁-C₈)alkyl)S(O)₂(C₁-C₅)alkyl groups, —(C₁-C₈)alkyl-O—(C₁-C₈)alkyl groups, —O—(C₁-C₅)alkyl-O—(C₁-C₈)alkyl groups, —C(O)OH, —C(O)O(C₁-C₈)alkyl groups, NHOH, NHO(C₁-C₈)alkyl groups, O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₅)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₅)alkyl groups, or —C(═N—O—(C₁-C₅)alkyl)-(C₁-C₆)alkyl groups.

Exemplary carbon atom substituents include, but are not limited to, deuterium, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(CI-6 alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, heteroalkyl, heteroalkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from deuterium, F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from deuterium, F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from deuterium, F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C-4 alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from deuterium, F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. As used herein, and unless otherwise specified, the term “pharmaceutically acceptable salt” includes both acid and base addition salts.

Examples of pharmaceutically acceptable acid addition salts include, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, capiylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Examples of pharmaceutically acceptable base addition salt include, but are not limited to, salts prepared from addition of an inorganic base or an organic base to a free acid compound. Salts derived from inorganic bases include, but are not limited to, the sodium, potassium, lithium, ammonium, calcium, magnesium, iron, zinc, copper, manganese, aluminum salts and the like. In one embodiment, the inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. In one embodiment, the organic bases are isopropylamine, diethylamine, ethanolamine, trimethylamine, dicyclohexylamine, choline and caffeine.

The term “tautomers” or “tautomeric” refers to two or more interconvertible compounds resulting from tautomerization. The exact ratio of the tautomers depends on several factors, including for example temperature, solvent, and pH. Tautomerizations are known to those skilled in the art. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, and unless otherwise specified, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a compound described herein to a subject in need of such treatment.

As used herein, and unless otherwise specified, the terms “prevent,” “preventing,” “prevention,” and the like refer to reducing the probability of the onset of a disease or condition, of redeveloping a disease or condition, or of a recurrence of a previously-controlled disease or condition, in a subject who does not have, but is at risk of or is susceptible to, developing or redeveloping a disease or condition or a recurrence of the disease or condition.

The term “effective amount” refers to that amount of a compound or combination of compounds as described herein that is sufficient to effect the intended application including, but not limited to, prophylaxis or treatment of diseases. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells and/or tissues. The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the singular form “a”, “an”, and “the”, includes plural references unless it is expressly stated or is unambiguously clear from the context that such is not intended.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It should be noted that if there is a discrepancy between a depicted structure and a name for that structure, the depicted structure is to be accorded more weight.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of embodiments herein can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. The abbreviations used in the Examples section should be understood as having their ordinary meanings in the art unless specifically indicated otherwise or obviously contrary from context. The examples are illustrative only and do not limit the claimed invention in any way.

Exemplary embodiments of steps for performing the synthesis of products described herein are described in greater detail infra. Some of the Examples discussed herein can be prepared by separating from the corresponding racemic mixtures. As would be understood by a person of ordinary skill in the art, the compounds described in the Examples section immediately prior to the chiral separation step, e.g., by supercritical fluid chromatography (SFC), exist in racemic and/or stereoisomeric mixture forms, the bolded but not wedged bonds are used in the chemical structure drawings to indicate relative stereochemistry. It should be understood that the enantiomeric excesses (“ee”) and/or diastereomeric excesses (“de”) reported for these examples are only representative from the exemplified procedures herein and not limiting; those skilled in the art would understand that such enantiomers with a different ee and/or de, such as a higher ee and/or higher de, can be obtained in view of the present disclosure.

In some illustrative examples, the synthesis of a deuterated compound is shown. To the extent applicable, it should be understood that the corresponding non-deuterated (i.e., with natural abundance) compound was prepared through the same method except by using a corresponding non-deuterated starting material or intermediate.

### Synthesis of Borate Intermediates

### 4,4,7-trifluoro-3,3-dimethyl-9-(4,4,5,5-tetramethyl-1,3,2-dioxaborolan-2-yl)-1,2,3,4-tetrahydrobenzo[c][2,6]naphthyridine (Int-1)

As the starting material, S—1.1 is obtained according to the procedures of illustration 24 in the example section, and it goes through substitution, hydrolysis, fluorination to provide S—1.4. Then Int-1 is synthesized by reduction and borylation from S—1.4.

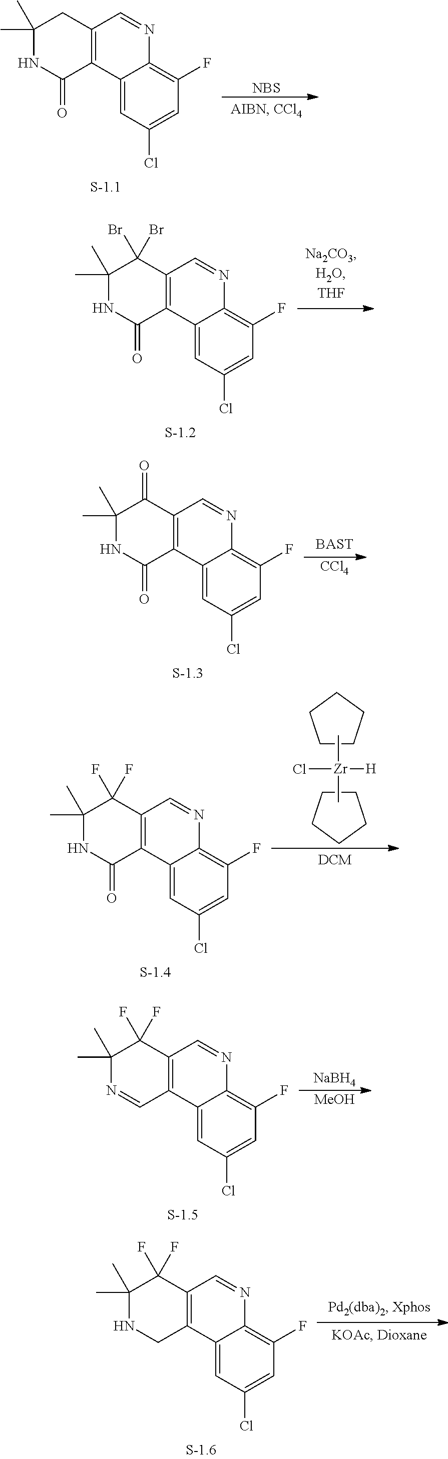

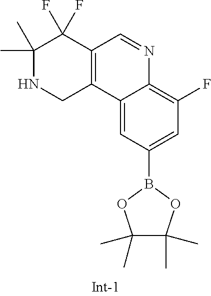 [SMILES: CC1(C)NCc2c(cnc3c(F)cc(B4OC(C)(C)C(C)(C)O4)cc23)C1(F)F]

### 7-fluoro-9-(4,4,5,5-tetramethyl-1,3,2-dioxaborolan-2-yl)-1,4-dihydro-2H-spiro[benzo[c][2,6]naphthyridine-3,1′-cyclopropane]-1,1-d₂ (Int-2)

Int-2 is synthesized starting from S—2.1 according to the synthetic route below. Firstly, S—2.9 is obtained via the similar procedures of illustration 24 in the example section. Then Int-2 is obtained by reduction and borylation from S—2.9 via the similar procedures of illustration 27 in the example section.

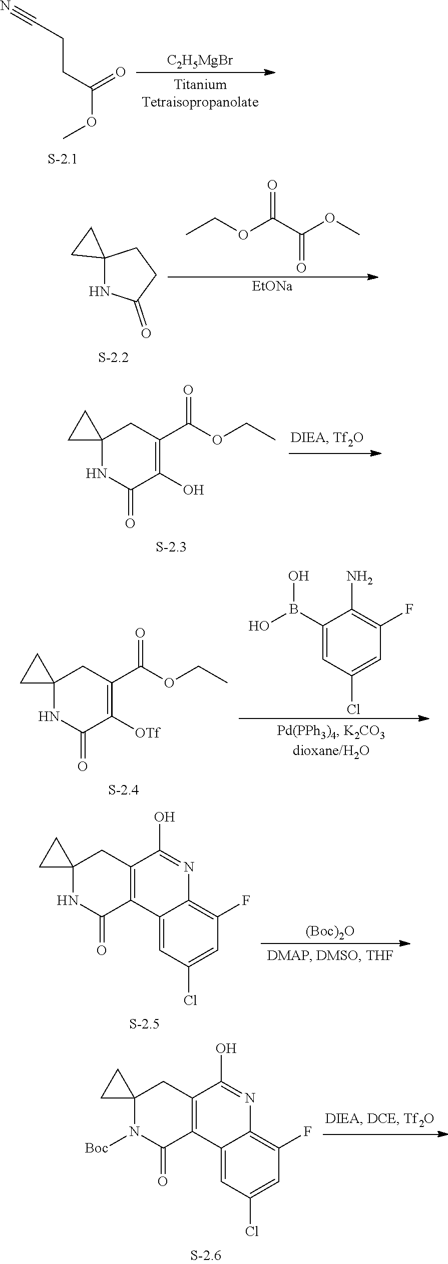

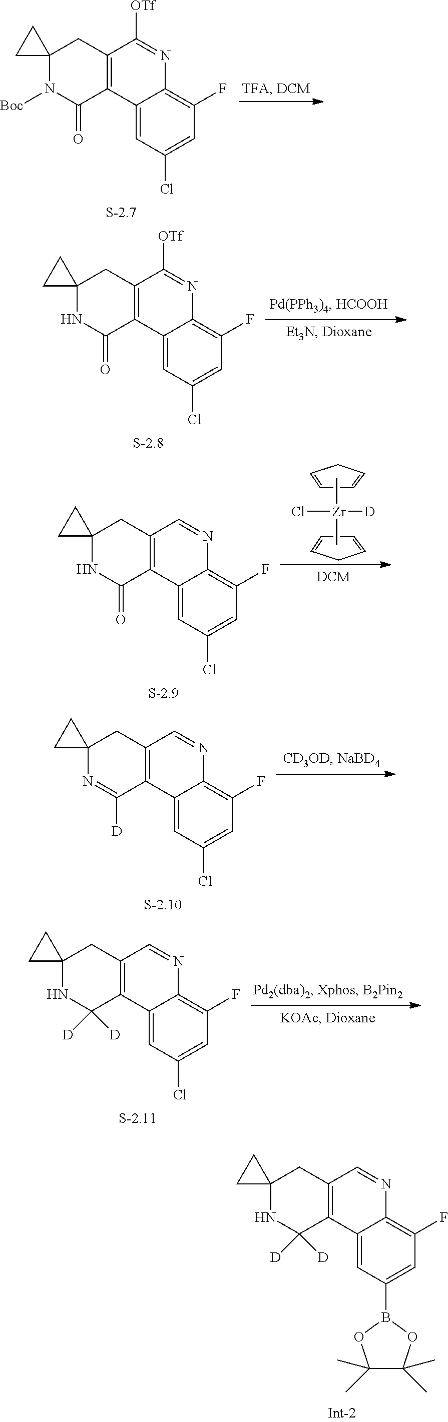

### 4,7-difluoro-3,3-dimethyl-9-(4,4,5,5-tetramethyl-1,3,2-dioxaborolan-2-yl)-1,2,3,4-tetrahydrobenzo[c][2,6]naphthyridine (Int-3)

Int-3 is synthesized starting from S—1.3 according to the synthetic route below.

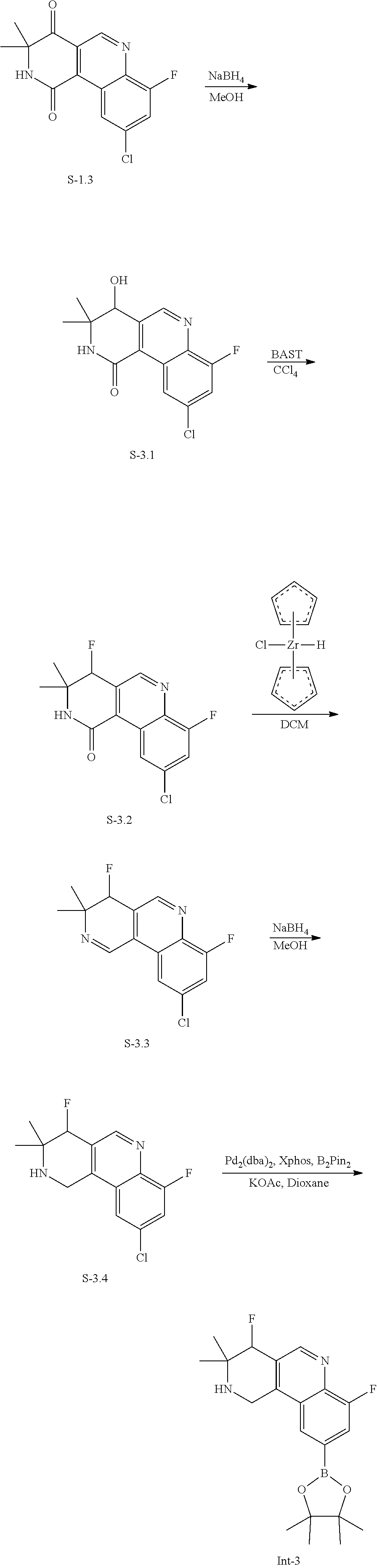

### 4,7-difluoro-9-(4,4,5,5-tetramethyl-1,3,2-dioxaborolan-2-yl)-1,4-dihydro-2H-spiro[benzo[c][2,6]naphthyridine-3,1′-cyclopropane](Int-4)

Int-4 is synthesized starting from S—2.9 according to the synthetic route below.

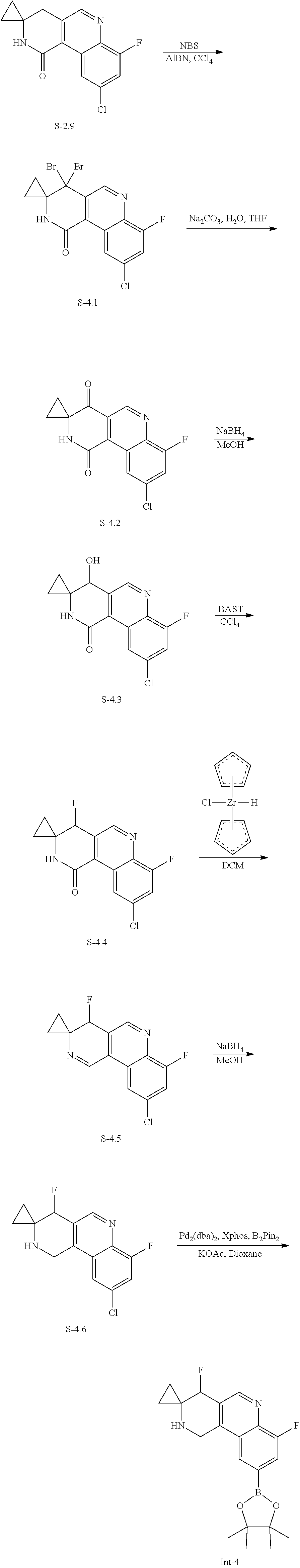

### 4,4,7-trifluoro-9-(4,4,5,5-tetramethyl-1,3,2-dioxaborolan-2-yl)-1,4-dihydro-2H-spiro[benzo[c][2,6]naphthyridine-3,1′-cyclopropane](Int-5)

Int-5 is synthesized starting from S—4.2 according to the synthetic route below.

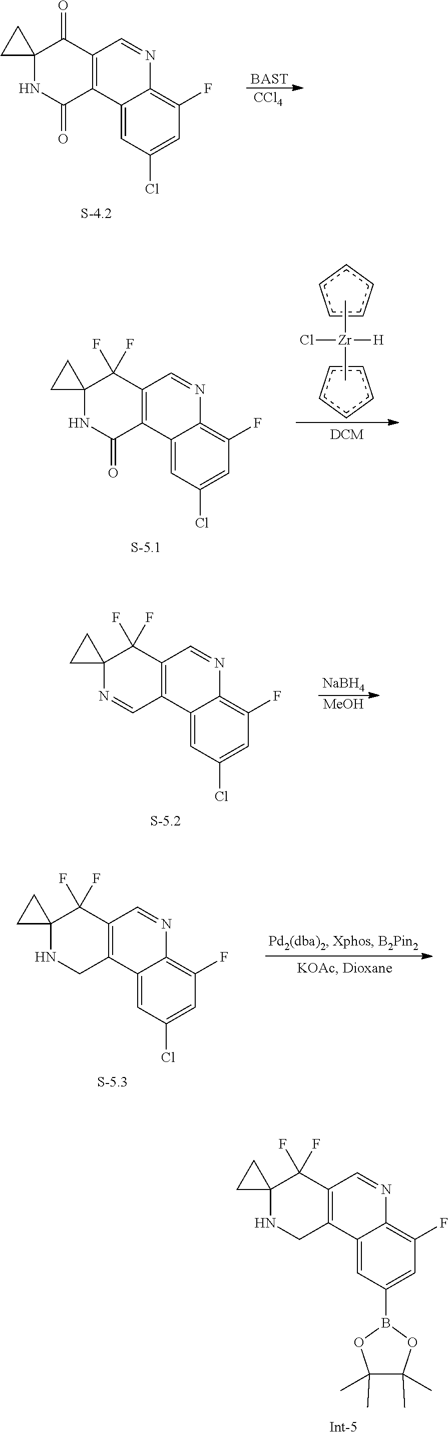

### Illustration 1. Synthesis of(3S,4R)-4-((5-chloro-4-(8-fluoro-3-(2-hydroxypropan-2-yl)quinolin-6-yl)pyrimidin-2-yl)amino)tetrahydro-2H-pyran-3-ol (1)

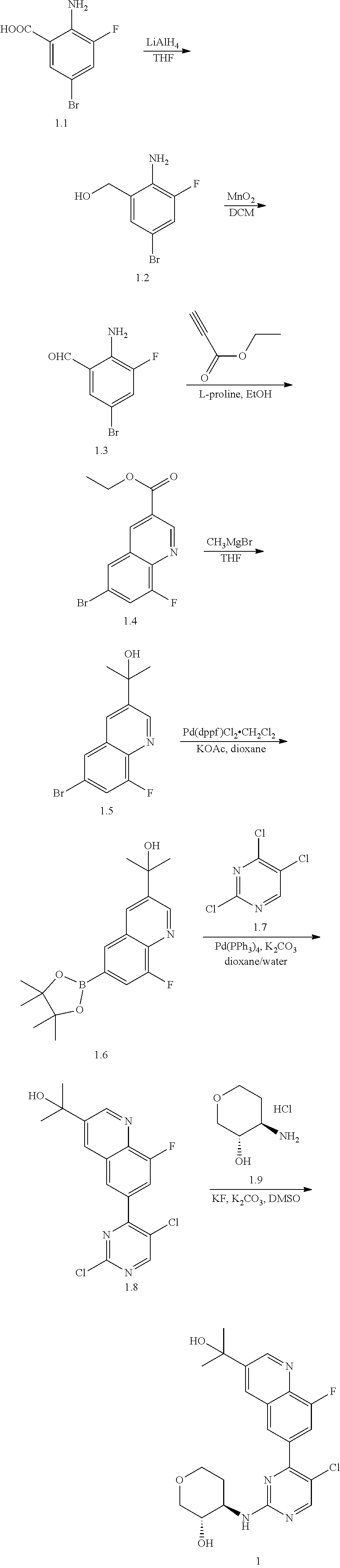

To a solution of 2-amino-5-bromo-3-fluorobenzoic acid (1.1, 1.00 g, 4.27 mmol) in tetrahydrofuran (20 mL) was added lithium aluminum hydride (0.32 g, 8.55 mmol) in portions at 0° C., and the mixture was stirred at 25° C. for 2 hrs. The reaction mixture was quenched with sodium sulfate decahydrate (1.20 g) slowly and diluted with ethyl acetate (20 mL). The resulting mixture was filtered and the filtrate was concentrated under reduced pressure. The residue was subjected to silica gel column chromatography to afford (2-amino-5-bromo-3-fluorophenyl)methanol (1.2, 750 mg, 80%) as a yellow solid. LC-MS (ESI): m/z 220.0 [M+H]⁺. ¹H NMR (400 MHz, CDCl₃) δ 7.16 (dd, J=10.3, 2.2 Hz, 1H), 7.07-7.04 (m, 1H), 4.70 (s, 2H), 4.28 (brs, 2H).

A mixture of (2-amino-5-bromo-3-fluorophenyl)methanol (1.2, 750 mg, 3.41 mmol) and manganese dioxide (5.93 g, 68.2 mmol) in dichloromethane (20 mL) was stirred at 25° C. for 12 hrs. The reaction mixture was filtered and the filter cake was washed with dichloromethane (30 mL). The filtrate was concentrated under reduced pressure and the residue was subjected to silica gel column chromatography to afford 2-amino-5-bromo-3-fluorobenzene-1-carbaldehyde (1.3, 560 mg, 75%) as a yellow solid. ¹H NMR (400 MHz, CDCl₃) δ 9.87 (d, J=2.0 Hz, 1H), 7.51-7.45 (m, 1H), 7.33 (dd, J=10.6, 2.2 Hz, 1H), 6.20 (brs, 2H).

A mixture of 2-amino-5-bromo-3-fluorobenzene-1-carbaldehyde (1.3, 470 mg, 2.16 mmol), ethyl prop-2-ynoate (254 mg, 2.59 mmol) and L-proline (74.5 mg, 0.65 mmol) in ethanol (20 mL) was stirred at 90° C. for 12 hrs. The reaction mixture was concentrated under reduced pressure and the residue was subjected to silica gel column chromatography to afford ethyl 6-bromo-8-fluoroquinoline-3-carboxylate (1.4, 620 mg, 96%) as a yellow solid. LC-MS (ESI): m/z 298.0 [M+H]⁺. ¹H NMR (400 MHz, CDCl₃) δ 9.52 (d, J=1.9 Hz, 1H), 8.84-8.78 (m, 1H), 7.98-7.94 (m, 1H), 7.69 (dd, J=9.5, 1.9 Hz, 1H), 4.53 (q, J=7.1 Hz, 2H), 1.50 (t, J=7.2 Hz, 3H).

To a solution of ethyl 6-bromo-8-fluoroquinoline-3-carboxylate (1.4, 300 mg, 1.06 mmol) in tetrahydrofuran (10 mL) was added methylmagnesium bromide (1.68 mL, 5.03 mmol, 3 M in 2-Methyltetrahydrofuran) dropwise under nitrogen atmosphere at 0° C., and the mixture was stirred at 25° C. for 4 hrs. The reaction mixture was quenched with saturated ammonium chloride solution (5 mL), diluted with water (20 mL) and extracted with ethyl acetate (20 mL×3). The combined organic layers were washed with brine (20 mL), dried over anhydrous sodium sulfate and concentrated under reduced pressure. The residue was subjected to silica gel column chromatography to afford 2-(6-bromo-8-fluoroquinolin-3-yl)propan-2-ol (1.5, 270 mg, 94%) as a yellow solid. LC-MS (ESI): m/z 284.0 [M+H]+.

A mixture of 2-(6-bromo-8-fluoroquinolin-3-yl)propan-2-ol (1.5, 200 mg, 0.70 mmol), 4,4,5,5-tetramethyl-2-(4,4,5,5-tetramethyl-1,3,2-dioxaborolan-2-yl)-1,3,2-dioxaborolane (215 mg, 0.84 mmol), potassium acetate (207 mg, 2.11 mmol) and 1,1′-bis(diphenylphosphino)ferrocene-palladium(II)dichloride dichloromethane complex (57.6 mg, 0.07 mmol) in dioxane (4 mL) was stirred at 90° C. for 4 hrs under nitrogen atmosphere. The reaction mixture was cooled to room temperature, diluted with ethyl acetate (30 mL) and filtered. The filtrate was concentrated under reduced pressure and the residue was subjected to silica gel column chromatography to afford 2-(8-fluoro-6-(4,4,5,5-tetramethyl-1,3,2-dioxaborolan-2-yl)quinolin-3-yl)propan-2-ol (1.6, 160 mg, 68%) as a yellow solid. LC-MS (ESI): m/z 332.2 [M+H]⁺.

A mixture of 2-(8-fluoro-6-(4,4,5,5-tetramethyl-1,3,2-dioxaborolan-2-yl) quinolin-3-yl)propan-2-ol (1.6, 80.0 mg, 0.24 mmol), 2,4,5-trichloropyrimidine (1.7, 40.0 mg, 0.22 mmol), tetrakis(triphenylphosphine)palladium (25.2 mg, 0.02 mmol) and potassium carbonate (60.3 mg, 0.43 mmol) in dioxane (1 mL) and water (0.25 mL) was stirred at 90° C. for 3 hrs under nitrogen atmosphere. The reaction mixture was concentrated under reduced pressure and the residue was subjected to silica gel column chromatography to afford 2-(6-(2,5-dichloropyrimidin-4-yl)-8-fluoroquinolin-3-yl)propan-2-ol (1.8, 40.0 mg, 52%) as a yellow solid. LC-MS (ESI): m/z 352.0 [M+H]⁺.

A mixture of 2-(6-(2,5-dichloropyrimidin-4-yl)-8-fluoroquinolin-3-yl)propan-2-ol (1.8, 40.0 mg, 0.11 mmol), (3S,4R)-4-aminotetrahydropyran-3-ol hydrogen chloride (1.9, 34.9 mg, 0.23 mmol), potassium fluoride (26.4 mg, 0.45 mmol), 18-crown-6 (30.0 mg, 0.11 mmol) and potassium carbonate (30.0 mg, 0.11 mmol) in dimethyl sulfoxide (2 mL) was stirred at 120° C. for 2 hrs. The reaction mixture was filtered and the filtrate was subjected to prep-HPLC to afford (3S,4R)-4-((5-chloro-4-(8-fluoro-3-(2-hydroxypropan-2-yl)quinolin-6-yl)pyrimidin-2-yl)amino)tetrahydro-2H-pyran-3-ol (1, 16.2 mg, 33%) as a white solid. LC-MS (ESI): m/z 433.1 [M+H]⁺. ¹H NMR (400 MHz, DMSO-d₆) δ 9.21 (d, J=2.1 Hz, 1H), 8.56 (s, 1H), 8.49 (s, 1H), 8.30 (s, 1H), 7.87 (d, J=11.6 Hz, 1H), 7.57 (d, J=8.1 Hz, 1H), 5.51 (s, 1H), 4.95 (d, J=5.4 Hz, 1H), 3.94-3.75 (m, 3H), 3.57-3.47 (m, 1H), 3.40-3.38 (m, 1H), 3.08 (t, J=10.4 Hz, 1H), 2.04-1.92 (m, 1H), 1.61 (s, 6H), 1.58-1.46 (m, 1H).

### Illustration 2. Synthesis of 4-(8-fluoro-3-(2-hydroxypropan-2-yl)quinolin-6-yl)-2-(((3S,4R)-3-hydroxytetrahydro-2H-pyran-4-yl)amino)pyrimidine-5-carbonitrile (2)

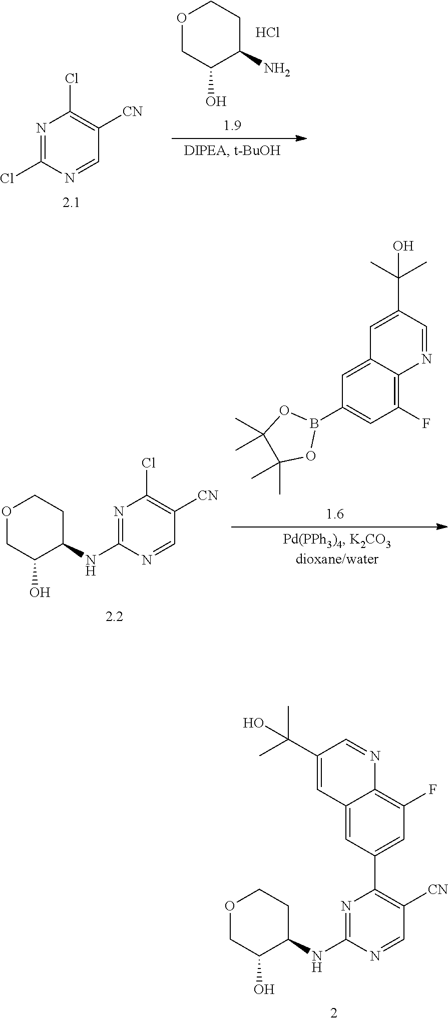 [SMILES: N#Cc1cnc(Cl)nc1Cl ; N[C@@H]1CCOC[C@H]1O ; Cl ; N#Cc1cnc(N[C@@H]2CCOC[C@H]2O)nc1Cl ; CC(C)(O)c1cnc2c(F)cc(B3OC(C)(C)C(C)(C)O3)cc2c1 ; CC(C)(O)c1cnc2c(F)cc(-c3nc(N[C@@H]4CCOC[C@H]4O)ncc3C#N)cc2c1]

To a solution of (3S,4R)-4-aminotetrahydro-2H-pyran-3-ol hydrochloride (1.9, 500 mg, 3.26 mmol) and N,N-diisopropylethylamine (841 mg, 6.51 mmol) in tert-butanol (10 mL) was added 2,4-dichloropyrimidine-5-carbonitrile (2.1, 849 mg, 4.88 mmol) at 25° C., and the mixture was stirred at 80° C. for 12 hrs. The reaction mixture was concentrated under reduced pressure and the residue was subjected to prep-HPLC to afford 4-chloro-2-(((3S,4R)-3-hydroxytetrahydro-2H-pyran-4-yl)amino)pyrimidine-5-carbonitrile (2.2, 480 mg, 58%) as a yellow solid. LC-MS (ESI): m/z 255.1 [M+H]⁺.

A mixture of 2-(8-fluoro-6-(4,4,5,5-tetramethyl-1,3,2-dioxaborolan-2-yl)quinolin-3-yl)propan-2-ol (1.6, 28.6 mg, 0.08 mmol), 4-chloro-2-(((3S,4R)-3-hydroxytetrahydro-2H-pyran-4-yl)amino)pyrimidine-5-carbonitrile (2.2, 20.0 mg, 0.08 mmol), tetrakis(triphenylphosphine)palladium (18.2 mg, 0.02 mmol) and potassium carbonate (21.7 mg, 0.16 mmol) in dioxane (1 mL) and water (0.25 mL) was stirred at 90° C. for 4 hrs under nitrogen atmosphere. The resulting mixture was diluted with water (40 mL) and extracted with ethyl acetate (20 mL×3). The combined organic layers were washed with brine (15 mL), dried over anhydrous sodium sulfate and concentrated under reduced pressure. The residue was subjected to prep-HPLC to afford 4-(8-fluoro-3-(2-hydroxypropan-2-yl)quinolin-6-yl)-2-(((3S,4R)-3-hydroxytetrahydro-2H-pyran-4-yl)amino)pyrimidine-5-carbonitrile (2, 5.50 mg, 16%) as a white solid. LC-MS (ESI): m/z 424.0 [M+H]⁺. ¹H NMR (400 MHz, DMSO-d₆) δ 9.24 (d, J=2.1 Hz, 1H), 8.85 (d, J=24.4 Hz, 1H), 8.58 (d, J=7.9 Hz, 1H), 8.55-8.40 (m, 2H), 8.05-7.91 (m, 1H), 5.55 (d, J=3.0 Hz, 1H), 5.06-4.96 (m, 1H), 4.14-3.97 (m, 1H), 3.90-3.79 (m, 2H), 3.59-3.52 (m, 1H), 3.43-3.40 (m, 1H), 3.16-3.03 (m, 1H), 2.02-1.87 (m, 1H), 1.61 (s, 6H), 1.60-1.53 (m, 1H).

### Illustration 3. Synthesis of (3S,4R)-4-((5-chloro-4-(4-(2-hydroxypropan-2-yl)quinolin-6-yl)pyrimidin-2-yl)amino)tetrahydro-2H-pyran-3-ol (3)

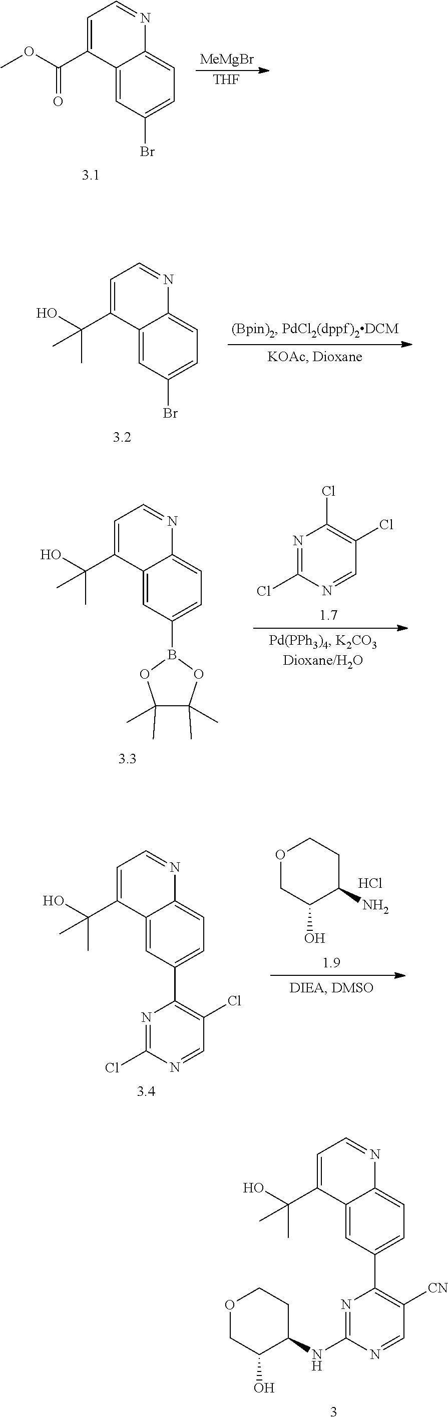

To a solution of methyl 6-bromoquinoline-4-carboxylate (3.1, 800 mg, 3.00 mmol) in anhydrous tetrahydrofuran (15 mL) was added methylmagnesium bromide (4 mL, 3 M in diethyl ether) dropwise under nitrogen atmosphere at 0° C., and the mixture was stirred at room temperature for 2 hrs under nitrogen atmosphere. The reaction mixture was quenched with sat. NH₄Cl (20 mL) solution at 0° C. and extracted with ethyl acetate (15 mL×2). The combined organic layers were washed with brine (10 mL), dried over anhydrous sodium sulfate and then concentrated under reduced pressure. The residue was subjected to silica gel column chromatography to afford 2-(6-bromoquinolin-4-yl)propan-2-ol (3.2, 564 mg, 71%). LC-MS (ESI): m/z 266.0 [M+H]⁺.

To a mixture of 2-(6-bromoquinolin-4-yl)propan-2-ol (3.2, 230 mg, 0.87 mmol) and 4,4,5,5-tetramethyl-2-(tetramethyl-1,3,2-dioxaborolan-2-yl)-1,3,2-dioxaborolane (209 mg, 0.82 mmol) in dioxane (4 mL) were added [1,1′-bis(diphenylphosphino)ferrocene]dichloropalladium(II) (36.0 mg, 0.04 mmol) and potassium acetate (255 mg, 2.60 mmol), and the mixture was stirred at 90° C. under nitrogen atmosphere for 2 hrs. The reaction mixture was cooled to room temperature, diluted with ethyl acetate (30 mL) and filtered. The filtrate was concentrated under reduced pressure and the residue was subjected to silica gel column chromatography to afford 2-(6-(4,4,5,5-tetramethyl-1,3,2-dioxaborolan-2-yl)quinolin-4-yl)propan-2-ol (3.3, 168 mg, 62%) as a yellow oil. LC-MS (ESI): m/z 314.2 [M+H]⁺.

To a mixture of 2-(6-(4,4,5,5-tetramethyl-1,3,2-dioxaborolan-2-yl)quinolin-4-yl)propan-2-ol (3.3, 168 mg, 0.54 mmol) and 2,4,5-trichloropyrimidine (1.7, 76.0 mg, 0.41 mmol) in dioxane/water (4 mL/1 mL) were added tetrakis(triphenylphosphine)palladium(0) (48.0 mg, 0.04 mmol) and potassium carbonate (114 mg, 0.83 mmol), and the mixture was stirred at 90° C. under nitrogen atmosphere for 2 hrs. The reaction mixture was cooled to room temperature, diluted with water (20 mL) and extracted with ethyl acetate (10 mL×2). The combined organic layers were washed with brine (10 mL), dried over anhydrous sodium sulfate and concentrated under reduced pressure. The residue was subjected to silica gel column chromatography to afford 2-(6-(2,5-dichloropyrimidin-4-yl)quinolin-4-yl)propan-2-ol (3.4, 103 mg, 75%). LC-MS (ESI): m/z 334.0 [M+H]⁺.

To a mixture of 2-(6-(2,5-dichloropyrimidin-4-yl)quinolin-4-yl)propan-2-ol (3.4, 50.0 mg, 0.15 mmol) and (3S,4R)-4-aminotetrahydro-2H-pyran-3-ol hydrochloride (1.9, 69.0 mg, 0.45 mmol) in anhydrous dimethylsulfoxide (2 mL) was added N,N-diisopropylethylamine (125 uL, 0.75 mmol), and the mixture was stirred at 110° C. for 2 hrs. The reaction mixture was cooled to room temperature and filtered. The filtrate was subjected to prep-HPLC to afford (3S,4R)-4-((5-chloro-4-(4-(2-hydroxypropan-2-yl)quinolin-6-yl)pyrimidin-2-yl)amino)tetrahydro-2H-pyran-3-ol (3, 10.6 mg, 17%) as a white solid. LC-MS (ESI): m/z 415.1 [M+H]⁺. ¹H NMR (400 MHz, DMSO-d₆) δ 9.31 (s, 1H), 8.89 (d, J=4.6 Hz, 1H), 8.45 (s, 1H), 8.13 (d, J=8.6 Hz, 1H), 8.09-8.00 (m, 1H), 7.59 (d, J=4.7 Hz, 1H), 7.49 (d, J=6.5 Hz, 1H), 5.60 (s, 1H), 4.93 (d, J=5.3 Hz, 1H), 3.88-3.76 (m, 3H), 3.56-3.46 (m, 1H), 3.31-3.29 (m, 1H), 3.02 (t, J=10.4 Hz, 1H), 2.05-1.91 (m, 1H), 1.72 (s, 6H), 1.57-1.43 (m, 1H).

### Illustration 4. Synthesis of (3S,4R)-4-((5-chloro-4-(4-(2-hydroxypropan-2-yl)-2-methylquinolin-6-yl)pyrimidin-2-yl)amino)tetrahydro-2H-pyran-3-ol (4)

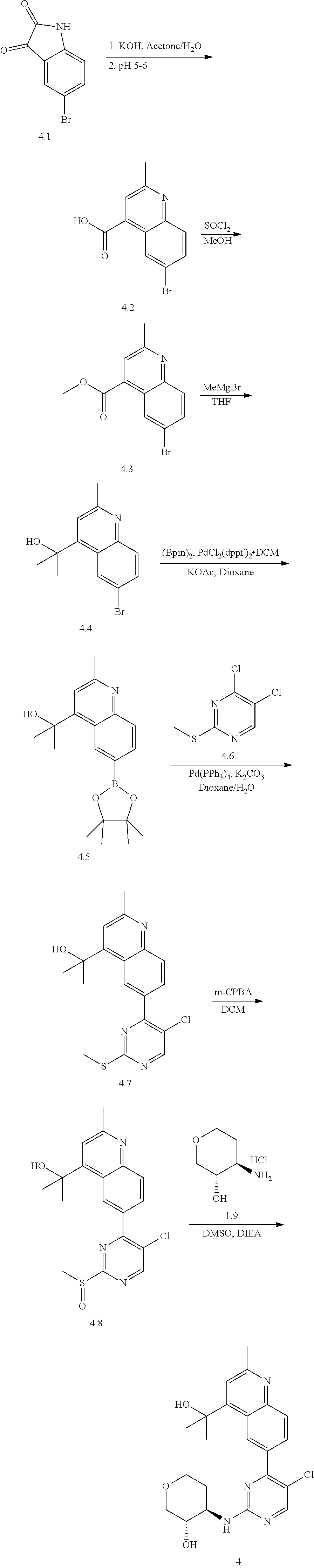

To a solution of 5-bromoindoline-2,3-dione (4.1, 2.00 g, 8.80 mmol) in acetone/water (10 mL/8 mL) was added potassium hydroxide (4.00 g, 71.0 mmol), and the mixture was stirred at 60° C. for 8 hrs. The reaction mixture was cooled to room temperature, adjusted to pH 5-6 with 10% HCl and then filtered. The filter cake was washed with warm water (10 mL) and dried in vacuum to afford 6-bromo-2-methylquinoline-4-carboxylic acid (4.2, 1.76 g, 75%). LC-MS (ESI): m/z 266.0 [M+H]⁺.

To a solution of 6-bromo-2-methylquinoline-4-carboxylic acid (4.2, 1.76 g, 6.63 mmol) in anhydrous methanol (25 mL) was added thionyl chloride (963 uL, 13.2 mmol) dropwise at 0° C., and the mixture was then stirred at 60° C. for 5 hrs. The reaction mixture was cooled to room temperature and concentrated under reduced pressure to afford methyl 6-bromo-2-methylquinoline-4-carboxylate (4.3, 1.90 g, crude). LC-MS (ESI): m/z 280.0 [M+H]⁺.

To a solution of methyl 6-bromo-2-methylquinoline-4-carboxylate (4.3, 1.90 g, crude) in anhydrous tetrahydrofuran (20 mL) was added methylmagnesium bromide (8.84 mL, 26.5 mmol, 3 M in diethyl ether) at 0° C. under nitrogen atmosphere, and the mixture was stirred at room temperature overnight. The reaction mixture was quenched with sat. NH₄Cl (20 mL) solution at 0° C. and extracted with ethyl acetate (15 mL×2). The combined organic layers were washed with brine (10 mL), dried over anhydrous sodium sulfate, filtered and concentrated under reduced pressure. The residue was subjected to silica gel column chromatography to afford 2-(6-bromo-2-methylquinolin-4-yl)propan-2-ol (4.4, 1.07 g). LC-MS (ESI): m/z 280.0 [M+H]⁺.

To a mixture of 2-(6-bromo-2-methylquinolin-4-yl)propan-2-ol (4.4, 600 mg, 2.15 mmol) and 4,4,5,5-tetramethyl-2-(tetramethyl-1,3,2-dioxaborolan-2-yl)-1,3,2-dioxaborolane (655 mg, 2.58 mmol) in dioxane (10 mL) were added [1,1′-bis(diphenylphosphino)ferrocene]dichloropalladium(II) (88.0 mg, 0.11 mmol) and potassium acetate (632 mg, 6.45 mmol). The mixture was stirred at 90° C. under nitrogen atmosphere for 2 hrs. The resulting mixture was cooled to room temperature and filtered. The filter cake was washed with ethyl acetate (10 mL) and the filtrate was concentrated under reduced pressure. The residue was subjected to silica gel column chromatography to afford 2-(2-methyl-6-(4,4,5,5-tetramethyl-1,3,2-dioxaborolan-2-yl)quinolin-4-yl)propan-2-ol (4.5, 583 mg, 83%). LC-MS (ESI): m/z 328.2 [M+H]⁺.

To a mixture of 2-(2-methyl-6-(4,4,5,5-tetramethyl-1,3,2-dioxaborolan-2-yl)quinolin-4-yl)propan-2-ol (4.5, 425 mg, 1.30 mmol) and 4,5-dichloro-2-(methylthio)pyrimidine (4.6, 195 mg, 1.00 mmol) in dioxane/water (4 mL/1 mL) were added tetrakis(triphenylphosphine)palladium(0) (115 mg, 0.10 mmol) and potassium carbonate (276 mg, 2.00 mmol). The mixture was stirred at 90° C. under nitrogen atmosphere for 2 hrs. The resulting mixture was diluted with water (15 mL) and extracted with ethyl acetate (10 mL×2). The combined organic layers were washed with brine (10 mL), dried over anhydrous sodium sulfate and concentrated under reduced pressure. The residue was subjected to silica gel column chromatography to afford 2-(6-(5-chloro-2-(methylthio)pyrimidin-4-yl)-2-methylquinolin-4-yl)propan-2-ol (4.7, 237 mg, 66%). LC-MS (ESI): m/z 360.1 [M+H]+.

To a solution of 2-(6-(5-chloro-2-(methylthio)pyrimidin-4-yl)-2-methylquinolin-4-yl)propan-2-ol (4.7, 237 mg, 0.66 mmol) in dichloromethane (5 mL) was added 3-chloroperbenzoic acid (137 mg, 0.79 mmol) at 0° C., and the mixture was stirred at room temperature for 3 hrs. The resulting mixture was diluted with dichloromethane (30 mL), washed with sat. sodium carbonate solution (10 mL×2) and brine (10 mL), dried over anhydrous sodium sulfate and then concentrated under reduced pressure to give 2-(6-(5-chloro-2-(methylsulfinyl)pyrimidin-4-yl)-2-methylquinolin-4-yl)propan-2-ol (4.8, 143 mg, crude) as a brown oil. LC-MS (ESI): m/z 376.1 [M+H]⁺.

To a mixture of 2-(6-(5-chloro-2-(methylsulfinyl)pyrimidin-4-yl)-2-methylquinolin-4-yl)propan-2-ol (4.8, 60.0 mg, crude) and (3S,4R)-4-aminotetrahydro-2H-pyran-3-ol hydrochloride (1.9, 74.0 mg, 0.48 mmol) in anhydrous dimethylsulfoxide (2 mL) was added N,N-diisopropylethylamine (130 uL, 0.80 mmol), and the resulting mixture was stirred at 110° C. for 2 hrs. The reaction mixture was cooled to room temperature and filtered, and the filtrate was subjected to prep-HPLC to afford (3S,4R)-4-((5-chloro-4-(4-(2-hydroxypropan-2-yl)-2-methylquinolin-6-yl)pyrimidin-2-yl)amino)tetrahydro-2H-pyran-3-ol (4, 5.40 mg, 8%) as a white solid. LC-MS (ESI): m/z 429.2 [M+H]⁺. ¹H NMR (400 MHz, DMSO-d₆) δ 9.23 (s, 1H), 8.44 (s, 1H), 8.01 (d, J=6.5 Hz, 2H), 7.50-7.44 (m, 2H), 5.54 (s, 1H), 4.93 (d, J=5.3 Hz, 1H), 3.86-3.77 (m, 3H), 3.55-3.48 (m, 1H), 3.34-3.32 (m, 1H), 3.02 (t, J=10.4 Hz, 1H), 2.67 (s, 3H), 2.04-1.94 (m, 1H), 1.71 (s, 6H), 1.56-1.43 (m, 1H).

### Illustration 5. Synthesis of(3S,4R)-4-((5-chloro-4-(8-fluoro-3-(2-hydroxypropan-2-yl)-2-methylquinolin-6-yl)pyrimidin-2-yl)amino)tetrahydro-2H-pyran-3-ol (5)

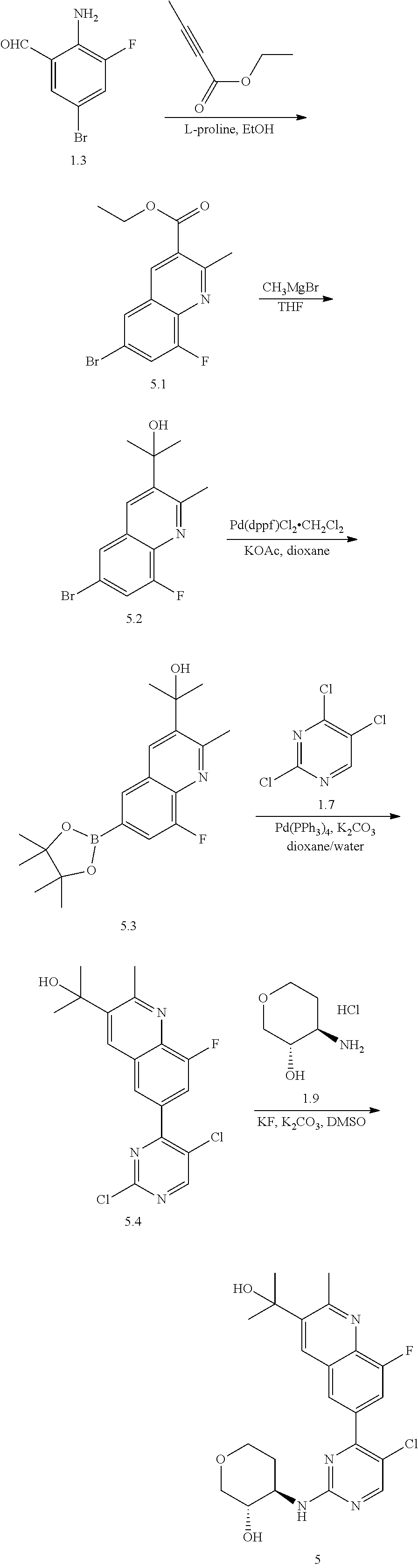

A mixture of 2-amino-5-bromo-3-fluorobenzene-1-carbaldehyde (1.3, 400 mg, 1.83 mmol), ethyl but-2-ynoate (617 mg, 5.51 mmol) and L-proline (127 mg, 1.10 mmol) in ethyl alcohol (10 mL) was stirred at 90° C. for 24 hrs. The reaction mixture was concentrated under reduced pressure and the residue was subjected to silica gel column chromatography to afford ethyl 6-bromo-8-fluoro-2-methylquinoline-3-carboxylate (5.1, 544 mg, 95%) as a yellow solid. LC-MS (ESI): m/z 312.0 [M+H]⁺. ¹H NMR (400 MHz, CDCl₃) δ 8.67 (s, 1H), 7.88 (s, 1H), 7.63 (dd, J=9.8, 2.0 Hz, 1H), 4.49 (q, J=7.1 Hz, 2H), 3.04 (s, 3H), 1.49 (t, J=7.1 Hz, 3H).

To a solution of ethyl 6-bromo-8-fluoro-2-methylquinoline-3-carboxylate (5.1, 600 mg, 1.92 mmol) in tetrahydrofuran (10 mL) was added methylmagnesium bromide (3.20 mL, 9.61 mmol, 3 M in 2-Methyltetrahydrofuran) dropwise under nitrogen atmosphere at 0° C., and the mixture was stirred at 25° C. for 4 hrs under nitrogen atmosphere. The reaction mixture was quenched with saturated ammonium chloride solution (5 mL), diluted with water (20 mL) and extracted with ethyl acetate (15 mL×3). The combined organic layers were washed with brine (20 mL), dried over anhydrous sodium sulfate and concentrated under reduced pressure. The residue was subjected to silica gel column chromatography to afford 2-(6-bromo-8-fluoro-2-methylquinolin-3-yl)propan-2-ol (5.2, 360 mg, 63%) as a yellow solid. LC-MS (ESI): m/z 298.0 [M+H]⁺. ¹H NMR (400 MHz, CDCl₃) δ 8.19 (s, 1H), 7.78 (s, 1H), 7.51 (dd, J=9.8, 2.0 Hz, 1H), 3.04 (s, 3H), 1.81 (s, 6H).

A mixture of 2-(6-bromo-8-fluoro-2-methylquinolin-3-yl)propan-2-ol (5.2, 340 mg, 1.14 mmol), 4,4,5,5-tetramethyl-2-(4,4,5,5-tetramethyl-1,3,2-dioxaborolan-2-yl)-1,3,2-dioxaborolane (347 mg, 1.37 mmol), potassium acetate (336 mg, 3.42 mmol) and 1,1′-bis(diphenylphosphino)ferrocene-palladium(II)dichloride dichloromethane complex (93.0 mg, 0.11 mmol) in dioxane (10 mL) was stirred at 90° C. for 1 hr under nitrogen atmosphere. The reaction mixture was diluted with ethyl acetate (30 mL) and filtered. The filtrate was concentrated under reduced pressure and the residue was subjected to silica gel column chromatography to afford 2-(8-fluoro-2-methyl-6-(4,4,5,5-tetramethyl-1,3,2-dioxaborolan-2-yl)quinolin-3-yl)propan-2-ol (5.3, 360 mg, 91%) as a yellow solid. LC-MS (ESI): m/z 346.2 [M+H]⁺. ¹H NMR (400 MHz, CDCl₃) δ 8.27 (s, 1H), 8.11 (s, 1H), 7.72 (d, J=11.1, 1H), 3.08 (s, 3H), 1.80 (s, 6H), 1.42 (s, 12H).

A mixture of 2-(8-fluoro-2-methyl-6-(4,4,5,5-tetramethyl-1,3,2-dioxaborolan-2-yl)quinolin-3-yl)propan-2-ol (5.3, 122 mg, 0.35 mmol), 2,4,5-trichloropyrimidine (1.7, 59.0 mg, 0.32 mmol), tetrakis(triphenylphosphine)palladium (37.2 mg, 0.03 mmol) and potassium carbonate (88.9 mg, 0.64 mmol) in dioxane/water (1 mL/0.25 mL) was stirred at 90° C. for 2 hrs under nitrogen atmosphere. The resulting mixture was concentrated under reduced pressure and the residue was subjected to silica gel column chromatography to afford 2-(6-(2,5-dichloropyrimidin-4-yl)-8-fluoro-2-methylquinolin-3-yl)propan-2-ol (5.4, 100 mg, 85%) as a yellow solid. LC-MS (ESI): m/z 366.0 [M+H]⁺. ¹H NMR (400 MHz, CDCl₃) δ 8.75 (s, 1H), 8.39 (s, 1H), 8.30 (s, 1H), 7.97 (d, J=11.3 Hz, 1H), 3.11 (s, 3H), 1.84 (s, 6H).

A mixture of 2-(6-(2,5-dichloropyrimidin-4-yl)-8-fluoro-2-methylquinolin-3-yl)propan-2-ol (5.4, 50.0 mg, 0.14 mmol), (3S,4R)-4-aminotetrahydropyran-3-ol hydrogen chloride (1.9, 41.9 mg, 0.27 mmol), potassium fluoride (31.7 mg, 0.55 mmol), 18-crown-6 (36.1 mg, 0.14 mmol) and potassium carbonate (18.9 mg, 0.14 mmol) in dimethyl sulfoxide (2 mL) was stirred at 120° C. for 2 hrs. The reaction mixture was filtered and the filtrate was subjected to prep-HPLC to afford (3S,4R)-4-((5-chloro-4-(8-fluoro-3-(2-hydroxypropan-2-yl)-2-methylquinolin-6-yl)pyrimidin-2-yl)amino)tetrahydro-2H-pyran-3-ol (5, 21.1 mg, 33%) as a white solid. LC-MS (ESI): m/z 447.2 [M+H]⁺. ¹H NMR (400 MHz, DMSO-d₆) δ 8.50 (s, 1H), 8.48 (s, 1H), 8.27 (s, 1H), 7.83 (d, J=11.7 Hz, 1H), 7.60-7.48 (m, 1H), 5.35 (s, 1H), 4.94 (d, J=5.4 Hz, 1H), 3.94-3.78 (m, 3H), 3.56-3.47 (m, 1H), 3.40-3.34 (m, 1H), 3.12-3.03 (m, 1H), 2.98 (s, 3H), 2.04-1.91 (m, 1H), 1.67 (s, 6H), 1.58-1.48 (m, 1H).

### Illustration 6. Synthesis of(3S,4R)-4-((5-chloro-4-(8-fluoro-3-(2-hydroxypropan-2-yl)-2-methylquinolin-6-yl)pyrimidin-2-yl)amino)tetrahydro-2H-pyran-3-ol (6)

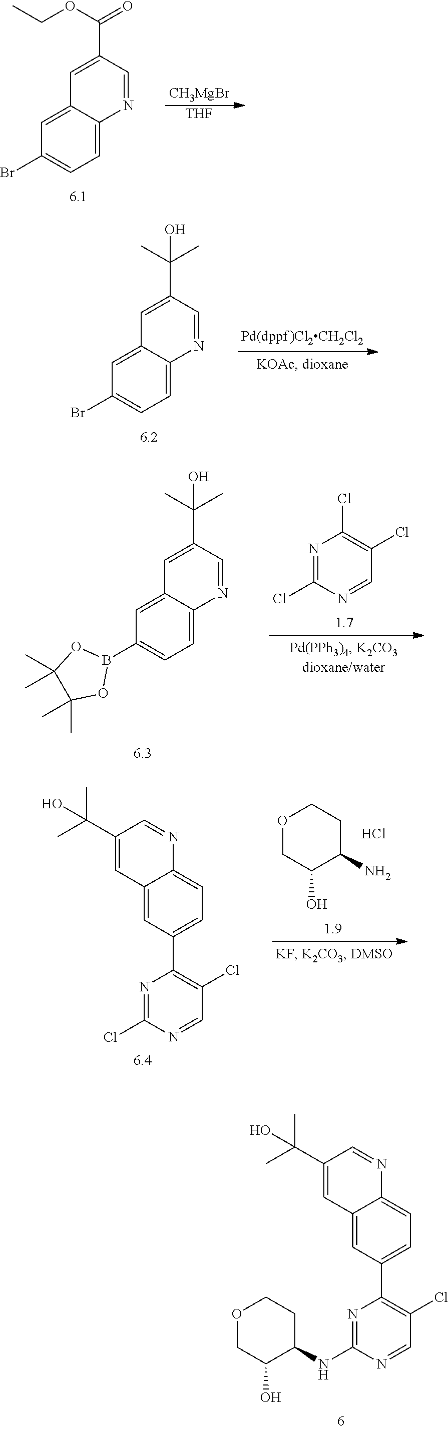

To a solution of ethyl 6-bromoquinoline-3-carboxylate (6.1, 400 mg, 1.43 mmol) in tetrahydrofuran (10 mL) was added methylmagnesium bromide (2.38 mL, 7.14 mmol, 3 M in 2-Methyltetrahydrofuran) dropwise under nitrogen atmosphere at 0° C., and the mixture was stirred at 25° C. for 2 hrs. The reaction mixture was quenched with saturated ammonium chloride solution (10 mL), diluted with water (20 mL) and extracted with ethyl acetate (15 mL×3). The combined organic layers were washed with brine (20 mL), dried over anhydrous sodium sulfate and concentrated under reduced pressure. The residue was subjected to silica gel column chromatography to afford 2-(6-bromoquinolin-3-yl)propan-2-ol (6.2, 360 mg, 1.35 mmol, 95%) as a yellow oil. LC-MS (ESI): m/z 266.0 [M+H]⁺. ¹H NMR (400 MHz, CDCl₃) δ 9.08 (d, J=2.2 Hz, 1H), 8.19 (d, J=2.2 Hz, 1H), 8.02 (d, J=2.2 Hz, 1H), 8.00 (d, J=9.0 Hz, 1H), 7.79 (dd, J=9.0, 2.2 Hz, 1H), 1.74 (s, 6H).

A mixture of 2-(6-bromoquinolin-3-yl)propan-2-ol (6.2, 360 mg, 1.35 mmol), 4,4,5,5-tetramethyl-2-(4,4,5,5-tetramethyl-1,3,2-dioxaborolan-2-yl)-1,3,2-dioxaborolane (412 mg, 1.62 mmol), potassium acetate (398 mg, 4.06 mmol) and 1,1′-bis(diphenylphosphino)ferrocene-palladium(II)dichloride dichloromethane complex (111 mg, 0.13 mmol) in dioxane (10 mL) was stirred at 90° C. for 12 hrs under nitrogen atmosphere. The reaction mixture was diluted with ethyl acetate (30 mL) and filtered. The filtrate was concentrated under reduced pressure and the residue was subjected to silica gel column chromatography to afford 2-(6-(4,4,5,5-tetramethyl-1,3,2-dioxaborolan-2-yl)quinolin-3-yl)propan-2-ol (6.3, 310 mg, 73%) as yellow oil. LC-MS (ESI): m/z 314.2 [M+H]⁺.

A mixture of 2-(6-(4,4,5,5-tetramethyl-1,3,2-dioxaborolan-2-yl)quinolin-3-yl)propan-2-ol (6.3, 310 mg, 0.99 mmol), 2,4,5-trichloropyrimidine (1.7, 150 mg, 0.82 mmol), tetrakis(triphenylphosphine)palladium (94.5 mg, 0.08 mmol) and potassium carbonate (226 mg, 1.64 mmol) in dioxane (0.8 mL) and water (0.2 mL) was stirred at 90° C. for 2 hrs under nitrogen atmosphere. The reaction mixture was concentrated under reduced pressure and the residue was subjected to silica gel column chromatography to afford 2-(6-(2,5-dichloropyrimidin-4-yl)quinolin-3-yl)propan-2-ol (6.4, 220 mg, 0.66 mmol, 88%) as a yellow oil. LC-MS (ESI): m/z 334.1 [M+H]⁺.

A mixture of 2-(6-(2,5-dichloropyrimidin-4-yl)quinolin-3-yl)propan-2-ol (6.4, 50.0 mg, 0.14 mmol), (3S,4R)-4-aminotetrahydropyran-3-ol hydrogen chloride (1.9, 41.9 mg, 0.27 mmol), potassium fluoride (31.7 mg, 0.54 mmol), 18-crown-6 (36.1 mg, 0.14 mmol) and potassium carbonate (18.9 mg, 0.14 mmol) in dimethyl sulfoxide (2 mL) was stirred at 120° C. for 2 hrs. The reaction mixture was filtered and the filtrate was subjected to prep-HPLC to afford (3S,4R)-4-((5-chloro-4-(3-(2-hydroxypropan-2-yl)quinolin-6-yl)pyrimidin-2-yl)amino)tetrahydro-2H-pyran-3-ol (6, 16.4 mg, 26%) as a white solid. LC-MS (ESI): m/z 415.2 [M+H]⁺. ¹H NMR (400 MHz, DMSO-d₆) δ 9.16 (d, J=2.3 Hz, 1H), 8.48 (s, 1H), 8.47 (d, J=2.2 Hz, 1H), 8.40 (s, 1H), 8.12 (d, J=8.7 Hz, 1H), 8.03 (d, J=8.8 Hz, 1H), 7.53 (s, 1H), 5.43 (s, 1H), 4.94 (d, J=5.4 Hz, 1H), 3.93-3.76 (m, 3H), 3.59-3.46 (m, 1H), 3.40-3.35 (m, 1H), 3.07 (t, J=10.3 Hz, 1H), 2.02-1.92 (m, 1H), 1.60 (s, 6H), 1.57-1.47 (m, 1H).

### Illustration 7. Synthesis of(3S,4R)-4-((5-chloro-4-(8-fluoro-3-(2-hydroxypropan-2-yl)-4-methylquinolin-6-yl)pyrimidin-2-yl)amino)tetrahydro-2H-pyran-3-ol (7)

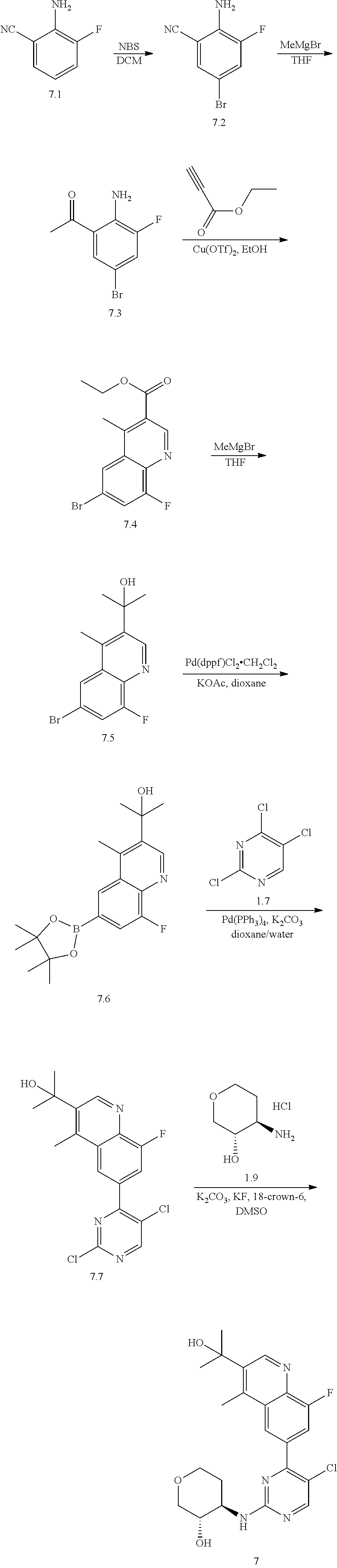

To a solution of 2-amino-3-fluorobenzene-1-carbonitrile (7.1, 1.00 g, 7.35 mmol) in dichloromethane (20 mL) was added N-bromosuccinimide (1.37 g, 7.71 mmol), and the mixture was stirred at 25° C. for 12 hrs. The reaction mixture was concentrated under reduced pressure and the residue was subjected to silica gel column chromatography to afford 2-amino-5-bromo-3-fluorobenzene-1-carbonitrile (7.2, 1.40 g, 89%) as a purple solid. LC-MS (ESI): m/z 215.0 [M+H]⁺. ¹H NMR (400 MHz, CDCl₃) δ 7.40-7.31 (m, 2H), 4.57 (brs, 2H).

To a solution of 2-amino-5-bromo-3-fluorobenzene-1-carbonitrile (7.2, 400 mg, 1.86 mmol) in tetrahydrofuran (5 mL) was added methylmagnesium bromide (3.10 mL, 9.30 mmol, 3 M in 2-Methyltetrahydrofuran) dropwise under nitrogen atmosphere at 0° C., and the mixture was stirred at 25° C. for 12 hrs under nitrogen atmosphere. The reaction mixture was quenched with saturated ammonium chloride solution (5 mL), diluted with water (20 mL) and extracted with ethyl acetate (20 mL×3). The combined organic layers were washed with brine (10 mL), dried over anhydrous sodium sulfate and concentrated under reduced pressure. The residue was subjected to silica gel column chromatography to afford 1-(2-amino-5-bromo-3-fluorophenyl)ethan-1-one (7.3, 330 mg, 76%) as a yellow solid. LC-MS (ESI): m/z 232.0 [M+H]⁺. ¹H NMR (400 MHz, DMSO-d₆) δ 7.78 (s, 1H), 7.57 (dd, J=11.0, 2.2 Hz, 1H), 7.21 (s, 2H), 2.57 (s, 3H).

A mixture of 1-(2-amino-5-bromo-3-fluorophenyl)ethan-1-one (7.3, 300 mg, 1.29 mmol), ethyl prop-2-ynoate (1.02 g, 10.34 mmol) and cupric bis(trifluoromethanesulfonate) (187 mg, 0.52 mmol) in ethanol (4 mL) was stirred at 90° C. for 12 hrs. The reaction mixture was concentrated under reduced pressure and the residue was subjected to silica gel column chromatography to afford ethyl 6-bromo-8-fluoro-4-methylquinoline-3-carboxylate (7.4, 120 mg, 30%) as a yellow solid. LC-MS (ESI): m/z 312.0 [M+H]⁺.

To a solution of ethyl 6-bromo-8-fluoro-4-methylquinoline-3-carboxylate (7.4, 300 mg, 1.07 mmol) in tetrahydrofuran (5 mL) was added methylmagnesium bromide (1.44 mL, 4.33 mmol, 3 M in 2-Methyltetrahydrofuran) dropwise under nitrogen atmosphere at 0° C., and the mixture was stirred at 25° C. for 1 hr under nitrogen atmosphere. The reaction mixture was quenched with saturated ammonium chloride solution (5 mL), diluted with water (20 mL) and extracted with ethyl acetate (20 mL×3). The combined organic layers were washed with brine (10 mL), dried over anhydrous sodium sulfate and concentrated under reduced pressure. The residue was subjected to silica gel column chromatography to afford 2-(6-bromo-8-fluoro-4-methylquinolin-3-yl)propan-2-ol (7.5, 70 mg, 27%) as a yellow oil. LC-MS (ESI): m/z 298.0 [M+H]⁺.

A mixture of 2-(6-bromo-8-fluoro-4-methylquinolin-3-yl)propan-2-ol (7.5, 70.0 mg, 0.23 mmol), 4,4,5,5-tetramethyl-2-(4,4,5,5-tetramethyl-1,3,2-dioxaborolan-2-yl)-1,3,2-dioxaborolane (71.6 mg, 0.28 mmol), potassium acetate (69 mg, 0.704 mmol) and 1,1′-bis(diphenylphosphino)ferrocene-palladium(II)dichloride dichloromethane complex (19.2 mg, 0.02 mmol) in dioxane (10 mL) was stirred at 90° C. for 1 hr under nitrogen atmosphere. The reaction mixture was cooled to room temperature, diluted with ethyl acetate (30 mL) and filtered. The filtrate was concentrated under reduced pressure and the residue was subjected to silica gel column chromatography to afford 2-(8-fluoro-4-methyl-6-(4,4,5,5-tetramethyl-1,3,2-dioxaborolan-2-yl)quinolin-3-yl)propan-2-ol (7.6, 40.0 mg, 49%) as a yellow oil. LC-MS (ESI): m/z 346.2 [M+H]⁺.

A mixture of 2-(8-fluoro-4-methyl-6-(4,4,5,5-tetramethyl-1,3,2-dioxaborolan-2-yl)quinolin-3-yl)propan-2-ol (7.6, 37.6 mg, 0.11 mmol), 2,4,5-trichloropyrimidine (1.7, 20.0 mg, 0.11 mmol), tetrakis(triphenylphosphine)palladium (12.6 mg, 0.01 mmol) and potassium carbonate (30.1 mg, 0.22 mmol) in dioxane (1 mL) and water (0.2 mL) was stirred at 90° C. for 2 hrs under nitrogen atmosphere. The reaction mixture was concentrated under reduced pressure and the residue was subjected to silica gel column chromatography to afford 2-(6-(2,5-dichloropyrimidin-4-yl)-8-fluoro-4-methylquinolin-3-yl)propan-2-ol (7.7, 30.0 mg, 75%) as a yellow oil. LC-MS (ESI): m/z 366.1 [M+H]⁺.

A mixture of 2-(6-(2,5-dichloropyrimidin-4-yl)-8-fluoro-4-methylquinolin-3-yl)propan-2-ol (7.7, 30.0 mg, 0.08 mmol), (3S,4R)-4-aminotetrahydropyran-3-ol hydrogen chloride (1.9, 19.0 mg, 0.12 mmol), potassium fluoride (19.0 mg, 0.33 mmol), 18-crown-6 (21.7 mg, 0.08 mmol) and potassium carbonate (11.3 mg, 0.08 mmol) in dimethyl sulfoxide (2 mL) was stirred at 120° C. for 2 hrs. The reaction mixture was filtered and the filtrate was subjected to prep-HPLC to afford (3S,4R)-4-((5-chloro-4-(8-fluoro-3-(2-hydroxypropan-2-yl)-4-methylquinolin-6-yl)pyrimidin-2-yl)amino)tetrahydro-2H-pyran-3-ol (7, 5.50 mg, 15%) as a white solid. LC-MS (ESI): m/z 447.2 [M+H]⁺. ¹H NMR (400 MHz, DMSO-d₆) δ 9.14 (s, 1H), 8.56-8.38 (m, 2H), 7.88 (d, J=11.1 Hz, 1H), 7.58 (s, 1H), 5.43 (s, 1H), 4.96 (d, J=5.4 Hz, 1H), 3.95-3.77 (m, 3H), 3.61-3.47 (m, 1H), 3.44-3.37 (m, 1H), 3.07 (t, J=10.4 Hz, 1H), 2.98 (s, 3H), 2.07-1.87 (m, 1H), 1.70 (s, 6H), 1.61-1.46 (m, 1H).

### Illustration 8. Synthesis of (3S,4R)-4-((5-chloro-4-(7-fluoro-1,2,3,4-tetrahydrobenzo[c][2,7]naphthyridin-9-yl)pyrimidin-2-yl)amino)tetrahydro-2H-pyran-3-ol (8)

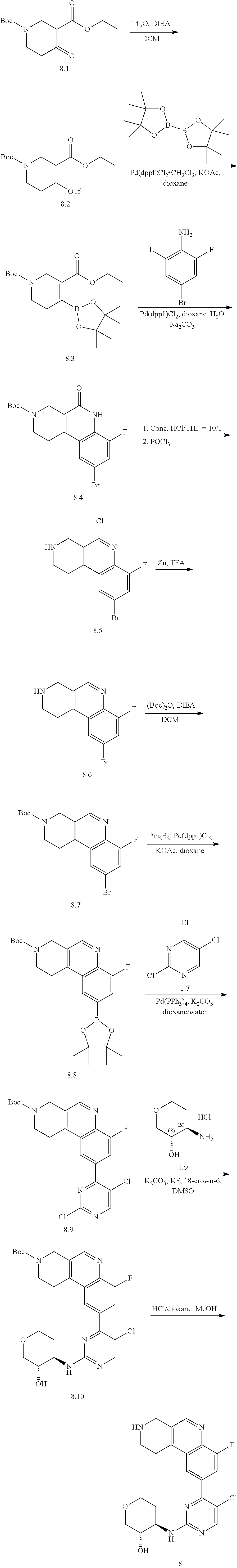

To a mixture of 1-(tert-butyl) 3-ethyl 4-oxopiperidine-1,3-dicarboxylate (8.1, 15.0 g, 55.3 mmol) and N,N-diisopropylethylamine (21.4 g, 166 mmol, 28.9 mL) in dichloromethane (400 mL) was added a solution of trifluoromethanesulfonic anhydride (18.7 g, 66.4 mmol, 11 mL) in dichloromethane (50 mL) dropwise at 0° C., and the resulting mixture was stirred at 0° C. for 2 hrs under nitrogen atmosphere. The reaction mixture was quenched with saturated sodium bicarbonate solution (200 mL) and extracted with dichloromethane (100 mL×3). The combined organic layers were dried over anhydrous sodium sulfate, filtered and concentrated under reduced pressure. The residue was subjected to silica gel column chromatography to afford 1-(tert-butyl) 3-ethyl 4-(((trifluoromethyl)sulfonyl)oxy)-5,6-dihydropyridine-1,3(2H)-dicarboxylate (8.2, 17.0 g, 69%) as a yellow oil. LC-MS (ESI): m/z 404.1 [M+H]⁺.

To a solution of 1-(tert-butyl) 3-ethyl 4-(((trifluoromethyl)sulfonyl)oxy)-5,6-dihydropyridine-1,3(2H)-dicarboxylate (8.2, 17.0 g, 42.1 mmol) in dioxane (300 mL) were added 4,4,5,5-tetramethyl-2-(4,4,5,5-tetramethyl-1,3,2-dioxaborolan-2-yl)-1,3,2-dioxaborolane (11.8 g, 46.4 mmol), potassium acetate (12.4 g, 126 mmol) and Pd(dppf)Cl₂·CH₂Cl₂ (3.44 g, 4.21 mmol), and the mixture was stirred at 110° C. for 3 hrs under nitrogen atmosphere. The reaction mixture was diluted with water (300 mL) and extracted with ethyl acetate (100 mL×3). The combined organic layers were dried over anhydrous sodium sulfate, filtered and concentrated under reduced pressure. The residue was subjected to silica gel column chromatography to afford 1-(tert-butyl) 3-ethyl 4-(4,4,5,5-tetramethyl-1,3,2-dioxaborolan-2-yl)-5,6-dihydropyridine-1,3(2H)-dicarboxylate (8.3, 11.0 g, 58%) as a yellow oil. LC-MS (ESI): m/z 382.2 [M+H]⁺.

To a solution of 4-bromo-2-fluoro-6-iodoaniline (9.11 g, 28.9 mmol) and 1-(tert-butyl) 3-ethyl 4-(4,4,5,5-tetramethyl-1,3,2-dioxaborolan-2-yl)-5,6-dihydro-pyridine-1,3(2H)-dicarboxylate (8.3, 11.0 g, 28.9 mmol) in dioxane (150 mL) and water (15 mL) were added sodium carbonate (9.17 g, 86.6 mmol) and Pd(dppf)Cl₂ (1.06 g, 1.44 mmol), and the resulting mixture was stirred at 50° C. for 3 hrs under nitrogen atmosphere. The reaction mixture was diluted with water (300 mL) and extracted with dichloromethane (100 mL×3). The combined organic layers were dried over anhydrous sodium sulfate, filtered and concentrated under reduced pressure. The residue was triturated with ethyl alcohol (60 mL) at 25° C. for 1 hr to give tert-butyl 9-bromo-7-fluoro-5-oxo-1,4,5,6-tetrahydrobenzo[c][2,7]naphthyridine-3(2H)-carboxylate (8.4, 4.50 g, 33%) as a white solid. LC-MS (ESI): m/z 397.1 [M+H]⁺. ¹H NMR (400 MHz CDCl₃) δ 9.96-9.50 (m, 1H), 7.56 (s, 1H), 7.40 (d, J=12.5 Hz, 1H), 4.48 (s, 2H), 3.85-3.75 (m, 2H), 2.89 (s, 2H), 1.49 (s, 9H).

To a solution of tert-butyl 9-bromo-7-fluoro-5-oxo-1,4,5,6-tetrahydrobenzo[c][2,7]naphthyridine-3(2H)-carboxylate (8.4, 4.50 g, 11.3 mmol) in dioxane (55 mL) were added phosphorus oxychloride (8.68 g, 56.6 mmol) and N,N-diisopropylethylamine (7.32 g, 56.6 mmol), and the mixture was stirred at 100° C. for 2 hrs. The reaction mixture was concentrated under reduced pressure, and the residue was diluted with tetrahydrofuran (4 mL) and conc. HCl (40 mL, 12 M). The resulting mixture was then stirred at 100° C. for 30 mins. The mixture was concentrated under reduced pressure and the residue was triturated with saturated sodium bicarbonate solution (50 mL). The filter cake was dried in vacuum, diluted with phosphorus oxychloride (20 mL) and then stirred at 100° C. for another 2 hrs. The mixture was concentrated under reduced pressure and triturated with acetonitrile/methyl tert-butyl ether=10/1 (50 mL) at 25° C. for 1 hr. The filter cake was diluted with saturated sodium bicarbonate solution (50 mL) and extracted with dichloromethane (30 mL×3). The combined organic layers were dried over anhydrous sodium sulfate, filtered and concentrated under reduced pressure. The residue was triturated with methyl tert-butyl ether (20 mL) at 25° C. for 1 hr and the filter cake was dried in vacuum to afford 9-bromo-5-chloro-7-fluoro-1,2,3,4-tetrahydrobenzo[c][2,7]naphthyridine (8.5, 2.50 g, 56%) as a yellow solid. LC-MS (ESI): m/z 315.0 [M+H]⁺.

To a solution of 9-bromo-5-chloro-7-fluoro-1,2,3,4-tetrahydrobenzo[c][2,7]naphthyridine (8.5, 1.00 g, 3.17 mmol) in trifluoroacetic acid (10 mL) was added zinc dust (2.07 g, 31.7 mmol) in portions, and the mixture was stirred at 40° C. for 1 hr. The reaction mixture was diluted with water (50 mL) and dichloromethane (30 mL), adjusted to pH 11 with sodium hydroxide (solid) and then filtered. The filtrate was extracted with dichloromethane (30 mL×3). The combined organic layers were dried over anhydrous sodium sulfate, filtered and concentrated under reduced pressure to afford 9-bromo-7-fluoro-1,2,3,4-tetrahydrobenzo[c][2,7]naphthyridine (8.6, 750 mg, crude) as a yellow solid. LC-MS (ESI): m/z 281.1 [M+H]⁺.

To a solution of 9-bromo-7-fluoro-1,2,3,4-tetrahydrobenzo[c][2,7]naphthyridine (8.6, 3.00 g, crude) in dichloromethane (20 mL) were added N,N-diisopropylethylamine (4.14 g, 32.0 mmol) and di-tert-butyl dicarbonate (2.79 g, 12.8 mmol), and the mixture was stirred at 25° C. for 1 hr. The reaction mixture was diluted with water (50 mL) and extracted with dichloromethane (30 mL×3). The combined organic layers were dried over anhydrous sodium sulfate, filtered and concentrated under reduced pressure. The residue was subjected to silica gel column chromatography and further purified by prep-TLC to afford tert-butyl 9-bromo-7-fluoro-1,4-dihydrobenzo[c][2,7]naphthyridine-3(2H)-carboxylate (8.7, 0.50 g) as a light yellow solid. LC-MS (ESI): m/z 381.1 [M+H]⁺.

To a solution of tert-butyl 9-bromo-7-fluoro-1,4-dihydrobenzo[c][2,7]naphthyridine-3(2H)-carboxylate (8.7, 0.50 g, 1.31 mmol) in dioxane (1 mL) were added 4,4,5,5-tetramethyl-2-(4,4,5,5-tetramethyl-1,3,2-dioxaborolan-2-yl)-1,3,2-dioxaborolane (366 mg, 1.44 mmol), potassium acetate (386 mg, 3.93 mmol), and Pd(dppf)Cl₂ (96.0 mg, 131 umol), and the mixture was stirred at 90° C. for 1 hr under nitrogen atmosphere. The reaction mixture was diluted with water (20 mL) and extracted with ethyl acetate (10 mL×3). The combined organic layers were dried over anhydrous sodium sulfate, filtered and concentrated under reduced pressure. The residue was subjected to silica gel column chromatography to afford tert-butyl 7-fluoro-9-(4,4,5,5-tetramethyl-1,3,2-dioxaborolan-2-yl)-1,4-dihydrobenzo[c][2,7]naphthyridine-3(2H)-carboxylate (8.8, 0.35 g, 58%) as a white solid. LC-MS (ESI): m/z 429.0 [M+H]⁺.

To a mixture of tert-butyl 7-fluoro-9-(4,4,5,5-tetramethyl-1,3,2-dioxaborolan-2-yl)-1,4-dihydrobenzo[c][2,7]naphthyridine-3(2H)-carboxylate (8.8, 100 mg, 0.23 mmol) and 2,4,5-trichloropyrimidine (1.7, 26.8 μL, 0.23 mmol) in dioxane (1 mL) and water (0.2 mL) were added Pd(PPh₃)₄ (54.0 mg, 0.05 mmol) and potassium carbonate (64.5 mg, 0.47 mmol), and the reaction was stirred at 90° C. for 4 hrs under nitrogen atmosphere. The mixture was diluted with water (10 mL), extracted with ethyl acetate (10 mL×2). The organic layer was dried over anhydrous sodium sulfate, filtered and concentrated under reduced pressure. The residue was subjected to silica gel column chromatography to afford tert-butyl 9-(2,5-dichloropyrimidin-4-yl)-7-fluoro-1,4-dihydrobenzo[c][2,7]naphthyridine-3(2H)-carboxylate (8.9, 72.0 mg, 68%) as a white solid. LC-MS (ESI): m/z 449.1 [M+H]⁺.

A mixture of tert-butyl 9-(2,5-dichloropyrimidin-4-yl)-7-fluoro-1,4-dihydrobenzo[c][2,7]naphthyridine-3(2H)-carboxylate (8.9, 110 mg, 0.25 mmol), (3S,4R)-4-aminotetrahydropyran-3-ol hydrochloride (1.9, 37.6 mg, 0.25 mmol), potassium carbonate (33.8 mg, 0.25 mmol), potassium fluoride (42.7 mg, 0.73 mmol) and 18-crown-6 (6.47 mg, 0.02 mmol) in dimethylsulfoxide (1 mL) was stirred at 120° C. for 1 hr. The mixture was diluted with water (10 mL) and extracted with ethyl acetate (10 mL×2). The combined organic layers were washed with water (10 mL), dried over anhydrous sodium sulfate and concentrated under reduced pressure. The residue was subjected to silica gel column chromatography to afford tert-butyl 9-(5-chloro-2-(((3S,4R)-3-hydroxytetrahydro-2H-pyran-4-yl)amino)pyrimidin-4-yl)-7-fluoro-1,4-dihydrobenzo[c][2,7]naphthyridine-3(2H)-carboxylate (8.10, 100 mg, 77%) as a white solid. LC-MS (ESI): m/z 530.2 [M+H]⁺.

To a solution of tert-butyl 9-(5-chloro-2-(((3S,4R)-3-hydroxytetrahydro-2H-pyran-4-yl)amino)pyrimidin-4-yl)-7-fluoro-1,4-dihydrobenzo[c][2,7]naphthyridine-3(2H)-carboxylate (8.10, 100 mg, 0.19 mmol) in methanol (1 mL) was added hydrochloric acid (1 mL, 4 M in dioxane), and the mixture was stirred at room temperature for 1 hr. The reaction mixture was concentrated under reduced pressure, adjusted to pH 7 with saturated sodium bicarbonate solution (5 mL) and extracted with ethyl acetate (20 mL×2). The combined organic layers were dried over anhydrous sodium sulfate, filtered and concentrated under reduced pressure. The residue was subjected to prep-HPLC to afford (3S,4R)-4-((5-chloro-4-(7-fluoro-1,2,3,4-tetrahydrobenzo[c][2,7]naphthyridin-9-yl)pyrimidin-2-yl)amino)tetrahydro-2H-pyran-3-ol (8, 52.0 mg, 64%) as a white solid. LC-MS (ESI): m/z 430.3 [M+H]⁺. ¹H NMR (400 MHz, DMSO-d₆) δ 8.76 (s, 1H), 8.47 (s, 1H), 8.21 (s, 1H), 7.87 (d, J=11.4 Hz, 1H), 7.64-7.49 (m, 1H), 4.16 (s, 2H), 3.83-3.79 (m, 3H), 3.53-3.50 (m, 1H), 3.37-3.30 (m, 1H), 3.24-3.11 (m, 4H), 3.05 (t, J=10.4 Hz, 1H), 2.02-1.89 (m, 1H), 1.58-1.44 (m, 1H).

### Illustration 9. Synthesis of (3S,4R)-4-((5-chloro-4-(7-fluoro-1,2,3,4-tetrahydrobenzo[c][2,6]naphthyridin-9-yl)pyrimidin-2-yl)amino)tetrahydro-2H-pyran-3-ol (9)

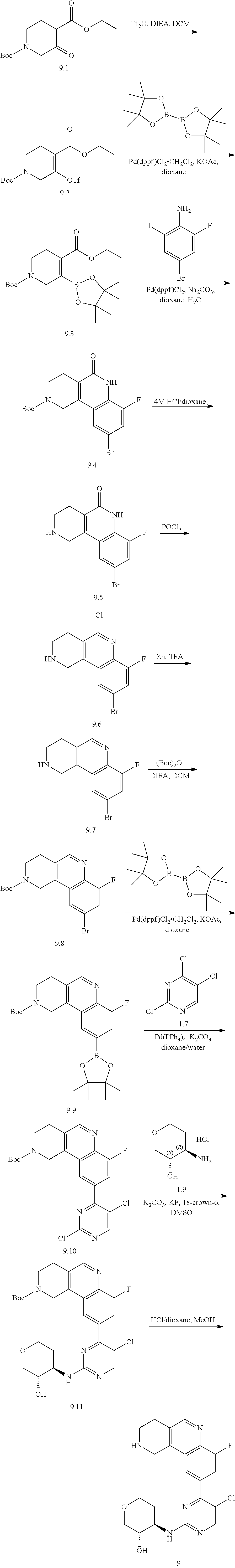

To a mixture of 1-(tert-butyl) 4-ethyl 3-oxopiperidine-1,4-dicarboxylate (9.1, 25.0 g, 92.2 mmol) and N,N-diisopropylethylamine (35.7 g, 276 mmol, 48.2 mL) in dichloromethane (600 mL) was added a solution of trifluoromethanesulfonic anhydride (31.2 g, 111 mmol, 18.2 mL) in dichloromethane (70 mL) dropwise at 0° C. The resulting mixture was stirred at 0° C. for 2 hrs under nitrogen atmosphere. The reaction mixture was diluted with water (500 mL) and extracted with dichloromethane (200 mL×3). The combined organic layers were dried over anhydrous sodium sulfate, filtered and concentrated under reduced pressure. The residue was subjected to silica gel column chromatography to afford 1-(tert-butyl) 4-ethyl 5-(((trifluoromethyl)sulfonyl)oxy)-3,6-dihydropyridine-1,4(2H)-dicarboxylate (9.2, 35.0 g, 85%) as a yellow oil. LC-MS (ESI): m/z 304.1 [M-100+H]⁺.

To a solution of 1-(tert-butyl) 4-ethyl 5-(((trifluoromethyl)sulfonyl)oxy)-3,6-dihydropyridine-1,4(2H)-dicarboxylate (9.2, 35.0 g, 86.8 mmol) in dioxane (500 mL) were added 4,4,5,5-tetramethyl-2-(4,4,5,5-tetramethyl-1,3,2-dioxaborolan-2-yl)-1,3,2-dioxaborolane (24.2 g, 95.5 mmol), potassium acetate (25.6 g, 260 mmol) and Pd(dppf)Cl₂·CH₂Cl₂ (7.09 g, 8.68 mmol), and the mixture was stirred at 110° C. for 3 hrs under nitrogen atmosphere. The reaction mixture was diluted with water (700 mL) and extracted with ethyl acetate (300 mL×3). The combined organic layers were dried over anhydrous sodium sulfate, filtered and concentrated under reduced pressure. The residue was subjected to silica gel column chromatography to afford 1-(tert-butyl) 4-ethyl 5-(4,4,5,5-tetramethyl-1,3,2-dioxaborolan-2-yl)-3,6-dihydropyridine-1,4(2H)-dicarboxylate (9.3, 30.0 g, 77%) as a yellow oil. LC-MS (ESI): m/z 282.2 [M-100+H]⁺.

To a mixture of 4-bromo-2-fluoro-6-iodoaniline (8.29 g, 26.2 mmol) and 1-(tert-butyl) 4-ethyl 5-(4,4,5,5-tetramethyl-1,3,2-dioxaborolan-2-yl)-3,6-dihydropyridine-1,4(2H)-dicarboxylate (9.3, 10.0 g, 26.2 mmol) in dioxane (100 mL) and water (10 mL) were added sodium carbonate (8.34 g, 78.7 mmol) and Pd(dppf)Cl₂ (960 mg, 1.31 mmol) under nitrogen atmosphere, and the resulting mixture was stirred at 50° C. for 3 hrs under nitrogen atmosphere. The reaction mixture was diluted with water (200 mL) and extracted with dichloromethane (100 mL×3). The combined organic layers were dried over anhydrous sodium sulfate, filtered and concentrated under reduced pressure. The residue was triturated with ethyl alcohol (50 mL) at 25° C. for 1 hr and then filtered. The filter cake was dried in vacuum to afford tert-butyl 9-bromo-7-fluoro-5-oxo-3,4,5,6-tetrahydrobenzo[c][2,6]naphthyridine-2(1H)-carboxylate (9.4, 8.00 g, 65%) as an off-white solid. LC-MS (ESI): m/z 397.0 [M+H]⁺.

A mixture of tert-butyl 9-bromo-7-fluoro-5-oxo-3,4,5,6-tetrahydrobenzo[c][2,6]naphthyridine-2(1H)-carboxylate (9.4, 8.00 g, 20.1 mmol) and hydrochloric acid (100 mL, 4 M in dioxane) was stirred at 25° C. for 2 hrs. The reaction mixture was concentrated under reduced pressure, diluted with saturated sodium bicarbonate solution (100 mL) and extracted with dichloromethane (50 mL×3). The combined organic layers were washed with water (50 mL), dried over anhydrous sodium sulfate and filtered. The filtrate was concentrated under reduced pressure to give 9-bromo-7-fluoro-2,3,4,6-tetrahydrobenzo[c][2,6]naphthyridin-5(1H)-one (9.5, 5.00 g, crude) as a yellow solid. LC-MS (ESI): m/z 297.0 [M+H]⁺.

A mixture of 9-bromo-7-fluoro-2,3,4,6-tetrahydrobenzo[c][2,6]naphthyridin-5(1H)-one (9.5, 5.00 g, crude) and phosphorus oxychloride (50 mL) was stirred at 100° C. for 2 hrs. The reaction mixture was concentrated under reduced pressure, and the residue was diluted with acetonitrile (10 mL) and then triturated with methyl tert-butyl ether (100 mL) at 25° C. for 30 mins. The resulting mixture was filtered and the filter cake was dried in vacuum to give a residue. The residue was diluted with saturated sodium bicarbonate solution (200 mL) and extracted with dichloromethane/methanol=10/1 (100 mL×3). The combined organic layers were dried over anhydrous sodium sulfate, filtered and concentrated under reduced pressure to afford 9-bromo-5-chloro-7-fluoro-1,2,3,4-tetrahydrobenzo[c][2,6]naphthyridine (9.6, 2.70 g, crude) as a yellow solid. LC-MS (ESI): m/z 314.9 [M+H]⁺.

To a solution of 9-bromo-5-chloro-7-fluoro-1,2,3,4-tetrahydrobenzo[c][2,6]naphthyridine (9.6, 50.0 mg, crude) in trifluoroacetic acid (1 mL) was added zinc dust (73.0 mg, 1.11 mmol), and the mixture was stirred at 40° C. for 1 hr. The reaction mixture was diluted with water (100 mL) and dichloromethane (100 mL) and then adjusted to pH 11 with 10 M sodium hydroxide solution. The resulting mixture was filtered and the filtrate was extracted with dichloromethane (50 mL×3). The combined organic layers were dried over anhydrous sodium sulfate, filtered and concentrated under reduced pressure to afford 9-bromo-7-fluoro-1,2,3,4-tetrahydrobenzo[c][2,6]naphthyridine (9.7, 1.60 g, crude) as a yellow solid. LC-MS (ESI): m/z 281.1 [M+H]⁺.

To a solution of 9-bromo-7-fluoro-1,2,3,4-tetrahydrobenzo[c][2,6]naphthyridine (9.7, 1.60 g, crude) in dichloromethane (20 mL) were added di-tert-butyl dicarbonate (1.49 g, 6.83 mmol, 1.57 mL) and N,N-diisopropylethylamine (2.21 g, 17.1 mmol, 2.97 mL), and the mixture was stirred at 25° C. for 2 hrs. The reaction mixture was diluted with water (50 mL) and extracted with dichloromethane (20 mL×3). The combined organic layers were dried over anhydrous sodium sulfate, filtered and concentrated under reduced pressure. The residue was subjected to silica gel column chromatography and further purified by prep-TLC to afford tert-butyl 9-bromo-7-fluoro-3,4-dihydrobenzo[c][2,6]naphthyridine-2(1H)-carboxylate (9.8, 0.45 g) as a white solid. LC-MS (ESI): m/z 381.0 [M+H]⁺.

To a solution of tert-butyl 9-bromo-7-fluoro-3,4-dihydrobenzo[c][2,6]naphthyridine-2(1H)-carboxylate (9.8, 0.44 g, 1.15 mmol) in dioxane (5 mL) were added 4,4,5,5-tetramethyl-2-(4,4,5,5-tetramethyl-1,3,2-dioxaborolan-2-yl)-1,3,2-dioxaborolane (322 mg, 1.27 mmol), potassium acetate (340 mg, 3.46 mmol) and Pd(dppf)Cl₂ (84.5 mg, 115 umol). The resulting mixture was stifred at 90° C. for 2 hrs under nitrogen atmosphere. The reaction mixture was diluted with water (20 mL) and extracted with ethyl acetate (10 mL×3). The combined organic layers were dried over anhydrous sodium sulfate, filtered and concentrated under reduced pressure. The residue was subjected to silica gel column chromatography to afford tert-butyl 7-fluoro-9-(4,4,5,5-tetramethyl-1,3,2-dioxaborolan-2-yl)-3,4-dihydrobenzo[c][2,6]naphthyridine-2(1H)-carboxylate (9.9, 390 mg, 63%) as a yellow solid. LC-MS (ESI): m/z 429.2 [M+H]⁺.

To a mixture of tert-butyl 7-fluoro-9-(4,4,5,5-tetramethyl-1,3,2-dioxaborolan-2-yl)-3,4-dihydrobenzo[c][2,6]naphthyridine-2(1H)-carboxylate (9.9, 100 mg, 0.29 mmol) and 2,4,5-trichloropyrimidine (1.7, 33 μL, 0.29 mmol) in water (0.2 mL) and dioxane (1 mL) were added Pd(PPh₃)₄ (67.0 mg, 0.06 mmol) and potassium carbonate (120 mg, 0.87 mmol). The reaction mixture was stirred at 90° C. for 4 hrs under nitrogen atmosphere. The mixture was diluted with water (20 mL) and extracted with ethyl acetate (10 mL×2). The combined organic layers were dried over anhydrous sodium sulfate, filtered and concentrated under reduced pressure. The residue was subjected to silica gel column chromatography to afford tert-butyl 9-(2,5-dichloropyrimidin-4-yl)-7-fluoro-3,4-dihydrobenzo[c][2,6]naphthyridine-2(1H)-carboxylate (9.10, 130 mg, crude) as a yellow solid. LC-MS (ESI): m/z 449.3 [M+H]⁺.

A mixture of tert-butyl 9-(2,5-dichloropyrimidin-4-yl)-7-fluoro-3,4-dihydrobenzo[c][2,6]naphthyridine-2(1H)-carboxylate (9.10, 130 mg, crude), (3S,4R)-4-aminotetrahydro-2H-pyran-3-ol hydrochloride (1.9, 44.0 mg, 0.29 mmol), potassium carbonate (40.0 mg, 0.29 mmol), potassium fluoride (50.0 mg, 0.87 mmol) and 18-crown-6 (8 mg, 0.03 mmol) in dimethylsulfoxide (2 mL) was stirred at 120° C. for 1 hr. The mixture was diluted with water (20 mL) and extracted with ethyl acetate (10 mL×2). The combined organic layers were dried over anhydrous sodium sulfate, filtered and concentrated under reduced pressure. The residue was subjected to silica gel column chromatography to afford tert-butyl 9-(5-chloro-2-(((3S,4R)-3-hydroxytetrahydro-2H-pyran-4-yl)amino)pyrimidin-4-yl)-7-fluoro-3,4-dihydrobenzo[c][2,6]naphthyridine-2(1H)-carboxylate (9.11, 150 mg, crude) as a white solid was afforded. LC-MS (ESI): m/z 530.2 [M+H]⁺.

To a solution of tert-butyl 9-(5-chloro-2-(((3S,4R)-3-hydroxytetrahydro-2H-pyran-4-yl)amino)pyrimidin-4-yl)-7-fluoro-3,4-dihydrobenzo[c][2,6]naphthyridine-2(1H)-carboxylate (9.11, 150 mg, crude) in methanol (2 mL) was added hydrochloric acid (1 mL, 4 M in dioxane), and the mixture was stirred at room temperature for 1 hr. The mixture was concentrated under reduced pressure, adjusted to pH 8 with saturated sodium bicarbonate solution (5 mL) and then extracted with ethyl acetate (20 mL×2). The combined organic layers were dried over anhydrous sodium sulfate, filtered and concentrated under reduced pressure. The residue was subjected to prep-HPLC to afford (3S,4R)-4-((5-chloro-4-(7-fluoro-1,2,3,4-tetrahydrobenzo[c][2,6]naphthyridin-9-yl)pyrimidin-2-yl)amino)tetrahydro-2H-pyran-3-ol (9, 19.2 mg) as a white solid. LC-MS (ESI): m/z 430.1 [M+H]⁺. ¹H NMR (400 MHz, DMSO-d₆) δ 8.75 (s, 1H), 8.47 (s, 1H), 8.16 (s, 1H), 7.84 (d, J=11.5 Hz, 1H), 7.56 (s, 1H), 4.92 (d, J=5.4 Hz, 1H), 4.29 (s, 2H), 3.89-3.76 (m, 3H), 3.54-3.47 (m, 1H), 3.38-3.35 (m, 1H), 3.08-3.00 (m, 3H), 2.92-2.84 (m, 2H), 2.02-1.88 (m, 1H), 1.58-1.44 (m, 1H).

### Illustration 10. Synthesis of (3S,4R)-4-((5-chloro-4-(2-methyl-3H-imidazo[4,5-c]quinolin-8-yl)pyrimidin-2-yl)amino)tetrahydro-2H-pyran-3-ol (10)

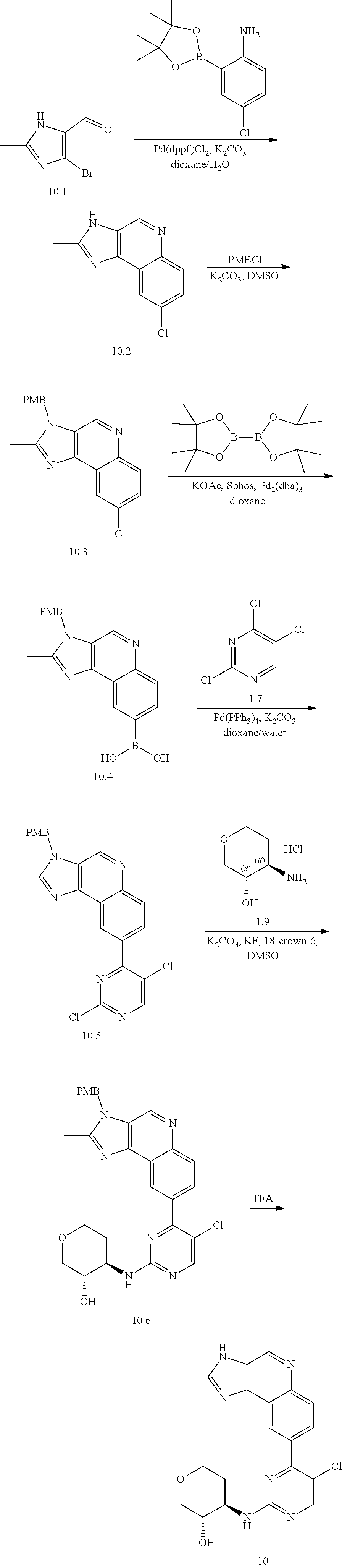

A mixture of 4-bromo-2-methyl-1H-imidazole-5-carbaldehyde (10.1, 1.00 g, 5.29 mmol), 4-chloro-2-(4,4,5,5-tetramethyl-1,3,2-dioxaborolan-2-yl)aniline (1.34 g, 5.29 mmol), potassium carbonate (2.19 g, 15.9 mmol) and Pd(dppf)Cl₂ (387 mg, 529 umol) in dioxane (10 mL) and water (1 mL) was stirred at 90° C. for 2 hrs under nitrogen atmosphere. The reaction mixture was concentrated under reduced pressure and the residue was subjected to silica gel column chromatography to afford 8-chloro-2-methyl-3H-imidazo[4,5-c]quinoline (10.2, 680 mg, 54%) as a yellow solid. LC-MS (ESI): m/z 218.1 [M+H]⁺.

To a solution of 8-chloro-2-methyl-3H-imidazo[4,5-c]quinoline (10.2, 580 mg, 2.66 mmol) in dimethylsulfoxide (5 mL) were added potassium carbonate (368 mg, 2.66 mmol) and 1-(chloromethyl)-4-methoxybenzene (417 mg, 2.66 mmol), and the mixture was stirred at 25° C. for 2 hrs. The reaction mixture was quenched with water (30 mL) and extracted with ethyl acetate (15 mL×3). The combined organic layers were washed with brine (20 mL×3), dried over anhydrous sodium sulfate and concentrated under reduced pressure. The residue was subjected to silica gel column chromatography to afford 8-chloro-3-(4-methoxybenzyl)-2-methyl-3H-imidazo[4,5-c]quinoline (10.3, 510 mg, 57%) as a white solid. LC-MS (ESI): m/z 338.1 [M+H]⁺.

A mixture of 8-chloro-3-(4-methoxybenzyl)-2-methyl-3H-imidazo[4,5-c]quinoline (10.3, 510 mg, 1.51 mmol), 4,4,4′,4′,5,5,5′,5′-octamethyl-2,2′-bi(1,3,2-dioxaborolane) (958 mg, 3.77 mmol), tris(dibenzylideneacetone)dipalladium (138 mg, 151 umol,) 2-dicyclohexylphosphino-2′,6′-dimethoxybiphenyl (124 mg, 302 umol) and potassium acetate (445 mg, 4.53 mmol) in dioxane (5 mL) was stirred at 100° C. for 12 hrs under nitrogen atmosphere. The reaction mixture was filtered and the filtrate was concentrated under reduced pressure. The residue was subjected to prep-HPLC to afford (3-(4-methoxybenzyl)-2-methyl-3H-imidazo[4,5-c]quinolin-8-yl)boronic acid (10.4, 315 mg, 60%) as a white solid. LC-MS (ESI): m/z 348.3 [M+H]⁺. ¹H NMR (400 MHz, DMSO-d₆) δ 9.16 (s, 1H), 8.96 (s, 1H), 8.27 (s, 2H), 8.00 (s, 2H), 7.20 (d, J=8.4 Hz, 2H), 6.91 (d, J=8.4 Hz, 2H), 5.62 (s, 2H), 3.70 (s, 3H), 2.69 (s, 3H).

To a mixture of (3-(4-methoxybenzyl)-2-methyl-3H-imidazo[4,5-c]quinolin-8-yl)boronic acid (10.4, 100 mg, 0.23 mmol) and 2,4,5-trichloropyrimidine (1.7, 27 μL, 0.23 mmol) in dioxane (1 mL) and water (0.2 mL) were added Pd(PPh₃)₄ (54 mg, 0.05 mmol) and potassium carbonate (64.0 mg, 0.46 mmol), and the mixture was stirred at 90° C. for 4 hrs. The mixture was diluted with water (20 mL) and extracted with ethyl acetate (10 mL×2). The combined organic layers were dried over anhydrous sodium sulfate and concentrated under reduced pressure. The residue was subjected to silica gel column chromatography to afford 8-(2,5-dichloropyrimidin-4-yl)-3-(4-methoxybenzyl)-2-methyl-3H-imidazo[4,5-c]quinoline (10.5, 100 mg, crude) as a yellow solid. LC-MS (ESI): m/z 450.2 [M+H]⁺.

A mixture of 8-(2,5-dichloropyrimidin-4-yl)-3-(4-methoxybenzyl)-2-methyl-3H-imidazo[4,5-c]quinoline (10.5, 100 mg, crude), (3S,4R)-4-aminotetrahydropyran-3-ol hydrochloride (1.9, 38.0 mg, 0.24 mmol), potassium carbonate (31.0 mg, 0.22 mmol), potassium fluoride (39.0 mg, 0.66 mmol) and 18-crown-6 (6.00 mg, 0.02 mmol) in dimethylsulfoxide (2 mL) was stirred at 120° C. for 1 hr. The mixture was diluted with water (20 mL) and extracted with ethyl acetate (10 mL×2). The combined organic layers were washed with brine (10 mL×2), dried over anhydrous sodium sulfate and concentrated under reduced pressure to afford (3S,4R)-4-((5-chloro-4-(3-(4-methoxybenzyl)-2-methyl-3H-imidazo[4,5-c]quinolin-8-yl)pyrimidin-2-yl)amino)tetrahydro-2H-pyran-3-ol (10.6, 100 mg, crude) as a yellow solid. LC-MS (ESI): m/z 531.2 [M+H]⁺.

A mixture of (3S,4R)-4-((5-chloro-4-(3-(4-methoxybenzyl)-2-methyl-3H-imidazo[4,5-c]quinolin-8-yl)pyrimidin-2-yl)amino)tetrahydro-2H-pyran-3-ol (10.6, 100 mg, crude) and trifluoroacetic acid (1 mL) was stirred at 100° C. for 10 mins. The reaction mixture was concentrated under reduced pressure. The residue was diluted with water (15 mL), adjusted to pH 8 with saturated sodium carbonate solution and then extracted with dichloromethane (10 mL×3). The combined organic layers were dried over anhydrous sodium sulfate and concentrated under reduced pressure. The residue was subjected to prep-HPLC to afford (3S,4R)-4-((5-chloro-4-(2-methyl-3H-imidazo[4,5-c]quinolin-8-yl)pyrimidin-2-yl)amino)tetrahydro-2H-pyran-3-ol (10, 27.1 mg) as a white solid. LC-MS (ESI): m/z 411.2 [M+H]⁺. ¹H NMR (400 MHz, DMSO-d₆) δ 9.13 (s, 1H), 8.75 (s, 1H), 8.45 (s, 1H), 8.16 (d, J=8.5 Hz, 1H), 7.96 (d, J=8.7 Hz, 1H), 7.48 (s, 1H), 4.93 (s, 1H), 3.88-3.75 (m, 3H), 3.52-3.45 (m, 2H), 3.03 (t, J=10.4 Hz, 1H), 2.63 (s, 3H), 2.02-1.90 (m, 1H), 1.57-1.41 (m, 1H).

### Illustration 11. Synthesis of (3S,4R)-4-((5-chloro-4-(1-methyl-3H-pyrazolo[3,4-c]quinolin-8-yl)pyrimidin-2-yl)amino)tetrahydro-2H-pyran-3-ol (11)

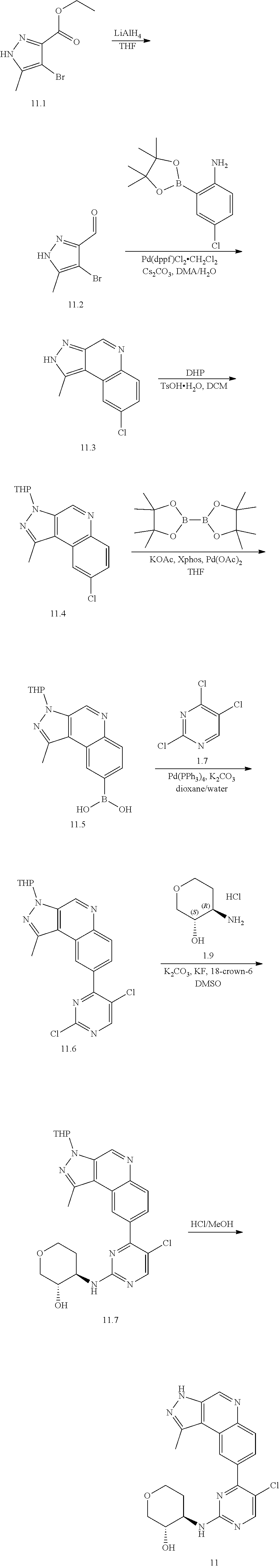

To a mixture of ethyl 4-bromo-5-methyl-1H-pyrazole-3-carboxylate (11.1, 7.85 g, 33.7 mmol) in tetrahydrofuran (80 mL) was added lithium aluminum hydride (1.28 g, 33.7 mmol) in portions at 0° C., and the mixture was then stirred at 0° C. for 3 hrs under nitrogen atmosphere. The reaction mixture was quenched with saturated sodium chloride solution (6 mL) slowly at 0° C. and stirred for 10 mins. The resulting mixture was filtered and the filtrate was concentrated under reduced pressure. The residue was subjected to silica gel column chromatography to afford 4-bromo-5-methyl-1H-pyrazole-3-carbaldehyde (11.2, 1.80 g, 28%) as a white solid. LC-MS (ESI): m/z 189.1 [M+H]⁺.

To a mixture of 4-bromo-5-methyl-1H-pyrazole-3-carbaldehyde (11.2, 0.90 g, 4.76 mmol), 4-chloro-2-(4,4,5,5-tetramethyl-1,3,2-dioxaborolan-2-yl)aniline (1.21 g, 4.76 mmol) and caesium carbonate (4.65 g, 14.3 mmol) in N,N-dimethylacetamide (70 mL) and water (15 mL) was added 1,1′-bis(diphenylphosphino)ferrocene-palladium(II)dichloride dichloromethane complex (389 mg, 476 umol), and the resulting mixture was stirred at 100° C. for 2 hrs under nitrogen atmosphere. The reaction mixture was cooled to room temperature, poured into water (100 mL) and extracted with ethyl acetate (50 mL×3). The combined organic layers were washed with brine (50 mL), dried over anhydrous sodium sulfate and concentrated under reduced pressure. The residue was subjected to silica gel column chromatography to afford 8-chloro-1-methyl-2H-pyrazolo[3,4-c]quinoline (11.3, 470 mg, 45%) as a yellow solid. LC-MS (ESI): m/z 218.2 [M+H]⁺.

To a mixture of 8-chloro-1-methyl-2H-pyrazolo[3,4-c]quinoline (11.3, 455 mg, 2.09 mmol) in dichloromethane (10 mL) were added 3,4-dihydro-2H-pyran (352 mg, 4.18 mmol) and p-toluenesulfonic acid monohydrate (79.5 mg, 418 umol), and the mixture was stirred at 40° C. for 2 hrs. The reaction mixture was cooled to room temperature, poured into water (30 mL) and extracted with dichloromethane (15 mL×3). The combined organic layers were washed with brine (20 mL), dried over anhydrous sodium sulfate and concentrated under reduced pressure. The residue was subjected to silica gel column chromatography to afford 8-chloro-1-methyl-3-(tetrahydro-2H-pyran-2-yl)-3H-pyrazolo[3,4-c]quinoline (11.4, 530 mg, 84%) as a yellow solid. LC-MS (ESI): m/z 302.2 [M+H]⁺.

To a mixture of 8-chloro-1-methyl-3-(tetrahydro-2H-pyran-2-yl)-3H-pyrazolo[3,4-c]quinoline (11.4, 510 mg, 1.69 mmol), 4,4,4′,4′,5,5,5′,5′-octamethyl-2,2′-bi(1,3,2-dioxaborolane) (858 mg, 3.38 mmol), potassium acetate (498 mg, 5.07 mmol) and dicyclohexyl(2′,4′,6′-triisopropyl-[1,1′-biphenyl]-2-yl)phosphane (161 mg, 338 umol) in tetrahydrofuran (10 mL) was added palladium(II) acetate (38.0 mg, 169 umol), and the resulting mixture was stirred at 70° C. for 2 hrs under nitrogen atmosphere. The reaction mixture was concentrated under reduced pressure and the residue was subjected to prep-HPLC to afford (1-methyl-3-(tetrahydro-2H-pyran-2-yl)-3H-pyrazolo[3,4-c]quinolin-8-yl)boronic acid (11.5, 169 mg, 29%) as a white solid. LC-MS (ESI): m/z 312.1 [M+H]⁺.

To a mixture of (1-methyl-3-(tetrahydro-2H-pyran-2-yl)-3H-pyrazolo[3,4-c]quinolin-8-yl)boronic acid (11.5, 80.0 mg, 0.26 mmol) and 2,4,5-trichloropyrimidine (1.7, 30 μL, 0.26 mmol) in water (0.2 mL) and dioxane (1 mL) were added tetrakis(triphenylphosphine) palladium(0) (59.0 mg, 0.05 mmol) and potassium carbonate (107 mg, 0.77 mmol), and the resulting mixture was stirred at 90° C. for 4 hrs under nitrogen atmosphere. The reaction mixture was diluted with water (20 mL) and extracted with ethyl acetate (10 mL×2). The combined organic layers were dried over anhydrous sodium sulfate and concentrated under reduced pressure. The residue was subjected to silica gel column chromatography to afford 8-(2,5-dichloropyrimidin-4-yl)-1-methyl-3-(tetrahydro-2H-pyran-2-yl)-3H-pyrazolo[3,4-c]quinoline (11.6, 100 mg, crude) as a white solid. LC-MS (ESI): m/z 414.1 [M+H]⁺.

A mixture of 8-(2,5-dichloropyrimidin-4-yl)-1-methyl-3-(tetrahydro-2H-pyran-2-yl)-3H-pyrazolo[3,4-c]quinoline (11.6, 100 mg, crude), (3S,4R)-4-aminotetrahydropyran-3-ol hydrochloride (1.9, 37.0 mg, 0.24 mmol), potassium carbonate (34.0 mg, 0.24 mmol), potassium fluoride (42.0 mg, 0.72 mmol) and 18-crown-6 (6.40 mg, 0.02 mmol) in dimethyl sulfoxide (2 mL) was stirred at 120° C. for 1 hr. The reaction mixture was diluted with water (20 mL) and extracted with ethyl acetate (10 mL×2). The combined organic layers were dried over anhydrous sodium sulfate and concentrated under reduced pressure to afford (3S,4R)-4-((5-chloro-4-(1-methyl-3-(tetrahydro-2H-pyran-2-yl)-3H-pyrazolo[3,4-c]quinolin-8-yl)pyrimidin-2-yl)amino)tetrahydro-2H-pyran-3-ol (11.7, 119 mg, crude) as a white solid. LC-MS (ESI): m/z 495.2 [M+H]⁺.

A mixture of (3S,4R)-4-((5-chloro-4-(1-methyl-3-(tetrahydro-2H-pyran-2-yl)-3H-pyrazolo[3,4-c]quinolin-8-yl)pyrimidin-2-yl)amino)tetrahydro-2H-pyran-3-ol (11.7, 119 mg, crude) and hydrogen chloride (2 mL, 4 M in methanol) was stirred at room temperature for 30 mins. The reaction mixture was concentrated under reduced pressure. The residue was diluted with water (15 mL), adjusted to pH 8 with saturated sodium carbonate solution and then extracted with dichloromethane (10 mL×3). The combined organic layers were dried over anhydrous sodium sulfate and concentrated under reduced pressure. The residue was subjected to prep-HPLC to afford (3S,4R)-4-((5-chloro-4-(1-methyl-3H-pyrazolo[3,4-c]quinolin-8-yl)pyrimidin-2-yl)amino)tetrahydro-2H-pyran-3-ol (11, 39.9 mg) as a white solid. LC-MS (ESI): m/z 411.1 [M+H]⁺. ¹H NMR (400 MHz, DMSO-d₆) δ 13.72 (s, 1H), 9.28 (s, 1H), 8.98-8.60 (m, 1H), 8.47 (s, 1H), 8.32-8.16 (m, 1H), 8.15-7.83 (m, 1H), 7.52 (s, 1H), 4.94 (d, J=5.3 Hz, 1H), 3.94-3.76 (m, 3H), 3.58-3.48 (m, 1H), 3.39-3.33 (m, 1H), 3.05 (t, J=10.4 Hz, 1H), 2.85 (s, 3H), 2.10-1.88 (m, 1H), 1.62-1.43 (m, 1H).

### Illustration 12. Synthesis of (3S,4R)-4-((5-chloro-4-(4-fluoro-2-(2-hydroxypropan-2-yl)-1-isopropyl-1H-benzo[d]imidazol-6-yl)pyridin-2-yl)amino)tetrahydro-2H-pyran-3-ol (12)

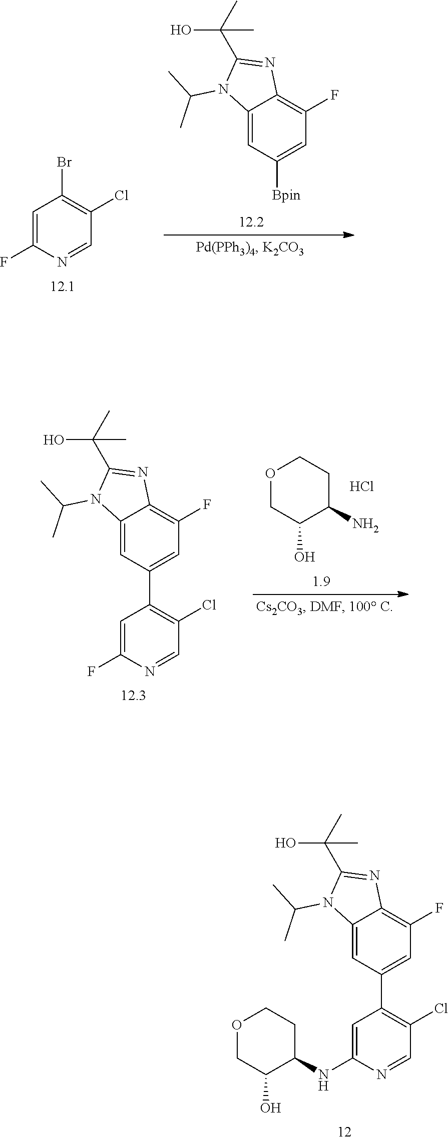

To a mixture of 4-bromo-5-chloro-2-fluoropyridine (12.1, 58.1 mg, 0.28 mmol) in dioxane (4 mL) and water (1 mL) were added 2-(4-fluoro-1-isopropyl-6-(4,4,5,5-tetramethyl-1,3,2-dioxaborolan-2-yl)-1H-benzo[d]imidazol-2-yl)propan-2-ol (12.2, 100 mg, 0.27 mmol), Pd(PPh₃)₄ (31.9 mg, 0.03 mmol) and potassium carbonate (76.3 mg, 0.55 mmol), and the resulting mixture was stirred at 110° C. for 3 hrs under nitrogen atmosphere. The reaction mixture was diluted with ethyl acetate (30 mL), washed with brine (15 mL×3), dried over anhydrous sodium sulfate and then concentrated under reduced pressure. The residue was subjected to silica gel column chromatography to afford 2-(6-(5-chloro-2-fluoropyridin-4-yl)-4-fluoro-1-isopropyl-1H-benzo[d]imidazol-2-yl)propan-2-ol (12.3, 98.0 mg, 97%) as a white solid. LC-MS (ESI): m/z 366.1 [M+H]⁺.

To a solution of 2-(6-(5-chloro-2-fluoropyridin-4-yl)-4-fluoro-1-isopropyl-1H-benzo[d]imidazol-2-yl)propan-2-ol (12.3, 98.0 mg, 0.28 mmol) in N,N-dimethyl formamide (3 mL) were added cesium carbonate (180 mg, 0.55 mmol) and (3S,4R)-4-aminotetrahydro-2H-pyran-3-ol hydrochloride (1.9, 85.0 mg, 0.55 mmol), and the mixture was stirred at 100° C. for 12 hrs. The reaction mixture was diluted with water (15 mL) and extracted with dichloromethane (20 mL×3). The combined organic layers were washed with brine (10 mL×3), dried over anhydrous sodium sulfate and concentrated under reduced pressure. The residue was subjected to prep-HPLC to afford (3S,4R)-4-((5-chloro-4-(4-fluoro-2-(2-hydroxypropan-2-yl)-1-isopropyl-1H-benzo[d]imidazol-6-yl)pyridin-2-yl)amino)tetrahydro-2H-pyran-3-ol (12, 15.0 mg, 12%) as a white solid. LC-MS (ESI): m/z 463.2 [M+H]⁺. ¹H NMR (400 MHz, DMSO-d₆) δ 8.06 (s, 1H), 7.56 (s, 1H), 7.05 (d, J=8.0 Hz, 1H), 6.80 (d, J=8.0 Hz, 1H), 6.64 (s, 1H), 5.79 (s, 1H), 5.76-5.71 (m, 1H), 5.03 (d, J=8.0 Hz, 1H), 3.83-3.77 (m, 3H), 3.54-3.43 (m, 2H), 3.08 (t, J=12.0 Hz, 1H), 2.07-1.96 (m, 1H), 1.66 (s, 6H), 1.58 (d, J=7.2 Hz, 7H).

### Illustration 13. Synthesis of (3S,4R)-4-((5-chloro-4-(1-methyl-3,4-dihydrochromeno[3,4-c]pyrazol-8-yl)pyrimidin-2-yl)amino)tetrahydro-2H-pyran-3-ol (13)

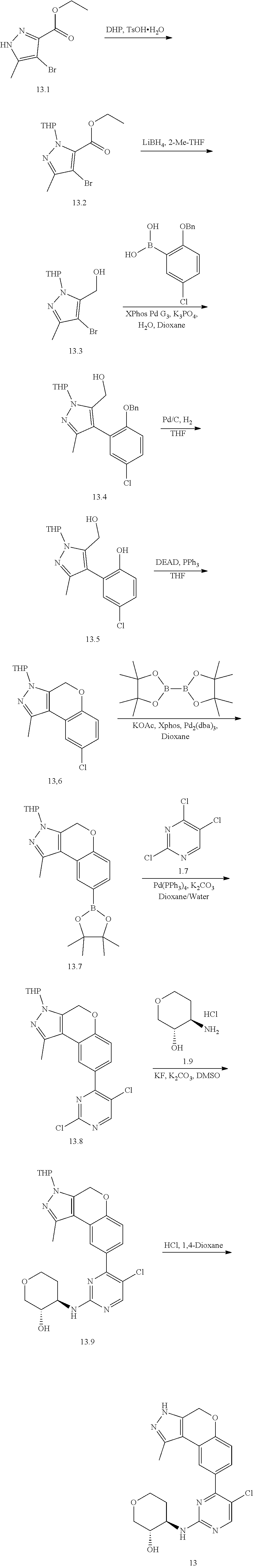

To a mixture of ethyl 4-bromo-5-methyl-1H-pyrazole-3-carboxylate (13.1, 5.00 g, 21.5 mmol) and p-toluenesulfonic acid monohydrate (816 mg, 4.29 mmol) in dichloromethane (85 mL) was added 3,4-dihydro-2H-pyran (4.00 mL, 42.9 mmol), and the mixture was stirred at 25° C. for 12 hrs. The reaction mixture was concentrated under reduced pressure and the residue was subjected to silica gel column chromatography to afford ethyl 4-bromo-3-methyl-1-(tetrahydro-2H-pyran-2-yl)-1H-pyrazole-5-carboxylate (13.2, 5.40 g, 79%) as a yellow solid. LC-MS (ESI): m/z 317.1 [M+H]⁺.

To a mixture of ethyl 4-bromo-3-methyl-1-(tetrahydro-2H-pyran-2-yl)-1H-pyrazole-5-carboxylate (13.2, 4.20 g, 13.2 mmol) in 2-methyltetrahydrofuran (75 mL) was added lithium borohydride (1.15 g, 53.0 mmol) at 0° C. under nitrogen atmosphere, and the mixture was stirred at 25° C. for 14 hrs under nitrogen atmosphere. The reaction mixture was poured into ice water (200 mL), and it was stirred for 10 mins and then extracted with ethyl acetate (100 mL×3). The combine organic layers were washed with brine (100 mL), dried over anhydrous sodium sulfate and concentrated under reduced pressure to give (4-bromo-3-methyl-1-(tetrahydro-2H-pyran-2-yl)-1H-pyrazol-5-yl)methanol (13.3, 3.60 g, crude) as a white solid. LC-MS (ESI): m/z 275.2 [M+H]⁺.

To a mixture of (4-bromo-3-methyl-1-(tetrahydro-2H-pyran-2-yl)-1H-pyrazol-5-yl)methanol (13.3, 3.60 g, crude), (2-(benzyloxy)-5-chlorophenyl)boronic acid (3.43 g, 13.1 mmol) and potassium phosphate (8.33 g, 39.3 mmol) in dioxane (90 mL) and water (18 mL) was added XPhos Pd G3 (1.11 g, 1.31 mmol), and the resulting mixture was stirred at 90° C. for 2 hrs under nitrogen atmosphere. The reaction mixture was cooled to room temperature, diluted with water (200 mL), and extracted with ethyl acetate (100 mL×3). The combine organic layers were washed with brine (100 mL), dried over anhydrous sodium sulfate and concentrated under reduced pressure. The residue was subjected to silica gel column chromatography to afford (4-(2-(benzyloxy)-5-chlorophenyl)-3-methyl-1-(tetrahydro-2H-pyran-2-yl)-1H-pyrazol-5-yl)methanol (13.4, 3.80 g, 70%) as a yellow oil. LC-MS (ESI): m/z 413.2 [M+H]⁺.

To a solution of (4-(2-(benzyloxy)-5-chlorophenyl)-3-methyl-1-(tetrahydro-2H-pyran-2-yl)-1H-pyrazol-5-yl)methanol (13.4, 1.00 g, 2.42 mmol) in tetrahydrofuran (20 mL) was added palladium (1.00 g, 10% on carbon). The resulting mixture was degassed and backfilled with hydrogen three times, and then it was stirred at room temperature for 15 mins under hydrogen atmosphere (1 atm). The reaction mixture was filtered and the filtrate was concentrated under reduced pressure to give 4-chloro-2-(5-(hydroxymethyl)-3-methyl-1-(tetrahydro-2H-pyran-2-yl)-1H-pyrazol-4-yl)phenol (13.5, 770 mg, crude) as a yellow solid. LC-MS (ESI): 323.2 [M+H]⁺.

To a mixture of 4-chloro-2-(5-(hydroxymethyl)-3-methyl-1-(tetrahydro-2H-pyran-2-yl)-1H-pyrazol-4-yl)phenol (13.5, 770 mg, crude) and triphenylphosphine (939 mg, 3.58 mmol) in tetrahydrofuran (20 mL) was added diethyl azodicarboxylate (0.66 mL, 3.58 mmol) at 0° C., and the mixture was stirred at 25° C. for 2 hrs under nitrogen atmosphere. The reaction mixture was poured into water (50 mL) and extracted with ethyl acetate (30 mL×3). The combine organic layers were washed with brine (30 mL), dried over anhydrous sodium sulfate and concentrated under reduced pressure. The residue was subjected to silica gel column chromatography to afford 8-chloro-1-methyl-3-(tetrahydro-2H-pyran-2-yl)-3,4-dihydrochromeno[3,4-c]pyrazole (13.6, 520 mg) as a yellow solid. LC-MS (ESI): 305.2 [M+H]⁺.

To a mixture of 8-chloro-1-methyl-3-(tetrahydro-2H-pyran-2-yl)-3,4-dihydrochromeno[3,4-c]pyrazole (13.6, 450 mg, 1.48 mmol), 4,4,4′,4′,5,5,5′,5′-octamethyl-2,2′-bi(1,3,2-dioxaborolane) (750 mg, 2.95 mmol), potassium acetate (435 mg, 4.43 mmol) and XPhos (141 mg, 0.30 mmol) in dioxane (15.0 mL) was added Pd₂(dba)₃ (136 mg, 0.15 mmol), and the resulting mixture was stirred at 100° C. for 2 hrs under nitrogen atmosphere. The reaction mixture was filtered and the filtrate was concentrated under reduced pressure. The residue was subjected to silica gel column chromatography to afford 1-methyl-3-(tetrahydro-2H-pyran-2-yl)-8-(4,4,5,5-tetramethyl-1,3,2-dioxaborolan-2-yl)-3,4-dihydrochromeno[3,4-c]pyrazole (13.7, 366 mg, 26%) as a yellow solid. LC-MS (ESI): 397.3 [M+H]⁺.

A mixture of 1-methyl-3-(tetrahydro-2H-pyran-2-yl)-8-(4,4,5,5-tetramethyl-1,3,2-dioxaborolan-2-yl)-3,4-dihydrochromeno[3,4-c]pyrazole (13.7, 100 mg, 0.25 mmol), 2,4,5-trichloropyrimidine (1.7, 55.5 mg, 0.30 mmol), tetrakis(triphenylphosphine)palladium (29.2 mg, 0.02 mmol) and potassium carbonate (69.8 mg, 0.50 mmol) in dioxane (1 mL) and water (0.25 mL) was stirred at 90° C. for 3 hrs under nitrogen atmosphere. The reaction mixture was concentrated under reduced pressure and the residue was subjected to silica gel column chromatography to afford 8-(2,5-dichloropyrimidin-4-yl)-1-methyl-3-(3,4,5,6-tetrahydro-2H-pyran-2-yl)-4H-chromeno[3,4-c]pyrazole (13.8, 102 mg, 96%) as a yellow solid. LC-MS (ESI): m/z 417.2 [M+H]⁺.

A mixture of 8-(2,5-dichloropyrimidin-4-yl)-1-methyl-3-(3,4,5,6-tetrahydro-2H-pyran-2-yl)-4H-chromeno[3,4-c]pyrazole (13.8, 100 mg, 0.24 mmol), (3S,4R)-4-aminotetrahydropyran-3-ol hydrogen chloride (1.9, 73.6 mg, 0.48 mmol), potassium fluoride (55.7 mg, 0.96 mmol), 18-crown-6 (63.3 mg, 0.24 mmol) and potassium carbonate (33.1 mg, 0.24 mmol) in dimethyl sulfoxide (2 mL) was stirred at 120° C. for 2 hrs. The reaction mixture was concentrated under reduced pressure and the residue was subjected to silica gel column chromatography to afford (3S,4R)-4-((5-chloro-4-(1-methyl-3-(tetrahydro-2H-pyran-2-yl)-3,4-dihydrochromeno[3,4-c]pyrazol-8-yl)pyrimidin-2-yl)amino)tetrahydro-2H-pyran-3-ol (13.9, 102 mg, 85%) as a white solid. LC-MS (ESI): m/z 498.3 [M+H]⁺.

A solution of (3S,4R)-4-((5-chloro-4-(1-methyl-3-(tetrahydro-2H-pyran-2-yl)-3,4-dihydrochromeno[3,4-c]pyrazol-8-yl)pyrimidin-2-yl)amino)tetrahydro-2H-pyran-3-ol (13.9, 102 mg, 0.205 mmol) in dioxane (2 mL) was added hydrochloric acid (1 mL, 4 M in dioxane), and the mixture was stirred at room temperature for 1 hr. The mixture was concentrated under reduced pressure, adjusted to pH 8 with saturated sodium bicarbonate solution (5 mL) and then extracted with ethyl acetate (20 mL×2). The combined organic layers were dried over anhydrous sodium sulfate, filtered and concentrated under reduced pressure. The residue was subjected to prep-HPLC to afford (3S,4R)-4-((5-chloro-4-(1-methyl-3,4-dihydrochromeno[3,4-c]pyrazol-8-yl)pyrimidin-2-yl)amino)tetrahydro-2H-pyran-3-ol (13, 42.0 mg, 49%) as a white solid. LC-MS (ESI): m/z 414.2 [M+H]⁺. ¹H NMR (400 MHz, DMSO-d₆) δ 12.75 (s, 1H), 8.36 (s, 1H), 7.91 (s, 1H), 7.57 (s, 1H), 7.37 (d, J=7.8 Hz, 1H), 7.04 (d, J=8.4 Hz, 1H), 5.25 (s, 2H), 4.92 (d, J=5.3 Hz, 1H), 3.91-3.72 (m, 3H), 3.57-3.44 (m, 1H), 3.37-3.25 (m, 1H), 3.03 (t, J=10.4 Hz, 1H), 2.47 (s, 3H), 2.06-1.89 (m, 1H), 1.58-1.43 (m, 1H).

### Illustration 14. Synthesis of (3S,4R)-4-((5-chloro-4-(8-fluoro-3-((R)-1-hydroxyethyl)-2,4-dimethylquinolin-6-yl)pyrimidin-2-yl)amino)tetrahydro-2H-pyran-3-ol (14)

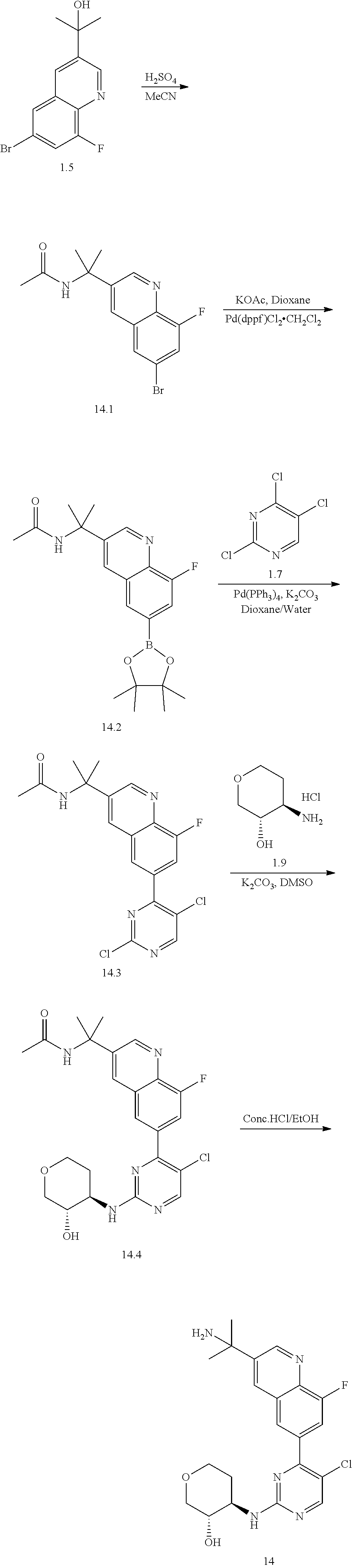

To a solution of 2-(6-bromo-8-fluoroquinolin-3-yl)propan-2-ol (1.5, 1.00 g, 3.52 mmol) in acetonitrile (15 mL) was added sulfuric acid (1.5 mL, 28.2 mmol) dropwise at 0° C., and the mixture was stirred at 25° C. for 12 hrs. The reaction mixture was poured into water (20 mL), adjusted to pH 9 with 1 M sodium hydroxide aqueous solution and then extracted with ethyl acetate (20 mL×3). The combined organic layers were washed with brine (20 mL), dried over anhydrous sodium sulfate and concentrated under reduced pressure. The residue was subjected to silica gel column chromatography to afford N-(2-(6-bromo-8-fluoroquinolin-3-yl)propan-2-yl)acetamide (14.1, 810 mg, 71%) as a white solid. LC-MS (EST): m/z 325.0 [M+H]⁺.

A mixture of N-(2-(6-bromo-8-fluoroquinolin-3-yl)propan-2-yl)acetamide (14.1, 670 mg, 2.06 mmol), 4,4,5,5-tetramethyl-2-(4,4,5,5-tetramethyl-1,3,2-dioxaborolan-2-yl)-1,3,2-dioxaborolane (628 mg, 2.47 mmol), potassium acetate (607 mg, 6.18 mmol) and 1,1′-bis(diphenylphosphino)ferrocene-palladium(II)dichloride dichloromethane complex (169 mg, 0.20 mmol) in dioxane (5 mL) was stirred at 90° C. for 1 hr under nitrogen atmosphere. The reaction mixture was cooled to room temperature, diluted with ethyl acetate (50 mL) and filtered. The filtrate was concentrated under reduced pressure and the residue was subjected to silica gel column chromatography to afford N-(2-(8-fluoro-6-(4,4,5,5-tetramethyl-1,3,2-dioxaborolan-2-yl)quinolin-3-yl)propan-2-yl)acetamide (14.2, 525 mg, 68%) as a yellow oil. LC-MS (ESI): m/z 373.2 [M+H]⁺.

A mixture of N-(2-(8-fluoro-6-(4,4,5,5-tetramethyl-1,3,2-dioxaborolan-2-yl) quinolin-3-yl)propan-2-yl)acetamide (14.2, 525 mg, 1.41 mmol), 2,4,5-trichloropyrimidine (1.7, 259 mg, 1.41 mmol), tetrakis(triphenylphosphine)palladium (163 mg, 0.14 mmol) and potassium carbonate (390 mg, 2.82 mmol) in dioxane (6 mL) and water (1 mL) was stirred at 90° C. for 2 hrs under nitrogen atmosphere. The reaction mixture was concentrated under reduced pressure and the residue was subjected to silica gel column chromatography to afford N-(2-(6-(2,5-dichloropyrimidin-4-yl)-8-fluoroquinolin-3-yl)propan-2-yl)acetamide (14.3, 450 mg, 81%) as a yellow oil. LC-MS (ESI): m/z 393.1 [M+H]f.

A mixture of N-(2-(6-(2,5-dichloropyrimidin-4-yl)-8-fluoroquinolin-3-yl)propan-2-yl)acetamide (14.3, 450 mg, 1.14 mmol), (3S,4R)-4-aminotetrahydropyran-3-ol hydrogen chloride (1.9, 264 mg, 1.72 mmol), potassium fluoride (266 mg, 4.57 mmol), potassium carbonate (237 mg, 1.72 mmol) and 18-crown-6 (302 mg, 1.14 mmol) in dimethyl sulfoxide (3 mL) was stirred at 120° C. for 2 hrs. The reaction mixture was diluted with water (20 mL) and extracted with ethyl acetate (10 mL×3). The combined organic layers were washed with brine (20 mL), dried over anhydrous sodium sulfate and concentrated under reduced pressure. The residue was subjected to silica gel column chromatography to afford N-(2-(6-(5-chloro-2-(((3S,4R)-3-hydroxytetrahydro-2H-pyran-4-yl)amino)pyrimidin-4-yl)-8-fluoroquinolin-3-yl)propan-2-yl)acetamide (14.4, 487 mg, 90%) as a white solid. LC-MS (ESI): m/z 474.2 [M+H]⁺.

A mixture of N-(2-(6-(5-chloro-2-(((3S,4R)-3-hydroxytetrahydro-2H-pyran-4-yl)amino)pyrimidin-4-yl)-8-fluoroquinolin-3-yl)propan-2-yl)acetamide (14.4, 487 mg, 1.03 mmol) and conc. hydrochloric acid (5 mL, 12 M) in ethanol (5 mL) was stirred at 90° C. for 48 hrs. The mixture was concentrated under reduced pressure to give a residue. The residue was diluted with water (10 mL) and adjusted to pH 9 with 1 M sodium hydroxide aqueous solution. The resulting mixture was extracted with ethyl acetate (20 mL×3). The combined organic layers were washed with brine (20 mL), dried over anhydrous sodium sulfate and concentrated under reduced pressure. The residue was subjected to silica gel column chromatography to afford (3S,4R)-4-((4-(3-(2-aminopropan-2-yl)-8-fluoroquinolin-6-yl)-5-chloropyrimidin-2-yl)amino)tetrahydro-2H-pyran-3-ol (14, 79.0 mg, 18%) as a white solid. LC-MS (ESI): m/z 432.2 [M+H]. ¹H NMR (500 MHz, DMSO-d₆) δ 9.29 (d, J=2.3 Hz, 1H), 8.57 (s, 1H), 8.48 (s, 1H), 8.26 (s, 1H), 7.84 (d, J=11.6 Hz, 1H), 7.56 (s, 1H), 4.93 (d, J=5.4 Hz, 1H), 3.98-3.75 (m, 3H), 3.58-3.44 (m, 1H), 3.38-3.33 (m, 1H), 3.12-3.01 (m, 1H), 2.07-1.87 (m, 1H), 1.61-1.43 (m, 7H).

### Illustration 15. Synthesis of (3S,4R)-4-((5-chloro-4-(8-fluoro-3-((R)-1-hydroxyethyl)-2,4-dimethylquinolin-6-yl)pyrimidin-2-yl)amino)tetrahydro-2H-pyran-3-ol (15a) and (3S,4R)-4-((5-chloro-4-(8-fluoro-3-((S)-1-hydroxyethyl)-2,4-dimethylquinolin-6-yl)pyrimidin-2-yl)amino)tetrahydro-2H-pyran-3-ol (15b)

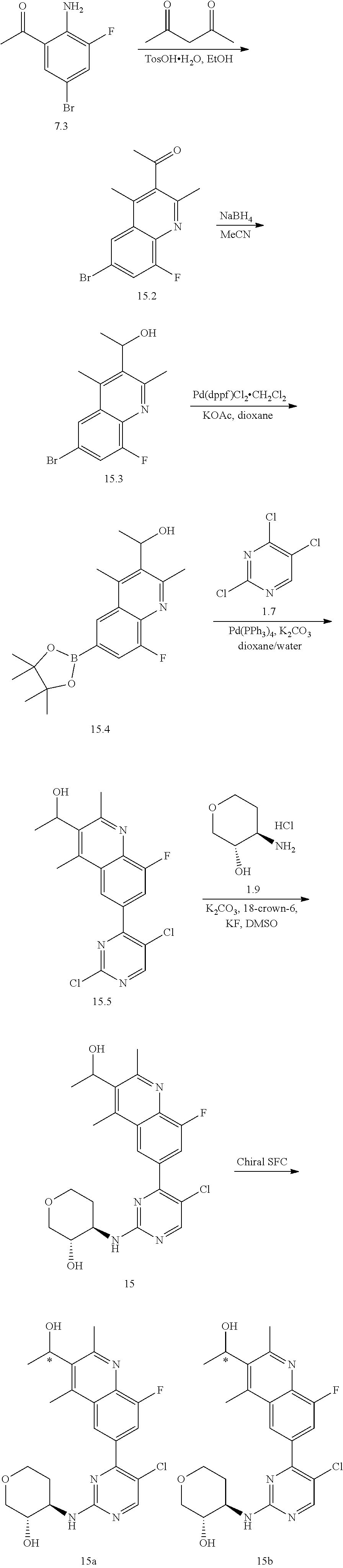

A mixture of 1-(2-amino-5-bromo-3-fluorophenyl)ethan-1-one (7.3, 300 mg, 1.29 mmol), pentane-2,4-dione (0.14 mL, 1.42 mmol) and p-toluenesulfonic acid monohydrate (49.2 mg, 0.26 mmol) in ethanol (5 mL) was stirred at 90° C. for 3 hrs. The reaction mixture was cooled to room temperature and concentrated under reduced pressure. The residue was subjected to silica gel column chromatography to afford 1-(6-bromo-8-fluoro-2,4-dimethylquinolin-3-yl)ethan-1-one (15.2, 270 mg, 64%) as a yellow solid. LC-MS (ESI): m/z 296.0 [M+H]⁺.

To a solution of 1-(6-bromo-8-fluoro-2,4-dimethylquinolin-3-yl)ethan-1-one (15.2, 200 mg, 0.67 mmol) in methanol (3 mL) was added sodium borohydride (204 mg, 5.40 mmol) at 0° C., and the mixture was stirred at 25° C. for 12 hrs. The mixture was quenched with water (20 mL) and extracted with ethyl acetate (20 mL×3). The combined organic layers were washed with brine (20 mL), dried over sodium sulfate and concentrated under reduced pressure. The residue was subjected to silica gel column chromatography to afford 1-(6-bromo-8-fluoro-2,4-dimethylquinolin-3-yl)ethan-1-ol (15.3, 190 mg, 94%) as a yellow oil. LC-MS (ESI): m/z 298.0 [M+H]⁺.

A mixture of 1-(6-bromo-8-fluoro-2,4-dimethylquinolin-3-yl)ethan-1-ol (15.3, 190 mg, 0.63 mmol), 4,4,5,5-tetramethyl-2-(4,4,5,5-tetramethyl-1,3,2-dioxaborolan-2-yl)-1,3,2-dioxaborolane (194 mg, 0.76 mmol), potassium acetate (187 mg, 1.91 mmol) and 1,1′-bis(diphenylphosphino)ferrocene-palladium(II)dichloride dichloromethane complex (52.2 mg, 0.06 mmol) in dioxane (4.0 mL) was stirred at 90° C. for 1 hr under nitrogen atmosphere. The reaction mixture was cooled to room temperature, diluted with ethyl acetate (50 mL) and filtered. The filtrate was concentrated under reduced pressure and the residue was subjected to silica gel column chromatography to afford 1-(8-fluoro-2,4-dimethyl-6-(4,4,5,5-tetramethyl-1,3,2-dioxaborolan-2-yl)quinolin-3-yl)ethan-1-ol (15.4, 210 mg, 95%) as a yellow oil. LC-MS (ESI): m/z 346.2 [M+H]⁺.

A mixture of 1-(8-fluoro-2,4-dimethyl-6-(4,4,5,5-tetramethyl-1,3,2-dioxaborolan-2-yl)quinolin-3-yl)ethan-1-ol (15.4, 210 mg, 0.61 mmol), 2,4,5-trichloropyrimidine (1.7, 112 mg, 0.61 mmol), tetrakis(triphenylphosphine)palladium (70.3 mg, 0.06 mmol) and potassium carbonate (168 mg, 1.22 mmol) in dioxane (1 mL) and water (0.2 mL) was stirred at 90° C. for 2 hrs under nitrogen atmosphere. The reaction mixture was concentrated under reduced pressure and the residue was subjected to silica gel column chromatography to afford 1-(6-(2,5-dichloropyrimidin-4-yl)-8-fluoro-2,4-dimethylquinolin-3-yl)ethan-1-ol (15.5, 110 mg, 46%) as a yellow oil. LC-MS (ESI): m/z 366.1 [M+H]⁺.

A mixture of 1-(6-(2,5-dichloropyrimidin-4-yl)-8-fluoro-2,4-dimethylquinolin-3-yl)ethan-1-ol (15.5, 200 mg, 0.54 mmol), (3S,4R)-4-aminotetrahydropyran-3-ol hydrogen chloride (1.9, 109 mg, 0.71 mmol), potassium fluoride (127 mg, 2.18 mmol), 18-crown-6 (144 mg, 0.54 mmol) and potassium carbonate (75.5 mg, 0.54 mmol) in dimethyl sulfoxide (2 mL) was stirred at 120° C. for 2 hrs. The reaction mixture was filtered and the filtrate was subjected to prep-HPLC to give (3S,4R)-4-((5-chloro-4-(8-fluoro-3-(1-hydroxyethyl)-2,4-dimethylquinolin-6-yl)pyrimidin-2-yl)amino)tetrahydro-2H-pyran-3-ol (15, 49.0 mg, 20%) as a white solid, which was further separated by chiral SFC to give:

Isomer 1: (15a, 100% ee); Retention time: 0.522 min. LC-MS (ESI): m/z 447.2 [M+H]⁺; ¹H NMR (500 MHz, DMSO-d₆) δ 8.47 (s, 1H), 8.37 (s, 1H), 7.83 (s, 1H), 7.56 (s, 1H), 5.47-5.36 (m, 2H), 4.94 (d, J=5.5 Hz, 1H), 3.92-3.77 (m, 3H), 3.51 (s, 1H), 3.39-3.33 (m, 1H), 3.11-3.02 (m, 1H), 2.83 (s, 3H), 2.81 (s, 3H), 1.97 (s, 1H), 1.59-1.44 (m, 4H).

Isomer 2: (15b, 100% ee); Retention time: 1.280 min; LC-MS (ESI): m/z 447.2 [M+H]⁺. ¹H NMR (500 MHz, DMSO-d₆) δ 8.47 (s, 1H), 8.37 (s, 1H), 7.83 (s, 1H), 7.56 (s, 1H), 5.48-5.37 (m, 2H), 4.94 (d, J=5.2 Hz, 1H), 3.94-3.75 (m, 3H), 3.51 (s, 1H), 3.39-3.33 (s, 1H), 3.10-3.00 (m, 1H), 2.83 (s, 3H), 2.80 (s, 3H), 1.96 (s, 1H), 1.60-1.43 (m, 4H).

Analytical separation method: column: CHIRALPAK IH-3, 4.6×50 mm, 3.0 um; A for MTBE (0.10% DEA) and B for MeOH; gradient: 3.6 min @B 50%; flow rate: 1.67 mL/min; high pressure: 76 bar; column temperature: 25° C.

Preparative separation method: instrument: GILSON-03; column: CHIRALPAK IH-3, 20×250 mm, 5 um; mobile phase: A for MTBE (0.10% DEA) and B for MeOH; gradient: B 50%; flow rate: 20 mL/min; high pressure: 78 bar; column temperature: 25° C.

### Illustration 16. Synthesis of (3S,4R)-4-((5-chloro-4-(8-fluoro-3-(1-hydroxycyclobutyl)quinolin-6-yl)pyrimidin-2-yl)amino)tetrahydro-2H-pyran-3-ol (16)

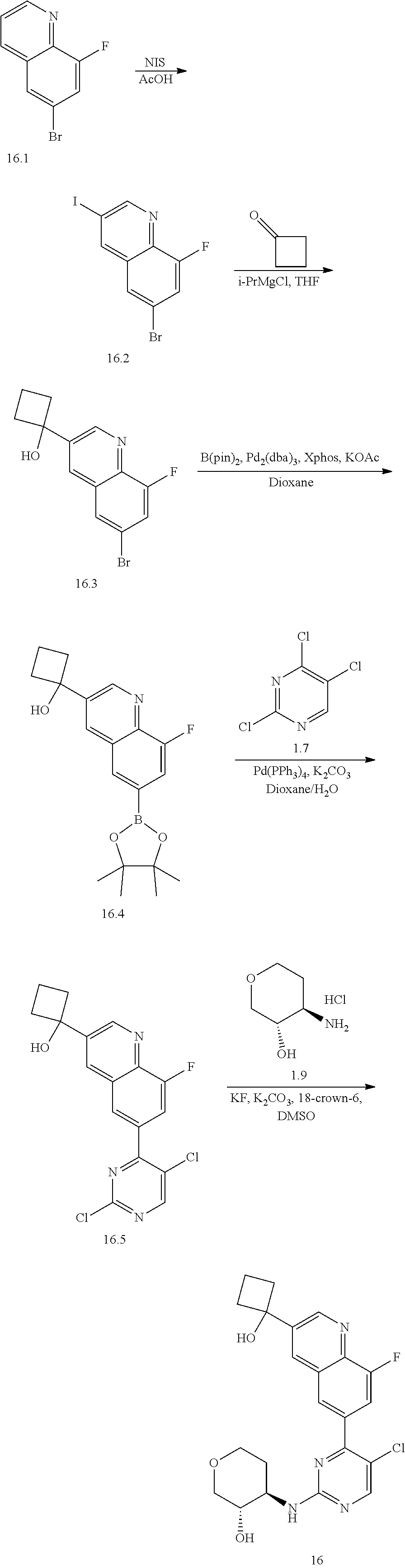

To a solution of 6-bromo-8-fluoroquinoline (16.1, 10.0 g, 44.2 mmol) in acetic acid (80 mL) was added N-iodosuccinimide (49.8 g, 221 mmol), and the mixture was stirred at 100° C. for 12 hrs. The reaction mixture was cooled to room temperature and then poured into water (100 mL). The resulting mixture was adjust to pH 8 with sat. sodium bicarbonate (300 mL) and extracted with ethyl acetate (50 mL×5). The combined organic layers were dried over anhydrous sodium sulfate, filtered and concentrated under reduced pressure. The residue was subjected to silica gel column chromatography to afford 6-bromo-8-fluoro-3-iodoquinoline (16.2, 9.00 g, 57%) as a yellow solid. ¹H NMR (400 MHz, DMSO-d₆) δ 9.12 (d, J=2.00 Hz, 1H), 8.94 (t, J=1.69 Hz, 1H), 8.10-8.05 (m, 1H), 7.93 (dd, J=10.19, 2.06 Hz, 1H).

To a solution of 6-bromo-8-fluoro-3-iodoquinoline (16.2, 2.00 g, 5.68 mmol) in tetrahydrofuran (30 mL) was added isopropylmagnesium chloride (5.25 mL, 1.30 M in tetrahydrofuran) dropwise at −78° C. under nitrogen atmosphere. The mixture was stirred at −78° C. for 1 hr followed by addition of a solution of cyclobutanone (597 mg, 8.52 mmol) in tetrahydrofuran (10 mL). The resulting mixture was stirred at −78° C. for 3 hrs, and it was then warmed to 25° C. slowly and stirred for another 8 hrs. The reaction mixture was poured into sat. ammonium chloride (100 mL) and extracted with ethyl acetate (40 mL×3). The combined organic layers were dried over anhydrous sodium sulfate, filtered and concentrated under reduced pressure. The residue was subjected to silica gel column chromatography to afford 1-(6-bromo-8-fluoroquinolin-3-yl)cyclobutan-1-ol (16.3, 950 mg, 45%) as a yellow solid. LC-MS (ESI): m/z 296.0 [M+H]⁺.

To a mixture of 1-(6-bromo-8-fluoroquinolin-3-yl)cyclobutan-1-ol (16.3, 880 mg, 2.97 mmol), bis(pinacolate)diboron (1.51 g, 5.94 mmol) and potassium acetate (875 mg, 8.91 mmol) in dioxane (10.0 mL) were added XPhos (283 mg, 0.59 mmol) and tris(dibenzylideneacetone)dipalladium (272 mg, 0.30 mmol), and the mixture was stirred 100° C. for 12 hrs under nitrogen atmosphere. The reaction mixture was filtered and the filter cake was washed with ethyl acetate (100 mL). The filtrate was concentrated under reduced pressure and the residue was subjected to silica gel column chromatography to afford 1-(8-fluoro-6-(4,4,5,5-tetramethyl-1,3,2-dioxaborolan-2-yl)quinolin-3-yl)cyclobutan-1-ol (16.4, 750 mg, 73%) as a purple oil. LC-MS (ESI): m/z 344.3 [M+H]⁺.

To a mixture of 1-(8-fluoro-6-(4,4,5,5-tetramethyl-1,3,2-dioxaborolan-2-yl)quinolin-3-yl)cyclobutan-1-ol (16.4, 260 mg, 0.76 mmol) and 2,4,5-trichloropyrimidine (1.7, 107 mg, 0.58 mmol) in dioxane/water (4 mL/l mL) were added tetrakis(triphenylphosphine)palladium (67.0 mg, 0.06 mmol) and potassium carbonate (160 mg, 1.16 mmol). The resulting mixture was stirred at 90° C. for 2 hrs under nitrogen atmosphere. The reaction mixture was diluted with water (20 mL) and extracted with ethyl acetate (10 mL×2). The combined organic layers were dried over anhydrous sodium sulfate, filtered and concentrated under reduced pressure. The residue was subjected to silica gel column chromatography to afford 1-(6-(2,5-dichloropyrimidin-4-yl)-8-fluoroquinolin-3-yl)cyclobutan-1-ol (16.5, 168 mg, 80%). LC-MS (ESI): m/z 364.0 [M+H]⁺.

To a mixture of 1-(6-(2,5-dichloropyrimidin-4-yl)-8-fluoroquinolin-3-yl)cyclobutan-1-ol (16.5, 168 mg, 0.46 mmol) and (3S,4R)-4-aminotetrahydro-2H-pyran-3-ol hydrochloride (1.9, 107 mg, 0.69 mmol) in anhydrous dimethylsulfoxide (2 mL) were added potassium fluoride (107 mg, 1.84 mmol), potassium carbonate (95.0 mg, 0.69 mmol) and 18-crown-6 (121 mg, 0.46 mmol). The resulting mixture was stirred at 110° C. for 1 hr. The reaction mixture was filtered and the filtrate was subjected to prep-HPLC to afford (3S,4R)-4-((5-chloro-4-(8-fluoro-3-(1-hydroxycyclobutyl)quinolin-6-yl)pyrimidin-2-yl)amino)tetrahydro-2H-pyran-3-ol (16, 40.0 mg, 20%) as white solid. LC-MS (ESI): m/z 445.1 [M+H]⁺. ¹H NMR (500 MHz, DMSO-d₆) δ 9.18 (d, J=2.2 Hz, 1H), 8.59 (t, J=1.9 Hz, 1H), 8.48 (s, 1H), 8.32 (s, 1H), 7.86 (d, J=12.6 Hz, 1H), 7.56 (s, 1H), 5.97 (s, 1H), 4.92 (d, J=5.5 Hz, 1H), 3.91-3.78 (m, 3H), 3.54-3.45 (m, 1H), 3.35 (dd, J=11.9, 2.3 Hz, 1H), 3.05 (dd, J=11.1, 9.6 Hz, 1H), 2.62-2.52 (m, 2H), 2.45-2.36 (m, 2H), 2.03-1.93 (m, 2H), 1.82-1.69 (m, 1H), 1.56-1.44 (m, 1H).

### Illustration 17. Synthesis of (3S,4R)-4-((5-chloro-4-(4-chloro-8-fluoro-3-(2-hydroxypropan-2-yl)quinolin-6-yl)pyrimidin-2-yl)amino)tetrahydro-2H-pyran-3-ol (17)

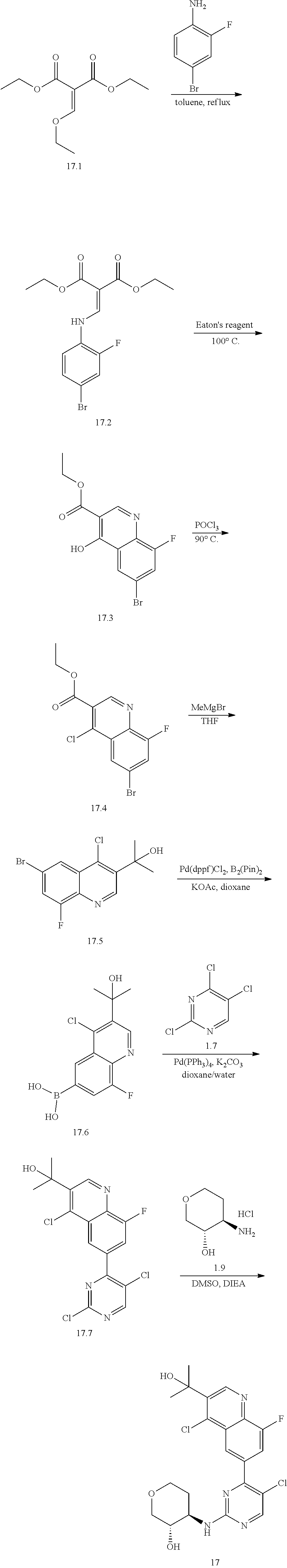

To a solution of 4-bromo-2-fluoroaniline (9.46 g, 49.8 mmol) in toluene (40 mL) was added diethyl 2-(ethoxymethylene)malonate (17.1, 11.6 g, 53.9 mmol), and the mixture was heated to reflux and stirred under nitrogen atmosphere overnight. The reaction mixture was concentrated under reduced pressure and the residue was subjected to silica gel column chromatography to afford diethyl 2-(((4-bromo-2-fluorophenyl)amino)methylene)malonate (17.2, 10.5 g, 59%). LC-MS (ESI): m/z 360.0 [M+H]⁺ and 362.0 [M+2+H]⁺.

A mixture of Eaton's reagent (6 mL) and diethyl 2-(((4-bromo-2-fluorophenyl)amino)methylene)malonate (17.2, 2.74 g, 7.61 mmol) was stirred at 100° C. under nitrogen atmosphere overnight. The reaction mixture was subjected to silica gel column chromatography to afford ethyl 6-bromo-8-fluoro-4-hydroxyquinoline-3-carboxylate (17.3, 400 mg, 17%). LC-MS (ESI): m/z 314.0 [M+H]⁺ and 316.1 [M+2+H]⁺.

A mixture of ethyl 6-bromo-8-fluoro-4-hydroxyquinoline-3-carboxylate (17.3, 400 mg, 1.27 mmol) and phosphorus oxychloride (3 mL) was stirred at 90° C. for 2 hrs. The reaction mixture was concentrated under reduced pressure and the residue was subjected to silica gel column chromatography to afford ethyl 6-bromo-4-chloro-8-fluoroquinoline-3-carboxylate (17.4, 350 mg, 83%). LC-MS (ESI): m/z 331.9 [M+H]⁺ and 334.0 [M+2+H]⁺.

To a solution of ethyl 6-bromo-4-chloro-8-fluoroquinoline-3-carboxylate (17.4, 190 mg, 0.57 mmol) in tetrahydrofuran (5 mL) was added methylmagnesium bromide (3 mL, 1 M in tetrahydrofuran) at 0° C., and the mixture was stirred at room temperature for 4 hrs. The reaction mixture was quenched with saturated ammonium chloride solution (5 mL), diluted with water (20 mL) and extracted with ethyl acetate (20 mL×3). The combined organic layers were washed with brine (20 mL), dried over anhydrous sodium sulfate and concentrated under reduced pressure. The residue was subjected to silica gel column chromatography to afford 2-(6-bromo-4-chloro-8-fluoroquinolin-3-yl)propan-2-ol (17.5, 182 mg, 52%). LC-MS (ESI): m/z 318.0 [M+H]⁺ and 319.9 [M+2+H]⁺.

To a solution of 2-(6-bromo-4-chloro-8-fluoroquinolin-3-yl)propan-2-ol (17.5, 85.0 mg, 0.27 mmol) in dioxane (2 mL) were added 4,4,5,5-tetramethyl-2-(4,4,5,5-tetramethyl-1,3,2-dioxaborolan-2-yl)-1,3,2-dioxaborolane (84.0 mg, 0.33 mmol), 1,1′-bis(diphenylphosphino)ferrocene-palladium(II) (7.7 mg, 0.01 mmol) and potassium acetate (110 mg, 1.12 mmol). The resulting mixture was stirred at 90° C. for 4 hrs under nitrogen atmosphere. The reaction mixture was cooled to room temperature, diluted with ethyl acetate (5 mL) and filtered. The filtrate was concentrated under reduced pressure and the residue was subjected to silica gel column chromatography to afford (4-chloro-8-fluoro-3-(2-hydroxypropan-2-yl)quinolin-6-yl)boronic acid (17.6, 36.0 mg, 48%). LC-MS (ESI): m/z 283.8 [M+H]⁺.

To a mixture of (4-chloro-8-fluoro-3-(2-hydroxypropan-2-yl)quinolin-6-yl)boronic acid (17.6, 36.0 mg, 0.10 mmol) and 2,4,5-trichloropyrimidine (1.7, 54.0 mg, 0.29 mmol) in dioxane (1 mL) and water (0.1 mL) were added tetrakis(triphenylphosphine)palladium (2.00 mg, 0.002 mmol) and potassium carbonate (35.0 mg, 0.25 mmol). The resulting mixture was stirred at 90° C. under nitrogen atmosphere for 2 hrs. The reaction mixture was cooled to room temperature, diluted with ethyl acetate (5 mL) and filtered. The filtrate was concentrated under reduced pressure and the residue was subjected to silica gel column chromatography to afford 2-(4-chloro-6-(2,5-dichloropyrimidin-4-yl)-8-fluoroquinolin-3-yl)propan-2-ol (17.7, 25.0 mg, 65%). LC-MS (ESI): m/z 386.0 [M+H]⁺ and 388.1 [M+2+H]⁺.

To a solution of 2-(4-chloro-6-(2,5-dichloropyrimidin-4-yl)-8-fluoroquinolin-3-yl)propan-2-ol (17.7, 24.6 mg, 0.06 mmol) and (3S,4R)-4-aminooxan-3-ol hydrochloride (1.9, 19.2 mg, 0.13 mmol) in anhydrous dimethyl sulfoxide (1.0 mL) was added N,N-diisopropylethylamine (0.15 mL), and the resulting mixture was stirred at 80° C. for 2 hours. The reaction mixture was subjected to prep-HPLC to afford (3S,4R)-4-((5-chloro-4-(4-chloro-8-fluoro-3-(2-hydroxypropan-2-yl)quinolin-6-yl)pyrimidin-2-yl)amino)tetrahydro-2H-pyran-3-ol (17, 14.0 mg, 47%). LC-MS (ESI): m/z 467.1 [M+H]⁺ and 469.0 [M+2+H]⁺. ¹H NMR (500 MHz, DMSO-d₆) δ 9.43 (s, 1H), 8.59 (s, 1H), 8.50 (s, 1H), 8.05 (d, J=10.0 Hz, 1H), 7.64 (s, 1H), 5.82 (s, 1H), 4.94 (d, J=5.5 Hz, 1H), 3.88-3.79 (m, 3H), 3.54-3.49 (m, 1H), 3.36-3.33 (m, 1H), 3.05 (t, J=10.0 Hz, 1H), 2.03-1.94 (m, 1H), 1.75 (s, 6H), 1.55-1.48 (m, 1H).

### Illustration 18. Synthesis of (3S,4R)-4-((5-chloro-4-(8-fluoro-3-(5-methyl-1H-imidazol-4-yl)quinolin-6-yl)pyrimidin-2-yl)amino)tetrahydro-2H-pyran-3-ol (18)

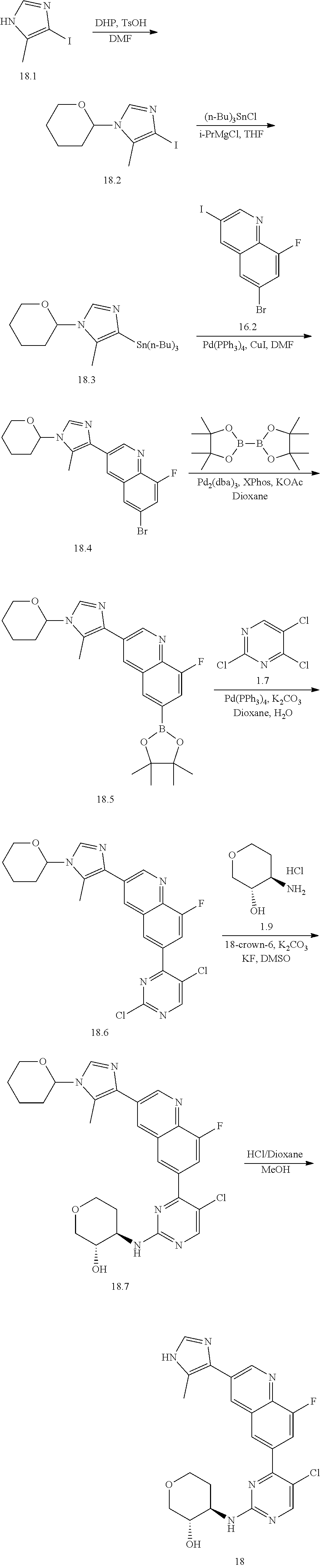

To a mixture of 4-iodo-5-methyl-1H-imidazole (18.1, 4.00 g, 19.2 mmol) and 3,4-dihydro-2H-pyran (4.85 g, 57.7 mmol) in N, N-dimethylformamide (50 mL) was added p-toluenesulfonic acid (331 mg, 1.92 mmol), and the mixture was stirred at 100° C. for 12 hrs. The reaction mixture was cooled to 25° C., then poured into water (50 mL) and extracted with ethyl acetate (50 mL×3). The combined organic layers were washed with brine (50 mL×3), dried over anhydrous sodium sulfate and concentrated under reduced pressure. The residue was subjected to silica gel column chromatography to afford 4-iodo-5-methyl-1-(tetrahydro-2H-pyran-2-yl)-1H-imidazole (18.2, 2.50 g, 44%) as a yellow oil. LC-MS (ESI): m/z 293.1 [M+H]⁺.

To a solution of 4-iodo-5-methyl-1-(tetrahydro-2H-pyran-2-yl)-1H-imidazole (18.2, 1.00 g, 3.42 mmol) in tetrahydrofuran (10 mL) was added isopropylmagnesium chloride (2.57 mL, 5.14 mmol) at 0° C. The mixture was stirred at 0° C. for 1 hr followed by addition of tributyl(chloro)stannane (1.25 mL, 4.64 mmol), and the resulting mixture was stirred at 25° C. for 12 hrs. The reaction mixture was poured into saturated aqueous ammonium chloride (20 mL) and extracted with ethyl acetate (20 mL×3). The combined organic layers were washed with brine (10 mL×2), dried over anhydrous sodium sulfate and concentrated under reduced pressure to give 5-methyl-1-(tetrahydro-2H-pyran-2-yl)-4-(tributylstannyl)-1H-imidazole (18.3, 1.50 g, crude) as a yellow oil. LC-MS (ESI): m/z 457.3 [M+H]⁺.

To a mixture of 6-bromo-8-fluoro-3-iodoquinoline (16.2, 900 mg, 2.56 mmol) and 5-methyl-1-(tetrahydro-2H-pyran-2-yl)-4-(tributylstannyl)-1H-imidazole (18.3, 1.50 g, crude) in N,N-dimethylformamide (15 mL) were added tetrakis(triphenylphosphine)palladium (296 mg, 0.26 mmol) and cuprous iodide (97.4 mg, 0.51 mmol), and the resulting mixture was stirred at 60° C. for 12 hrs under nitrogen atmosphere. The mixture was cooled to room temperature, then poured into water (20 mL) and extracted with ethyl acetate (20 mL×3). The combined organic layers were washed with brine (20 mL×2), dried over anhydrous sodium sulfate and concentrated under reduced pressure. The residue was subjected to silica gel column chromatography to afford 6-bromo-8-fluoro-3-(5-methyl-1-(tetrahydro-2H-pyran-2-yl)-1H-imidazol-4-yl)quinoline (18.4, 800 mg) as a yellow solid. LC-MS (ESI): m/z 390.2 [M+H]⁺.

To a mixture of 6-bromo-8-fluoro-3-(5-methyl-1-(tetrahydro-2H-pyran-2-yl)-1H-imidazol-4-yl)quinoline (18.4, 800 mg, 2.05 mmol), 4,4,5,5-tetramethyl-2-(4,4,5,5-tetramethyl-1,3,2-dioxaborolan-2-yl)-1,3,2-dioxaborolane (1.04 g, 4.10 mmol) and potassium acetate (604 mg, 6.15 mmol) in 1,4-dioxane (10 mL) were added 2-(dicyclohexylphosphino)-2′,4′,6′-tri-i-propyl-1,1′-biphenyl (XPhos, 195 mg, 0.41 mmol) and tris(dibenzylideneacetone)dipalladium (188 mg, 0.21 mmol). The mixture was stirred at 100° C. for 12 hrs under nitrogen atmosphere. The reaction mixture was filtered and the filter cake was washed with ethyl acetate (20 mL). The filtrate was concentrated under reduced pressure and the residue was subjected to silica gel column chromatography to afford 8-fluoro-3-(5-methyl-1-(tetrahydro-2H-pyran-2-yl)-1H-imidazol-4-yl)-6-(4,4,5,5-tetramethyl-1,3,2-dioxaborolan-2-yl)quinoline (18.5, 550 mg, 31%) as a yellow solid. LC-MS (ESI): m/z 438.2 [M+H]⁺.

To a mixture of 8-fluoro-3-(5-methyl-1-(tetrahydro-2H-pyran-2-yl)-1H-imidazol-4-yl)-6-(4,4,5,5-tetramethyl-1,3,2-dioxaborolan-2-yl)quinoline (18.5, 200 mg, 0.46 mmol) and 2,4,5-trichloropyrimidine (1.7, 251 mg, 1.37 mmol) in 1,4-dioxane (2 mL) and water (0.2 mL) were added tetrakis(triphenylphosphine)palladium (53.1 mg, 0.05 mmol) and potassium carbonate (127 mg, 0.92 mmol), and the reaction was stirred at 90° C. for 16 hrs under nitrogen atmosphere. The mixture was diluted with water (10 mL) and extracted with ethyl acetate (10 mL×2). The combined organic layers were dried over anhydrous sodium sulfate, filtered and concentrated under reduced pressure. The residue was subjected to silica gel column chromatography to afford 6-(2,5-dichloropyrimidin-4-yl)-8-fluoro-3-(5-methyl-1-(tetrahydro-2H-pyran-2-yl)-1H-imidazol-4-yl)quinoline (18.6, 190 mg, 91%) as a yellow solid. LC-MS (ESI): m/z 458.1 [M+H]⁺.

A mixture of 6-(2,5-dichloropyrimidin-4-yl)-8-fluoro-3-(5-methyl-1-(tetrahydro-2H-pyran-2-yl)-1H-imidazol-4-yl)quinoline (18.6, 190 mg, 0.42 mmol), (3S,4R)-4-aminotetrahydropyran-3-ol hydrochloride (1.9, 76.8 mg, 0.50 mmol), potassium carbonate (69.0 mg, 0.50 mmol), potassium fluoride (72.3 mg, 1.25 mmol) and 18-crown-6 (11.1 mg, 0.04 mmol) in dimethylsulfoxide (3 mL) was stirred at 120° C. for 2 hrs. The mixture was diluted with water (10 mL) and extracted with ethyl acetate (10 mL×2). The combined organic layers were washed with water (10 mL), dried over anhydrous sodium sulfate and concentrated under reduced pressure. The residue was subjected to silica gel column chromatography to afford (3S,4R)-4-((5-chloro-4-(8-fluoro-3-(5-methyl-1-(tetrahydro-2H-pyran-2-yl)-1H-imidazol-4-yl)quinolin-6-yl)pyrimidin-2-yl)amino)tetrahydro-2H-pyran-3-ol (18.7, 100 mg, 45%) as a yellow solid. LC-MS (ESI): m/z 539.2 [M+H]⁺.

To a solution of (3S,4R)-4-((5-chloro-4-(8-fluoro-3-(5-methyl-1-(tetrahydro-2H-pyran-2-yl)-1H-imidazol-4-yl)quinolin-6-yl)pyrimidin-2-yl)amino)tetrahydro-2H-pyran-3-ol (18.7, 100 mg, 0.19 mmol) in methanol (3 mL) was added hydrochloric acid (3 mL, 4 M in dioxane), and the mixture was stirred at room temperature for 24 hrs. The reaction mixture was concentrated under reduced pressure and the residue was subjected to prep-HPLC to afford (3S,4R)-4-((5-chloro-4-(8-fluoro-3-(5-methyl-1H-imidazol-4-yl)quinolin-6-yl)pyrimidin-2-yl)amino)tetrahydro-2H-pyran-3-ol (18, 21.7 mg, 26%) as a white solid. LC-MS (ESI): m/z 455.3 [M+H]⁺. ¹H NMR (500 MHz, DMSO-d₆) δ 12.29 (s, 1H), 9.42 (s, 1H), 8.64 (s, 1H), 8.48 (s, 11H), 8.31 (s, 11H), 7.81 (d, J=10.9 Hz, 1H), 7.73 (s, 11H), 7.56 (s, 1H), 4.93 (d, J=5.4 Hz, 1H), 3.86 (dd, J=10.6, 4.3 Hz, 1H), 3.82 (dd, J=10.9, 4.6 Hz, 2H), 3.53-3.48 (m, 1H), 3.36 (s, 1H), 3.05 (t, J=10.4 Hz, 1H), 2.54 (s, 3H), 1.96 (d, J=9.0 Hz, 1H), 1.50 (qd, J=12.2, 4.5 Hz, 1H).

### Illustration 19. Synthesis of (3S,4R)-4-((5-chloro-4-((R)-7-fluoro-1,2,3,4,4a,5-hexahydrobenzo[b]pyrazino[1,2-d][1,4]oxazin-9-yl)pyrimidin-2-yl)amino)tetrahydro-2H-pyran-3-ol (19)

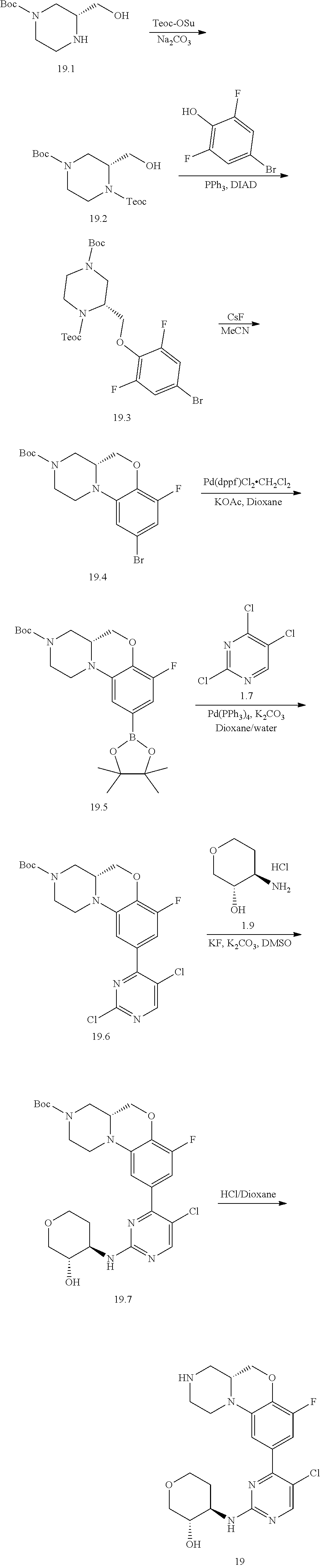

To a solution of tert-butyl (R)-3-(hydroxymethyl)piperazine-1-carboxylate (19.1, 5.00 g, 23.1 mmol) in tetrahydrofuran (80 mL) and water (80 mL) were added sodium carbonate (3.68 g, 34.7 mmol) and 2,5-dioxopyrrolidin-1-yl (2-(trimethylsilyl)ethyl) carbonate (5.39 g, 20.8 mmol), and the mixture was stirred at room temperature for 16 hrs. The reaction mixture was filtered and the filtrate was extracted with ethyl acetate (50 mL×3). The combined organic layers were dried over anhydrous sodium sulfate and concentrated under reduced pressure. The residue was subjected to silica gel column chromatography to afford 4-(tert-butyl) 1-(2,2,2-trichloroethyl) (R)-2-(hydroxymethyl)piperazine-1,4-dicarboxylate (19.2, 4.00 g, 48%) as a colorless oil. LC-MS (ESI): m/z 391.0 [M+H]⁺.

To a mixture of 4-(tert-butyl) 1-(2,2,2-trichloroethyl) (R)-2-(hydroxymethyl)piperazine-1,4-dicarboxylate (19.2, 4.00 g, 11.1 mmol), triphenylphosphine (3.78 g, 14.4 mmol) and 4-bromo-2,6-difluorophenol (2.32 g, 11.1 mmol) in tetrahydrofuran (60 mL) was added diisopropyl azodiformate (2.92 g, 14.4 mmol, 2.54 mL) dropwise at 0° C., and the resulting mixture was stirred at room temperature for 14 hrs under nitrogen atmosphere. The reaction mixture was diluted with water (100 mL) and extracted with ethyl acetate (50 mL×3). The combined organic layers were dried over anhydrous sodium sulfate and concentrated under reduced pressure. The residue was subjected to silica gel column chromatography to afford 4-(tert-butyl) 1-(2,2,2-trichloroethyl) (R)-2-((4-bromo-2,6-difluorophenoxy)methyl)piperazine-1,4-dicarboxylate (19.3, 3.20 g, 52%) as a colorless oil. LC-MS (ESI): m/z 525.0 [M-56+H]⁺.

To a solution of 4-(tert-butyl) 1-(2,2,2-trichloroethyl) (R)-2-((4-bromo-2,6-difluorophenoxy)methyl)piperazine-1,4-dicarboxylate (19.3, 3.20 g, 5.80 mmol) in acetonitrile (40 mL) was added cesium fluoride (4.40 g, 29.0 mmol), and the mixture was stirred at 90° C. for 15 hrs. The reaction mixture was filtered and the filtrate was concentrated under reduced pressure. The residue was subjected to silica gel column chromatography to afford tert-butyl (R)-9-bromo-7-fluoro-1,2,4a,5-tetrahydrobenzo[b]pyrazino[1,2-d][1,4]oxazine-3(4H)-carboxylate (19.4, 1.60 g, 71%) as a white solid. LC-MS (ESI): m/z 387.1 [M+H]⁺.

To a solution of tert-butyl (R)-9-bromo-7-fluoro-1,2,4a,5-tetrahydrobenzo[b]pyrazino[1,2-d][1,4]oxazine-3(4H)-carboxylate (19.4, 1.00 g, 2.58 mmol) in dioxane (5 mL) were added 4,4,5,5-tetramethyl-2-(4,4,5,5-tetramethyl-1,3,2-dioxaborolan-2-yl)-1,3,2-dioxaborolane (787 mg, 3.10 mmol), potassium acetate (760 mg, 7.74 mmol) and Pd(dppf)Cl₂ (191 mg, 0.25 mmol). The resulting mixture was stirred at 90° C. for 6 hrs under nitrogen atmosphere. The reaction mixture was diluted with water (20 mL) and extracted with ethyl acetate (10 mL×3). The combined organic layers were dried over anhydrous sodium sulfate, filtered and concentrated under reduced pressure. The residue was subjected to silica gel column chromatography to afford tert-butyl (R)-7-fluoro-9-(4,4,5,5-tetramethyl-1,3,2-dioxaborolan-2-yl)-1,2,4a,5-tetrahydrobenzo[b]pyrazino[1,2-d][1,4]oxazine-3(4R)-carboxylate (19.5, 340 mg, 30%) as a yellow solid. LC-MS (ESI): m/z 435.2 [M+H]⁺.

To a mixture of tert-butyl (R)-7-fluoro-9-(4,4,5,5-tetramethyl-1,3,2-dioxaborolan-2-yl)-1,2,4a,5-tetrahydrobenzo[b]pyrazino[1,2-d][1,4]oxazine-3(4H)-carboxylate (19.5, 340 mg, 0.78 mmol) and 2,4,5-trichloropyrimidine (1.7, 100 μL, 0.86 mmol) in water (0.5 mL) and dioxane (2 mL) were added Pd(PPh₃)₄ (135 mg, 0.11 mmol) and potassium carbonate (216 mg, 1.56 mmol). The reaction mixture was stirred at 90° C. for 15 hrs under nitrogen atmosphere. The mixture was diluted with water (20 mL) and extracted with ethyl acetate (10 mL×2). The combined organic layers were dried over anhydrous sodium sulfate, filtered and concentrated under reduced pressure. The residue was subjected to silica gel column chromatography to afford tert-butyl (R)-9-(2,5-dichloropyrimidin-4-yl)-7-fluoro-1,2,4a,5-tetrahydrobenzo[b]pyrazino[1,2-d][1,4]oxazine-3(4H)-carboxylate (19.6, 261 mg, 73%) as a yellow solid. LC-MS (ESI): m/z 399.1 [M-56+H]⁺.

A mixture of tert-butyl (R)-9-(2,5-dichloropyrimidin-4-yl)-7-fluoro-1,2,4a,5-tetrahydrobenzo[b]pyrazino[1,2-d][1,4]oxazine-3(4H)-carboxylate (19.6, 261 mg, 0.57 mmol), (3S,4R)-4-aminotetrahydro-2H-pyran-3-ol hydrochloride (1.9, 105 mg, 0.68 mmol), potassium carbonate (79.2 mg, 0.57 mmol), potassium fluoride (100 mg, 1.72 mmol) and 18-crown-6 (30.0 mg, 0.11 mmol) in dimethylsulfoxide (2 mL) was stirred at 120° C. for 1 hr. The mixture was diluted with water (20 mL) and extracted with ethyl acetate (10 mL×2). The combined organic layers were dried over anhydrous sodium sulfate, filtered and concentrated under reduced pressure. The residue was subjected to silica gel column chromatography to afford tert-butyl (R)-9-(5-chloro-2-(((3S,4R)-3-hydroxytetrahydro-2H-pyran-4-yl)amino)pyrimidin-4-yl)-7-fluoro-1,2,4a,5-tetrahydrobenzo[b]pyrazino[1,2-d][1,4]oxazine-3(4H)-carboxylate (19.7, 210 mg, 68%) as a white solid. LC-MS (ESI): m/z 536.2 [M+H]⁺.

To a solution of tert-butyl (R)-9-(5-chloro-2-(((3S,4R)-3-hydroxytetrahydro-2H-pyran-4-yl)amino)pyrimidin-4-yl)-7-fluoro-1,2,4a,5-tetrahydrobenzo[b]pyrazino[1,2-d][1,4]oxazine-3(4H)-carboxylate (19.7, 120 mg, 0.22 mmol) in methanol (2 mL) was added hydrochloric acid (1 mL, 4 M in dioxane), and the mixture was stirred at room temperature for 1 hr. The mixture was concentrated under reduced pressure, adjusted to pH 8 with saturated sodium bicarbonate solution (5 mL) and then extracted with ethyl acetate (20 mL×2). The combined organic layers were dried over anhydrous sodium sulfate, filtered and concentrated under reduced pressure. The residue was subjected to prep-HPLC to afford (3S,4R)-4-((5-chloro-4-((R)-7-fluoro-1,2,3,4,4a,5-hexahydrobenzo[b]pyrazino[1,2-d][1,4]oxazin-9-yl)pyrimidin-2-yl)amino)tetrahydro-2H-pyran-3-ol (19, 60.0 mg, 61%) as a white solid. LC-MS (ESI): m/z 436.1 [M+H]⁺. ¹H NMR (500 MHz, DMSO-d₆) δ 8.34 (s, 1H), 7.38 (d, J=8.0 Hz, 1H), 7.16-7.01 (m, 2H), 4.88 (brs, 1H), 4.38 (dd, J=10.8, 2.8 Hz, 1H), 3.98 (dd, J=10.8, 8.7 Hz, 1H), 3.82-3.76 (m, 3H), 3.67-3.63 (m, 1H), 3.51-3.47 (m, 1H), 3.31 (td, J=11.7, 2.3 Hz, 1H), 3.13-3.01 (m, 4H), 2.78 (td, J=12.1, 3.1 Hz, 1H), 2.67 (td, J=11.8, 3.2 Hz, 1H), 2.40 (t, J=11.4 Hz, 1H), 1.93 (d, J=13.2 Hz, 1H), 1.48 (qd, J=11.8, 4.5 Hz, 1H).

### Illustration 20. Synthesis of (3S,4R)-4-((5-chloro-4-(4-fluoro-8,9,10,11-tetrahydro-7H-8,11-epiminocyclohepta[c]quinolin-2-yl)pyrimidin-2-yl)amino)tetrahydro-2H-pyran-3-ol (20a) and (3S,4R)-4-((5-chloro-4-(4-fluoro-8,9,10,11-tetrahydro-7H-8,11-epiminocyclohepta[c]quinolin-2-yl)pyrimidin-2-yl)amino)tetrahydro-2H-pyran-3-ol (20b)

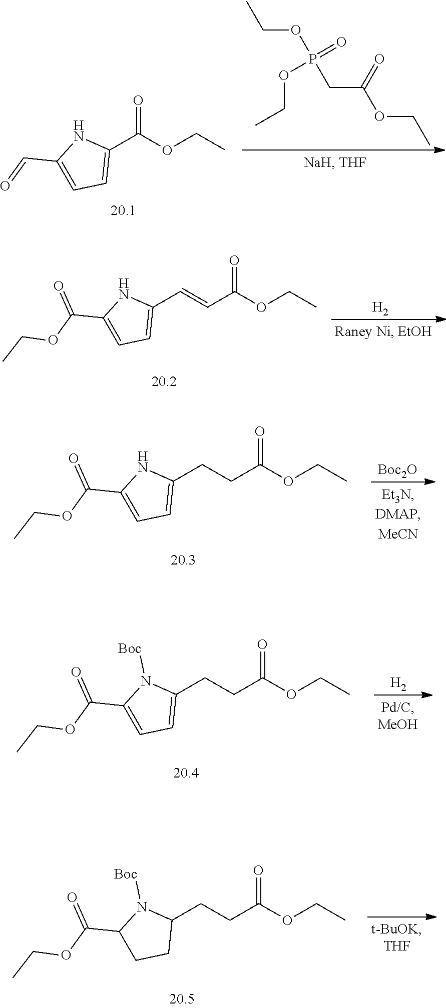

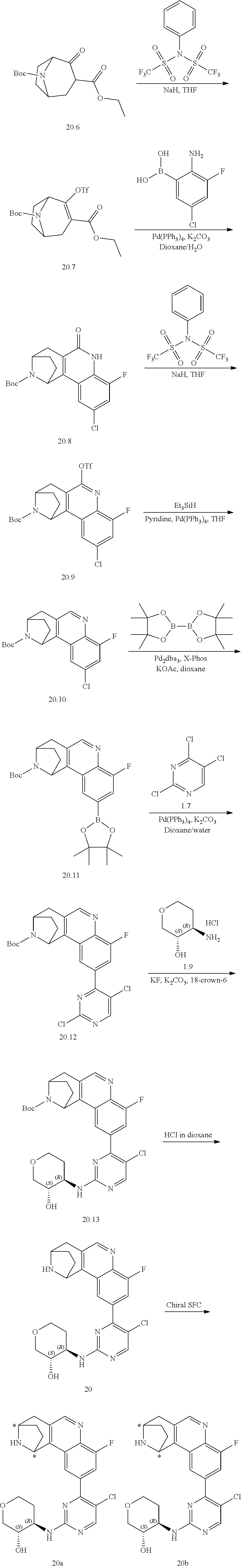

To a mixture of diethyl ethyl 2-(diethoxyphosphoryl) acetate (20.5 g, 91.5 mmol) in tetrahydrofuran (80 mL) was added sodium hydride (3.46 g, 86.4 mmol, 60% dispersion in mineral oil) in portions at 0° C. The mixture was stirred at 0° C. for 3 hrs under nitrogen atmosphere followed by addition of a solution of ethyl 5-formyl-1H-pyrrole-2-carboxylate (20.1, 8.50 g, 50.8 mmol) in tetrahydrofuran (30 mL). The resulting mixture was then stirred at room temperature for 18 hrs. The reaction mixture was quenched with saturated ammonium chloride solution (100 mL) and the aqueous layer was extracted with ethyl acetate (50 mL×3). The combined organic layers were washed with brine (100 mL), dried over anhydrous sodium sulfate, and concentrated under reduced pressure. The residue was purified by recrystallization from petroleum ether to afford ethyl (E)-5-(3-ethoxy-3-oxoprop-1-en-1-yl)-1H-pyrrole-2-carboxylate (20.2, 12.0 g, 99%). LC-MS (ESI): m/z 238.1 [M+H]⁺.

To a solution of ethyl (E)-5-(3-ethoxy-3-oxoprop-1-en-1-yl)-1H-pyrrole-2-carboxylate (20.2, 12.0 g, 50.5 mmol) in ethyl alcohol (100 mL) was added raney nickel (1.11 g, 5.06 mmol). The resulting mixture was degassed and backfilled with hydrogen three times, and then it was stirred at room temperature for 18 hrs under hydrogen atmosphere (1 atm). The reaction mixture was filtered and the filtrate was concentrated under reduced pressure to afford ethyl 5-(3-ethoxy-3-oxopropyl)-1H-pyrrole-2-carboxylate (20.3, 11.50 g, 95%) as a yellow oil. LC-MS (ESI): 240.1 [M+H]⁺.

To a mixture of ethyl 5-(3-ethoxy-3-oxopropyl)-1H-pyrrole-2-carboxylate (20.3, 11.5 g, 48.0 mmol), 4-dimethylaminopyridine (0.59 g, 4.81 mmol) and triethylamine (20 mL, 144 mmol) in acetonitrile (100 mL) was added di-tert-butyl dicarbonate (21.0 g, 96.2 mmol), and the mixture was stirred at 60° C. for 3 hrs. The reaction mixture was concentrated under reduced pressure. The residue was diluted with dichloromethane (50 mL) and washed with 1 N hydrochloric acid (25 mL×2). The organic phase was dried over anhydrous sodium sulfate, filtered and concentrated under reduced pressure. The residue was subjected to silica gel column chromatography to afford 1-(tert-butyl) 2-ethyl 5-(3-ethoxy-3-oxopropyl)-1H-pyrrole-1,2-dicarboxylate (20.4, 10.0 g, 61%) as a white solid. LC-MS (ESI): m/z 240.3[M-100+H]⁺.

To a solution of 1-(tert-butyl) 2-ethyl 5-(3-ethoxy-3-oxopropyl)-1H-pyrrole-1,2-dicarboxylate (20.4, 10.0 g, 29.4 mmol) in methanol (100 mL) was added palladium (1.00 g, 10% on carbon). The resulting mixture was degassed and backfilled with hydrogen three times, and then it was stirred at room temperature for 15 hrs under hydrogen atmosphere (1 atm). The reaction mixture was filtered and the filtrate was concentrated under reduced pressure to afford 1-(tert-butyl) 2-ethyl-5-(3-ethoxy-3-oxopropyl) pyrrolidine-1,2-dicarboxylate (20.5, 9.00 g, 89%) as a yellow oil. LC-MS (ESI): 244.1 [M-100+H]⁺.

To a solution of 1-(tert-butyl) 2-ethyl-5-(3-ethoxy-3-oxopropyl) pyrrolidine-1,2-dicarboxylate (20.5, 8.50 g, 24.7 mmol) in tetrahydrofuran (160 mL) was added potassium tert-butoxide (4.17 g, 37.1 mmol), and the mixture was stirred at 60° C. for 2 hrs. The reaction mixture was quenched with saturated ammonium chloride solution (100 mL) and the aqueous phase was extracted with ethyl acetate (80 mL×2). The combined organic layers were washed with brine (100 mL), dried over anhydrous sodium sulfate and concentrated under reduced pressure. The residue was subjected to silica gel column chromatography to afford 8-(tert-butyl) 3-ethyl 2-oxo-8-azabicyclo[3.2.1]octane-3,8-dicarboxylate (20.6, 5.50 g, 75%) as a white solid. LC-MS (ESI): 242.3 [M-56+H]⁺.

To a solution of 8-(tert-butyl) 3-ethyl 2-oxo-8-azabicyclo [3.2.1]octane-3,8-dicarboxylate (20.6, 5.00 g, 16.8 mmol) in tetrahydrofuran (50 mL) were added sodium hydride (2.02 g, 50.4 mmol, 60% dispersion in mineral oil) and N-phenyl-bis(trifluoromethanesulfonimide) (12.0 g, 33.6 mmol), and the mixture was stirred at 25° C. for 12 hrs. The reaction mixture was quenched with saturated ammonium chloride solution (100 mL) and the aqueous phase was extracted with ethyl acetate (50 mL×2). The combine organic layers were washed with brine (100 mL), dried over anhydrous sodium sulfate and concentrated under reduced pressure. The residue was subjected to silica gel column chromatography to afford 8-(tert-butyl) 3-ethyl 2-(((trifluoromethyl)sulfonyl) oxy)-8-azabicyclo[3.2.1]oct-2-ene-3,8-dicarboxylate (20.7, 6.30 g, crude). LC-MS (ESI): m/z 374.0 [M-56+H]⁺.

To a solution of 8-(tert-butyl) 3-ethyl 2-(((trifluoromethyl)sulfonyl) oxy)-8-azabicyclo[3.2.1]oct-2-ene-3,8-dicarboxylate (20.7, 6.30 g, crude) in dioxane (80 mL) and water (14 mL) were added (2-amino-5-chloro-3-fluorophenyl) boronic acid (3.43 g, 14.6 mmol), potassium carbonate (6.08 g, 44.0 mmol) and tetrakis (triphenylphosphine) palladium (1.70 g, 1.47 mmol). The mixture was stirred at 90° C. for 3 hrs under nitrogen atmosphere. The reaction mixture was diluted with water (50 mL) and extracted with ethyl acetate (50 mL×3). The combined organic layers were dried over anhydrous sodium sulfate, filtered and concentrated under reduced pressure. The residue was subjected to silica gel column chromatography to afford tert-butyl 2-chloro-4-fluoro-6-oxo-6,7,8,9,10,11-hexahydro-5H-8,11-epiminocyclohepta[c]quinoline-12-carboxylate (20.8, 1.30 g) as a yellow solid. LC-MS (ESI): 323.0 [M-56+H]⁺.

To a solution of tert-butyl 2-chloro-4-fluoro-6-oxo-6,7,8,9,10,11-hexahydro-5H-8,11-epiminocyclohepta[c]quinoline-12-carboxylate (20.8, 1.30 g, 3.43 mmol) in tetrahydrofuran (20 mL) were added sodium hydride (0.34 g, 8.58 mmol, 60% dispersion in paraffin liquid) and N-phenyl-bis(trifluoromethanesulfonimide)) (3.06 g, 8.58 mmol), and the mixture was stirred at 25° C. for 12 hrs. The reaction mixture was quenched with saturated ammonium chloride solution (50 mL) and the aqueous phase was extracted with ethyl acetate (50 mL×3). The combined organic layers were dried over anhydrous sodium sulfate, filtered and concentrated under reduced pressure. The residue was subjected to silica gel column chromatography to afford tert-butyl 2-chloro-4-fluoro-6-(((trifluoromethyl)sulfonyl)oxy)-8,9,10,11-tetrahydro-7H-8,11-epiminocyclohepta[c]quinoline-12-carboxylate (20.9, 1.74 g, 99%) as a yellow oil. LC-MS (ESI): m/z 511.2[M+H]⁺.

To a solution of tert-butyl 2-chloro-4-fluoro-6-(((trifluoromethyl)sulfonyl) oxy)-8,9,10,11-tetrahydro-7H-8,11-epiminocyclohepta[c]quinoline-12-carboxylate (20.9, 1.00 g, 1.96 mmol) in tetrahydrofuran (10 mL) were added tetrakis(triphenylphosphine)palladium (0.23 g, 0.20 mmol), pyridine (1.6 mL, 19.6 mmol) and triethylsilane (2.28 g, 19.6 mmol). The mixture was stirred at 50° C. for 3 hrs under nitrogen atmosphere. The reaction mixture was diluted with water (30 mL) and extracted with ethyl acetate (30 mL×3). The combined organic layers were dried over anhydrous sodium sulfate, filtered and concentrated under reduced pressure. The residue was subjected to silica gel column chromatography to afford tert-butyl 2-chloro-4-fluoro-8,9,10,11-tetrahydro-7H-8,11-epiminocyclohepta[c]quinoline-12-carboxylate (20.10, 0.30 g, 38%) as a white solid. LC-MS (ESI): m/z 363.0[M+H]⁺.

To a mixture of tert-butyl 2-chloro-4-fluoro-8,9,10,11-tetrahydro-7H-8,11-epiminocyclohepta[c]quinoline-12-carboxylate (20.10, 300 mg, 0.83 mmol), 4,4,4′,4′,5,5,5′,5′-octamethyl-2,2′-bi(1,3,2-dioxaborolane) (315 mg, 1.24 mmol), potassium acetate (243 mg, 2.48 mmol) and X-Phos (39 mg, 0.08 mmol) in dioxane (5 mL) was added tris(dibenzylideneacetone)dipalladium (76.0 mg, 0.08 mmol). The resulting mixture was stirred at 110° C. for 15 hrs under nitrogen atmosphere. The reaction mixture was filtered and the filtrate was concentrated under reduced pressure to afford tert-butyl 4-fluoro-2-(4,4,5,5-tetramethyl-1,3,2-dioxaborolan-2-yl)-8,9,10,11-tetrahydro-7H-8,11-epiminocyclohepta[c]quinoline-12-carboxylate (20.11, 0.38 g, crude). LC-MS (ESI): 455.1 [M+H]⁺.

A mixture of tert-butyl 4-fluoro-2-(4,4,5,5-tetramethyl-1,3,2-dioxaborolan-2-yl)-8,9,10,11-tetrahydro-7H-8,11-epiminocyclohepta[c]quinoline-12-carboxylate (20.11, 380 mg, crude), 2,4,5-trichloropyrimidine (1.7, 153 mg, 0.84 mmol), tetrakis(triphenylphosphine)palladium (97.0 mg, 0.08 mmol) and potassium carbonate (347 mg, 2.51 mmol) in dioxane (5 mL) and water (1 mL) was stirred at 90° C. for 3 hrs under nitrogen atmosphere. The reaction mixture was concentrated under reduced pressure and the residue was subjected to silica gel column chromatography to afford tert-butyl 2-(2,5-dichloropyrimidin-4-yl)-4-fluoro-8,9,10,11-tetrahydro-7H-8,11-epiminocyclohepta[c]quinoline-12-carboxylate (20.12, 364 mg, crude) as a white solid. LC-MS (ESI): m/z 475.0 [M+H]⁺.

A mixture of tert-butyl 2-(2,5-dichloropyrimidin-4-yl)-4-fluoro-8,9,10,11-tetrahydro-7H-8,11-epiminocyclohepta[c]quinoline-12-carboxylate (20.12, 364 mg, crude), (3S,4R)-4-aminotetrahydropyran-3-ol hydrochloride (1.9, 141 mg, 0.92 mmol), potassium carbonate (106 mg, 0.77 mmol), 18-crown-6 (20 mg, 0.08 mmol) and potassium fluoride (178 mg, 3.06 mmol) in dimethyl sulfoxide (2 mL) was stirred at 110° C. for 2 hrs. The reaction mixture was diluted with water (10 mL) and extracted with ethyl acetate (10 mL×2). The combined organic layers were washed with water (10 mL), dried over anhydrous sodium sulfate and concentrated under reduced pressure. The residue was subjected to silica gel column chromatography to afford tert-butyl 2-(5-chloro-2-(((3S,4R)-3-hydroxytetrahydro-2H-pyran-4-yl)amino)pyrimidin-4-yl)-4-fluoro-8,9,10,11-tetrahydro-7H-8,11-epiminocyclohepta[c]quinoline-12-carboxylate (20.13, 0.16 g) as a white solid. LC-MS (ESI): m/z 556.1 [M+H]⁺.

To a solution of tert-butyl 2-(5-chloro-2-(((3S,4R)-3-hydroxytetrahydro-2H-pyran-4-yl)amino)pyrimidin-4-yl)-4-fluoro-8,9,10,11-tetrahydro-7H-8,11-epiminocyclohepta[c]quinoline-12-carboxylate (20.13, 160 mg, 0.29 mmol) in dioxane (1 mL) was added hydrochloric acid (1 mL, 4 M in dioxane), and the mixture was stirred at room temperature for 1 hr. The mixture was concentrated under reduced pressure, adjusted to pH 8 with saturated sodium bicarbonate solution (5 mL) and then extracted with ethyl acetate (20 mL×2). The combined organic layers were dried over anhydrous sodium sulfate, filtered and concentrated under reduced pressure to afford (3S,4R)-4-((5-chloro-4-(4-fluoro-8,9,10,11-tetrahydro-7H-8,11-epiminocyclohepta[c]quinolin-2-yl)pyrimidin-2-yl)amino)tetrahydro-2H-pyran-3-ol (20, 118 mg, 91%) as a white solid, which was further separated by chiral SFC to give:

Isomer 1: (20a, 99.0% ee); Retention time: 0.763 min. LC-MS (ESI): m/z 456.2 [M+H]⁺; ¹H NMR (500 MHz, DMSO-d₆) δ 8.70 (s, 1H), 8.48 (s, 1H), 8.34 (s, 1H), 7.84 (s, 1H), 7.58 (s, 1H), 4.92 (dd, J=19.6, 5.8 Hz, 2H), 3.93-3.76 (m, 4H), 3.51 (s, 1H), 3.36 (s, 1H), 3.24 (dd, J=17.0, 4.6 Hz, 1H), 3.05 (t, J=10.4 Hz, 1H), 2.69 (d, J=17.1 Hz, 1H), 2.09 (dt, J=17.6, 5.7 Hz, 1H), 2.05-1.90 (m, 2H), 1.84 (t, J=9.8 Hz, 1H), 1.55 (dt, J=20.0, 9.7 Hz, 2H).

Isomer 2: (20b, 99.3% ee); Retention time: 1.130 min; LC-MS (ESI): m/z 456.2 [M+H]⁺. ¹H NMR (500 MHz, DMSO-d₆) δ 8.70 (s, 1H), 8.48 (s, 1H), 8.34 (s, 1H), 7.84 (s, 1H), 7.58 (s, 1H), 4.92 (dd, J=15.8, 5.8 Hz, 2H), 3.95-3.74 (m, 4H), 3.51 (s, 1H), 3.36 (s, 1H), 3.24 (dd, J=17.0, 4.5 Hz, 1H), 3.05 (t, J=10.4 Hz, 1H), 2.69 (d, J=17.0 Hz, 1H), 2.16-1.92 (m, 3H), 1.84 (t, J=9.7 Hz, 1H), 1.63-1.43 (m, 2H).

Analytical separation method: column: CHIRAL ART Cellulose-SB, 4.6×50 mm, 3.0 um; mobile phase: A for MTBE (0.1% DEA) and B for EtOH; gradient: B 30%; flow rate: 1.67 mL/min; high pressure: 114 bar; column temperature: 25° C.; wavelength: 254 nm.

Preparative separation method: column: CHIRAL ART Cellulose-SB, 20×250 mm, 5 um; mobile phase: A for MTBE (10 mM NH₃) and B for EtOH; gradient: B 30%; flow rate: 20 mL/min; high pressure: 63 bar; column temperature: 25° C. wavelength: 256 nm/234 nm; cycle time: ~12 mins.

### Illustration 21. Synthesis of (3S,4R)-4-((5-chloro-4-(7-fluoro-1,4-dihydro-2H-spiro[benzo[c][2,6]naphthyridine-3,1′-cyclopropan]-9-yl)pyrimidin-2-yl)amino)tetrahydro-2H-pyran-3-ol (21)

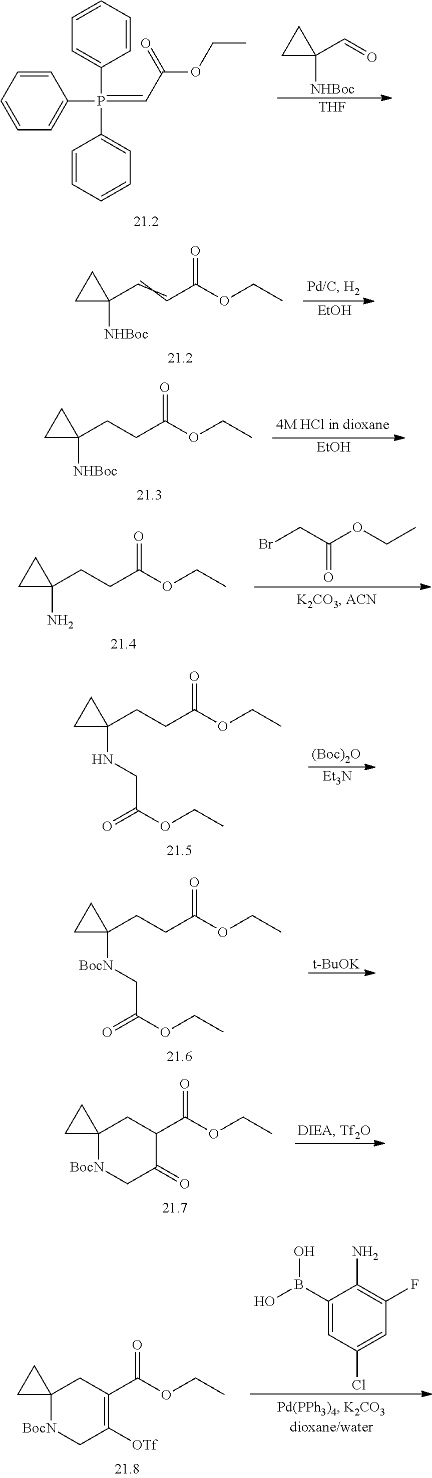

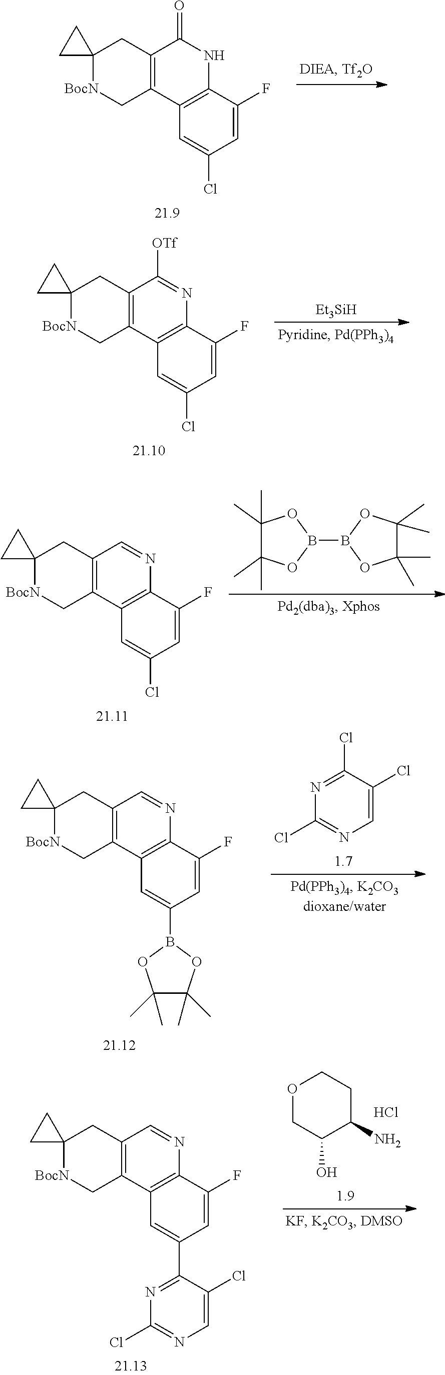

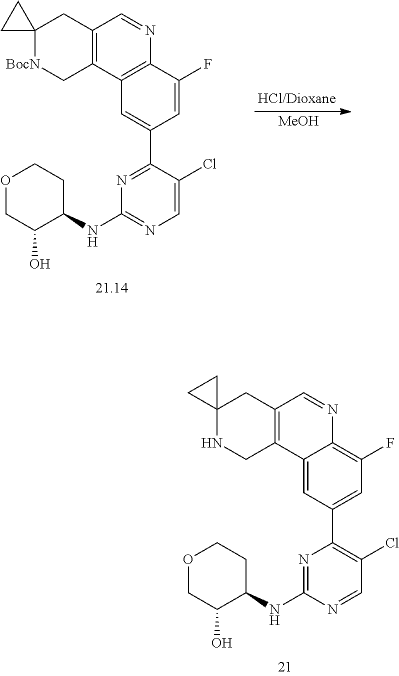 [SMILES: "MeOH" ; CC(C)(C)OC(=O)N1Cc2c(cnc3c(F)cc(-c4nc(N[C@@H]5CCOC[C@H]5O)ncc4Cl)cc23)CC12CC2 ; O[C@@H]1COCC[C@H]1Nc1ncc(Cl)c(-c2cc(F)c3ncc4c(c3c2)CNC2(CC2)C4)n1]

To a suspension of tert-butyl (1-formylcyclopropyl)carbamate (10.0 g, 54.0 mmol) in tetrahydrofuran (200 mL) was added ethyl 2-(triphenyl-λ⁵-phosphaneylidene)acetate (21.1, 37.6 g, 108 mmol) in portions, and the mixture was stirred at room temperature for 20 hrs. The reaction mixture was concentrated in vacuum to remove the solvent and the residue was triturated with petroleum ether (150 mL). Precipitation occurred after 10 mins and the mixture was filtered. The filtrate was concentrated under reduced pressure and the residue was subjected to silica gel column chromatography to afford ethyl 3-(1-((tert-butoxycarbonyl)amino)cyclopropyl)acrylate (21.2, 12.0 g, 87%) as a white solid. LC-MS (ESI): m/z 156.2 [M-100+H]⁺.

To a solution of ethyl 3-(1-((tert-butoxycarbonyl)amino)cyclopropyl)acrylate (21.2, 12.00 g, 47.0 mmol) in ethyl acetate (80 mL) was added palladium (1.00 g, 10% on carbon). The resulting mixture was degassed and backfilled with hydrogen three times, and then it was stirred at room temperature for 6 hrs under hydrogen atmosphere (1 atm). The reaction mixture was filtered and the filtrate was concentrated under reduced pressure to give ethyl 3-(1-((tert-butoxycarbonyl)amino)cyclopropyl)propanoate (21.3, 11.0 g, crude) as a white solid. LC-MS (ESI): m/z 158.2 [M-100+H]⁺.

To a solution of ethyl 3-(1-((tert-butoxycarbonyl)amino)cyclopropyl)propanoate (21.3, 11.0 g, crude) in ethanol (60 mL) was added hydrochloric acid (50 mL, 4 M in dioxane), and the mixture was stirred at 40° C. for 3 hrs. The reaction mixture was concentrated under reduced pressure to afford ethyl 3-(1-aminocyclopropyl)propanoate hydrogen chloride (21.4, 8.56 g, crude) as an off-white solid. LC-MS (ESI): m/z 158.1 [M+H]⁺.

To a mixture of ethyl 3-(1-aminocyclopropyl)propanoate hydrogen chloride (21.4, 8.56 g, crude) and potassium carbonate (12.2 g, 88.4 mmol) in acetonitrile (80 mL) was added ethyl 2-bromoacetate (8.85 g, 53.0 mmol), and the resulting mixture was stirred at room temperature for 16 hrs. The reaction mixture was filtered and the filtrate was concentrated under reduced pressure. The residue was subjected to silica gel column chromatography to afford ethyl 3-(1-((2-ethoxy-2-oxoethyl)amino)cyclopropyl)propanoate (21.5, 5.74 g) as a colorless oil. LC-MS (ESI): m/z 244.2 [M+H]⁺.

To a mixture of ethyl 3-(1-((2-ethoxy-2-oxoethyl)amino)cyclopropyl)propanoate (21.5, 5.74 g, 23.6 mmol) and triethylamine (4.92 mL, 35.4 mmol) in dichloromethane (50 mL) were added 4-dimethylaminopyridine (144 mg, 1.18 mmol) and di-tert-butyl dicarbonate (6.18 g, 28.3 mmol). The reaction mixture was stirred at room temperature for 3 hrs. The mixture was concentrated under reduced pressure and the residue was subjected to silica gel column chromatography to afford ethyl 3-(1-((tert-butoxycarbonyl)(2-ethoxy-2-oxoethyl)amino)cyclopropyl)propanoate (21.6, 3.81 g, 47%) as a colorless oil. LC-MS (ESI): m/z 244.2 [M-100+H]⁺.

To a solution of ethyl 3-(1-((tert-butoxycarbonyl)(2-ethoxy-2-oxoethyl)amino)cyclopropyl)propanoate (21.6, 1.50 g, 4.37 mmol) in tetrahydrofuran (15 mL) was added potassium tert-butoxide (637 mg, 5.68 mmol) at 0° C., and the mixture was stirred at 0° C. for 30 mins. The mixture was poured into sat. ammonium chloride aqueous solution (30 mL) and extracted with ethyl acetate (15 mL×3). The combined organic layers were dried over anhydrous sodium sulfate, filtered and concentrated under reduced pressure. The residue was subjected to silica gel column chromatography to afford 4-(tert-butyl) 7-ethyl 6-oxo-4-azaspiro[2.5]octane-4,7-dicarboxylate (21.7, 1.20 g, 92%) as a colorless oil. LC-MS (ESI): m/z 198.2 [M-100+H]⁺.

To a mixture of 4-(tert-butyl) 7-ethyl 6-oxo-4-azaspiro[2.5]octane-4,7-dicarboxylate (21.7, 1.20 g, 4.03 mmol) and N,N-diisopropylethylamine (7.03 mL, 40.3 mmol) in dichloromethane (20 mL) was added trifluoromethanesulfonic anhydride (1.70 mL, 10.1 mmol) dropwise at 0° C., and the mixture was stirred at 0° C. for 30 mins. The mixture was concentrated under reduced pressure and the residue was subjected to silica gel column chromatography to afford 4-(tert-butyl) 7-ethyl 6-(((trifluoromethyl)sulfonyl)oxy)-4-azaspiro[2.5]oct-6-ene-4,7-dicarboxylate (21.8, 1.34 g, 77%) as a colorless oil. LC-MS (ESI): m/z 330.1 [M-100+H]⁺.

To a solution of 4-(tert-butyl) 7-ethyl 6-(((trifluoromethyl)sulfonyl)oxy)-4-azaspiro[2.5]oct-6-ene-4,7-dicarboxylate (21.8, 1.34 g, 3.12 mmol) in dioxane (10 mL) and water (2 mL) were added (2-amino-5-chloro-3-fluorophenyl) boronic acid (1.02 g, 3.74 mmol), potassium carbonate (862 mg, 6.24 mmol) and tetrakis (triphenylphosphine) palladium (541 mg, 0.47 mmol). The mixture was stirred at 90° C. for 3 hrs under nitrogen atmosphere. The reaction mixture was cooled to room temperature, diluted with water (50 mL) and extracted with ethyl acetate (30 mL×3). The combined organic layers were dried over anhydrous sodium sulfate, filtered and concentrated under reduced pressure. The residue was subjected to silica gel column chromatography to afford tert-butyl 9-chloro-7-fluoro-5-oxo-1,4,5,6-tetrahydro-2H-spiro[benzo[c][2,6]naphthyridine-3,1′-cyclopropane]-2-carboxylate (21.9, 860 mg, 73%) as a white solid. LC-MS (ESI): 323.1 [M-56+H]⁺.

To a mixture of tert-butyl 9-chloro-7-fluoro-5-oxo-1,4,5,6-tetrahydro-2H-spiro[benzo[c][2,6]naphthyridine-3,1′-cyclopropane]-2-carboxylate (21.9, 860 mg, 2.27 mmol) and N,N-diisopropylethylamine (3.95 mL, 22.7 mmol) in dichloromethane (10 mL) was added trifluoromethanesulfonic anhydride (0.95 mL, 5.67 mmol) at 0° C., and the mixture was stirred at 0° C. for 45 mins. The mixture was concentrated under reduced pressure and the residue was subjected to silica gel column chromatography to afford tert-butyl 9-chloro-7-fluoro-5-(((trifluoromethyl)sulfonyl)oxy)-1,4-dihydro-2H-spiro[benzo[c][2,6]naphthyridine-3,1′-cyclopropane]-2-carboxylate (21.10, 730 mg, 63%) as a white solid. LC-MS (ESI): m/z 511.1 [M+H]⁺.

To a solution of tert-butyl 9-chloro-7-fluoro-5-(((trifluoromethyl)sulfonyl)oxy)-1,4-dihydro-2H-spiro[benzo[c][2,6]naphthyridine-3,1′-cyclopropane]-2-carboxylate (21.10, 730 mg, 1.43 mmol) in tetrahydrofuran (10 mL) were added tetrakis(triphenylphosphine)palladium (0.33 g, 0.28 mmol), pyridine (1.15 mL, 14.3 mmol) and triethylsilane (1.66 g, 14.3 mmol).

The mixture was stirred at 70° C. for 2 hrs under nitrogen atmosphere. The reaction mixture was cooled to room temperature, diluted with water (30 mL) and extracted with ethyl acetate (30 mL×3). The combined organic layers were dried over anhydrous sodium sulfate, filtered and concentrated under reduced pressure. The residue was subjected to silica gel column chromatography to afford tert-butyl 9-chloro-7-fluoro-1,4-dihydro-2H-spiro[benzo[c][2,6]naphthyridine-3,1′-cyclopropane]-2-carboxylate (21.11, 0.43 g, 83%) as a white solid. LC-MS (EST): m/z 363.2[M+H]⁺.

To a mixture of tert-butyl 9-chloro-7-fluoro-1,4-dihydro-2H-spiro[benzo[c][2,6]naphthyridine-3,1′-cyclopropane]-2-carboxylate (21.11, 430 mg, 1.18 mmol), 4,4,4′,4′,5,5,5′,5′-octamethyl-2,2′-bi(1,3,2-dioxaborolane) (391 mg, 1.54 mmol), potassium acetate (349 mg, 3.55 mmol) and X-Phos (113 mg, 0.24 mmol) in dioxane (6 mL) was added tris(dibenzylideneacetone)dipalladium (217 mg, 0.24 mmol). The resulting mixture was stirred at 100° C. for 2 hrs under nitrogen atmosphere. The reaction mixture was cooled to room temperature and filtered. The filtrate was concentrated under reduced pressure and the residue was subjected to silica gel column chromatography to afford tert-butyl 7-fluoro-9-(4,4,5,5-tetramethyl-1,3,2-dioxaborolan-2-yl)-1,4-dihydro-2H-spiro[benzo[c][2,6]naphthyridine-3,1′-cyclopropane]-2-carboxylate (21.12, 452 mg, 84%) as a yellow solid. LC-MS (ESI): 455.2 [M+H]⁺.

A mixture of tert-butyl 7-fluoro-9-(4,4,5,5-tetramethyl-1,3,2-dioxaborolan-2-yl)-1,4-dihydro-2H-spiro[benzo[c][2,6]naphthyridine-3,1′-cyclopropane]-2-carboxylate (21.12, 150 mg, 0.33 mmol), 2,4,5-trichloropyrimidine (1.7, 109 mg, 0.59 mmol), tetrakis(triphenylphosphine)palladium (57.2 mg, 0.05 mmol) and potassium carbonate (91.2 mg, 0.66 mmol) in dioxane (3 mL) and water (0.6 mL) was stirred at 90° C. for 3 hrs under nitrogen atmosphere. The reaction mixture was cooled to room temperature and concentrated under reduced pressure. The residue was subjected to silica gel column chromatography to afford tert-butyl 9-(2,5-dichloropyrimidin-4-yl)-7-fluoro-1,4-dihydro-2H-spiro[benzo[c][2,6]naphthyridine-3,1′-cyclopropane]-2-carboxylate (21.13, 144 mg, 92%) as a yellow solid. LC-MS (ESI): m/z 475.2 [M+H]⁺.

A mixture of tert-butyl 9-(2,5-dichloropyrimidin-4-yl)-7-fluoro-1,4-dihydro-2H-spiro[benzo[c][2,6]naphthyridine-3,1′-cyclopropane]-2-carboxylate (21.13, 144 mg, 0.30 mmol), (3S,4R)-4-aminotetrahydropyran-3-ol hydrochloride (1.9, 55.8 mg, 0.36 mmol), potassium carbonate (41.8 mg, 0.30 mmol), 18-crown-6 (16.0 mg, 0.06 mmol) and potassium fluoride (52.8 mg, 0.91 mmol) in dimethyl sulfoxide (2 mL) was stirred at 110° C. for 1 hr. The reaction mixture was cooled to room temperature, diluted with water (10 mL) and extracted with ethyl acetate (10 mL×2). The combined organic layers were washed with water (10 mL×3), dried over anhydrous sodium sulfate and concentrated under reduced pressure. The residue was subjected to silica gel column chromatography to afford tert-butyl 9-(5-chloro-2-(((3S,4R)-3-hydroxytetrahydro-2H-pyran-4-yl)amino)pyrimidin-4-yl)-7-fluoro-1,4-dihydro-2H-spiro[benzo[c][2,6]naphthyridine-3,1′-cyclopropane]-2-carboxylate (21.14, 0.13 g, 77%) as a white solid. LC-MS (ESI): m/z 556.2 [M+H]⁺.

To a solution of tert-butyl 9-(5-chloro-2-(((3S,4R)-3-hydroxytetrahydro-2H-pyran-4-yl)amino)pyrimidin-4-yl)-7-fluoro-1,4-dihydro-2H-spiro[benzo[c][2,6]naphthyridine-3,1′-cyclopropane]-2-carboxylate (21.14, 130 mg, 0.23 mmol) in methanol (1 mL) was added hydrochloric acid (1 mL, 4 M in dioxane), and the mixture was stirred at room temperature for 1 hr. The mixture was concentrated under reduced pressure, adjusted to pH 8 with saturated sodium bicarbonate aqueous solution (0.6 mL) and then subjected to prep-HPLC to afford (3S,4R)-4-((5-chloro-4-(7-fluoro-1,4-dihydro-2H-spiro[benzo[c][2,6]naphthyridine-3,1′-cyclopropan]-9-yl)pyrimidin-2-yl)amino)tetrahydro-2H-pyran-3-ol (21, 73.0 mg, 68%) as a yellow solid. LC-MS (ESI): m/z 456.2 [M+H]⁺. ¹H NMR (500 MHz, DMSO-d₆) δ 8.73 (s, 1H), 8.48 (s, 1H), 8.22 (s, 1H), 7.86 (d, J=11.4 Hz, 1H), 7.57 (s, 1H), 4.94 (s, 1H), 4.29 (s, 2H), 3.92-3.77 (m, 3H), 3.52-3.50 (m, 1H), 3.35 (td, J=11.7, 2.3 Hz, 1H), 3.06 (dd, J=11.1, 9.6 Hz, 1H), 2.88 (s, 2H), 2.01-1.92 (m, 1H), 1.57-1.45 (m, 1H), 0.69-0.62 (m, 2H), 0.55-0.49 (m, 2H).

### Illustration 22. Synthesis of (3S,4R)-4-((4-(4-chloro-8-fluoro-3-(2-hydroxypropan-2-yl)quinolin-6-yl)-5-fluoropyrimidin-2-yl)amino)tetrahydro-2H-pyran-3-ol (22)

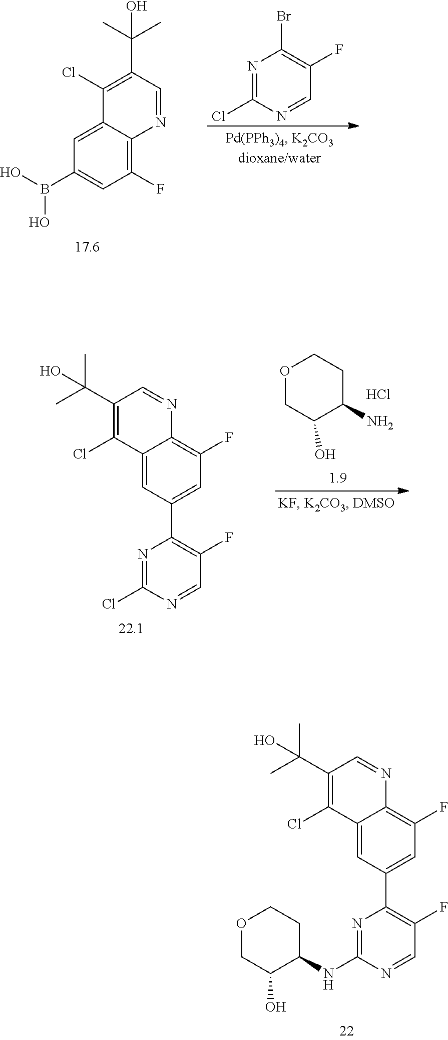 [SMILES: CC(C)(O)c1cnc2c(F)cc(B(O)O)cc2c1Cl ; Fc1cnc(Cl)nc1Br ; CC(C)(O)c1cnc2c(F)cc(-c3nc(Cl)ncc3F)cc2c1Cl ; N[C@@H]1CCOC[C@H]1O ; Cl ; CC(C)(O)c1cnc2c(F)cc(-c3nc(N[C@@H]4CCOC[C@H]4O)ncc3F)cc2c1Cl]

To a mixture of (4-chloro-8-fluoro-3-(2-hydroxypropan-2-yl)quinolin-6-yl)boronic acid (17.6, 50.0 mg, 0.17 mmol) and 4-bromo-2-chloro-5-fluoropyrimidine (48.5 mg, 0.23 mmol) in dioxane (1 mL) and water (0.2 mL) were added tetrakis(triphenylphosphine)palladium (30.6 mg, 0.03 mmol) and potassium carbonate (48.7 mg, 0.35 mmol). The resulting mixture was stirred at 90° C. under nitrogen atmosphere for 2 hrs. The reaction mixture was cooled to room temperature, diluted with ethyl acetate (5 mL) and filtered. The filtrate was concentrated under reduced pressure and the residue was subjected to silica gel column chromatography to afford 2-(4-chloro-6-(2-chloro-5-fluoropyrimidin-4-yl)-8-fluoroquinolin-3-yl)propan-2-ol (22.1, 50.0 mg, 76%) as a white solid. LC-MS (ESI): m/z 370.0 [M+H]⁺.

To a solution of 2-(4-chloro-6-(2-chloro-5-fluoropyrimidin-4-yl)-8-fluoroquinolin-3-yl)propan-2-ol (22.1, 50.0 mg, 0.13 mmol) and (3S,4R)-4-aminooxan-3-ol hydrochloride (1.9, 27.0 mg, 0.17 mmol) in anhydrous dimethyl sulfoxide (1.0 mL) were added potassium carbonate (18.6 mg, 0.13 mmol), potassium fluoride (23.5 mg, 0.40 mmol) and 18-crown-6 (7.19 mg, 0.03 mmol). The resulting mixture was stirred at 80° C. for 2 hours. The reaction mixture was cooled to room temperature, filtered and then subjected to prep-HPLC to afford (3S,4R)-4-((4-(4-chloro-8-fluoro-3-(2-hydroxypropan-2-yl)quinolin-6-yl)-5-fluoropyrimidin-2-yl)amino)tetrahydro-2H-pyran-3-ol (22, 12.0 mg, 19%) as a white solid. LC-MS (ESI): m/z 451.1 [M+H]. ¹H NMR (500 MHz, DMSO-d₆) δ 9.44 (s, 1H), 8.85 (s, 1H), 8.54 (d, J=3.7 Hz, 1H), 8.21 (d, J=13 Hz, 1H), 7.37 (d, J=7.8 Hz, 1H), 5.82 (s, 1H), 4.94 (d, J=5.5 Hz, 1H), 3.87-3.82 (m, 3H), 3.57-3.50 (m, 1H), 3.38 (t, J=11.8 Hz, 1H), 3.09 (t, J=10.4 Hz, 1H), 2.07-1.98 (m, 1H), 1.75 (s, 6H), 1.58-1.46 (m, 1H).

### Illustration 23. Synthesis of(3S,4R)-4-((5-fluoro-4-(8-fluoro-3-(2-hydroxypropan-2-yl)-4-methylquinolin-6-yl)pyrimidin-2-yl)amino)tetrahydro-2H-pyran-3-ol (23)

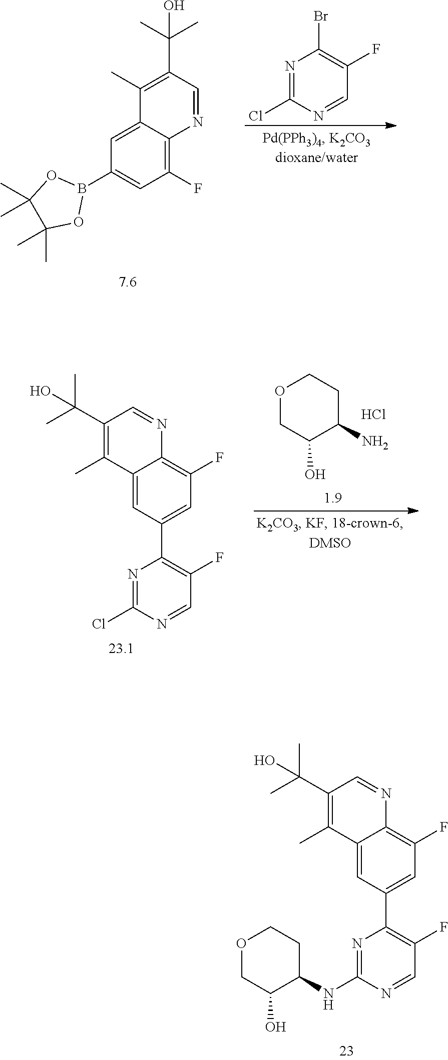 [SMILES: Cc1c(C(C)(C)O)cnc2c(F)cc(B3OC(C)(C)C(C)(C)O3)cc12 ; Fc1cnc(Cl)nc1Br ; Cc1c(C(C)(C)O)cnc2c(F)cc(-c3nc(Cl)ncc3F)cc12 ; N[C@@H]1CCOC[C@H]1O ; Cl ; Cc1c(C(C)(C)O)cnc2c(F)cc(-c3nc(N[C@@H]4CCOC[C@H]4O)ncc3F)cc12]

A mixture of 2-(8-fluoro-4-methyl-6-(4,4,5,5-tetramethyl-1,3,2-dioxaborolan-2-yl)quinolin-3-yl)propan-2-ol (7.6, 342 mg, 0.99 mmol), 4-bromo-2-chloro-5-fluoropyrimidine (210 mg, 0.99 mmol), tetrakis(triphenylphosphine)palladium (115 mg, 0.099 mmol) and potassium carbonate (275 mg, 1.99 mmol) in dioxane (3 mL) and water (0.6 mL) was stirred at 90° C. for 2 hrs under nitrogen atmosphere. The reaction mixture was cooled to room temperature and concentrated under reduced pressure. The residue was subjected to silica gel column chromatography to afford 2-(6-(2-chloro-5-fluoropyrimidin-4-yl)-8-fluoro-4-methylquinolin-3-yl)propan-2-ol (23.1, 220 mg, 63%) as a yellow oil. LC-MS (EST): m/z 350.1 [M+H]⁺.

A mixture of 2-(6-(2-chloro-5-fluoropyrimidin-4-yl)-8-fluoro-4-methylquinolin-3-yl)propan-2-ol (23.1, 100 mg, 0.27 mmol), (3S,4R)-4-aminotetrahydropyran-3-ol hydrogen chloride (1.9, 53.0 mg, 0.34 mmol), potassium fluoride (66.0 mg, 1.14 mmol), 18-crown-6 (75.6 mg, 0.29 mmol) and potassium carbonate (79.0 mg, 0.58 mmol) in dimethyl sulfoxide (2 mL) was stirred at 80° C. for 6 hrs. The reaction mixture was cooled to room temperature and filtered. The filtrate was subjected to prep-HPLC to afford (3S,4R)-4-((5-fluoro-4-(8-fluoro-3-(2-hydroxypropan-2-yl)-4-methylquinolin-6-yl)pyrimidin-2-yl)amino)tetrahydro-2H-pyran-3-ol (23, 12.4 mg, 10%)_as a white solid. LC-MS (ESI): m/z 431.2 [M+H]⁺. ¹H NMR (500 MHz, DMSO-d₆) δ 9.13 (s, 1H), 8.68 (s, 1H), 8.52 (d, J=3.7 Hz, 1H), 8.07 (d, J=10.7 Hz, 1H), 7.31 (d, J=7.8 Hz, 11H), 5.44 (s, 1H), 4.96 (d, J=5.4 Hz, 11H), 3.94-3.75 (m, 3H), 3.59-3.48 (m, 1H), 3.40-3.30 (m, 1H), 3.13-3.05 (m, 1H), 2.99 (s, 3H), 2.10-1.94 (m, 1H), 1.69 (s, 6H), 1.57-1.47 (m, 1H).

### Illustration 24. Synthesis of (3S,4R)-4-((5-fluoro-4-(7-fluoro-3,3-dimethyl-1,2,3,4-tetrahydrobenzo[c][2,6]naphthyridin-9-yl)pyrimidin-2-yl)amino)tetrahydro-2H-pyran-3-ol (24)

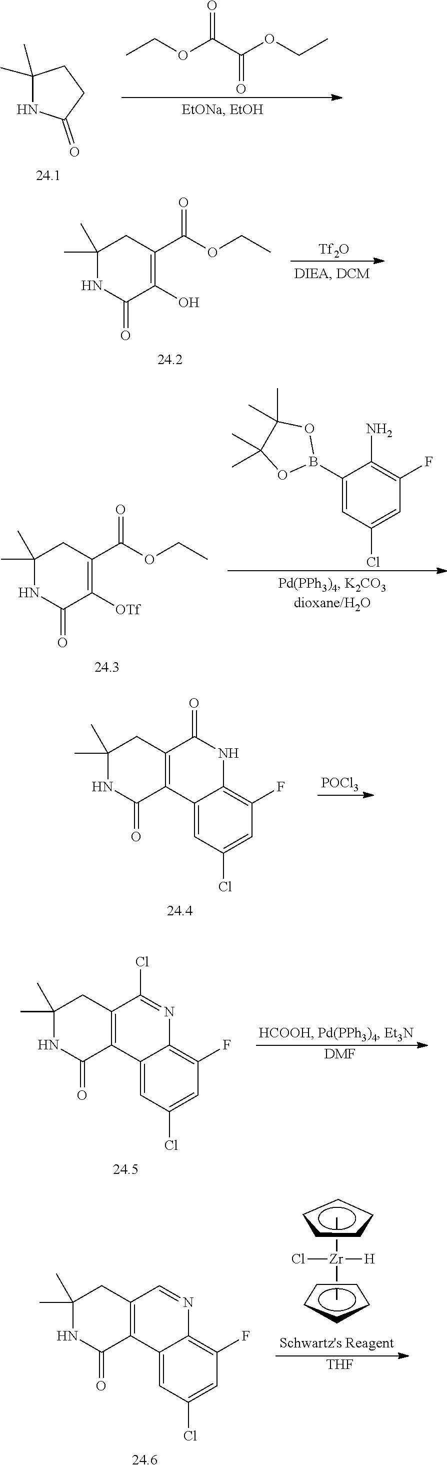

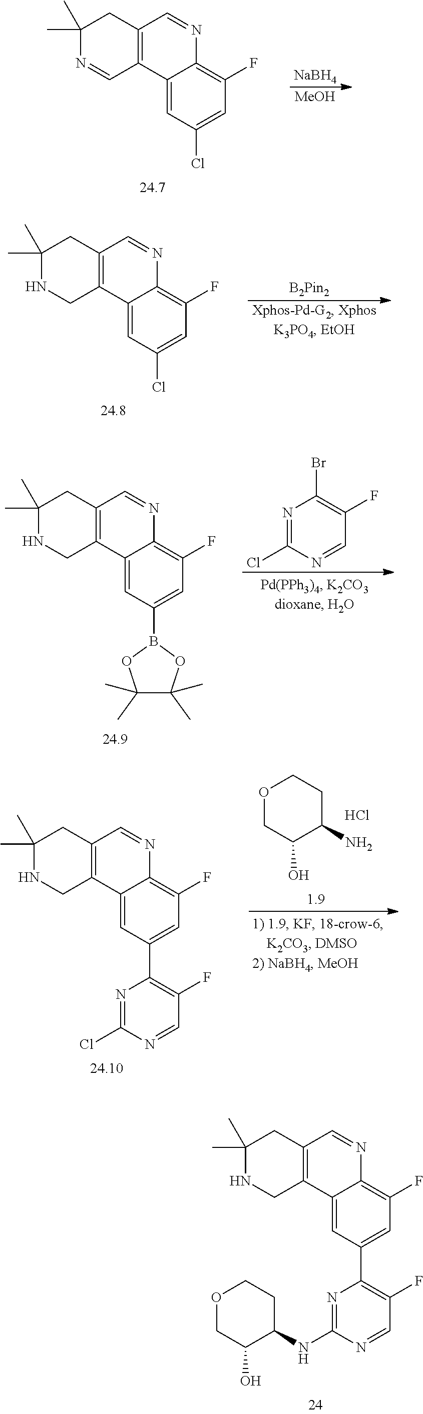

To a solution of 5,5-dimethyltetrahydropyrrol-2-one (24.1, 10.0 g, 88.4 mmol) and ethyl 2-ethoxy-2-oxoacetate (13.2 mL, 97.2 mmol) in ethanol (100 mL) was added sodium ethoxide (50.1 mL, 133 mmol, 20% in ethanol) dropwise at 0° C., and the mixture was stirred at 80° C. for 48 hrs. The resulting mixture was cooled to room temperature, diluted with water (50 mL) and adjusted to pH 3-4 with 2 N hydrochloric acid. The mixture was then extracted with petroleum ether/ethyl acetate (3/1, 30 mL) to remove impurities. The remaining suspension was filtered and the filter cake was redissolved in dichloromethane (100 mL), and then it was dried over anhydrous sodium sulfate, filtered and concentrated under reduced pressure to afford ethyl 5-hydroxy-2,2-dimethyl-6-oxo-1,2,3,6-tetrahydropyridine-4-carboxylate (24.2, 5.00 g, crude) as a white solid.

To a solution of ethyl 5-hydroxy-2,2-dimethyl-6-oxo-1,2,3,6-tetrahydropyridine-4-carboxylate (24.2, 5.00 g, crude) and N,N-diisopropylethylamine (11.6 mL, 70.3 mmol) in dichloromethane (100 mL) was added trifluoromethanesulfonic anhydride (3.95 mL, 23.4 mmol) dropwise at −78° C., and the mixture was stirred at −78° C. for 1 hr before it was warmed to room temperature. The resulting mixture was diluted with water (50 mL) and extracted with dichloromethane (50 mL×2). The combined organic layers were dried over anhydrous sodium sulfate, filtered and concentrated under reduced pressure. The residue was subjected to silica gel column chromatography to afford 4-(ethoxycarbonyl)-6,6-dimethyl-2-oxo-1,2,5,6-tetrahydropyridine-3-yl trifluoromethanesulfonate (24.3, 5.00 g) as a white solid. ¹H NMR (500 MHz, Chloroform-d₃) δ 6.52 (s, 1H), 4.35 (q, J=7.1 Hz, 2H), 2.80 (s, 2H), 1.43-1.31 (m, 9H).

To a solution of 4-(ethoxycarbonyl)-6,6-dimethyl-2-oxo-1,2,5,6-tetrahydropyridine-3-yl trifluoromethanesulfonate (24.3, 2.00 g, 5.79 mmol) and 4-chloro-6-fluoro-2-(4,4,5,5-tetramethyl-1,3,2-dioxaborolan-2-yl)aniline (1.73 g, 6.37 mmol) in dioxane (30 mL) and water (6 mL) were added tetrakis(triphenylphosphine)palladium (0.67 g, 0.58 mmol) and potassium carbonate (2.40 g, 17.4 mmol). The mixture was stirred at 110° C. for 2 hrs under nitrogen atmosphere. After cooled to room temperature, the resulting mixture was concentrated under reduced pressure and the residue was triturated with water (30 mL) and ethyl acetate (30 mL). The suspension was filtered and the filter cake was washed with water (10 mL) and ethyl acetate (20 mL) in turn. The filter cake was then dried in vacuum to afford 9-chloro-7-fluoro-3,3-dimethyl-2,3,4,6-tetrahydrobenzo[c][2,6]naphthyridine-1,5-dione (24.4, 1.40 g, 82%) as an off-white solid. LC-MS (ESI): m/z 295.2 [M+H]⁺.

A mixture of 9-chloro-7-fluoro-3,3-dimethyl-2,3,4,6-tetrahydrobenzo[c][2,6]naphthyridine-1,5-dione (24.4, 1.40 g, 4.75 mmol) and phosphorus oxychloride (10 mL) was stirred at 100° C. for 16 hrs under nitrogen atmosphere. After cooled to room temperature, the mixture was concentrated under reduced pressure to remove most of phosphorus oxychloride. The residue was quenched with water (20 mL), adjusted to pH 7 with sat. sodium bicarbonate aqueous solution and extracted with dichloromethane (50 mL×2). The combined organic layers were washed with water (20 mL), dried over anhydrous sodium sulfate and concentrated under reduced pressure to afford 5,9-dichloro-7-fluoro-3,3-dimethyl-3,4-dihydrobenzo[c][2,6]naphthyridin-1(2H)-one (24.5, 1.40 g, crude) as a yellow solid. LC-MS (ESI): m/z 313.3 [M+H]⁺.

A mixture of 5,9-dichloro-7-fluoro-3,3-dimethyl-3,4-dihydrobenzo[c][2,6]naphthyridin-1(2H)-one (24.5, 1.20 g, crude), triethylamine (1.60 mL, 11.5 mmol), formic acid (723 μL, 19.2 mmol) and tetrakis(triphenylphosphine)palladium (0.44 g, 0.38 mmol) in dimethylformamide (10 mL) was stirred at 70° C. for 16 hrs. The reaction mixture was cooled to room temperature, diluted with water (20 mL) and filtered. The filter cake was subjected to silica gel chromatography to afford a crude product and it was triturated with petroleum ether/ethyl acetate (1/1, 2 mL). The suspension was filtered, and the filter cake was washed with petroleum ether/ethyl acetate (1/1, 2 mL) and dried in vacuum to afford 9-chloro-7-fluoro-3,3-dimethyl-3,4-dihydrobenzo[c][2,6]naphthyridin-1(2H)-one (24.6, 700 mg) as a yellow solid. LC-MS (ESI): m/z 279.1 [M+H]⁺.

To a solution of 9-chloro-7-fluoro-3,3-dimethyl-3,4-dihydrobenzo[c][2,6]naphthyridin-1(2H)-one (24.6, 100 mg, 0.36 mmol) in tetrahydrofuran (3 mL) was added bis(cyclopentadienyl)zirconium chloride hydride (463 mg, 1.80 mmol) in portions at 0° C., and the mixture was stirred at 0° C. for 1 hr. Then to this reaction mixture was added methanol (10 mL) followed by addition of sodium borohydride (57.0 mg, 1.51 mmol) in portions at 0° C. The resulting mixture was stirred at 0° C. for 10 mins and then concentrated under reduced pressure. The residue was diluted with water (5 mL) and saturated sodium bicarbonate aqueous solution (1 mL), and the mixture was then extracted with dichloromethane (20 mL×3). The combined organic layers were dried over anhydrous sodium sulfate, filtered and concentrated under reduced pressure. The residue was subjected to silica gel chromatography to afford 9-chloro-7-fluoro-3,3-dimethyl-1,2,3,4-tetrahydrobenzo[c][2,6]naphthyridine (24.8, 300 mg, two steps yield 90%) as a yellow solid. LC-MS (ESI): m/z 265.2 [M+H]⁺.

A mixture of 9-chloro-7-fluoro-3,3-dimethyl-1,2,3,4-tetrahydrobenzo[c][2,6]naphthyridine (24.8, 30.0 mg, 0.11 mmol), 4,4,4′,4′,5,5,5′,5′-octamethyl-2,2′-bi(1,3,2-dioxaborolane) (43.0 mg, 0.17 mmol), tris(dibenzylideneacetone)dipalladium (10.3 mg, 0.01 mmol), dicyclohexyl(2′,4′,6′-triisopropyl-[1,1′-biphenyl]-2-yl)phosphane (10.8 mg, 0.02 mmol) and potassium acetate (33.3 mg, 0.34 mmol) in dioxane (1 mL) was stirred at 90° C. for 16 hrs under nitrogen atmosphere. The reaction mixture was cooled to room temperature and to this reaction mixture were added water (0.2 mL), 4-bromo-2-chloro-5-fluoropyrimidine (47.5 mg, 0.23 mmol), tetrakis(triphenylphosphine)palladium (13.0 mg, 0.01 mmol) and potassium carbonate (46.6 mg, 0.34 mmol). The resulting mixture was stirred at 90° C. for 4 hrs under nitrogen atmosphere. The reaction mixture was cooled to room temperature, diluted with water (2 mL) and extracted with ethyl acetate (5 mL×2). The combined organic layers were dried over anhydrous sodium sulfate, filtered and concentrated under reduced pressure. The residue was subjected to silica gel chromatography to afford 9-(2-chloro-5-fluoropyrimidin-4-yl)-7-fluoro-3,3-dimethyl-1,2,3,4-tetrahydrobenzo[c][2,6]naphthyridine (24.10, 13.0 mg, two steps yield 32%) as a brown solid. LC-MS (ESI): m/z 361.2 [M+H]⁺.

A mixture of 9-(2-chloro-5-fluoropyrimidin-4-yl)-7-fluoro-3,3-dimethyl-1,2,3,4-tetrahydrobenzo[c][2,6]naphthyridine (24.10, 10.0 mg, 0.03 mmol), (3S,4R)-4-aminotetrahydropyran-3-ol hydrochloride (1.9, 12.8 mg, 0.08 mmol), potassium fluoride (4.80 mg, 0.08 mmol), 18-crown-6 (0.70 mg, 0.003 mmol) and potassium carbonate (11.5 mg, 0.08 mmol) in dimethyl sulfoxide (100 μL) was stirred at 120° C. for 2 hrs. The reaction mixture was cooled to room temperature before methanol (200 μL) and sodium borohydride (1.00 mg, 0.03 mmol) were added in turn. The resulting mixture was stirred at room temperature for another 10 mins before it was filtered and subjected to prep-HPLC to afford (3S,4R)-4-((5-fluoro-4-(7-fluoro-3,3-dimethyl-1,2,3,4-tetrahydrobenzo[c][2,6]naphthyridin-9-yl)pyrimidin-2-yl)amino)tetrahydro-2H-pyran-3-ol (24, 3.10 mg, two steps yield 25%) as a yellow solid. LC-MS (EST): m/z 442.3 [M+H]⁺. ¹H NMR (500 MHz, DMSO-d₆) δ 8.73 (s, 1H), 8.52 (d, J=3.7 Hz, 1H), 8.39 (s, 1H), 8.05 (d, J=12.1 Hz, 1H), 7.33 (d, J=7.6 Hz, 1H), 4.96 (d, J=5.5 Hz, 1H), 4.34 (s, 2H), 3.88-3.75 (m, 4H), 3.47-3.44 (m, 1H), 3.08 (t, J=9.8 Hz, 1H), 2.76 (s, 2H), 2.10-1.86 (m, 1H), 1.58-1.40 (m, 1H), 1.14 (s, 6H).

### Illustration 25. Synthesis of (3S,4R)-4-((5-chloro-4-(8-fluoro-3-(2-hydroxypropan-2-yl-1,1,1,3,3,3-d)-2-methylquinolin-6-yl)pyrimidin-2-yl)amino)tetrahydro-2H-pyran-3-ol (60)

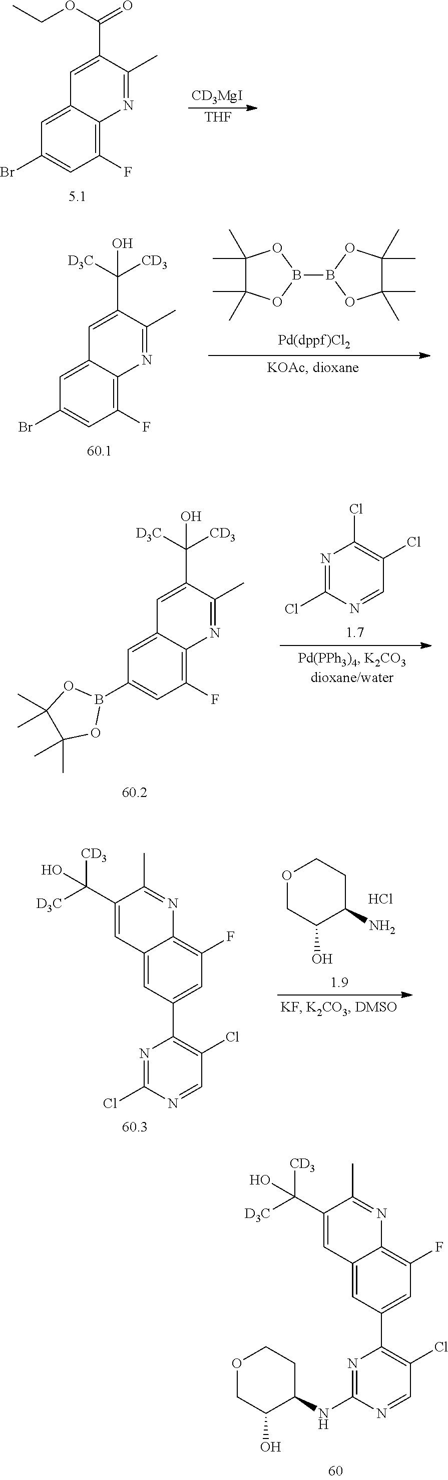

To a solution of ethyl 6-bromo-8-fluoro-2-methylquinoline-3-carboxylate (5.1, 4.00 g, 12.8 mmol) in anhydrous tetrahydrofuran (80 mL) was added methyl-d₃-magnesium iodide (64 mL, 64 mmol, 1.0 M in diethyl ether) dropwise under nitrogen atmosphere at 0° C., and the mixture was stifred at 25° C. for 3 hrs under nitrogen atmosphere. The reaction mixture was quenched with saturated ammonium chloride solution (20 mL), diluted with water (50 mL) and extracted with ethyl acetate (50 mL×3). The combined organic layers were washed with brine (20 mL), dried over anhydrous sodium sulfate and concentrated under reduced pressure. The residue was subjected to silica gel column chromatography to afford 2-(6-bromo-8-fluoro-2-methylquinolin-3-yl)propan-1,1,1,3,3,3-d₆₋₂-ol (60.1, 2.5 g, 64%) as a yellow solid. LC-MS (ESI): m/z 304.0 [M+H]⁺.

A mixture of 2-(6-bromo-8-fluoro-2-methylquinolin-3-yl)propan-1,1,1,3,3,3-d₆₋₂-ol (60.1, 2.20 g, 7.23 mmol), 4,4,5,5-tetramethyl-2-(4,4,5,5-tetramethyl-1,3,2-dioxaborolan-2-yl)-1,3,2-dioxaborolane (2.75 g, 10.8 mmol), potassium acetate (2.13 g, 21.7 mmol) and 1,1′-bis(diphenylphosphino)ferrocene-palladium(II)dichloride (525 mg, 0.72 mmol) in dioxane (40 mL) was stirred at 90° C. for 3 hrs under nitrogen atmosphere. The reaction mixture was diluted with ethyl acetate (30 mL) and filtered. The filtrate was concentrated under reduced pressure and the residue was subjected to silica gel column chromatography to afford 2-(8-fluoro-2-methyl-6-(4,4,5,5-tetramethyl-1,3,2-dioxaborolan-2-yl)quinolin-3-yl)propan-1,1,1,3,3,3-d₆-2-ol (60.2, 2.20 g, 86%) as a yellow solid. LC-MS (ESI): m/z 352.2 [M+H]⁺.

A mixture of 2-(8-fluoro-2-methyl-6-(4,4,5,5-tetramethyl-1,3,2-dioxaborolan-2-yl)quinolin-3-yl)propan-1,1,1,3,3,3-d₆-2-ol (60.2, 2.00 g, 5.60 mmol), 2,4,5-trichloropyrimidine (1.7, 1.34 g, 7.28 mmol), tetrakis(triphenylphosphine)palladium (650 mg, 0.56 mmol) and potassium carbonate (2.32 g, 16.8 mmol) in dioxane/water (20 mL/4 mL) was stirred at 90° C. for 4 hrs under nitrogen atmosphere. The resulting mixture was concentrated under reduced pressure and the residue was subjected to silica gel column chromatography to afford 2-(6-(2,5-dichloropyrimidin-4-yl)-8-fluoro-2-methylquinolin-3-yl)propan-1,1,1,3,3,3-d₆-2-ol (60.3, 1.20 g, 56%) as a yellow solid. LC-MS (ESI): m/z 372.0 [M+H]⁺.

A mixture of 2-(6-(2,5-dichloropyrimidin-4-yl)-8-fluoro-2-methylquinolin-3-yl)propan-1,1,1,3,3,3-d₆-2-ol (60.3, 1.00 g, 2.70 mmol), (3S,4R)-4-aminotetrahydropyran-3-ol hydrogen chloride (1.9, 590 mg, 3.80 mmol), potassium fluoride (620 mg, 10.7 mmol), 18-crown-6 (710 mg, 2.70 mmol) and potassium carbonate (370 mg, 2.70 mmol) in dimethyl sulfoxide (2 mL) was stirred at 120° C. for 2.5 hrs. The reaction mixture was filtered and the filtrate was subjected to prep-HPLC to afford (3S,4R)-4-((5-chloro-4-(8-fluoro-3-(2-hydroxypropan-2-yl-1,1,1,3,3,3-d₆)-2-methylquinolin-6-yl)pyrimidin-2-yl)amino)tetrahydro-2H-pyran-3-ol (60, 180 mg, 15%) as a white solid. LC-MS (ESI): m/z 453.2 [M+H]⁺. ¹H NMR (400 MHz, DMSO-d₆) δ 8.46 (s, 2H), 8.24 (s, 1H), 7.80 (d, J=11.5 Hz, 1H), 7.53 (s, 1H), 5.30 (d, J=6.8 Hz, 1H), 4.92 (d, J=5.4 Hz, 1H), 3.89-3.79 (m, 3H), 3.53-3.45 (m, 1H), 3.36-3.33 (m, 1H), 3.08-3.03 (m, 1H), 2.95 (s, 3H), 2.01-1.91 (m, 1H), 1.55-1.45 (m, 1H).

### Illustration 26. Synthesis of (3S,4R)-4-((5-chloro-4-(4-ethyl-8-fluoro-3-(2-hydroxypropan-2-yl)cinnolin-6-yl)pyrimidin-2-yl)amino)tetrahydro-2H-pyran-3-ol (61)

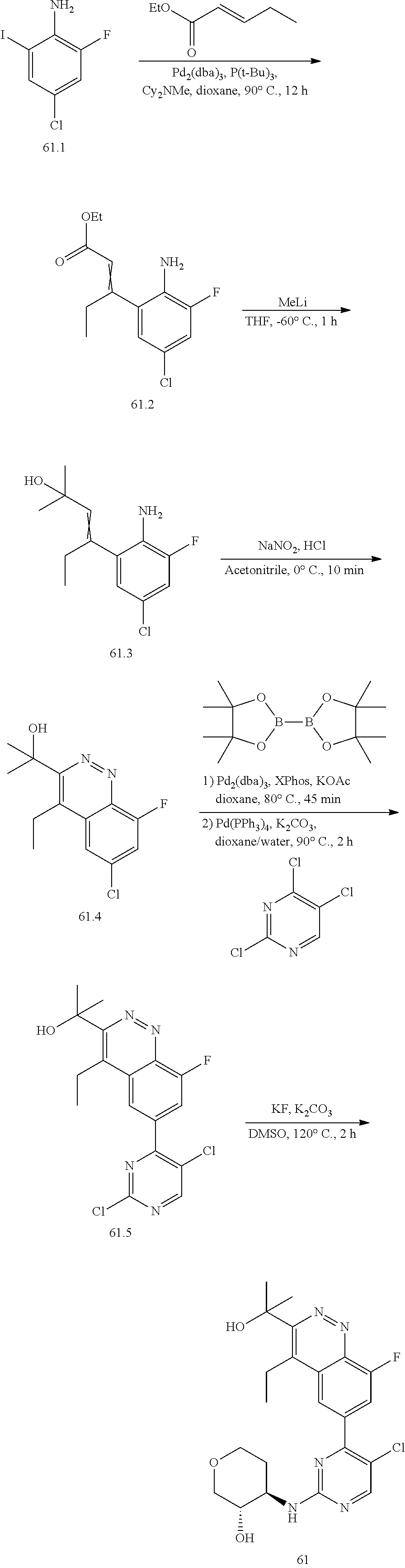

A mixture of 4-chloro-2-fluoro-6-iodoaniline (61.1, 2.00 g, 7.37 mmol), ethyl (E)-pent-2-enoate (1.35 mL, 9.58 mmol), tris(dibenzylideneacetone)dipalladium (0.10 g, 0.11 mmol), N,N-dicyclohexylmethylamine (1.74 mL, 8.10 mmol) and tri-tert-butylphosphine (0.05 mL, 0.22 mmol) in dioxane (20 mL) was stirred at 90° C. for 12 hrs under nitrogen atmosphere. The resulting mixture was concentrated under reduced pressure and the residue was subjected to silica gel column chromatography to afford ethyl 3-(2-amino-5-chloro-3-fluorophenyl)pent-2-enoate (61.2, 530 mg, crude) as a yellow oil. LC-MS (ESI): m/z 272.1 [M+H]⁺.

To a solution of ethyl 3-(2-amino-5-chloro-3-fluorophenyl)pent-2-enoate (61.2, 500 mg, crude) in anhydrous tetrahydrofuran (10.0 mL) was added methyllithium (9.75 mL, 15.6 mmol, 1.6 M in diethyl ether) dropwise at −60° C. under nitrogen atmosphere, and the mixture was stirred at −60° C. for 1 hr. The reaction mixture was quenched with saturated aqueous solution of sodium bicarbonate (25 mL) at −60° C. The resulting mixture was warmed up to room temperature and the aqueous phase was extracted with ethyl acetate (3×20 mL). The combined organic layers were washed with brine, dried over anhydrous sodium sulfate and concentrated under reduced pressure. The residue was subjected to silica gel column chromatography to afford 4-(2-amino-5-chloro-3-fluorophenyl)-2-methylhex-3-en-2-ol (61.3, 200 mg) as a yellow oil. LC-MS (ESI): m/z 258.1 [M+H]⁺.

To a solution of 4-(2-amino-5-chloro-3-fluorophenyl)-2-methylhex-3-en-2-ol (61.3, 200 mg, 0.77 mmol) in acetone (8.0 mL) was added hydrochloric acid (4.0 mL, 0.08 mmol, wt %=18%) and sodium nitrite (80.3 mg, 1.16 mmol) at 0° C. The mixture was stirred at 0° C. for 10 mins before it was quenched with water (100 mL) and extracted with ethyl acetate (3×20 mL). The combined organic layers were washed with brine, dried over anhydrous sodium sulfate and concentrated under reduced pressure. The residue was subjected to silica gel column chromatography to afford 2-(6-chloro-4-ethyl-8-fluorocinnolin-3-yl)propan-2-ol (61.4, 200 mg, 96%) as a yellow solid. LC-MS (ESI): m/z 269.1 [M+H]⁺.

A mixture of 2-(6-chloro-4-ethyl-8-fluorocinnolin-3-yl)propan-2-ol (61.4, 200 mg, 0.74 mmol), 4,4,4′,4′,5,5,5′,5′-octamethyl-2,2′-bi(1,3,2-dioxaborolane) (284 mg, 1.12 mmol), tris(dibenzylideneacetone)dipalladium (68.2 mg, 0.07 mmol), potassium acetate (146 mg, 1.49 mmol) and 2-dicyclohexylphosphino-2′,4′,6′-tri-i-propyl-1,1′-biphenyl (71.0 mg, 0.15 mmol) in dioxane (3.0 mL) was stirred at 80° C. for 45 mins under nitrogen atmosphere. The reaction mixture was cooled to room temperature. To the cooled mixture were added 2,4,5-trichloropyrimidine (1.7, 164 mg, 0.89 mmol), tetrakis(triphenylphosphine)palladium (43.0 mg, 0.04 mmol), potassium carbonate (206 mg, 1.49 mmol) and water (0.70 mL). The resulting mixture was stirred at 90° C. for 2 hrs under nitrogen atmosphere before it was concentrated under reduced pressure. The residue was subjected to silica gel column chromatography to afford 2-(6-(2,5-dichloropyrimidin-4-yl)-4-ethyl-8-fluorocinnolin-3-yl)propan-2-ol (61.5, 210 mg, 74%) as a yellow oil. LC-MS (EST): m/z 381.1 [M+H]⁺.

A mixture of 2-(6-(2,5-dichloropyrimidin-4-yl)-4-ethyl-8-fluorocinnolin-3-yl)propan-2-ol (61.5, 210 mg, 0.551 mmol), ((3S,4R)-4-aminotetrahydropyran-3-ol hydrogen chloride (1.9, 127 mg, 0.82 mmol), potassium fluoride (128 mg, 2.20 mmol) and potassium carbonate (152 mg, 1.10 mmol) in dimethyl sulfoxide (2.0 mL) was stirred at 120° C. for 2 hrs. The reaction mixture was cooled to room temperature and filtered. The filtrate was subjected to prep-HPLC to afford (3S,4R)-4-((5-chloro-4-(4-ethyl-8-fluoro-3-(2-hydroxypropan-2-yl)cinnolin-6-yl)pyrimidin-2-yl)amino)tetrahydro-2H-pyran-3-ol (61, 18.8 mg, 7%) as a yellow solid. LC-MS (ESI): m/z 462.2 [M+H]⁺. ¹H NMR (400 MHz, DMSO-d₆) δ 8.54 (s, 1H), 8.53-8.39 (m, 1H), 8.00 (s, 1H), 7.68 (s, 1H), 5.64 (s, 1H), 4.96 (d, J=5.5 Hz, 1H), 3.94-3.79 (m, 3H), 3.66-3.47 (m, 3H), 3.41-3.37 (m, 1H), 3.07 (t, J=10.3 Hz, 1H), 2.05-1.98 (m, 1H), 1.81 (s, 6H), 1.59-1.48 (m, 1H), 1.37 (t, J=7.4 Hz, 3H).

### Illustration 27. Synthesis of (3S,4R)-4-((5-fluoro-4-(7-fluoro-3,3-dimethyl-1,2,3,4-tetrahydrobenzo[c][2,6]naphthyridin-9-yl-1,1-d₂)pyrimidin-2-yl)amino)tetrahydro-2H-pyran-3-ol (62)

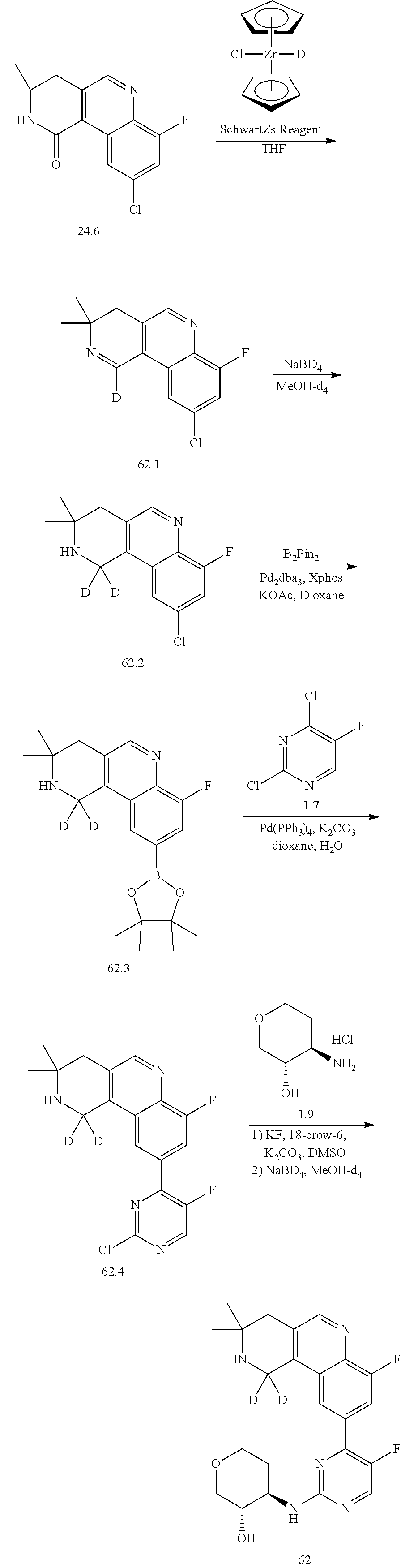

To a solution of 9-chloro-7-fluoro-3,3-dimethyl-3,4-dihydrobenzo[c][2,6]naphthyridin-1(2H)-one (24.6, 360 mg, 1.29 mmol) in anhydrous tetrahydrofuran (5 mL) was added bis(cyclopentadienyl)zirconium chloride deuteride (1.61 g, 6.46 mmol) in portions at 0° C., and the mixture was stirred at 0° C. for 1 hr. To the resulting mixture was added methanol-d₄ (0.5 mL) followed by addition of sodium borohydride-d₄ (162 mg, 3.87 mmol) in portions at 0° C. The mixture was stirred at 0° C. for 10 mins and then concentrated under reduced pressure. The residue was diluted with water (5 mL) and saturated sodium bicarbonate aqueous solution (1 mL), and then it was extracted with dichloromethane (20 mL×3). The combined organic layers were dried over anhydrous sodium sulfate, filtered and concentrated under reduced pressure. The residue was subjected to silica gel chromatography to afford 9-chloro-7-fluoro-3,3-dimethyl-1,2,3,4-tetrahydrobenzo[c][2,6]naphthyridine-1,1-d₂ (62.2, 250 mg, two steps yield 72%) as a yellow solid. LC-MS (ESI): m/z 267.1 [M+H]⁺.

A mixture of 9-chloro-7-fluoro-3,3-dimethyl-1,2,3,4-tetrahydrobenzo[c][2,6]naphthyridine-1,1-d₂ (62.2, 120 mg, 0.45 mmol), 4,4,4′,4′,5,5,5′,5′-octamethyl-2,2′-bi(1,3,2-dioxaborolane) (228 mg, 0.90 mmol), tris(dibenzylideneacetone)dipalladium (41.2 mg, 0.04 mmol), dicyclohexyl(2′,4′,6′-triisopropyl-[1,1′-biphenyl]-2-yl)phosphane (21.5 mg, 0.04 mmol) and potassium acetate (132 mg, 1.35 mmol) in dioxane (5 mL) was stirred at 100° C. for 3 hrs under nitrogen atmosphere. The reaction mixture was cooled to room temperature. To the mixture were added water (1 mL), 2,4-dichloro-5-fluoropyrimidine (1.7, 74.6 mg, 0.45 mmol), tetrakis(triphenylphosphine)palladium (51.6 mg, 0.04 mmol) and potassium carbonate (185 mg, 1.34 mmol), and the resulting mixture was stirred at 90° C. for 4 hrs under nitrogen atmosphere. The reaction mixture was cooled to room temperature, diluted with water (2 mL) and extracted with ethyl acetate (5 mL×2). The combined organic layers were dried over anhydrous sodium sulfate, filtered and concentrated under reduced pressure. The residue was subjected to silica gel chromatography to afford 9-(2-chloro-5-fluoropyrimidin-4-yl)-7-fluoro-3,3-dimethyl-1,2,3,4-tetrahydrobenzo[c][2,6]naphthyridine-1,1-d₂ (62.4, 120 mg, two steps yield 75%) as a yellow solid. LC-MS (ESI): m/z 363.1 [M+H]⁺.

A mixture of 9-(2-chloro-5-fluoropyrimidin-4-yl)-7-fluoro-3,3-dimethyl-1,2,3,4-tetrahydrobenzo[c][2,6]naphthyridine-1,1-d₂ (62.4, 120 mg, 0.33 mmol), (3S,4R)-4-aminotetrahydropyran-3-ol hydrochloride (1.9, 61.0 mg, 0.40 mmol), potassium fluoride (76.9 mg, 1.32 mmol), 18-crown-6 (8.70 mg, 0.03 mmol) and potassium carbonate (45.7 mg, 0.33 mmol) in dimethyl sulfoxide (1 mL) was stirred at 120° C. for 2 hrs. The reaction mixture was cooled to room temperature, and to it were added methanol-d₄ (200 μL) and sodium borohydride-d₄ (12.0 mg, 0.33 mmol) in turn. The resulting mixture was stirred at room temperature for another 10 mins. The reaction mixture was filtered and subjected to prep-HPLC to afford (3S,4R)-4-((5-fluoro-4-(7-fluoro-3,3-dimethyl-1,2,3,4-tetrahydrobenzo[c][2,6]naphthyridin-9-yl-1,1-d₂)pyrimidin-2-yl)amino)tetrahydro-2H-pyran-3-ol (62, 32.0 mg, two steps yield 21%) as a yellow solid. LC-MS (ESI): m/z 444.2 [M+H]⁺; ¹H NMR (500 MHz, DMSO-d₆) δ 8.73 (s, 1H), 8.52 (d, J=3.2 Hz, 1H), 8.40 (s, 1H), 8.06 (d, J=11.8 Hz, 1H), 7.33 (d, J=7.6 Hz, 1H), 4.97 (d, J=4.7 Hz, 1H), 3.88-3.80 (m, 3H), 3.57-3.49 (m, 1H), 3.43-3.37 (m, 1H), 3.09 (t, J=10.1 Hz, 1H), 2.76 (s, 2H), 2.38 (brs, 1H), 2.07-1.98 (m, 1H), 1.56-1.46 (m, 1H), 1.14 (s, 6H).

LC-MS and ¹H NMR, and de and/or ee values as applicable, for additional exemplary compounds which were prepared using similar synthetic methodology and routes are provided in Table A, below.

TABLE A
Exemplary Characterization of Additional Compounds of the Present disclosure

| Compound | | LC-MS; ¹H NMR (ppm); de value; separation |
|---|---|---|
| No. | Structure | conditions |
| 25 | 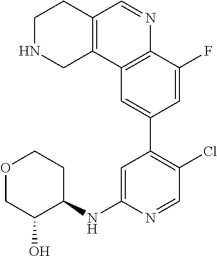 [SMILES: O[C@@H]1COCC[C@H]1Nc1cc(-c2cc(F)c3ncc4c(c3c2)CNCC4)c(Cl)cn1] | LC-MS (ESI): m/z 429.2 [M + H]⁺; ¹H NMR (500 MHz, DMSO-d₆) δ 8.71 (s, 1H), 8.24 (s, 1H), 7.84 (d, J = 1.8 Hz, 1H), 7.62 (dd, J = 11.3, 1.7 Hz, 1H), 6.86 (d, J = 7.4 Hz, 1H), 6.70 (s, 1H), 5.02 (s, 1H), 4.05 (s, 2H), 3.85-3.76 (m, 3H), 3.52-3.43 (m, 2H), 3.13-3.04 (m, 5H), 2.00-2.05 (m, 1H), 1.47-1.36 (m, 1H). |
| 26 | 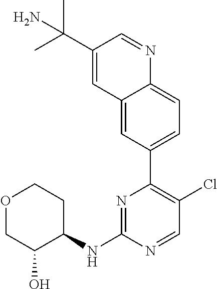 [SMILES: CC(C)(N)c1cnc2ccc(-c3nc(N[C@@H]4CCOC[C@H]4O)ncc3Cl)cc2c1] | LC-MS (ESI): m/z 414.2 [M + H]⁺; ¹H NMR (400 MHz, DMSO-d₆) δ 9.25 (d, J = 2.4 Hz, 1H), 8.51 (d, J = 2.4 Hz, 1H), 8.48 (s, 1H), 8.39 (s, 1H), 8.15-8.09 (m, 1H), 8.08-8.00 (m, 1H), 7.62-7.42 (m, 1H), 4.95 (s, 1H), 3.96-3.75 (m, 3H), 3.60-3.28 (m, 4H), 3.12-3.02 (m, 1H), 2.07-1.90 (m, 1H), 1.63-1.45 (m, 7H). |
| 27 | 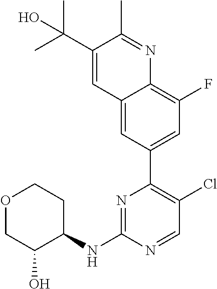 | LC-MS (ESI): m/z 461.2 [M + H]⁺; ¹H NMR (500 MHz, DMSO-d₆) δ 8.47 (s, 2H), 8.25 (s, 1H), 7.81 (d, J = 11.2 Hz, 1H), 7.54 (s, 1H), 5.37 (s, 1H), 4.94 (d, J = 5.5 Hz, 1H), 3.90- 3.75 (m, 3H), 3.55-3.45 (m, 1H), 3.40-3.25 (m, 3H), 3.10-3.01 (m, 1H), 2.05-1.86 (m, 1H), 1.66 (s, 6H), 1.57-1.46 (m, 1H), 1.36 (t, J = 7.4 Hz, 3H). |
| 28 | 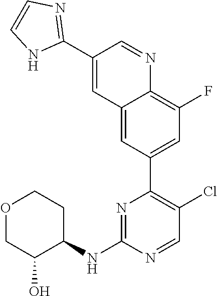 [SMILES: O[C@@H]1COCC[C@H]1Nc1ncc(Cl)c(-c2cc(F)c3ncc(-c4ncc[nH]4)cc3c2)n1] | LC-MS (ESI): m/z 441.2 [M + H]⁺; ¹H NMR (500 MHz, DMSO-d₆) δ 13.03 (s, 1H), 9.61 (d, J = 2.1 Hz, 1H), 8.95 (s, 1H), 8.50 (s, 1H), 8.29 (s, 1H), 7.91 (d, J = 11.4 Hz, 1H), 7.60 (s, 1H), 7.45 (s, 1H), 7.20 (s, 1H), 4.94 (s, 1H), 3.93- 3.77 (m, 3H), 3.52 (s, 2H), 3.06 (t, J = 10.4 Hz, 1H), 2.04-1.89 (m, 1H), 1.60-1.45 (m, 1H). |
| 29 | 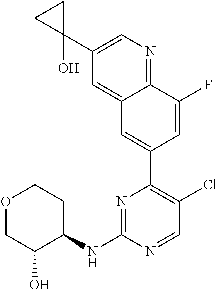 [SMILES: O[C@@H]1COCC[C@H]1Nc1ncc(Cl)c(-c2cc(F)c3ncc(C4(O)CC4)cc3c2)n1] | LC-MS (ESI): m/z 431.2 [M + H]⁺; ¹H NMR (500 MHz, DMSO-d₆) δ 8.87 (d, J = 2.2 Hz, 1H), 8.47 (s, 1H), 8.39 (s, 1H), 8.24 (s, 1H), 7.82 (d, J = 10.9 Hz, 1H), 7.55 (s, 1H), 6.37 (s, 1H), 4.92 (d, J = 5.4 Hz, 1H), 3.91-3.75 (m, 3H), 3.57-3.45 (m, 1H), 3.38-3.34 (m, 1H), 3.10-2.99 (m, 1H), 2.02-1.87 (m, 1H), 1.55- 1.44 (m, 1H), 1.33-1.18 (m, 4H). |
| 30 | 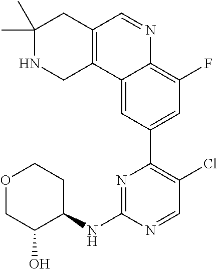 [SMILES: CC1(C)Cc2cnc3c(F)cc(-c4nc(N[C@@H]5CCOC[C@H]5O)ncc4Cl)cc3c2CN1] | LC-MS (ESI): m/z 458.2 [M + H]⁺; ¹H NMR (500 MHz, DMSO-d₆) δ 8.74 (s, 1H), 8.48 (s, 1H), 8.20 (s, 1H), 7.86 (d, J = 11.3 Hz, 1H), 7.58 (s, 1H), 4.37 (s, 2H), 3.87-3.78 (m, 3H), 3.53-3.51 (m, 1H), 3.34 (td, J = 11.7, 2.3 Hz, 1H), 3.06 (dd, J = 11.0, 9.7 Hz, 1H), 2.80 (s, 2H), 2.02-1.90 (m, 1H), 1.56-1.46 (m, 1H), 1.17 (s, 6H). |
| 31a | 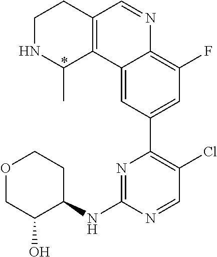 [SMILES: CC1NCCc2cnc3c(F)cc(-c4nc(N[C@@H]5CCOC[C@H]5O)ncc4Cl)cc3c21] Isomer 1 (earlier eluting isomer) | LC-MS (ESI): m/z 444.2 [M + H]⁺; ¹H NMR (500 MHz, DMSO-d₆) δ 8.78 (s, 1H), 8.48 (s, 1H), 8.25 (s, 1H), 7.85 (s, 1H), 7.57 (s, 1H), 4.94-4.87 (m, 2H), 3.87-3.79 (m, 3H), 3.54- 3.47 (m, 1H), 3.32-3.26 (m, 2H), 3.20-3.16 (m, 1H), 3.07-3.03 (m, 1H), 2.99-2.93 (m, 2H), 1.96-1.92 (m, 1H), 1.59-1.44 (m, 4H). de: 100%; Retention time: 0.762 min; instrument: Agilent- 1260; column: CHIRALART Cellulose-SB, 4.6 * 50 mm, 3.0 um; mobile phase: A for MtBE (0.1% DEA) and B for EtOH; gradient: B 30%; flow rate: 1.67 mL/min; high pressure: 104 bar; column temperature: 25° C.; wavelength: 254 nm. |
| 31b | 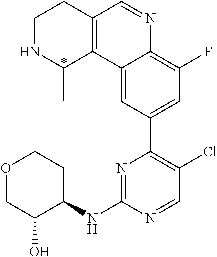 [SMILES: CC1NCCc2cnc3c(F)cc(-c4nc(N[C@@H]5CCOC[C@H]5O)ncc4Cl)cc3c21] Isomer 2 (later eluting isomer) | LC-MS (ESI): m/z 444.2 [M + H]⁺; ¹H NMR (500 MHz, DMSO-d₆) δ 8.75 (s, 1H), 8.48 (s, 1H), 8.23 (s, 1H), 7.82 (s, 1H), 7.56 (s, 1H), 4.92 (d, J = 5.5 Hz, 1H), 4.78-4.70 (m, 1H), 3.83-3.78 (m 3H), 3.54-3.46 (m, 1H), 3.35- 3.32 (m, 2H), 3.25-3.17 (m, 1H), 3.08-3.03 (m, 1H), 2.92-2.85 (m, 2H), 2.11-1.84 (m, 1H), 1.56-1.45 (m, 4H). de: 100%; Retention time: 1.581 min; instrument: Agilent- 1260; column: CHIRALART Cellulose-SB, 4.6 * 50 mm, 3.0 um; mobile phase: A for MtBE (0.1% DEA) and B for EtOH; gradient: B 30%; flow rate: 1.67 mL/min; high pressure: 104 bar; column temperature: 25° C.; wavelength: 254 nm. |
| 32a | 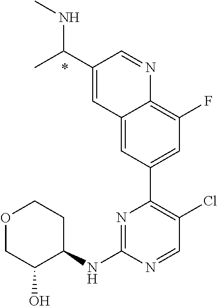 [SMILES: CNC(C)c1cnc2c(F)cc(-c3nc(N[C@@H]4CCOC[C@H]4O)ncc3Cl)cc2c1] Isomer 1 (earlier eluting isomer) | LC-MS (ESI): m/z 432.2 [M + H]⁺; ¹H NMR (500 MHz, DMSO-d₆) δ 9.03 (d, J = 2.1 Hz, 1H), 8.48 (s, 1H), 8.43 (s, 1H), 8.24 (s, 1H), 7.86 (d, J = 11.5 Hz, 1H), 7.56 (s, 1H), 4.94 (s, 1H), 3.90-3.78 (m, 4H), 3.53-3.48 (m, 1H), 3.39-3.36 (m, 1H), 3.06 (dd, J = 11.1, 9.7 Hz, 1H), 2.20 (s, 3H), 1.96 (d, J = 13.1 Hz, 1H), 1.57-1.45 (m, 1H), 1.39 (d, J = 6.6 Hz, 3H). de: 100%; Retention time: 1.175 min; instrument: Agilent- 1260; column: CHIRALPAK IA-3, 4.6 * 50 mm, 3.0 um; mobile phase: A for MtBE (0.1% DEA) and B for EtOH; gradient: B 20%; flow rate: 1.67 mL/min; high pressure: 134 bar; column temperature: 25° C.; wavelength: 254 nm. |
| 32b | 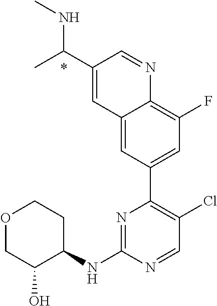 [SMILES: CNC(C)c1cnc2c(F)cc(-c3nc(N[C@@H]4CCOC[C@H]4O)ncc3Cl)cc2c1] Isomer 2 (later eluting isomer) | LC-MS (ESI): m/z 432.2 [M + H]⁺; ¹H NMR (500 MHz, DMSO-d₆) δ 9.03 (d, J = 2.0 Hz, 1H), 8.48 (s, 1H), 8.43 (s, 1H), 8.24 (s, 1H), 7.86 (d, J = 11.5 Hz, 1H), 7.56 (s, 1H), 4.94 (s, 1H), 3.91-3.79 (m, 4H), 3.53-3.49 (m, 1H), 3.38-3.35 (m, 1H), 3.06 (dd, J = 11.1, 9.7 Hz, 1H), 2.19 (s, 3H), 1.96 (d, J = 13.2 Hz, 1H), 1.57-1.45 (m, 1H), 1.39 (d, J = 6.6 Hz, 3H). de: 100%; Retention time: 1.639 min; instrument: Agilent- 1260; column: CHIRALPAK IA-3, 4.6 * 50 mm, 3.0 um; mobile phase: A for MtBE (0.1% DEA) and B for EtOH; gradient: B 20%; flow rate: 1.67 mL/min; high pressure: 134 bar; column temperature: 25° C.; wavelength: 254 nm. |
| 33 | 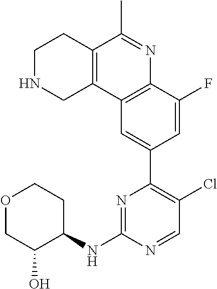 [SMILES: Cc1nc2c(F)cc(-c3nc(N[C@@H]4CCOC[C@H]4O)ncc3Cl)cc2c2c1CCNC2] | LC-MS (ESI): m/z 444.1 [M + H]⁺; ¹H NMR (500 MHz, DMSO-d₆) δ 8.43 (s, 1H), 7.96 (s, 1H), 7.57-7.38 (m, 2H), 5.99 (s, 1H), 4.93 (d, J = 5.3 Hz, 1H), 3.88-3.75 (m, 3H), 3.57-3.46 (m, 1H), 3.44-3.35 (m, 2H), 3.05 (t, J = 10.4 Hz, 1H), 2.97 (t, J = 6.1 Hz, 2H), 2.50 (s, 3H), 2.03-1.86 (m, 3H), 1.56-1.46 (m, 1H). |
| 34a | 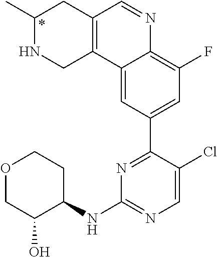 [SMILES: CC1Cc2cnc3c(F)cc(-c4nc(N[C@@H]5CCOC[C@H]5O)ncc4Cl)cc3c2CN1] Isomer 1 (earlier eluting isomer) | LC-MS (ESI): m/z 444.1 [M + H]⁺; ¹H NMR (500 MHz, DMSO-d₆) δ 8.74 (s, 1H), 8.47 (s, 1H), 8.16 (s, 1H), 7.85 (d, J = 11.3 Hz, 1H), 7.58 (s, 1H), 4.93 (d, J = 5.5 Hz, 1H), 4.47 (d, J = 17.7 Hz, 1H), 4.23 (d, J = 17.7 Hz, 1H), 3.88- 3.76 (m, 3H), 3.57-3.45 (m, 1H), 3.35-3.33 (m, 1H), 3.06 (dd, J = 11.1, 9.7 Hz, 1H), 3.01- 2.88 (m, 2H), 2.59-2.54 (m, 1H), 1.97 (s, 1H), 1.62-1.41 (m, 1H), 1.22 (d, J = 6.1 Hz, 3H). de: 100%; Retention time: 3.411 min; instrument: Agilent- 1260; column: CHIRALPAK IG-3, 3.0 * 100 mm, 3.0 um; mobile phase: A for CO₂ and B for IPA (20 mMNH₃); gradient: B 50%; flow rate: 2 mL/min; high pressure: 110 bar; column temperature: 35° C.; wavelength: 220 nm. |
| 34b | 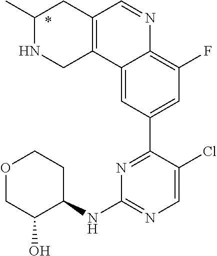 [SMILES: CC1Cc2cnc3c(F)cc(-c4nc(N[C@@H]5CCOC[C@H]5O)ncc4Cl)cc3c2CN1] Isomer 2 (later eluting isomer) | LC-MS (ESI): m/z 444.1 [M + H]⁺; ¹H NMR (500 MHz, DMSO-d₆) δ 8.74 (s, 1H), 8.48 (s, 1H), 8.16 (s, 1H), 7.85 (s, 1H), 7.58 (s, 1H), 4.93 (d, J = 5.5 Hz, 1H), 4.46 (d, J = 17.7 Hz, 1H), 4.23 (d, J = 17.8 Hz, 1H), 3.91-3.78 (m, 3H), 3.56-3.44 (m, 1H), 3.36-3.33 (m, 1H), 3.06 (td, J = 11.1, 9.7 Hz, 1H), 3.00-2.89 (m, 2H), 2.60-2.53 (m, 1H), 1.96 (s, 1H), 1.61- 1.43 (m, 1H), 1.22 (d, J = 6.1 Hz, 3H). de: 100%; Retention time: 5.235 min; instrument: Agilent- 1260; column: CHIRALPAK IG-3, 3.0 * 100 mm, 3.0 um; mobile phase: A for CO₂ and B for IPA (20 mMNH₃); gradient: B 50%; flow rate: 2 mL/min; high pressure: 110 bar; column temperature: 35° C.; wavelength: 220 nm. |
| 35a | 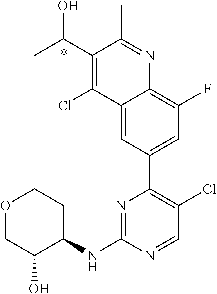 [SMILES: Cc1nc2c(F)cc(-c3nc(N[C@@H]4CCOC[C@H]4O)ncc3Cl)cc2c(Cl)c1C(C)O] Isomer 1 (earlier eluting isomer) | LC-MS (ESI): m/z 467.1 [M + H]⁺; ¹H NMR (500 MHz, DMSO-d₆) δ 8.49 (s, 2H), 7.99 (d, J = 9.5 Hz, 1H), 7.62 (s, 1H), 5.76-5.61 (m, 2H), 4.94 (d, J = 5.4 Hz, 1H), 3.94-3.74 (m, 3H), 3.58-3.32 (m, 2H), 3.06 (t, J = 10.4 Hz, 1H), 2.95 (s, 3H), 1.96 (s, 1H), 1.63-1.41 (m, 4H). de: 100%; Retention time: 0.571 min; instrument: Agilent- 1260; column: CHIRALPAK IH-3, 4.6 * 50 mm, 3.0 um; mobile phase: A for Hex (0.1% DEA) and B for EtOH; gradient: B 50%; flow rate: 1.67 mL/min; high pressure: 110 bar; column temperature: 25° C.; wavelength: 254 nm. |
| 35b | 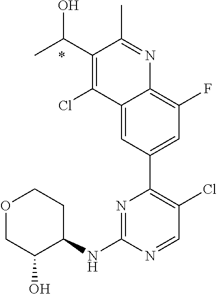 [SMILES: Cc1nc2c(F)cc(-c3nc(N[C@@H]4CCOC[C@H]4O)ncc3Cl)cc2c(Cl)c1C(C)O] Isomer 2 (later eluting isomer) | LC-MS (ESI): m/z 467.1 [M + H]⁺; ¹H NMR (500 MHz, DMSO-d₆) δ 8.49 (s, 2H), 8.00 (d, J = 8.9 Hz, 1H), 7.62 (s, 1H), 5.74-5.60 (m, 2H), 4.93 (d, J = 5.4 Hz, 1H), 3.92-3.77 (m, 3H), 3.56-3.34 (m, 2H), 3.11-3.00 (m, 1H), 2.95 (s, 3H), 1.96 (s, 1H), 1.63-1.43 (m, 4H). de: 98.6%; Retention time: 1.335 min; instrument: Agilent- 1260; column: CHIRALPAK IH-3, 4.6 * 50 mm, 3.0 um; mobile phase: A for Hex (0.1% DEA) and B for EtOH; gradient: B 50%; flow rate: 1.67 mL/min; high pressure: 110 bar; column temperature: 25° C.; wavelength: 254 nm. |
| 36 | 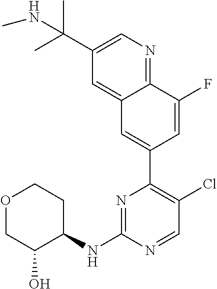 [SMILES: CNC(C)(C)c1cnc2c(F)cc(-c3nc(N[C@@H]4CCOC[C@H]4O)ncc3Cl)cc2c1] | LC-MS (ESI): m/z 446.2 [M + H]⁺; ¹H NMR (500 MHz, DMSO-d₆) δ 9.24 (d, J = 2.2 Hz, 1H), 8.56 (s, 1H), 8.49 (s, 1H), 8.30 (s, 1H), 7.89 (d, J = 11.4 Hz, 1H), 7.56 (s, 1H), 4.94 (d, J = 5.4 Hz, 1H), 3.93-3.76 (m, 3H), 3.53-3.37 (m, 2H), 3.09-3.07 (m, 1H), 2.15 (s, 3H), 2.02- 1.90 (m, 1H), 1.59 (s, 6H), 1.56-1.44 (m, 1H). |
| 37 | 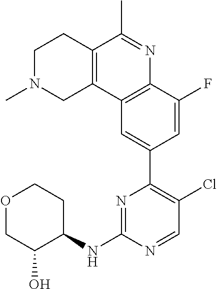 [SMILES: Cc1nc2c(F)cc(-c3nc(N[C@@H]4CCOC[C@H]4O)ncc3Cl)cc2c2c1CCN(C)C2] | LC-MS (ESI): m/z 458.3 [M + H]⁺; ¹H NMR (500 MHz, DMSO-d₆) δ 8.45 (s, 1H), 8.13 (s, 1H), 7.68 (d, J = 8.0 Hz, 1H), 7.54 (s, 1H), 4.95 (s, 1H), 3.81 (dt, J = 8.9, 5.7 Hz, 3H), 3.50 (dd, J = 12.0, 7.3 Hz, 1H), 3.43-3.39 (m, 1H), 3.16- 3.09 (m, 2H), 3.09-3.01 (m, 3H), 2.78 (s, 3H), 2.66 (s, 3H), 2.06-1.85 (m, 3H), 1.57-1.42 (m, 1H). |
| 38a | 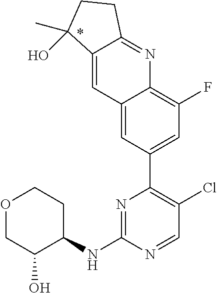 [SMILES: CC1(O)CCc2nc3c(F)cc(-c4nc(N[C@@H]5CCOC[C@H]5O)ncc4Cl)cc3cc21] Isomer 1 (earlier eluting isomer) | LC-MS (ESI): m/z 445.1 [M + H]⁺; ¹H NMR (500 MHz, DMSO-d₆) δ 8.47 (s, 1H), 8.37 (s, 1H), 8.27 (s, 1H), 7.83 (d, J = 11.8 Hz, 1H), 7.54 (s, 1H), 5.43 (s, 1H), 4.92 (d, J = 5.5 Hz, 1H), 3.90-3.78 (m, 3H), 3.50-3.47 (m, 1H), 3.38-3.34 (m, 1H), 3.23-3.17 (m, 1H), 3.09- 3.02 (m, 2H), 2.21 (t, J = 7.4 Hz, 2H), 2.02- 1.88 (m, 1H), 1.56 (s, 3H), 1.54-1.44 (m, 1H). de: 100%; Retention time: 0.762 min; instrument: Agilent- 1260; column: CHIRALPAK IA-3, 4.6 * 50 mm, 3.0 um; mobile phase: A for MtBE (0.1% DEA) and B for EtOH; gradient: B 30%; flow rate: 1.67 mL/min; high pressure: 114 bar; column temperature: 25° C.; wavelength: 254 nm. |
| 38b | 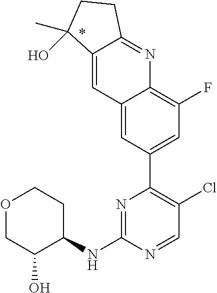 [SMILES: CC1(O)CCc2nc3c(F)cc(-c4nc(N[C@@H]5CCOC[C@H]5O)ncc4Cl)cc3cc21] Isomer 2 (later eluting isomer) | LC-MS (ESI): m/z 445.1 [M + H]⁺; ¹H NMR (500 MHz, DMSO-d₆) δ 8.47 (s, 1H), 8.37 (s, 1H), 8.27 (s, 1H), 7.83 (d, J = 11.8 Hz, 1H), 7.54 (s, 1H), 5.43 (s, 1H), 4.92 (d, J = 5.5 Hz, 1H), 3.89-3.80 (m, 3H), 3.50-3.48 (m, 1H), 3.38-3.33 (m, 1H), 3.23-3.20 (m, 1H), 3.09- 3.03 (m, 2H), 2.21 (t, J = 7.4 Hz, 2H), 2.02- 1.88 (m, 1H), 1.56 (s, 3H), 1.53-1.44 (m, 1H). de: 100%; Retention time: 1.581 min; instrument: Agilent- 1260; column: CHIRALPAK IA-3, 4.6*50 mm, 3.0 um; mobile phase: A for MtBE (0.1% DEA) and B for EtOH; gradient: B 30%; flow rate: 1.67 mL/min; high pressure: 114 bar; column temperature: 25° C.; wavelength: 254 nm. |
| 39a | 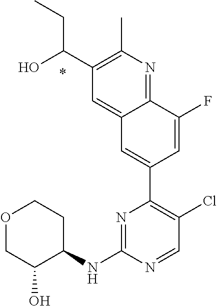 [SMILES: CCC(O)c1cc2cc(-c3nc(N[C@@H]4CCOC[C@H]4O)ncc3Cl)cc(F)c2nc1C] Isomer 1 (earlier eluting isomer) | LC-MS (ESI): m/z 447.2 [M + H]⁺; ¹H NMR (500 MHz, DMSO-d₆) δ 8.47 (s, 1H), 8.45 (s, 1H), 8.25 (s, 1H), 7.81 (d, J = 11.6 Hz, 1H), 7.54 (s, 1H), 5.45 (d, J = 4.4 Hz, 1H), 4.92 (d, J = 5.5 Hz, 1H), 4.88-4.83 (m, 1H), 3.90-3.77 (m, 3H), 3.55-3.45 (m, 1H), 3.39-3.30 (m, 1H), 3.11-3.01 (m, 1H), 2.74 (s, 3H), 2.04- 1.92 (m, 1H), 1.86-1.76 (m, 1H), 1.70-1.60 (m, 1H), 1.56-1.44 (m, 1H), 0.96 (t, J = 7.3 Hz, 3H). de: 100%; Retention time: 0.699 min; instrument: Agilent- 1260; column: CHIRALPAK IH-3, 4.6 * 50 mm, 3.0 um; mobile phase: A for MtBE (0.1% DEA) and B for EtOH; gradient: 3.6 min @B 30%; flow rate: 1.67 mL/min; high pressure: 114 bar; column temperature: 25° C.; wavelength: 254 nm. |
| 39b | 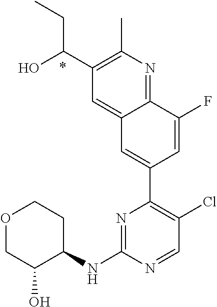 [SMILES: CCC(O)c1cc2cc(-c3nc(N[C@@H]4CCOC[C@H]4O)ncc3Cl)cc(F)c2nc1C] Isomer 2 (later eluting isomer) | LC-MS (ESI): m/z 447.2 [M + H]⁺; ¹H NMR (500 MHz, DMSO-d₆) δ 8.47 (s, 1H), 8.45 (s, 1H), 8.25 (s, 1H), 7.81 (d, J = 11.6 Hz, 1H), 7.54 (s, 1H), 5.45 (d, J = 4.5 Hz, 1H), 4.92 (d, J = 5.5 Hz, 1H), 4.88-4.83 (m, 1H), 3.90-3.77 (m, 3H), 3.55-3.45 (m, 1H), 3.39-3.30(m, 1H), 3.11-3.01 (m, 1H), 2.74 (s, 3H), 2.04- 1.92 (s, 1H),1.86-1.76 (m, 1H), 1.70-1.60 (m, 1H), 1.56-1.44 (m, 1H), 0.96 (t, J = 7.3 Hz, 3H). de: 100%; Retention time: 1.235 min; instrument: Agilent- 1260; column: CHIRALPAK IH-3, 4.6*50 mm, 3.0 um; mobile phase: A for MtBE (0.1% DEA) and B for EtOH; gradient: 3.6 min @B 30%; flow rate: 1.67 mL/min; high pressure: 114 bar; column temperature: 25° C.; wavelength: 254 nm. |
| 40a | 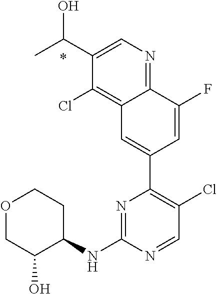 [SMILES: CC(O)c1cnc2c(F)cc(-c3nc(N[C@@H]4CCOC[C@H]4O)ncc3Cl)cc2c1Cl] Isomer 1 (earlier eluting isomer) | LC-MS (ESI): m/z 453.1 [M + H]⁺; ¹H NMR (500 MHz, DMSO-d₆) δ 9.19 (s, 1H) 8.51 (s, 2H), 8.06 (d, J = 11.0 Hz, 1H), 7.64 (s, 1H), 5.86 (d, J = 4.0 Hz, 1H), 5.34-5.31 (m, 1H), 4.94 (d, J = 5.5 Hz, 1H), 3.88-3.79 (m, 3H), 3.54-3.49 (m, 1H), 3.37-3.35 (m, 1H), 3.05 (t, J = 10.0 Hz, 1H), 2.03-1.98 (m, 1H), 1.55- 1.52 (m, 1H), 1.49 (d, J = 6.5 Hz, 3H). de: 100%; Retention time: 2.380 min; instrument: Agilent- 1260; column: CHIRALPAK IH-3, 3.0 × 100 mm, 3.0 um; mobile phase: A for CO₂ and B for MeOH (0.1% DEA); gradient: B 10% to 50% in 4 min, hold 2 min at 50%; flow rate: 2.0 mL/min; high pressure: 110 bar; column temperature: 35° C.; wavelength: 254 nm. |
| 40b | 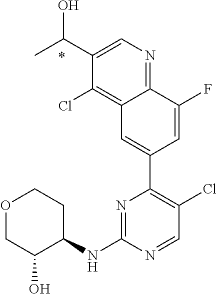 [SMILES: CC(O)c1cnc2c(F)cc(-c3nc(N[C@@H]4CCOC[C@H]4O)ncc3Cl)cc2c1Cl] Isomer 2 (later eluting isomer) | LC-MS (ESI): m/z 453.1 [M + H]⁺; ¹H NMR (500 MHz, DMSO-d₆) δ 9.19 (s, 1H) 8.51 (s, 2H), 8.06 (d, J = 11.0 Hz, 1H), 7.65 (s, 1H), 5.86 (d, J = 4.0 Hz, 1H), 5.34-5.31 (m, 1H), 4.93 (d, J = 5.5 Hz, 1H), 3.88-3.79 (m, 3H), 3.54-3.49 (m, 1H), 3.37-3.35 (m, 1H), 3.05 (t, J = 10.0 Hz, 1H), 2.03-1.98 (m, 1H), 1.55- 1.52 (m, 1H), 1.49 (d, J = 6.5 Hz, 3H). de: 100%; Retention time: 4.289 min; instrument: Agilent- 1260; column: CHIRALPAK IH-3, 3.0 × 100 mm, 3.0 um; mobile phase: A for CO₂ and B for MeOH (0.1% DEA); gradient: B 10% to 50% in 4 min, hold 2 min at 50%; flow rate: 2.0 mL/min; high pressure: 110 bar; column temperature: 35° C. |
| 41 | 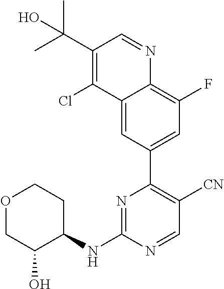 [SMILES: CC(C)(O)c1cnc2c(F)cc(-c3nc(N[C@@H]4CCOC[C@H]4O)ncc3C#N)cc2c1Cl] | LC-MS (ESI): m/z 458.1 [M + H]⁺; ¹H NMR (500 MHz, DMSO-d₆) δ 9.46 (s, 1H), 8.91- 8.86 (m, 1H), 8.84-8.76 (m, 1H), 8.53 (dd, J = 43.5, 8.4 Hz, 1H), 8.15 (ddd, J = 28.7, 11.1, 1.8 Hz, 1H), 5.84 (s, 1H), 5.02 (dd, J = 11.2, 5.6 Hz, 1H), 4.11-3.97 (m, 1H), 3.87-3.81 (m, 2H), 3.60-3.51 (m, 1H), 3.42-3.34 (m, 1H), 3.11- 3.03 (m, 1H), 2.04-1.86 (m, 1H), 1.75 (s, 6H), 1.65-1.52 (m, 1H). |
| 42 | 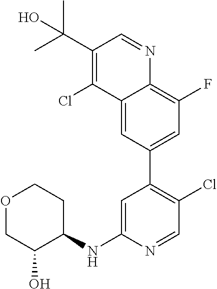 [SMILES: CC(C)(O)c1cnc2c(F)cc(-c3cc(N[C@@H]4CCOC[C@H]4O)ncc3Cl)cc2c1Cl] | LC-MS (ESI): m/z 466.1 [M + H]⁺; ¹H NMR (500 MHz, DMSO-d₆) δ 9.42 (s, 1H), 8.15- 8.13 (m, 1H), 8.12 (s, 1H), 7.81 (dd, J = 10.9, 1.8 Hz, 1H), 6.90 (d, J = 7.4 Hz, 1H), 6.71 (s, 1H), 5.81 (s, 1H), 5.01 (d, J = 5.3 Hz, 1H), 3.85- 3.75 (m, 3H), 3.44-3.35 (m, 2H), 3.09 (dd, J = 11.0, 9.4 Hz, 1H), 2.06-1.99 (m, 1H), 1.75 (s, 6H), 1.47-1.34 (m, 1H). |
| 43 | 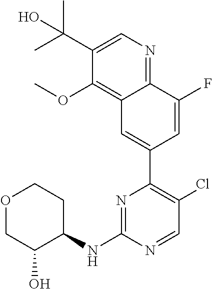 [SMILES: COc1c(C(C)(C)O)cnc2c(F)cc(-c3nc(N[C@@H]4CCOC[C@H]4O)ncc3Cl)cc12] | LC-MS (ESI): m/z 463.1 [M + H]⁺; ¹H NMR (500 MHz, DMSO-d₆) δ 9.28 (s, 1H) 8.49 (s, 1H), 8.37 (s, 1H), 7.92 (s, 1H), 7.58 (s, 1H), 5.51 (s, 1H), 4.94 (d, J = 5.0 Hz, 1H), 4.08 (s, 3H), 3.88-3.79 (m, 3H), 3.54-3.49 (m, 1H), 3.37-3.35 (m, 1H), 3.05 (t, J = 10.0 Hz, 1H), 2.03-1.98 (m, 1H), 1.64 (s, 6H), 1.55-1.52 (m, 1H). |
| 44 | 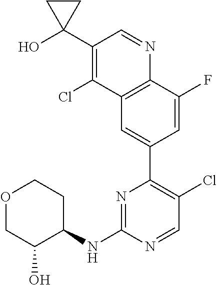 [SMILES: O[C@@H]1COCC[C@H]1Nc1ncc(Cl)c(-c2cc(F)c3ncc(C4(O)CC4)c(Cl)c3c2)n1] | LC-MS (ESI): m/z 465.1 [M + H]⁺; ¹H NMR (500 MHz, DMSO-d₆) δ 9.06 (s, 1H), 8.54 (s, 1H), 8.50 (s, 1H), 8.06 (d, J = 10.2 Hz, 1H), 7.64 (s, 1H), 6.21 (s, 1H), 4.94 (d, J = 5.4 Hz, 1H), 3.90-3.77 (m, 3H), 3.55-3.47 (m, 1H), 3.40-3.36 (m, 1H), 3.05 (dd, J = 10.9, 9.9 Hz, 1H), 2.04-1.87 (m, 1H), 1.56-1.47 (m, 1H), 1.17 (dd, J = 7.3, 5.3 Hz, 2H), 1.05 (dd, J = 7.1, 5.4 Hz, 2H). |
| 45a | 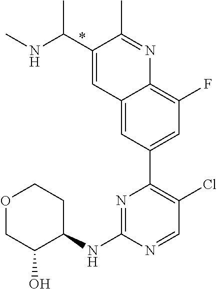 [SMILES: CNC(C)c1cc2cc(-c3nc(N[C@@H]4CCOC[C@H]4O)ncc3Cl)cc(F)c2nc1C] Isomer 1 (earlier eluting isomer) | LC-MS (ESI): m/z 446.2 [M + H]⁺; ¹H NMR (500 MHz, DMSO-d₆) δ 8.49-8.45 (m, 2H), 8.20 (s, 1H), 7.80 (d, J = 11.5 Hz, 1H), 7.55 (s, 1H), 4.93 (d, J = 5.4 Hz, 1H), 3.99 (q, J = 6.4 Hz, 1H), 3.89-3.78 (m, 3H), 3.52-3.44 (m, 1H), 3.38-3.34 (m, 1H), 3.05 (t, J = 10.4 Hz, 1H), 2.76 (s, 3H), 2.21 (s, 3H), 2.01-1.92 (m, 1H), 1.54-1.43 (m, 1H), 1.32 (d, J = 6.5 Hz, 3H). de: 96.8%; Retention time: 0.646 min; instrument: Agilent- 1260; column: CHIRALPAK IA-3, 4.6*50 mm, 3.0 um; mobile phase: A for MtBE (0.1% DEA) and B for MeOH; gradient: B 30%; flow rate: 1.67 mL/min; high pressure: 110 bar; column temperature: 25° C.; wavelength: 254 nm. |
| 45b | 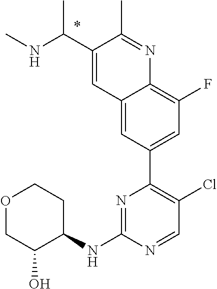 [SMILES: CNC(C)c1cc2cc(-c3nc(N[C@@H]4CCOC[C@H]4O)ncc3Cl)cc(F)c2nc1C] Isomer 2 (later eluting isomer) | LC-MS (ESI): m/z 446.2 [M + H]⁺; ¹H NMR (500 MHz, DMSO-d₆) δ 8.51-8.44 (m, 2H), 8.21 (s, 1H), 7.81 (d, J = 11.6 Hz, 1H), 7.56 (s, 1H), 4.93 (d, J = 5.5 Hz, 1H), 4.01-3.97 (m, J = 6.4 Hz, 1H), 3.89-3.77 (m, 3H), 3.51-3.49 (m, 1H), 3.38-3.36 (m, 1H), 3.06 (t, J = 10.4 Hz, 1H), 2.76 (s, 3H), 2.22 (s, 3H), 2.01-1.90 (m, 1H), 1.55-1.46 (m, 1H), 1.32 (d, J = 6.6 Hz, 3H). de: 98.5%; Retention time: 1.029 min; Instrument: Agilent-1260; column: CHIRALPAK IA-3, 4.6*50 mm, 3.0 um; mobile phase: A for MtBE (0.1% DEA) and B for MeOH; gradient: B 30%; flow rate: 1.67 mL/min; high pressure: 110 bar; column temperature: 25° C.; Wavelength: 254 nm. |
| 46 | 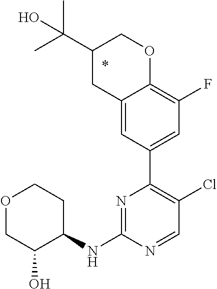 [SMILES: CC(C)(O)C1COc2c(F)cc(-c3nc(N[C@@H]4CCOC[C@H]4O)ncc3Cl)cc2C1] | LC-MS (ESI): m/z 438.2 [M + H]⁺; ¹H NMR (500 MHz, DMSO-d₆) δ 8.35 (s, 1H), 7.46- 7.41 (m, 2H), 7.39 (d, J = 8.0 Hz, 1H), 4.89 (d, J = 5.5 Hz, 1H), 4.59-4.56 (m, 1H), 4.55 (s, 1H), 3.86 (t, J = 10.8 Hz, 1H), 3.82-3.75 (m, 3H), 3.53-3.42 (m, 1H), 3.37-3.33 (m, 1H), 3.04 (t, J = 10.4 Hz, 1H), 2.87-2.69 (m, 2H), 1.98- 1.89 (m, 2H), 1.53-1.41 (m, 1H), 1.17 (d, J = 17.2 Hz, 6H). |
| 47 | 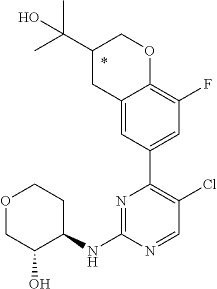 [SMILES: CC(C)(O)C1COc2c(F)cc(-c3nc(N[C@@H]4CCOC[C@H]4O)ncc3Cl)cc2C1] | LC-MS (ESI): m/z 438.2 [M + H]⁺; ¹H NMR (500 MHz, DMSO-d₆) δ 8.36 (s, 1H), 7.47- 7.42 (m, 2H), 7.39 (d, J = 8.0 Hz, 1H), 4.90 (d, J = 5.4 Hz, 1H), 4.59-4.57 (m, 1H), 4.56 (s, 1H), 3.87 (t, J = 10.8 Hz, 1H), 3.84-3.76 (m, 3H), 3.51-3.44 (m, 1H), 3.37-3.33 (m, 1H), 3.09- 3.01 (m, 1H), 2.88-2.72 (m, 2H), 1.98-1.90 (m, 2H), 1.52-1.44 (m, 1H), 1.17 (d, J = 17.2 Hz, 6H). |
| 48a | 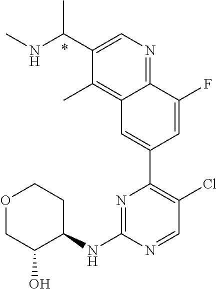 [SMILES: CNC(C)c1cnc2c(F)cc(-c3nc(N[C@@H]4CCOC[C@H]4O)ncc3Cl)cc2c1C] Isomer 1 (earlier eluting isomer) | LC-MS (ESI): m/z 446.2 [M + H]⁺; ¹H NMR (500 MHz, DMSO-d₆) δ 9.09 (s, 1H), 8.48 (s, 1H), 8.38 (s, 1H), 7.87 (s, 1H), 7.59 (s, 1H), 4.95 (d, J = 5.4 Hz, 1H), 4.18 (q, J = 6.3 Hz, 1H), 3.89-3.76 (m, 3H), 3.51-3.45 (m, 1H), 3.37-3.34 (m, 1H), 3.06 (t, J = 10.4 Hz, 1H), 2.71 (s, 3H), 2.18 (s, 3H), 1.98-1.89 (m, 1H), 1.54-1.43 (m, 1H), 1.34 (d, J = 6.5 Hz, 3H). de: 100%; Retention time: 1.450 min; instrument: Agilent- 1260; column: CHIRALPAK IH-3 , 4.6*50 mm, 3.0 um; mobile phase: A for Hex (0.1% DEA) and B for EtOH; gradient: B 50%; flow rate: 1 mL/min; high pressure: 110 bar; column temperature: 25° C.; wavelength: 254 nm. |
| 48b | 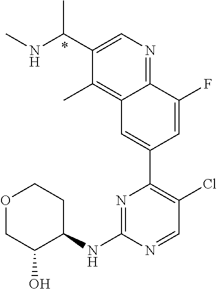 [SMILES: CNC(C)c1cnc2c(F)cc(-c3nc(N[C@@H]4CCOC[C@H]4O)ncc3Cl)cc2c1C] Isomer 2 (later eluting isomer) | LC-MS (ESI): m/z 446.2 [M + H]⁺; ¹H NMR (500 MHz, DMSO-d₆) δ 9.09 (s, 1H), 8.48 (s, 1H), 8.38 (s, 1H), 7.87 (s, 1H), 7.59 (s, 1H), 4.95 (d, J = 5.4 Hz, 1H), 4.18 (q, J = 6.5 Hz, 1H), 3.92-3.75 (m, 3H), 3.51-3.44 (m, 1H), 3.37-3.33 (m, 1H), 3.06 (t, J = 10.4 Hz, 1H), 2.71 (s, 3H), 2.18 (s, 3H), 2.01-1.90 (m, 1H), 1.57-1.45 (m, 1H), 1.33 (d, J = 6.5 Hz, 3H). de: 100%; Retention time: 3.647 min; Instrument: Agilent-1260; column: CHIRALPAK IH-3 , 4.6*50 mm, 3.0 um; mobile phase: A for Hex(0.1% DEA) and B for EtOH; gradient: B 50%; flow rate: 1 mL/min; high pressure: 110 bar; column temperature: 25° C.; Wavelength: 254 nm. |
| 49 | 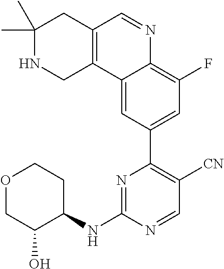 [SMILES: CC1(C)Cc2cnc3c(F)cc(-c4nc(N[C@@H]5CCOC[C@H]5O)ncc4C#N)cc3c2CN1] | LC-MS (ESI): m/z 449.2 [M + H]⁺; ¹H NMR (500 MHz, DMSO-d₆) δ 8.84 (d, J = 28.4 Hz, 1H), 8.76 (s, 1H), 8.54-8.49 (m, 1H), 8.46- 8.42 (m, 1H), 8.03-7.94 (m, 1H), 5.02 (s, 1H), 4.36 (d, J = 4.7 Hz, 2H), 4.09-3.96 (m, 1H), 3.86-3.80 (m, 2H), 3.58-3.49 (m, 1H), 3.41- 3.37 (m, 1H), 3.12-3.03 (m, 1H), 2.78 (s, 2H), 1.99-1.85 (m, 1H), 1.61-1.53 (m, 1H), 1.14 (s, 6H). |
| 50 | 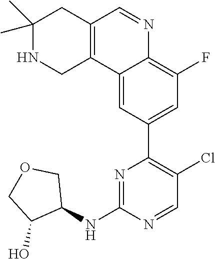 [SMILES: CC1(C)Cc2cnc3c(F)cc(-c4nc(N[C@@H]5COC[C@H]5O)ncc4Cl)cc3c2CN1] | LC-MS (ESI): m/z 444.2 [M + H]⁺; ¹H NMR (500 MHz, DMSO-d₆) δ 8.72 (s, 1H), 8.52 (s, 1H), 8.24 (s, 1H), 7.93-7.81 (m, 2H), 5.22 (s, 1H), 4.32 (s, 2H), 4.24 (s, 1H), 4.15 (s, 1H), 4.06-4.00 (m, 1H), 3.91 (dd, J = 9.3, 4.5 Hz, 1H), 3.67 (s, 1H), 3.55 (dd, J = 9.3, 2.2 Hz, 1H), 2.75 (s, 2H), 1.13 (s, 6H). |
| 51 | 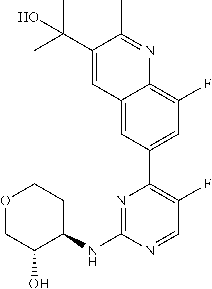 [SMILES: Cc1nc2c(F)cc(-c3nc(N[C@@H]4CCOC[C@H]4O)ncc3F)cc2cc1C(C)(C)O] | LC-MS (ESI): m/z 431.2 [M + H]⁺; ¹H NMR (500 MHz, DMSO-d₆) δ 8.50 (d, J = 3.7 Hz, 2H), 8.45 (s, 1H), 8.06 (dd, J = 12.2, 1.7 Hz, 1H), 7.27 (d, J = 7.9 Hz, 1H), 5.34 (s, 1H), 4.93 (d, J = 5.4 Hz, 1H), 3.89-3.78 (m, 3H), 3.54- 3.47 (m, 1H), 3.38 (t, J = 11.6 Hz, 1H), 3.09 (t, J = 10.3 Hz, 1H), 2.96 (s, 3H), 2.04-1.95 (m, 1H), 1.65 (s, 6H), 1.55-1.44 (m, 1H). |
| 52 | 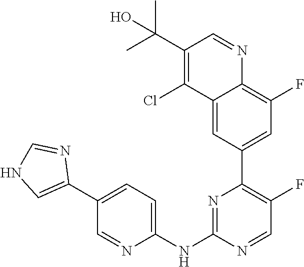 [SMILES: CC(C)(O)c1cnc2c(F)cc(-c3nc(Nc4ccc(-c5c[nH]cn5)cn4)ncc3F)cc2c1Cl] | LC-MS (ESI): m/z 494.1 [M + H]⁺; ¹H NMR (500 MHz, DMSO-d₆) δ 12.23 (s, 1H), 10.21 (s, 1H), 9.46 (s, 1H), 8.98 (d, J = 1.7 Hz, 1H), 8.83 (d, J = 3.5 Hz, 1H), 8.76 (s, 1H), 8.32 (dd, J = 11.5, 1.8 Hz, 1H), 8.26 (d, J = 8.7 Hz, 1H), 8.16 (s, 1H), 7.75 (s, 1H), 7.69 (s, 1H), 5.85 (s, 1H), 1.77 (s, 6H). |
| 53 | 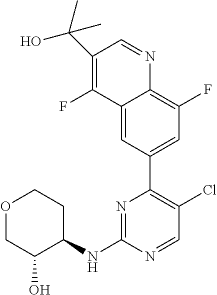 [SMILES: CC(C)(O)c1cnc2c(F)cc(-c3nc(N[C@@H]4CCOC[C@H]4O)ncc3Cl)cc2c1F] | LC-MS (ESI): m/z 451.1 [M + H]⁺; ¹H NMR (500 MHz, DMSO-d₆) δ 9.28 (d, J = 10.3 Hz, 1H), 8.50 (s, 1H), 8.35 (s, 1H), 8.06-7.98 (m, 1H), 7.64 (s, 1H), 5.79 (s, 1H), 4.95 (d, J = 5.4 Hz, 1H), 3.96-3.75 (m, 3H), 3.54-3.47 (m, 1H), 3.38-3.34 (m, 1H), 3.06 (t, J = 10.4 Hz, 1H), 1.95 (d, J = 10.3 Hz, 1H), 1.65 (s, 6H), 1.53-1.46 (m, 1H). |
| 54a | 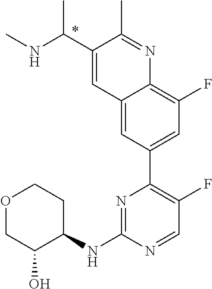 [SMILES: CNC(C)c1cc2cc(-c3nc(N[C@@H]4CCOC[C@H]4O)ncc3F)cc(F)c2nc1C] Isomer 1 (earlier eluting isomer) | LC-MS (ESI): m/z 430.2 [M + H]⁺; ¹H NMR (500 MHz, DMSO-d₆) δ 8.52-8.49 (m, 2H), 8.41 (s, 1H), 8.06 (d, J = 12.4 Hz, 1H), 7.27 (d, J = 7.8 Hz, 1H), 4.92 (d, J = 5.5 Hz, 1H), 3.99 (q, J = 6.7 Hz, 1H), 3.86-3.79 (m, 3H), 3.55- 3.47 (m, 1H), 3.43-3.36 (m, 1H), 3.09 (t, J = 10.1 Hz, 1H), 2.76 (s, 3H), 2.21 (s, 3H), 2.04- 1.94 (m, 1H), 1.56-1.44 (m, 1H), 1.33 (d, J = 6.6 Hz, 3H). de: 99.5%; Retention time: 0.686 min; Instrument: Agilent-1260; column: CHIRALPAK IA-3, 4.6 * 50 mm, 3.0 um; mobile phase: A for MtBE (0.1% DEA) and B for MeOH; gradient: B 15%; flow rate: 1.67 mL/min; high pressure: 114 bar; column temperature: 25° C.; Wavelength: 254 nm. |
| 54b | 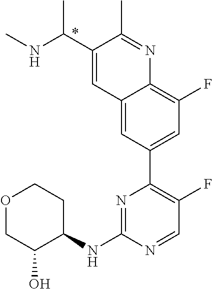 [SMILES: CNC(C)c1cc2cc(-c3nc(N[C@@H]4CCOC[C@H]4O)ncc3F)cc(F)c2nc1C] Isomer 2 (later eluting isomer) | LC-MS (ESI): m/z 430.2 [M + H]⁺; ¹H NMR (500 MHz, DMSO-d₆) δ 8.52-8.49 (m, 2H), 8.41 (s, 1H), 8.06 (d, J = 12.3 Hz, 1H), 7.27 (d, J = 7.8 Hz, 1H), 4.93 (d, J = 5.5 Hz, 1H), 4.00 (q, J = 6.7 Hz, 1H), 3.87-3.79 (m, 3H), 3.54- 3.47 (m, 1H), 3.42-3.36 (m, 1H), 3.09 (t, J = 10.4 Hz, 1H), 2.76 (s, 3H), 2.22 (s, 3H), 2.03- 1.96 (m, 1H), 1.54-1.43 (m, 1H), 1.33 (d, J = 6.5 Hz, 3H). de: 97.4%; Retention time: 0.931 min; Instrument: Agilent-1260; column: CHIRALPAK IA-3, 4.6 * 50 mm, 3.0 um; mobile phase: A for MtBE (0.1% DEA) and B for MeOH; gradient: B 15%; flow rate: 1.67 mL/min; high pressure: 114 bar; column temperature: 25° C.; Wavelength: 254 nm. |
| 55 | 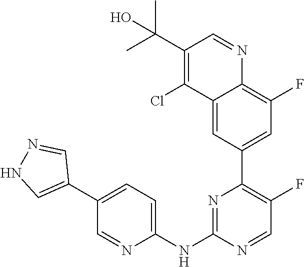 [SMILES: CC(C)(O)c1cnc2c(F)cc(-c3nc(Nc4ccc(-c5cn[nH]c5)cn4)ncc3F)cc2c1Cl] | LC-MS (ESI): m/z 494.1 [M + H]⁺; ¹H NMR (500 MHz, DMSO-d₆) δ 13.00 (s, 1H), 10.20 (s, 1H), 9.46 (s, 1H), 8.95 (s, 1H), 8.82 (d, J = 3.5 Hz, 1H), 8.62 (d, J = 2.5 Hz, 1H), 8.39-8.17 (m, 3H), 8.15-7.92 (m, 2H), 5.85 (s, 1H), 1.77 (s, 6H). |
| 56 | 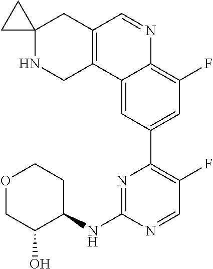 [SMILES: O[C@@H]1COCC[C@H]1Nc1ncc(F)c(-c2cc(F)c3ncc4c(c3c2)CNC2(CC2)C4)n1] | LC-MS (ESI): m/z 440.2 [M + H]⁺; ¹H NMR (500 MHz, DMSO-d₆) δ 8.74 (s, 1H), 8.52 (d, J = 3.5 Hz, 1H), 8.42 (s, 1H), 8.07 (d, J = 11.9 Hz, 1H), 7.32 (d, J = 7.8 Hz, 1H), 4.95 (d, J = 5.4 Hz, 1H), 4.32 (s, 2H), 3.90-3.81 (m, 3H), 3.58- 3.50 (m, 1H), 3.46-3.40 (m, 1H), 3.14-3.05 (m, 1H), 2.89 (s, 2H), 2.08-1.97 (m, 1H), 1.57- 1.46 (m, 1H), 0.66 (s, 2H), 0.53 (s, 2H). |
| 57a | 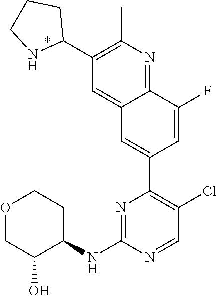 [SMILES: Cc1nc2c(F)cc(-c3nc(N[C@@H]4CCOC[C@H]4O)ncc3Cl)cc2cc1C1CCCN1] Isomer 1 (earlier eluting isomer) | LC-MS (ESI): m/z 458.2 [M + H]⁺; ¹H NMR (500 MHz, DMSO-d₆) δ 8.55 (s, 1H), 8.46 (s, 1H), 8.22 (s, 1H), 7.80 (d, J = 11.0 Hz, 1H), 7.53 (s, 1H), 4.93 (d, J = 5.5 Hz, 1H), 4.48 (t, J = 7.5 Hz, 1H), 3.88-3.79 (m, 3H), 3.54-3.49 (m, 1H), 3.37-3.35 (m, 1H), 3.12-3.01 (m, 3H), 2.75 (s, 3H), 2.36-2.32 (m, 1H), 1.98- 1.94 (m, 1H), 1.84-1.80 (m, 2H), 1.54-1.47 (m, 2H). de: 100%; Retention time: 2.583 min; Instrument: Agilent-1260; column: CHIRALPAK ID-3, 4.6 * 50 mm, 3.0 um; mobile phase: A for Hex: MtBE = 1:1 (0.1% DEA) and B for IPA; gradient: B 50%; flow rate: 1.0 mL/min; high pressure: 72 bar; column temperature: 25° C.; Wavelength: 254 nm. |
| 57b | 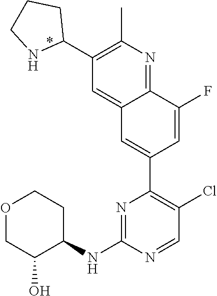 [SMILES: Cc1nc2c(F)cc(-c3nc(N[C@@H]4CCOC[C@H]4O)ncc3Cl)cc2cc1C1CCCN1] Isomer 2 (later eluting isomer) | LC-MS (ESI): m/z 458.2 [M + H]⁺; ¹H NMR (500 MHz, DMSO-d₆) δ 8.55 (s, 1H), 8.46 (s, 1H), 8.22 (s, 1H), 7.80 (d, J = 11.0 Hz, 1H), 7.53 (s, 1H), 4.93 (d, J = 5.5 Hz, 1H), 4.48 (t, J = 7.5 Hz, 1H), 3.88-3.79 (m, 3H), 3.54-3.49 (m, 1H), 3.37-3.35 (m, 1H), 3.12-3.02 (m, 3H), 2.75 (s, 3H), 2.37-2.32 (m, 1H), 1.98- 1.94 (m, 1H), 1.84-1.80 (m, 2H), 1.54-1.46 (m, 2H). de: 97.9%; Retention time: 3.301 min; Instrument: Agilent-1260; column: CHIRALPAK ID-3, 4.6 * 50 mm, 3.0 um; mobile phase: A for Hex: MtBE = 1:1 (0.1% DEA) and B for IPA; gradient: B 50%; flow rate: 1.0 mL/min; high pressure: 72 bar; column temperature: 25° C.; Wavelength: 254 nm. |
| 58 | 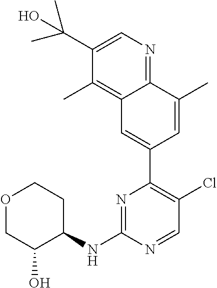 [SMILES: Cc1c(C(C)(C)O)cnc2c(C)cc(-c3nc(N[C@@H]4CCOC[C@H]4O)ncc3Cl)cc12] | LC-MS (ESI): m/z 443.2 [M + H]⁺; ¹H NMR (500 MHz, DMSO-d₆) δ 9.08 (s, 1H), 8.45 (s, 1H), 8.43 (s, 1H), 7.89 (s, 1H), 7.53 (s, 1H), 5.32 (s, 1H), 4.94 (s, 1H), 3.88-3.79 (m, 3H), 3.54-3.49 (m, 1H), 3.37-3.35 (m, 1H), 3.05 (t, J= 10.5 Hz, 1H), 2.95 (s, 3H), 2.74 (s, 3H), 1.98- 1.94 (m, 1H), 1.68 (s, 6H), 1.54-1.47 (m, 1H). |
| 59a | 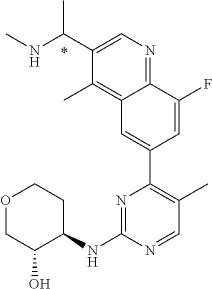 Isomer 1 (earlier eluting isomer) | LC-MS (ESI): m/z 430.2 [M + H]⁺; ¹H NMR (500 MHz, DMSO-d₆) δ 9.10 (s, 1H), 8.63 (s, 1H), 8.52 (d, J = 3.6 Hz, 1H), 8.08 (d, J = 11.6 Hz, 1H), 7.32 (d, J = 7.6 Hz, 1H), 4.96 (d, J = 5.0 Hz, 1H), 4.20 (q, J = 6.5 Hz, 1H), 3.89- 3.77 (m, 3H), 3.58-3.50 (m, 1H), 3.42-3.38 (m, 1H), 3.09 (t, J = 10.2 Hz, 1H), 2.74 (s, 3H), 2.19 (s, 3H), 2.07-1.98 (m, 1H), 1.57-1.47 (m, 1H), 1.36 (d, J = 6.5 Hz, 3H). de: 100%; Retention time: 1.985 min; Instrument: Agilent-1260; column: CHIRALPAK IH-3, 3.0 * 100 mm, 3.0 um; mobile phase: A for CO₂ and B for MeOH (0.1% DEA); gradient: B 10% to 50% in 4 min, hold 2 min at 50%; flow rate: 2 mL/min; high pressure: 110 bar; column temperature: 25° C.; Wavelength: 254 nm. |
| 59b | 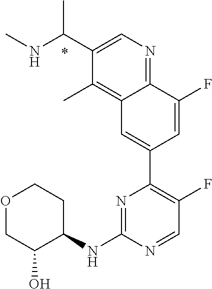 [SMILES: CNC(C)c1cnc2c(F)cc(-c3nc(N[C@@H]4CCOC[C@H]4O)ncc3F)cc2c1C] Isomer 2 (later eluting isomer) | LC-MS (ESI): m/z 430.2 [M + H]⁺; ¹H NMR (500 MHz, DMSO-d₆) δ 9.10 (s, 1H), 8.62 (s, 1H), 8.52 (d, J = 3.3 Hz, 1H), 8.07 (d, J = 11.6 Hz, 1H), 7.32 (d, J = 7.6 Hz, 1H), 4.96 (d, J = 5.3 Hz, 1H), 4.20 (q, J = 6.5 Hz, 1H), 3.88- 3.79 (m, 3H), 3.58-3.50 (m, 1H), 3.42-3.37 (m, 1H), 3.09 (t, J = 10.2 Hz, 1H), 2.74 (s, 3H), 2.19 (s, 3H), 2.07-1.99 (m, 1H), 1.57-1.46 (m, 1H), 1.36 (d, J = 6.5 Hz, 3H). de: 100%; Retention time: 3.740 min; Instrument: Agilent-1260; column: CHIRALPAK IH-3, 3.0 * 100 mm, 3.0 um; mobile phase: A for CO₂ and B for MeOH (0.1% DEA); gradient: B 10% to 50% in 4 min, hold 2 min at 50%; flow rate: 2 mL/min; high pressure: 110 bar; column temperature: 25° C.; Wavelength: 254 nm. |
| 63a | 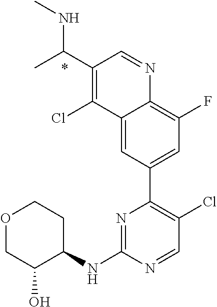 [SMILES: CNC(C)c1cnc2c(F)cc(-c3nc(N[C@@H]4CCOC[C@H]4O)ncc3Cl)cc2c1Cl] Isomer 1 (earlier eluting isomer) | LC-MS (ESI): m/z 466.1 [M + H]⁺; ¹H NMR (500 MHz, DMSO-d₆) δ 9.16 (s, 1H), 8.56-8.44 (m, 2H), 8.04 (s, 1H), 7.74-7.54 (m, 1H), 4.95 (d, J = 5.4 Hz, 1H), 4.29 (q, J = 6.7 Hz, 1H), 3.87-3.78 (m, 3H), 3.53-3.47 (m, 1H), 3.42- 3.37 (m, 1H), 3.04 (t, J = 10.4 Hz, 1H), 2.17 (s, 3H), 2.01-1.91 (m, 1H), 1.55-1.47 (m, 1H), 1.38 (d, J = 6.6 Hz, 3H). de: 100%; Retention time: 2.259 min; instrument: Agilent- 1260; column: CHIRALPAK IH-3, 3.0 * 100 mm, 3.0 um; mobile phase: A for CO₂ and B for MeOH (0.1% DEA); gradient: B 10% to 50% in 4 min, hold 2 min at 50%; flow rate: 2 mL/min; high pressure: 110 bar; column temperature: 25° C.; Wavelength: 254 nm. |
| 63b | 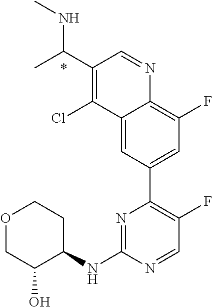 Isomer 2 (later eluting isomer) | LC-MS (ESI): m/z 466.1 [M + H]⁺; ¹H NMR (500 MHz, DMSO-d₆) δ 9.15 (s, 1H), 8.59-8.43 (m, 2H), 8.04 (s, 1H), 7.74-7.55 (m, 1H), 4.95 (d, J = 5.4 Hz, 1H), 4.28 (q, J = 6.7 Hz, 1H), 3.87-3.78 (m, 3H), 3.55-3.49 (m, 1H), 3.42- 3.38 (m, 1H), 3.04 (t, J = 10.4 Hz, 1H), 2.17 (s, 3H), 2.01-1.90 (m, 1H), 1.54-1.47 (m, 1H), 1.37 (d, J = 6.6 Hz, 3H). de: 98.8%; Retention time: 3.369 min; instrument: Agilent- 1260; column: CHIRALPAK IH-3, 3.0 * 100 mm, 3.0 um; mobile phase: A for CO₂ and B for MeOH (0.1% DEA); gradient: B 10% to 50% in 4 min, hold 2 min at 50%; flow rate: 2 mL/min; high pressure: 110 bar; column temperature: 25° C.; Wavelength: 254 nm. |
| 64a | 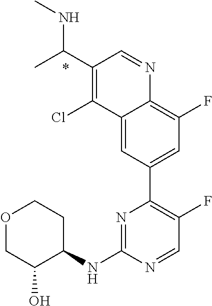 [SMILES: CNC(C)c1cnc2c(F)cc(-c3nc(N[C@@H]4CCOC[C@H]4O)ncc3F)cc2c1Cl] Isomer 1 (earlier eluting isomer) | LC-MS (ESI): m/z 450.2 [M + H]⁺; ¹H NMR (500 MHz, DMSO-d₆) δ 9.16 (s, 1H), 8.73 (s, 1H), 8.54 (d, J = 3.6 Hz, 1H), 8.21 (d, J = 11.5 Hz, 1H), 7.39 (d, J = 7.9 Hz, 1H), 4.96 (d, J = 5.4 Hz, 1H), 4.29 (q, J = 6.7 Hz, 1H), 3.87-3.80 (m, 3H), 3.55-3.50 (m, 1H), 3.41-3.37 (m, 1H), 3.07 (t, J = 10.4 Hz, 1H), 2.17 (s, 3H), 2.05- 1.97 (m, 1H), 1.54-1.47 (m, 1H), 1.38 (d, J = 6.6 Hz, 3H). de: 100%; Retention time: 2.901 min; instrument: Agilent- 1260; column: Lux 3um Cellulose-2, 4.6 * 50 mm, 3.0 um; mobile phase: A for Hex (0.1% DEA) and B for EtOH; gradient: B 50%; flow rate: 1 mL/min; high pressure: 110 bar; column temperature: 25° C.; wavelength: 254 nm. |
| 64b | 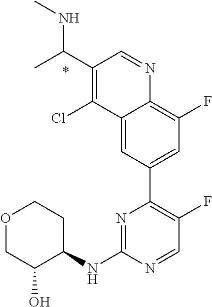 [SMILES: CNC(C)c1cnc2c(F)cc(-c3nc(N[C@@H]4CCOC[C@H]4O)ncc3F)cc2c1Cl] Isomer 2 (later eluting isomer) | LC-MS (ESI): m/z 450.2 [M + H]⁺; ¹H NMR (500 MHz, DMSO-d₆) δ 9.16 (s, 1H), 8.73 (s, 1H), 8.54 (d, J = 3.6 Hz, 1H), 8.21 (d, J = 11.5 Hz, 1H), 7.39 (d, J = 7.9 Hz, 1H), 4.96 (d, J = 5.3 Hz, 1H), 4.29 (q, J = 6.9 Hz, 1H), 3.87-3.81 (m, 3H), 3.57-3.51 (m, 1H), 3.46-3.43 (m, 1H), 3.07 (t, J = 10.4 Hz, 1H), 2.17 (s, 3H), 2.05- 1.97 (m, 1H), 1.55-1.46 (m, 1H), 1.38 (d, J = 6.6 Hz, 3H). de: 100%; Retention time: 4.133 min; instrument: Agilent- 1260; column: Lux 3 um Cellulose-2, 4.6 * 50 mm, 3.0 um; mobile phase: A for Hex (0.1% DEA) and B for EtOH; gradient: B 50%; flow rate: 1 mL/min; high pressure: 110 bar; column temperature: 25° C.; wavelength: 254 nm. |
| 65 | 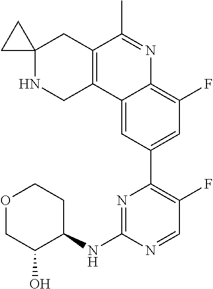 [SMILES: Cc1nc2c(F)cc(-c3nc(N[C@@H]4CCOC[C@H]4O)ncc3F)cc2c2c1CC1(CC1)NC2] | LC-MS (ESI): m/z 454.2 [M + H]⁺; ¹H NMR (500 MHz, DMSO-d₆) δ 8.50 (d, J = 3.6 Hz, 1H), 8.39 (s, 1H), 8.02 (d, J = 11.9 Hz, 1H), 7.30 (d, J = 7.8 Hz, 1H), 4.97 (s, 1H), 4.29 (d, J = 6.4 Hz, 2H), 3.86-3.79 (m, 3H), 3.54-3.49 (m, 1H), 3.40-3.37 (m, 1H), 3.08 (t, J = 10.4 Hz, 1H), 2.75 (s, 2H), 2.59 (s, 3H), 2.04-1.97 (m, 1H), 1.53-1.45 (m, 1H), 0.65-0.61 (m, 2H), 0.53-0.48 (m, 2H). |
| 66 | 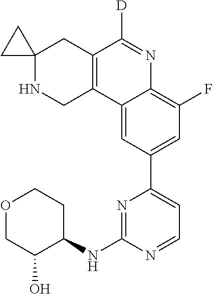 [SMILES: [2H]c1nc2c(F)cc(-c3ccnc(N[C@@H]4CCOC[C@H]4O)n3)cc2c2c1CC1(CC1)NC2] | LC-MS (ESI): m/z 423.2 [M + H]⁺; ¹H NMR (500 MHz, DMSO-d₆) δ 8.50 (s, 1H), 8.43 (d, J = 5.1 Hz, 1H), 8.21 (d, J = 12.0 Hz, 1H), 7.44 (d, J = 5.1 Hz, 1H), 7.24 (d, J = 7.7 Hz, 1H), 5.01 (s, 1H), 4.37 (d, J = 6.7 Hz, 2H), 3.96-3.82 (m, 3H), 3.59-3.52 (m, 1H), 3.29-3.23 (m, 1H), 3.14-3.03 (m, 1H), 2.87 (s, 2H), 2.12-1.92 (m, 1H), 1.55-1.48 (m, 1H), 0.66-0.61 (m, 2H), 0.54-0.48 (m, 2H). |
| 67 | 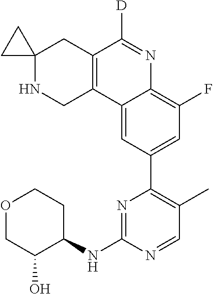 [SMILES: [2H]c1nc2c(F)cc(-c3nc(N[C@@H]4CCOC[C@H]4O)ncc3C)cc2c2c1CC1(CC1)NC2] | LC-MS (ESI): m/z 437.2 [M + H]⁺; ¹H NMR (500 MHz, DMSO-d₆) δ 8.28 (s, 1H), 7.98 (s, 1H), 7.77 (d, J = 11.4 Hz, 1H), 7.03 (d, J = 7.7 Hz, 1H), 4.99 (s, 1H), 4.29 (s, 2H), 3.85-3.79 (m, 3H), 3.54-3.48 (m, 1H), 3.45-3.42 (m, 1H), 3.06 (t, J = 10.4 Hz, 1H), 2.88 (s, 2H), 2.23 (s, 3H), 2.03-1.97 (m, 1H), 1.53-1.45 (m, 1H), 0.66-0.62 (m, 2H), 0.53-0.49 (m, 2H). |
| 68 | 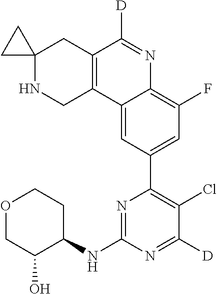 [SMILES: [2H]c1nc(N[C@@H]2CCOC[C@H]2O)nc(-c2cc(F)c3nc([2H])c4c(c3c2)CNC2(CC2)C4)c1Cl] | LC-MS (ESI): m/z 458.2 [M + H]⁺; ¹H NMR (500 MHz, DMSO-d₆) δ 8.22 (s, 1H), 7.86 (s, 1H), 7.59 (s, 1H), 4.95 (d, J = 5.5 Hz, 1H), 4.29 (s, 2H), 3.88-3.79 (m, 3H), 3.55-3.50 (m, 1H), 3.46-3.42 (m, 1H), 3.06 (t, J = 10.4 Hz, 1H), 2.88 (s, 2H), 2.01-1.92 (m, 1H), 1.55-1.47 (m, 1H), 0.67-0.63 (m, 2H), 0.54-0.49 (m, 2H). |
| 69 | 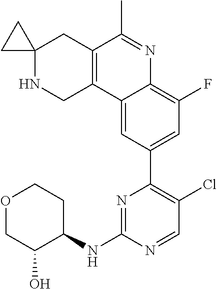 [SMILES: Cc1nc2c(F)cc(-c3nc(N[C@@H]4CCOC[C@H]4O)ncc3Cl)cc2c2c1CC1(CC1)NC2] | LC-MS (ESI): m/z 470.2 [M + H]⁺; ¹H NMR (500 MHz, DMSO-d₆) δ 8.47 (s, 1H), 8.19 (s, 1H), 7.83 (s, 1H), 7.58 (s, 1H), 4.95 (d, J = 5.4 Hz, 1H), 4.30 (s, 2H), 3.92-3.73 (m, 3H), 3.55- 3.50 (m, 1H), 3.46-3.43 (m, 1H), 3.06 (t, J = 10.3 Hz, 1H), 2.77 (s, 2H), 2.60 (s, 3H), 2.02- 1.92 (m, 1H), 1.56-1.46 (m, 1H), 0.70-0.65 (m, 2H), 0.58-0.53 (m, 2H). |
| 70 | 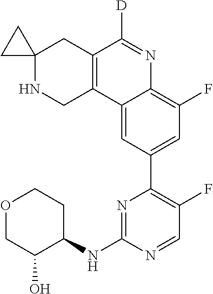 [SMILES: [2H]c1nc2c(F)cc(-c3nc(N[C@@H]4CCOC[C@H]4O)ncc3F)cc2c2c1CC1(CC1)NC2] | LC-MS (ESI): m/z 441.2 [M + H]⁺; ¹H NMR (500 MHz, DMSO-d₆) δ 8.53 (d, J = 3.5 Hz, 1H), 8.42 (s, 1H), 8.07 (d, J = 11.8 Hz, 1H), 7.33 (d, J = 7.8 Hz, 1H), 4.96 (d, J = 5.2 Hz, 1H), 4.31 (s, 2H), 3.89-3.80 (m, 3H), 3.60-3.52 (m, 1H), 3.48-3.45 (m, 1H), 3.09 (t, J = 10.5 Hz, 1H), 2.89 (s, 2H), 2.08-1.95 (m, 1H), 1.58- 1.43 (m, 1H), 0.68-0.64 (m, 2H), 0.55-0.50 (m, 2H). |
| 71 | 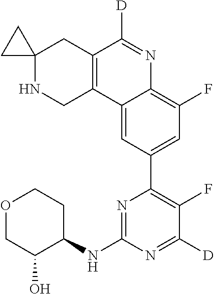 [SMILES: [2H]c1nc(N[C@@H]2CCOC[C@H]2O)nc(-c2cc(F)c3nc([2H])c4c(c3c2)CNC2(CC2)C4)c1F] | LC-MS (ESI): m/z 442.2 [M + H]⁺; ¹H NMR (500 MHz, DMSO-d₆) δ 8.42 (s, 1H), 8.07 (d, J = 11.9 Hz, 1H), 7.33 (d, J = 7.8 Hz, 1H), 4.96 (d, J = 5.4 Hz, 1H), 4.31 (s, 2H), 3.89-3.80 (m, 3H), 3.60-3.52 (m, 1H), 3.48-3.44 (m, 1H), 3.09 (t, J = 10.4 Hz, 1H), 2.89 (s, 2H), 2.08- 1.95 (m, 1H), 1.57-1.45 (m, 1H), 0.68-0.64 (m, 2H), 0.54-0.50 (m, 2H). |
| 72 | 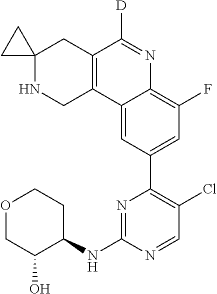 [SMILES: [2H]c1nc2c(F)cc(-c3nc(N[C@@H]4CCOC[C@H]4O)ncc3Cl)cc2c2c1CC1(CC1)NC2] | LC-MS (ESI): m/z 457.1 [M + H]⁺; ¹H NMR (500 MHz, DMSO-d₆) δ 8.48 (s, 1H), 8.22 (s, 1H), 7.86 (s, 1H), 7.59 (s, 1H), 4.95 (d, J = 5.3 Hz, 1H), 4.29 (s, 2H), 3.89-3.79 (m, 3H), 3.56- 3.49 (m, 1H), 3.45-3.40 (m, 1H), 3.06 (t, J = 10.3 Hz, 1H), 2.88 (s, 2H), 2.05-1.90 (m, 1H), 1.56-1.46 (m, 1H), 0.67-0.63 (m, 2H), 0.54- 0.50 (m, 2H). |
| 73a | 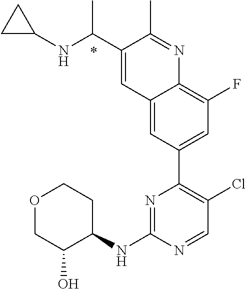 [SMILES: Cc1nc2c(F)cc(-c3nc(N[C@@H]4CCOC[C@H]4O)ncc3Cl)cc2cc1C(C)NC1CC1] Isomer 1 (earlier eluting isomer) | LC-MS (ESI): m/z 472.2 [M + H]⁺; ¹H NMR ¹H NMR (500 MHz, DMSO-d₆) δ 8.51 (s, 1H), 8.47 (s, 1H), 8.20 (s, 1H), 7.81 (d, J = 11.9 Hz, 1H), 7.56 (brs, 1H), 4.94 (d, J = 5.5 Hz, 1H), 4.22- 4.16 (m, 1H), 3.87-3.77 (m, 3H), 3.52-3.48 (m, 1H), 3.36-3.33 (m, 1H), 3.05 (t, J = 10.4 Hz, 1H), 2.78 (s, 3H), 2.00-1.89 (m, 2H), 1.55-1.45 (m, 1H), 1.35 (d, J = 6.6 Hz, 3H), 0.33-0.22 (m, 4H). de: 98.5 %; Retention time: 0.714 min; instrument: Agilent- 1260; column: CHIRALPAK IA-3, 4.6 * 50 mm, 3.0 um; mobile phase: A for MtBE (0.1% DEA) and B for MeOH; gradient: B 15%; flow rate: 1.67 mL/min; high pressure: 110 bar; column temperature: 25° C.; wavelength: 254 nm. |
| 73b | 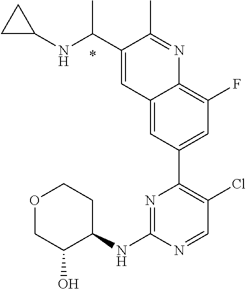 [SMILES: Cc1nc2c(F)cc(-c3nc(N[C@@H]4CCOC[C@H]4O)ncc3Cl)cc2cc1C(C)NC1CC1] Isomer 2 (later eluting isomer) | LC-MS (ESI): m/z 472.2 [M + H]⁺; ¹H NMR (500 MHz, DMSO-d₆) δ 8.52 (s, 1H), 8.48 (s, 1H), 8.21 (s, 1H), 7.82 (d, J = 11.4 Hz, 1H), 7.57 (brs, 1H), 4.95 (d, J = 5.4 Hz, 1H), 4.23-4.16 (m, 1H), 3.88- 3.78 (m, 3H), 3.52-3.46 (m, 1H), 3.40-3.37 (m, 1H), 3.06 (t, J = 10.2 Hz, 1H), 2.79 (s, 3H), 2.00-1.92 (m, 2H), 1.55-1.46 (m, 1H), 1.36 (d, J = 6.6 Hz, 3H), 0.32-0.23 (m, 4H). de: 100%; Retention time: 0.972 min; instrument: Agilent- 1260; column: CHIRALPAK IA-3, 4.6 * 50 mm, 3.0 um; mobile phase: A for MtBE (0.1% DEA) and B for MeOH; gradient: B 15%; flow rate: 1.67 mL/min; high pressure: 110 bar; column temperature: 25° C.; wavelength: 254 nm. |
| 74a | 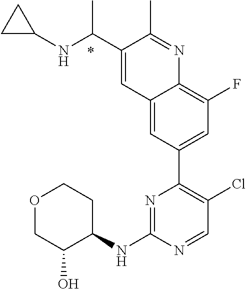 Isomer 1 (earlier eluting isomer) | LC-MS (ESI): m/z 456.2 [M + H]⁺; ¹H NMR (500 MHz, DMSO-d₆) δ 8.55 (s, 1H), 8.50 (d, J = 3.6 Hz, 1H), 8.41 (s, 1H), 8.06 (d, J = 12.1 Hz, 1H), 7.28 (d, J = 8.0 Hz, 1H), 4.93 (d, J = 5.4 Hz, 1H), 4.22-4.16 (m, 1H), 3.88-3.80 (m, 3H), 3.53- 3.47 (m, 1H), 3.42-3.36 (m, 1H), 3.12-3.06 (m, 1H), 2.78 (s, 3H), 2.03-1.93 (m, 2H), 1.54-1.45 (m, 1H), 1.36 (d, J = 6.5 Hz, 3H), 0.33-0.23 (m, 4H). de: 100%; Retention time: 2.369 min; instrument: Agilent- 1260; column: CHIRALPAK AD-3, 4.6 * 50 mm, 3.0 um; mobile phase: A for Hex (0.1% DEA) and B for EtOH: ACN = 5:1; gradient: B 50%; flow rate: 1.67 mL/min; high pressure: 110 bar; column temperature: 25° C.; wavelength: 254 nm. |
| 74b | 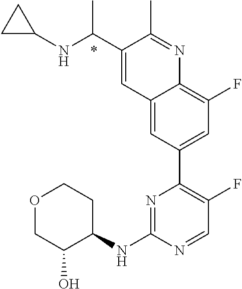 [SMILES: Cc1nc2c(F)cc(-c3nc(N[C@@H]4CCOC[C@H]4O)ncc3F)cc2cc1C(C)NC1CC1] Isomer 2 (later eluting isomer) | LC-MS (ESI): m/z 456.2 [M + H]⁺; ¹H NMR (500 MHz, DMSO-d₆) δ 8.55 (s, 1H), 8.50 (d, J = 3.7 Hz, 1H), 8.41 (s, 1H), 8.06 (d, J = 12.3 Hz, 1H), 7.28 (d, J = 7.9 Hz, 1H), 4.93 (d, J = 5.4 Hz, 1H), 4.22-4.17 (m, 1H), 3.88-3.79 (m, 3H), 3.54- 3.48 (m, 1H), 3.38-3.34 (m, 1H), 3.13-3.06 (m, 1H), 2.78 (s, 3H), 2.03-1.95 (m, 2H), 1.55-1.45 (m, 1H), 1.35 (d, J = 6.6 Hz, 3H), 0.32-0.23 (m, 4H). de: 97.9%; Retention time: 4.046 min; instrument: Agilent- 1260; column: CHIRALPAK AD-3, 4.6 * 50 mm, 3.0 um; mobile phase: A for Hex (0.1% DEA) and B for EtOH: ACN = 5:1; gradient: B 50%; flow rate: 1.67 mL/min; high pressure: 110 bar; column temperature: 25° C.; wavelength: 254 nm. |
| 75 | 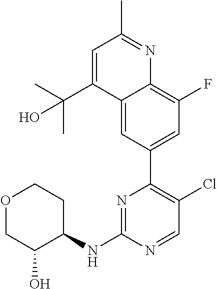 [SMILES: Cc1cc(C(C)(C)O)c2cc(-c3nc(N[C@@H]4CCOC[C@H]4O)ncc3Cl)cc(F)c2n1] | LC-MS (ESI): m/z 447.1 [M + H]⁺; ¹H NMR (500 MHz, DMSO-d₆) δ 9.12 (s, 1H), 8.46 (s, 1H), 7.92- 7.78 (m, 1H), 7.59 (s, 1H), 7.53 (s, 1H), 5.62 (s, 1H), 4.93 (d, J = 5.4 Hz, 1H), 3.88-3.77 (m, 3H), 3.55-3.45 (m, 1H), 3.31-3.28 (m, 1H), 3.03 (t, J = 10.4 Hz, 1H), 2.71 (s, 3H), 2.07-1.91 (m, 1H), 1.71 (s, 6H), 1.54-1.46 (m, 1H). |
| 76 | 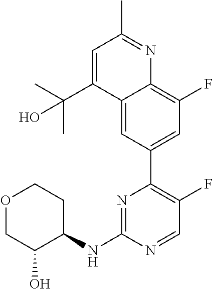 [SMILES: Cc1cc(C(C)(C)O)c2cc(-c3nc(N[C@@H]4CCOC[C@H]4O)ncc3F)cc(F)c2n1] | LC-MS (ESI): m/z 431.1; ¹H NMR (500 MHz, DMSO-d₆) δ 9.38 (s, 1H), 8.49 (d, J = 3.8 Hz, 1H), 8.03 (d, J = 11.5 Hz, 1H), 7.60 (s, 1H), 7.25 (d, J = 7.8 Hz, 1H), 5.65 (s, 1H), 4.94 (d, J = 5.4 Hz, 1H), 3.88-3.79 (m, 3H), 3.55-3.50 (m, 1H), 3.42-3.36 (m, 1H), 3.07 (t, J = 10.4 Hz, 1H), 2.71 (s, 3H), 2.08-1.98 (m, 1H), 1.72 (s, 6H), 1.54- 1.46 (m, 1H). |
| 77a | 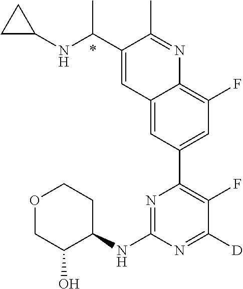 [SMILES: [2H]c1nc(N[C@@H]2CCOC[C@H]2O)nc(-c2cc(F)c3nc(C)c(C(C)NC4CC4)cc3c2)c1F] Isomer 1 (earlier eluting isomer) | LC-MS (ESI): m/z 457.2; ¹H NMR (500 MHz, DMSO-d₆) δ 8.55 (s, 1H), 8.41 (s, 1H), 8.06 (d, J = 12.1 Hz, 1H), 7.28 (d, J = 7.9 Hz, 1H), 4.93 (d, J = 5.4 Hz, 1H), 4.22-4.16 (m, 1H), 3.90-3.79 (m, 3H), 3.54-3.47 (m, 1H), 3.42-3.38 (m, 1H), 3.09 (t, J = 10.4 Hz, 1H), 2.78 (s, 3H), 2.04-1.93 (m, 2H), 1.55-1.45 (m, 1H), 1.36 (d, J = 6.6 Hz, 3H), 0.33-0.23 (m, 4H). de: 99.1%; Retention time: 2.41 min; instrument: Agilent- 1260; column: CHIRALPAK AD-3, 4.6 * 50 mm, 3.0 um; mobile phase: A for Hex (0.1% DEA) and B for EtOH: ACN = 5:1; gradient: B 50%; flow rate: 1.0 mL/min; high pressure: 110 bar; column temperature: 25° C.; wavelength: 254 nm. |
| 77b | 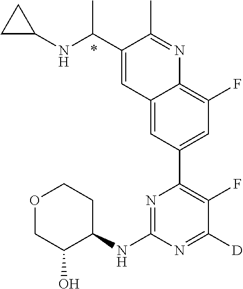 [SMILES: [2H]c1nc(N[C@@H]2CCOC[C@H]2O)nc(-c2cc(F)c3nc(C)c(C(C)NC4CC4)cc3c2)c1F] Isomer 2 (later eluting isomer) | LC-MS (ESI): m/z 457.2; ¹H NMR (500 MHz, DMSO-d₆) δ 8.55 (s, 1H), 8.41 (s, 1H), 8.06 (d, J = 12.1 Hz, 1H), 7.28 (d, J = 7.9 Hz, 1H), 4.93 (d, J = 5.4 Hz, 1H), 4.22-4.17 (m, 1H), 3.88-3.79 (m, 3H), 3.54-3.48 (m, 1H), 3.42-3.37 (m, 1H), 3.09 (t, J = 10.3 Hz, 1H), 2.78 (s, 3H), 2.04-1.93 (m, 2H), 1.54-1.45 (m, 1H), 1.35 (d, J = 6.5 Hz, 3H), 0.33-0.23 (m, 4H). de: 96.6%; Retention time: 4.589 min; instrument: Agilent- 1260; column: CHIRALPAK AD-3, 4.6 * 50 mm, 3.0 um; mobile phase: A for Hex (0.1% DEA) and B for EtOH: ACN = 5:1; gradient: B 50%; flow rate: 1.0 mL/min; high pressure: 110 bar; column temperature: 25° C.; wavelength: 254 nm. |
| 78 | 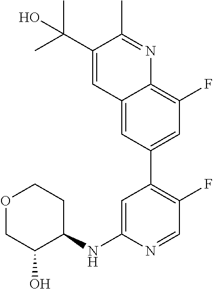 [SMILES: Cc1nc2c(F)cc(-c3cc(N[C@@H]4CCOC[C@H]4O)ncc3F)cc2cc1C(C)(C)O] | LC-MS (ESI): m/z 430.2; ¹H NMR (400 MHz, DMSO-d₆) δ 8.45 (d, J = 1.8 Hz, 1H), 8.06 (d, J = 2.6 Hz, 1H), 8.01 (s, 1H), 7.67 (d, J = 11.6 Hz, 1H), 6.76 (d, J = 5.4 Hz, 1H), 6.56 (d, J = 7.3 Hz, 1H), 5.34 (s, 1H), 5.07 (d, J = 5.2 Hz, 1H), 3.84- 3.67 (m, 4H), 3.44-3.39 (m, 1H), 3.12-3.05 (m, 1H), 2.95 (s, 3H), 2.08-1.98 (m, 1H), 1.64 (s, 6H), 1.47-1.35 (m, 1H). |
| 79a | 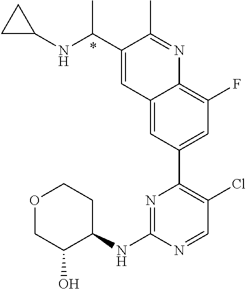 Isomer 1 (earlier eluting isomer) | LC-MS (ESI): m/z 458.2 [M + H]⁺; ¹H NMR (500 MHz, DMSO-d₆) δ 9.07 (s, 1H), 8.52-8.43 (m, 2H), 8.25 (s, 1H), 7.86 (d, J = 11.4 Hz, 1H), 7.57 (brs, 1H), 4.94 (d, J = 5.4 Hz, 1H), 4.15-4.01 (m, 1H), 3.89-3.77 (m, 3H), 3.54-3.47 (m, 1H), 3.39- 3.35 (m, 1H), 3.08-3.02 (m, 1H), 2.03-1.86 (m, 2H), 1.55-1.46 (m, 1H), 1.42 (brs, 3H), 0.39- 0.13 (m, 4H). de: 100%; Retention time: 1.396 min; instrument: Agilent- 1260; column: Lux 3 um Cellulose-2, 4.6 * 50 mm, 3.0 um; mobile phase: A for Hex (0.1% DEA) and B for EtOH; gradient: B 40%; flow rate: 1.67 mL/min; high pressure: 110 bar; column temperature: 25° C.; wavelength: 254 nm. |
| 79b | 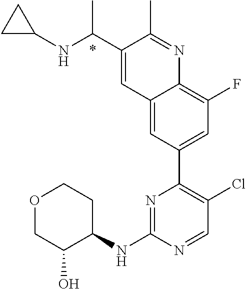 Isomer 2 (later eluting isomer) | LC-MS (ESI): m/z 458.2 [M + H]⁺; ¹H NMR (500 MHz, DMSO-d₆) δ 9.07 (s, 1H), 8.52-8.44 (m, 2H), 8.25 (s, 1H), 7.86 (d, J = 11.4 Hz, 1H), 7.57 (brs, 1H), 4.94 (d, J = 5.4 Hz, 1H), 4.16-4.02 (m, 1H), 3.88-3.77 (m, 3H), 3.54-3.46 (m, 1H), 3.39- 3.35 (m, 1H), 3.10-3.02 (m, 1H), 2.02-1.86 (m, 2H), 1.42 (brs, 3H), 1.55-1.46 (m, 1H), 0.40- 0.10 (m, 4H). de: 100%; Retention time: 1.761 min; instrument: Agilent-1260; column: Lux 3 um Cellulose-2, 4.6 * 50 mm, 3.0 um; mobile phase: A for Hex (0.1% DEA) and B for EtOH; gradient: B 40%; flow rate: 1.67 mL/min; high pressure: 110 bar; column temperature: 25° C.; wavelength: 254 nm. |
| 80 | 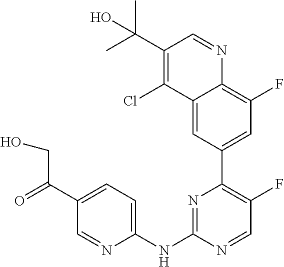 [SMILES: CC(C)(O)c1cnc2c(F)cc(-c3nc(Nc4ccc(C(=O)CO)cn4)ncc3F)cc2c1Cl] | LC-MS (ESI): m/z 486.1 [M + H]⁺; ¹H NMR (500 MHz, DMSO-d₆) δ 10.87 (s, 1H), 9.47 (s, 1H), 8.97 (s, 1H), 8.93-8.88 (m, 2H), 8.38 (d, J = 8.9 Hz, 1H), 8.34-8.27 (m, 2H), 5.87 (s, 1H), 5.16 (t, J = 5.9 Hz, 1H), 4.79 (d, J = 5.9 Hz, 2H), 1.77 (s, 6H). |
| 81a | 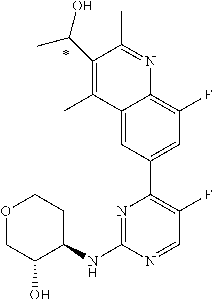 [SMILES: Cc1nc2c(F)cc(-c3nc(N[C@@H]4CCOC[C@H]4O)ncc3F)cc2c(C)c1C(C)O] Isomer 1 (earlier eluting isomer) | LC-MS (ESI): m/z 431.2 [M + H]⁺; ¹H NMR (500 MHz, DMSO-d₆) δ 8.60 (s, 1H), 8.51 (d, J = 3.6 Hz, 1H), 8.04 (d, J = 11.7 Hz, 1H), 7.31 (d, J = 7.8 Hz, 1H), 5.48-5.45 (m, 1H), 5.44-5.39 (m, 1H), 4.95 (d, J = 5.4 Hz, 1H), 3.90-3.80 (m, 3H), 3.58-3.49 (m, 1H), 3.44-3.35 (m, 1H), 3.09 (t, J = 10.4 Hz, 1H), 2.86 (s, 3H), 2.81 (s, 3H), 2.08-1.96 (m, 1H), 1.60-1.43 (m, 4H). de: 100%; Retention time: 1.204 min; instrument: CHIRALPAK AD-3, 4.6 * 50 mm, 3.0 um mobile phase: A for Hex (0.1% DEA) and B for IPA; gradient: B 30%; flow rate: 1.67 mL/min; high pressure: 110 bar; column temperature: 25° C.; wavelength: 254 nm. |
| 81b | 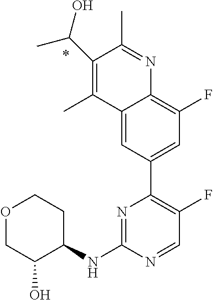 [SMILES: Cc1nc2c(F)cc(-c3nc(N[C@@H]4CCOC[C@H]4O)ncc3F)cc2c(C)c1C(C)O] Isomer 2 (later eluting isomer) | LC-MS (ESI): m/z 431.2 [M + H]⁺; ¹H NMR (500 MHz, DMSO-d₆) δ 8.60 (s, 1H), 8.51 (d, J = 3.6 Hz, 1H), 8.04 (d, J = 11.7 Hz, 1H), 7.31 (d, J = 7.8 Hz, 1H), 5.48-5.45 (m, 1H), 5.44-5.38 (m, 1H), 4.95 (d, J = 5.4 Hz, 1H), 3.90-3.80 (m, 3H), 3.58-3.49 (m, 1H), 3.43-3.35 (m, 1H), 3.09 (t, J = 10.4 Hz, 1H), 2.86 (s, 3H), 2.81 (s, 3H), 2.10-1.94 (m, 1H), 1.55-1.47 (m, 4H). de: 97.2%; Retention time: 1.594 min; instrument: CHIRALPAK AD-3, 4.6 * 50 mm, 3.0 um mobile phase: A for Hex (0.1% DEA) and B for IPA; gradient: B 30%; flow rate: 1.67 mL/min; high pressure: 110 bar; column temperature: 25° C.; wavelength: 254 nm. |
| 82a | 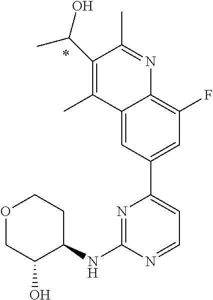 [SMILES: Cc1nc2c(F)cc(-c3ccnc(N[C@@H]4CCOC[C@H]4O)n3)cc2c(C)c1C(C)O] Isomer 1 (earlier eluting isomer) | LC-MS (ESI): m/z 413.2 [M + H]⁺; ¹H NMR (500 MHz, DMSO-d₆) δ 8.68 (s, 1H), 8.43 (d, J = 5.1 Hz, 1H), 8.18 (d, J = 11.8 Hz, 1H), 7.41 (d, J = 5.2 Hz, 1H), 7.23 (d, J = 7.7 Hz, 1H), 5.47- 5.38 (m, 2H), 5.00 (d, J = 5.3 Hz, 1H), 4.00- 3.78 (m, 3H), 3.63-3.49 (m, 1H), 3.47-3.35 (m, 1H), 3.20-3.02 (m, 1H), 2.90 (s, 3H), 2.80 (s, 3H), 2.20-1.92 (m, 1H), 1.62-1.43 (m, 4H). de: 100%; Retention time: 0.813 min; instrument: CHIRALPAK IH-3, 4.6 * 50 mm, 3.0 um mobile phase: A for Hex (0.1% DEA) and B for EtOH; gradient: B 30%; flow rate: 1.67 mL/min; high pressure: 110 bar; column temperature: 25° C.; wavelength: 254 nm. |
| 82b | 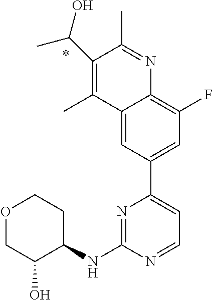 [SMILES: Cc1nc2c(F)cc(-c3ccnc(N[C@@H]4CCOC[C@H]4O)n3)cc2c(C)c1C(C)O] Isomer 2 (later eluting isomer) | LC-MS (ESI): m/z 413.2 [M + H]⁺; ¹H NMR (500 MHz, DMSO-d₆) δ 8.68 (s, 1H), 8.43 (d, J = 5.1 Hz, 1H), 8.18 (d, J = 11.8 Hz, 1H), 7.41 (d, J = 5.2 Hz, 1H), 7.23 (d, J = 7.7 Hz, 1H), 5.46- 5.40 (m, 2H), 5.00 (d, J = 5.3 Hz, 1H), 4.00- 3.78 (m, 3H), 3.63-3.49 (m, 1H), 3.46-3.35 (m, 1H), 3.20-3.01(m, 1H), 2.90 (s, 3H), 2.80 (s, 3H), 2.15-1.95 (s, 1H), 1.60-1.45 (m, 4H). de: 100%; Retention time: 1.369 min; instrument: CHIRALPAK IH-3, 4.6 * 50 mm, 3.0 um mobile phase: A for Hex (0.1% DEA) and B for EtOH; gradient: B 30%; flow rate: 1.67 mL/min; high pressure: 110 bar; column temperature: 25° C.; wavelength: 254 nm. |
| 83 | 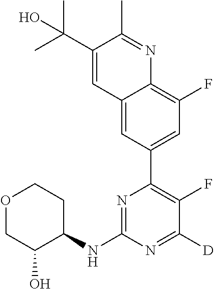 [SMILES: [2H]c1nc(N[C@@H]2CCOC[C@H]2O)nc(-c2cc(F)c3nc(C)c(C(C)(C)O)cc3c2)c1F] | LC-MS (ESI): m/z 432.2 [M + H]⁺; ¹H NMR (500 MHz, DMSO-d₆) δ 8.50 (s, 1H), 8.45 (s, 1H), 8.06 (dd, J = 12.2, 1.8 Hz, 1H), 7.26 (d, J = 7.9 Hz, 1H), 5.35 (s, 1H), 4.95 (d, J = 5.4 Hz, 1H), 3.96-3.74 (m, 3H), 3.57-3.47 (m, 1H), 3.44-3.40 (m, 1H), 3.10 (t, J = 10.3 Hz, 1H), 2.97 (s, 3H), 2.06-1.95 (m, 1H), 1.65 (s, 6H), 1.56-1.45 (m, 1H). |
| 84a | 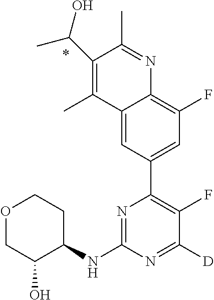 [SMILES: [2H]c1nc(N[C@@H]2CCOC[C@H]2O)nc(-c2cc(F)c3nc(C)c(C(C)O)c(C)c3c2)c1F] Isomer 1 (earlier eluting isomer) | LC-MS (ESI): m/z 432.2 [M + H]⁺; ¹H NMR (500 MHz, DMSO-d₆) δ 8.60 (s, 1H), 8.04 (d, J = 11.7 Hz, 1H), 7.31 (d, J = 7.9 Hz, 1H), 5.53- 5.37 (m, 2H), 4.95 (d, J = 5.4 Hz, 1H), 3.92- 3.77 (m, 3H), 3.60-3.48 (m, 1H), 3.45-3.35 (m, 1H), 3.09 (t, J = 10.3 Hz, 1H), 2.86 (s, 3H), 2.81 (s, 3H), 2.09-1.94 (m, 1H), 1.59-1.45 (m, 4H). de: 100%; Retention time: 1.067 min; instrument: CHIRALPAK IH-3, 4.6 * 50 mm, 3.0 um mobile phase: A for Hex (0.1% DEA) and B for EtOH; gradient: B 50%; flow rate: 1 mL/min; high pressure: 110 bar; column temperature: 25° C.; wavelength: 254 nm. |
| 84b | 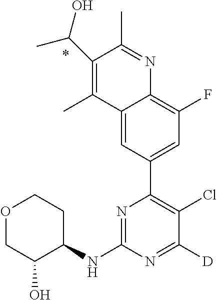 Isomer 2 (later eluting isomer) | LC-MS (ESI): m/z 432.2 [M + H]⁺; ¹H NMR (500 MHz, DMSO-d₆) δ 8.60 (s, 1H), 8.04 (d, J = 11.7 Hz, 1H), 7.31 (d, J = 7.9 Hz, 1H), 5.52- 5.39 (m, 2H), 4.95 (d, J = 5.4 Hz, 1H), 3.92- 3.78 (m, 3H), 3.58-3.49 (m, 1H), 3.45-3.35 (m, 1H), 3.09 (t, J = 10.3 Hz, 1H), 2.86 (s, 3H), 2.81 (s, 3H), 2.08-1.97 (m, 1H), 1.57-1.45 (m, 4H). de: 100%; Retention time: 2.14 min; instrument: CHIRALPAK IH-3, 4.6 * 50 mm, 3.0 um mobile phase: A for Hex (0.1% DEA) and B for EtOH; gradient: B 50%; flow rate: 1 mL/min; high pressure: 110 bar; column temperature: 25° C.; wavelength: 254 nm. |
| 85a | 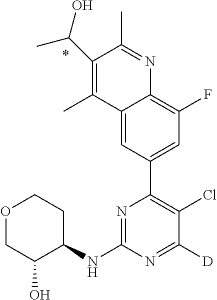 [SMILES: [2H]c1nc(N[C@@H]2CCOC[C@H]2O)nc(-c2cc(F)c3nc(C)c(C(C)O)c(C)c3c2)c1Cl] Isomer 1 (earlier eluting isomer) | LC-MS (ESI): m/z 448.2 [M + H]⁺; ¹H NMR (500 MHz, DMSO-d₆) δ 8.45-8.30 (m, 1H), 7.83 (s, 1H), 7.58 (s, 1H), 5.47-5.38 (m, 2H), 4.95 (d, J = 5.3 Hz, 1H), 3.92-3.76 (m, 3H), 3.56-3.46 (m, 1H), 3.40-3.35 (m, 1H), 3.06 (t, J = 10.4 Hz, 1H), 2.84 (s, 3H), 2.81 (s, 3H), 2.04- 1.90 (m, 1H), 1.57-1.44 (m, 4H). de: 100%; Retention time: 1.022 min; instrument: CHIRALPAK IH-3, 4.6 * 50 mm, 3.0 um mobile phase: A for Hex (0.1% DEA) and B for EtOH; gradient: B 50%; flow rate: 1.67 mL/min; high pressure: 110 bar; column temperature: 25° C.; wavelength: 254 nm. |
| 85b | 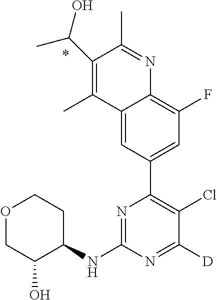 [SMILES: [2H]c1nc(N[C@@H]2CCOC[C@H]2O)nc(-c2cc(F)c3nc(C)c(C(C)O)c(C)c3c2)c1Cl] Isomer 2 (later eluting isomer) | LC-MS (ESI): m/z 448.2 [M + H]⁺; ¹H NMR (500 MHz, DMSO-d₆) δ 8.44-8.30 (m, 1H), 7.83 (s, 1H), 7.58 (s, 1H), 5.47-5.38 (m, 2H), 4.95 (d, J = 5.3 Hz, 1H), 3.92-3.76 (m, 3H), 3.56-3.46 (m, 1H), 3.40-3.35 (m, 1H), 3.06 (t, J = 10.4 Hz, 1H), 2.84 (s, 3H), 2.81 (s, 3H), 2.04- 1.90 (m, 1H), 1.56-1.43 (m, 4H). de: 98.4%; Retention time: 4.283 min; instrument: CHIRALPAK IH-3, 4.6 * 50 mm, 3.0 um mobile phase: A for Hex (0.1% DEA) and B for EtOH; gradient: B 50%; flow rate: 1.67 mL/min; high pressure: 110 bar; column temperature: 25° C.; wavelength: 254 nm. |
| 87 | 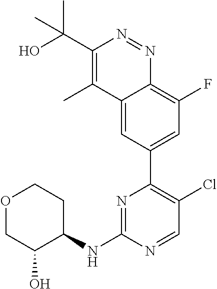 [SMILES: Cc1c(C(C)(C)O)nnc2c(F)cc(-c3nc(N[C@@H]4CCOC[C@H]4O)ncc3Cl)cc12] | LC-MS (ESI): m/z 448.2 [M + H]⁺; ¹H NMR (400 MHz, DMSO-d₆) δ 8.55 (s, 1H), 8.52-8.42 (m, 1H), 8.02 (d, J = 10.9 Hz, 1H), 7.70 (s, 1H), 5.61 (s, 1H), 4.97 (d, J = 5.4 Hz, 1H), 3.92-3.77 (m, 3H), 3.58-3.48 (m, 1H), 3.42-3.38 (m, 1H), 3.13-3.03 (m, 4H), 2.05-1.90 (m, 1H), 1.81 (s, 6H), 1.60-1.47 (m, 1H). |
| 88a | 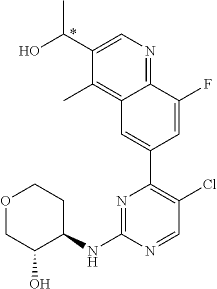 [SMILES: Cc1c(C(C)O)cnc2c(F)cc(-c3nc(N[C@@H]4CCOC[C@H]4O)ncc3Cl)cc12] Isomer 1 (earlier eluting isomer) | LC-MS (ESI): m/z 433.1 [M + H]⁺; ¹H NMR (500 MHz, DMSO-d₆) δ 9.10 (s, 1H), 8.48 (s, 1H), 8.46-8.34 (m, 1H), 7.94-7.81 (m, 1H), 7.63-7.50 (m, 1H), 5.54 (d, J = 3.8 Hz, 1H), 5.31-5.24 (m, 1H), 4.93 (d, J = 5.4 Hz, 1H), 3.93-3.78 (m, 3H), 3.57-3.46 (m, 1H), 3.39- 3.33 (m, 1H), 3.09-3.02 (m, 1H), 2.68 (s, 3H), 2.06-1.90 (m, 1H), 1.58-1.48 (m, 1H), 1.44 (d, J = 6.5 Hz, 3H). de: 100%; Retention time: 4.144 min; instrument: CHIRALART Cellulose-SB, 3.0 * 50 mm, 3.0 um; mobile phase: A for CO₂ and B for MeOH (0.1% DEA); gradient: B 5% to 20% in 4 min, hold 2 min at 20%; flow rate: 2 mL/min; high pressure: 110 bar; column temperature: 35° C.; wavelength: 254 nm. |
| 88b | 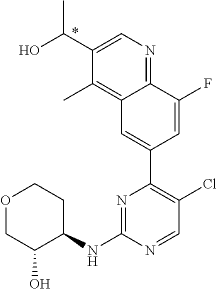 [SMILES: Cc1c(C(C)O)cnc2c(F)cc(-c3nc(N[C@@H]4CCOC[C@H]4O)ncc3Cl)cc12] Isomer 2 (later eluting isomer) | LC-MS (ESI): m/z 433.1 [M + H]⁺; ¹H NMR (500 MHz, DMSO-d₆) δ 9.10 (s, 1H), 8.48 (s, 1H), 8.45-8.34 (m, 1H), 7.98-7.81 (m, 1H), 7.70-7.46 (m, 1H), 5.54 (d, J = 3.8 Hz, 1H), 5.32-5.24 (m, 1H), 4.98-4.90 (m, 1H), 3.93- 3.78 (m, 3H), 3.57-3.46 (m, 1H), 3.39-3.33 (m, 1H), 3.10-3.02 (m, 1H), 2.68 (s, 3H), 2.06- 1.88 (m, 1H), 1.58-1.47 (m, 1H), 1.44 (d, J = 6.5 Hz, 3H). de: 100%; Retention time: 4.452 min; instrument: CHIRALART Cellulose-SB, 3.0 * 50 mm, 3.0 um; mobile phase: A for CO₂ and B for MeOH (0.1% DEA); gradient: B 5% to 20% in 4 min, hold 2 min at 20%; flow rate: 2 mL/min; high pressure: 110 bar; column temperature: 35° C.; wavelength: 254 nm. |
| 89 | 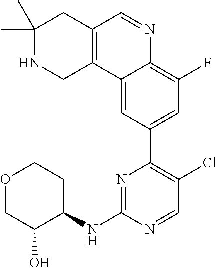 | LC-MS (ESI): m/z 501.1 [M + H]⁺; ¹H NMR (500 MHz, DMSO-d₆) δ 12.21 (s, 1H), 10.29 (s, 1H), 8.77 (s, 1H), 8.74 (s, 1H), 8.73 (d, J = 2.3 Hz, 1H), 8.33 (s, 1H), 8.22 (d, J = 8.7 Hz, 1H), 8.10 (dd, J = 8.8, 2.4 Hz, 1H), 7.98 (dd, J = 11.4, 1.7 Hz, 1H), 7.73 (s, 1H), 7.61 (brs, 1H), 4.36 (s, 2H), 2.78 (s, 2H), 1.25-1.22 (m, 1H), 1.15 (s, 6H). |
| 90a | 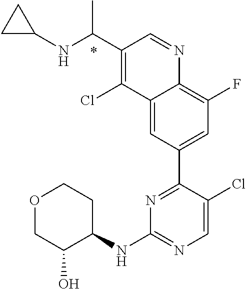 [SMILES: CC(NC1CC1)c1cnc2c(F)cc(-c3nc(N[C@@H]4CCOC[C@H]4O)ncc3Cl)cc2c1Cl] Isomer 1 (earlier eluting isomer) | LC-MS (ESI): m/z 492.1 [M + H]⁺; ¹H NMR (500 MHz, DMSO-d₆) δ 9.20 (s, 1H), 8.58-8.42 (m, 2H), 8.03 (d, J = 11.0 Hz, 1H), 7.65 (brs, 1H), 4.94 (d, J = 5.3 Hz, 1H), 4.54-4.47 (m, 1H), 3.87-3.77 (m, 3H), 3.54-3.45 (m, 1H), 3.36-3.32 (m, 1H), 3.25-3.18 (m, 1H), 3.04 (t, J = 10.4 Hz, 1H), 2.01-1.89 (m, 1H), 1.87- 1.83 (m, 1H), 1.56-1.45 (m, 1H), 1.38 (d, J = 6.7 Hz, 3H), 0.32-0.22 (m, 3H), 0.18-0.11 (m, 1H). de: 100%; Retention time: 1.350 min; instrument: Agilent- 1260; column: CHIRALPAK IH-3, 4.6 * 50 mm, 3.0 um; mobile phase: A for Hex (0.1% DEA) and B for EtOH; gradient: B 20%; flow rate: 1.67 mL/min; high pressure: 110 bar; column temperature: 25° C.; wavelength: 254 nm. |
| 90b | 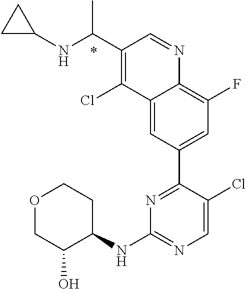 [SMILES: CC(NC1CC1)c1cnc2c(F)cc(-c3nc(N[C@@H]4CCOC[C@H]4O)ncc3Cl)cc2c1Cl] Isomer 2 (later eluting isomer) | LC-MS (ESI): m/z 492.1 [M + H]⁺; ¹H NMR (500 MHz, DMSO-d₆) δ 9.20 (s, 1H), 8.57-8.46 (m, 2H), 8.03 (d, J = 10.4 Hz, 1H), 7.65 (brs, 1H), 4.94 (d, J = 5.3 Hz, 1H), 4.52 (d, J = 7.1 Hz, 1H), 3.88-3.78 (m, 3H), 3.54-3.46 (m, 1H), 3.37-3.34 (m, 1H), 3.26-3.18 (m, 1H), 3.05 (t, J = 10.4 Hz, 1H), 2.03-1.90 (m, 1H), 1.88-1.83 (m, 1H), 1.55-1.46 (m, 1H), 1.38 (d, J = 7.0 Hz, 3H), 0.32-0.22 (m, 3H), 0.18-0.12 (m, 1H). de: 100%; Retention time: 1.920 min; instrument: Agilent- 1260; column: CHIRALPAK IH-3, 4.6 * 50 mm, 3.0 um; mobile phase: A for Hex (0.1% DEA) and B for EtOH; gradient: B 20%; flow rate: 1.67 mL/min; high pressure: 110 bar; column temperature: 25° C.; wavelength: 254 nm. |
| 91a | 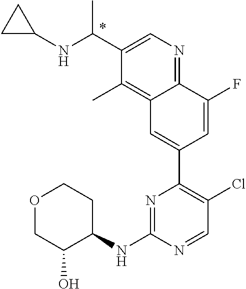 [SMILES: Cc1c(C(C)NC2CC2)cnc2c(F)cc(-c3nc(N[C@@H]4CCOC[C@H]4O)ncc3Cl)cc12] Isomer 1 (earlier eluting isomer) | LC-MS (ESI): m/z 472.2 [M + H]⁺; ¹H NMR (500 MHz, DMSO-d₆) δ 9.11 (s, 1H), 8.48 (s, 1H), 8.39 (brs, 1H), 7.91-7.82 (m, 1H), 7.59 (s, 1H), 4.94 (d, J = 5.4 Hz, 1H), 4.39 (q, J = 6.5 Hz, 1H), 3.94-3.75 (m, 3H), 3.55-3.47 (m, 1H), 3.40-3.35 (m, 1H), 3.09-2.90 (m, 2H), 2.73 (s, 3H), 2.04-1.91 (m, 1H), 1.90-1.85 (m, 1H), 1.56-1.44 (m, 1H), 1.36 (d, J = 6.7 Hz, 3H), 0.32-0.22 (m, 3H), 0.18-0.12 (m, 1H). de: 96.6%; Retention time: 1.710 min; instrument: Agilent- 1260; column: CHIRAL ART Amylose-C, 4.6 * 50 mm, 3.0 um; mobile phase: A for Hex (0.1% DEA) and B for EtOH; gradient: B 50%; flow rate: 1.67 mL/min; high pressure: 110 bar; column temperature: 25° C.; wavelength: 254 nm. |
| 91b | 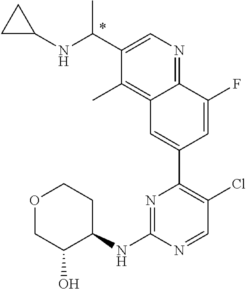 [SMILES: Cc1c(C(C)NC2CC2)cnc2c(F)cc(-c3nc(N[C@@H]4CCOC[C@H]4O)ncc3Cl)cc12] Isomer 2 (later eluting isomer) | LC-MS (ESI): m/z 472.2 [M + H]⁺; ¹H NMR (500 MHz, DMSO-d₆) δ 9.11 (s, 1H), 8.48 (s, 1H), 8.40 (brs, 1H), 7.91-7.82 (m, 1H), 7.58 (s, 1H), 4.95 (d, J = 5.4 Hz, 1H), 4.39 (q, J = 6.7 Hz, 1H), 3.90-3.75 (m, 3H), 3.55-3.48 (m, 1H), 3.40-3.36 (m, 1H), 3.11-2.90 (m, 2H), 2.73 (s, 3H), 2.04-1.92 (m, 1H), 1.90-1.84 (m, 1H), 1.55-1.44 (m, 1H), 1.36 (d, J = 6.7 Hz, 3H), 0.32-0.22 (m, 3H), 0.18-0.12 (m, 1H). de: 97.8%; Retention time: 3.908 min; instrument: Agilent- 1260; column: CHIRAL ART Amylose-C, 4.6 * 50 mm, 3.0 um; mobile phase: A for Hex (0.1% DEA) and B for EtOH; gradient: B 50%; flow rate: 1.67 mL/min; high pressure: 110 bar; column temperature: 25° C.; wavelength: 254 nm. |
| 92 | 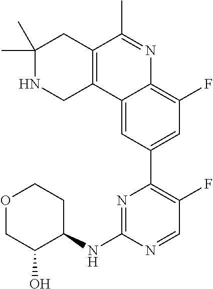 [SMILES: Cc1nc2c(F)cc(-c3nc(N[C@@H]4CCOC[C@H]4O)ncc3F)cc2c2c1CC(C)(C)NC2] | LC-MS (ESI): m/z 456.2 [M + H]⁺; ¹H NMR (500 MHz, DMSO-d₆) δ 8.50 (d, J = 3.7 Hz, 1H), 8.37 (s, 1H), 8.04 (d, J = 12.0 Hz, 1H), 7.30 (d, J = 7.7 Hz, 1H), 4.43 (s, 2H), 3.87-3.79 (m, 3H), 3.55-3.50 (m, 1H), 3.39 (t, J = 11.3 Hz, 1H), 3.10 (t, J = 9.8 Hz, 1H), 2.72 (s, 2H), 2.63 (s, 3H), 2.55 (s, 1H), 2.06-1.96 (m, 1H), 1.55- 1.46 (m, 1H), 1.22 (s, 6H). |
| 93a | 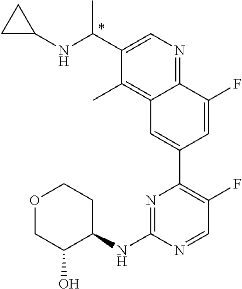 [SMILES: Cc1c(C(C)NC2CC2)cnc2c(F)cc(-c3nc(N[C@@H]4CCOC[C@H]4O)ncc3F)cc12] Isomer 1 (earlier eluting isomer) | LC-MS (ESI): m/z 456.2 [M + H]⁺; ¹H NMR (500 MHz, DMSO-d₆) δ 9.13 (s, 1H), 8.63 (s, 1H), 8.53 (s, 1H), 8.08 (d, J = 11.3 Hz, 1H), 7.34 (d, J = 6.6 Hz, 1H), 4.97 (d, J = 5.0 Hz, 1H), 4.43-4.36 (m, 1H), 3.90-3.79 (m, 3H), 3.58- 3.49 (m, 1H), 3.44-3.38 (m, 1H), 3.14-2.93 (m, 2H), 2.76 (s, 3H), 2.08-1.96 (m, 1H), 1.91- 1.83 (m, 1H), 1.56-1.45 (m, 1H), 1.37 (d, J = 5.4 Hz, 3H), 0.33-0.21 (m, 3H), 0.18-0.12 (m, 1H). de: 91.6%; Retention time: 1.467 min; instrument: Agilent- 1260; column: CHIRALPAK IH-3, 4.6 * 50 mm, 3.0 um; mobile phase: A for Hex (0.1% DEA) and B for EtOH; gradient: B 20%; flow rate: 1.67 mL/min; high pressure: 110 bar; column temperature: 25° C.; wavelength: 254 nm. |
| 93b | 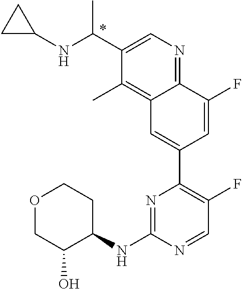 [SMILES: Cc1c(C(C)NC2CC2)cnc2c(F)cc(-c3nc(N[C@@H]4CCOC[C@H]4O)ncc3F)cc12] Isomer 2 (later eluting isomer) | LC-MS (ESI): m/z 456.2 [M + H]⁺; ¹H NMR (500 MHz, DMSO-d₆) δ 9.12 (s, 1H), 8.63 (s, 1H), 8.52 (d, J = 3.5 Hz, 1H), 8.07 (d, J = 11.7 Hz, 1H), 7.33 (d, J = 7.6 Hz, 1H), 4.96 (d, J = 5.4 Hz, 1H), 4.43-4.37 (m, 1H), 3.88-3.80 (m, 3H), 3.57-3.51 (m, 1H), 3.43-3.36 (m, 1H), 3.13-2.91 (m, 2H), 2.75 (s, 3H), 2.08-1.98 (m, 1H), 1.90-1.84 (m, 1H), 1.58-1.45 (m, 1H), 1.37 (d, J = 6.6 Hz, 3H), 0.33-0.23 (m, 3H), 0.18-0.12 (m, 1H). de: 100%; Retention time: 2.520 min; instrument: Agilent- 1260; column: CHIRALPAK IH-3, 4.6 * 50 mm, 3.0 um; mobile phase: A for Hex (0.1% DEA) and B for EtOH; gradient: B 20%; flow rate: 1.67 mL/min; high pressure: 110 bar; column temperature: 25° C.; wavelength: 254 nm. |
| 94 | 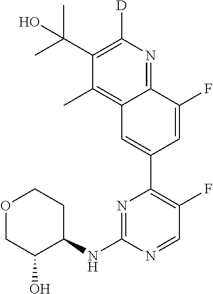 [SMILES: [2H]c1nc2c(F)cc(-c3nc(N[C@@H]4CCOC[C@H]4O)ncc3F)cc2c(C)c1C(C)(C)O] | LC-MS (ESI): m/z 432.2 [M + H]⁺; ¹H NMR (500 MHz, DMSO-d₆) δ 8.68 (s, 1H), 8.52 (d, J = 3.3 Hz, 1H), 8.08 (d, J = 11.6 Hz, 1H), 7.33 (d, J = 7.5 Hz, 1H), 5.44 (s, 1H), 4.96 (d, J = 5.3 Hz, 1H), 3.90-3.79 (m, 3H), 3.58-3.50 (m, 1H), 3.44-3.39 (m, 1H), 3.09 (t, J = 10.3 Hz, 1H), 2.99 (s, 3H), 2.07-1.98 (m, 1H), 1.69 (s, 6H), 1.57-1.47 (m, 1H). |
| 95 | 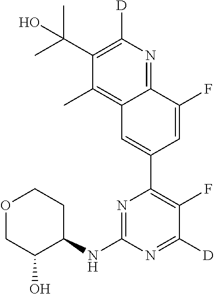 [SMILES: [2H]c1nc(N[C@@H]2CCOC[C@H]2O)nc(-c2cc(F)c3nc([2H])c(C(C)(C)O)c(C)c3c2)c1F] | LC-MS (ESI): m/z 433.2 [M + H]⁺; ¹H NMR (500 MHz, DMSO-d₆) δ 8.68 (s, 1H), 8.08 (d, J = 11.5 Hz, 1H), 7.33 (d, J = 7.4 Hz, 1H), 5.44 (s, 1H), 4.96 (d, J = 5.0 Hz, 1H), 3.89-3.80 (m, 3H), 3.57-3.50 (m, 1H), 3.42-3.37 (m, 1H), 3.09 (t, J = 10.4 Hz, 1H), 2.99 (s, 3H), 2.07- 1.97 (m, 1H), 1.69 (s, 6H), 1.57-1.46 (m, 1H). |
| 96 | 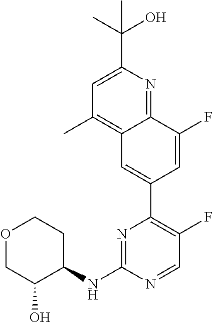 [SMILES: Cc1cc(C(C)(C)O)nc2c(F)cc(-c3nc(N[C@@H]4CCOC[C@H]4O)ncc3F)cc12] | LC-MS (ESI): m/z 431.2 [M + H]⁺; ¹H NMR (500 MHz, DMSO-d₆) δ 8.57 (s, 1H), 8.53 (d, J = 3.7 Hz, 1H), 8.13 (d, J = 11.9 Hz, 1H), 7.93 (s, 1H), 7.36 (d, J = 7.6 Hz, 1H), 5.53 (brs, 1H), 5.03 (brs, 1H), 3.90-3.79 (m, 3H), 3.59-3.51 (m, 1H), 3.43-3.40 (m, 1H), 3.10 (t, J = 10.2 Hz, 1H), 2.79 (s, 3H), 2.09-1.98 (m, 1H), 1.62- 1.46 (m, 7H). |
| 97 | 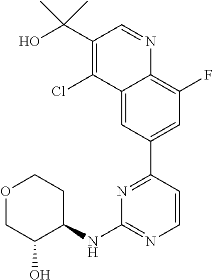 [SMILES: CC(C)(O)c1cnc2c(F)cc(-c3ccnc(N[C@@H]4CCOC[C@H]4O)n3)cc2c1Cl] | LC-MS (ESI): m/z 433.1 [M + H]⁺; ¹H NMR (500 MHz, DMSO-d₆) δ 9.39 (s, 1H), 8.93 (s, 1H), 8.46 (d, J = 5.1 Hz, 1H), 8.35 (d, J = 11.3 Hz, 1H), 7.41-7.27 (m, 2H), 5.82 (s, 1H), 4.99 (d, J = 5.2 Hz, 1H), 4.00-3.74 (m, 3H), 3.63- 3.49 (m, 1H), 3.44-3.40 (m, 1H), 3.17-2.99 (m, 1H), 2.20-1.89 (m, 1H), 1.74 (s, 6H), 1.58- 1.45 (m, 1H). |
| 98 | 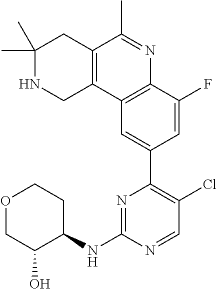 [SMILES: Cc1nc2c(F)cc(-c3nc(N[C@@H]4CCOC[C@H]4O)ncc3Cl)cc2c2c1CC(C)(C)NC2] | LC-MS (ESI): m/z 472.1 [M + H]⁺; ¹H NMR (500 MHz, DMSO-d₆) δ 8.46 (s, 1H), 8.16 (s, 1H), 7.82 (d, J = 9.3 Hz, 1H), 7.56 (s, 1H), 4.92 (brs, 1H), 4.35 (s, 2H), 3.85-3.79 (m, 3H), 3.51- 3.48 (m, 1H), 3.34 (td, J = 11.6, 1.9 Hz, 1H), 3.08-3.02 (m, 1H), 2.67 (s, 2H), 2.62 (s, 3H), 2.01-1.90 (m, 1H), 1.55-1.45 (m, 1H), 1.17 (s, 6H). |
| 99 | 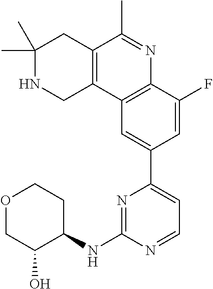 [SMILES: Cc1nc2c(F)cc(-c3ccnc(N[C@@H]4CCOC[C@H]4O)n3)cc2c2c1CC(C)(C)NC2] | LC-MS (ESI): m/z 438.2 [M + H]⁺; ¹H NMR (500 MHz, DMSO-d₆) δ 8.45 (s, 1H), 8.41 (d, J = 5.2 Hz, 1H), 8.15 (d, J = 12.0 Hz, 1H), 7.40 (d, J= 5.1 Hz, 1H), 7.21 (d, J = 7.7 Hz, 1H), 5.00 (d, J = 5.4 Hz, 1H), 4.37 (s, 2H), 4.00-3.79 (m, 3H), 3.59-3.49 (m, 1H), 3.43-3.39 (m, 1H), 3.15-3.05 (m, 1H), 2.61 (s, 2H), 2.59 (s, 3H), 2.12-1.94 (m, 1H), 1.57-1.47 (m, 1H), 1.15 (s, 6H). |
| 100 | 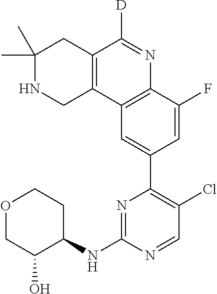 [SMILES: [2H]c1nc2c(F)cc(-c3nc(N[C@@H]4CCOC[C@H]4O)ncc3Cl)cc2c2c1CC(C)(C)NC2] | LC-MS (ESI): m/z 458.9 [M + H]⁺; ¹H NMR (500 MHz, DMSO-d₆) δ 8.48 (s, 1H), 8.19 (s, 1H), 7.86 (d, J = 10.3 Hz, 1H), 7.59 (s, 1H), 4.89 (brs, 1H), 4.36 (s, 2H), 3.90-3.76 (m, 3H), 3.55- 3.44 (m, 1H), 3.34 (t, J = 11.6 Hz, 1H), 3.05 (t, J = 10.3 Hz, 1H), 2.80 (s, 2H), 2.05-1.90 (m, 1H), 1.55-1.45 (m, 1H), 1.16 (s, 6H). |
| 101 | 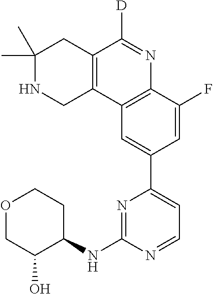 [SMILES: [2H]c1nc2c(F)cc(-c3ccnc(N[C@@H]4CCOC[C@H]4O)n3)cc2c2c1CC(C)(C)NC2] | LC-MS (ESI): m/z 425.2 [M + H]⁺; ¹H NMR (500 MHz, DMSO-d₆) δ 8.48 (s, 1H), 8.43 (d, J = 5.0 Hz, 1H), 8.21 (d, J = 12.0 Hz, 1H), 7.44 (d, J = 5.1 Hz, 1H), 7.25 (d, J = 7.5 Hz, 1H), 4.99 (s, 1H), 4.44 (s, 2H), 3.98-3.79 (m, 3H), 3.59- 3.51 (m, 1H), 3.45-3.32 (m, 1H), 3.14-3.05 (m, 1H), 2.78 (s, 2H), 2.14-1.94 (m, 1H), 1.57- 1.47 (m, 1H), 1.16 (s, 6H). |
| 102 | 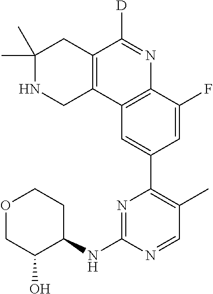 [SMILES: [2H]c1nc2c(F)cc(-c3nc(N[C@@H]4CCOC[C@H]4O)ncc3C)cc2c2c1CC(C)(C)NC2] | LC-MS (ESI): m/z 439.2 [M + H]⁺; ¹H NMR (500 MHz, DMSO-d₆) δ 8.27 (s, 1H), 7.96 (s, 1H), 7.76 (d, J = 11.5 Hz, 1H), 7.01 (d, J = 7.0 Hz, 1H), 4.97 (brs, 1H), 4.36 (s, 2H), 3.84-3.77 (m, 3H), 3.51-3.46 (m, 1H), 3.34 (t, J = 11.5 Hz, 1H), 3.05 (t, J = 10.3 Hz, 1H), 2.78 (s, 2H), 2.22 (s, 3H), 2.02-1.96 (m, 1H), 1.52-1.43 (m, 1H), 1.16 (s, 6H). |
| 103 | 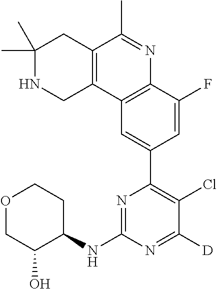 [SMILES: [2H]c1nc(N[C@@H]2CCOC[C@H]2O)nc(-c2cc(F)c3nc(C)c4c(c3c2)CNC(C)(C)C4)c1Cl] | LC-MS (ESI): m/z 473.2 [M + H]⁺; ¹H NMR (500 MHz, DMSO-d₆) δ 8.16 (s, 1H), 7.82 (d, J = 5.6 Hz, 1H), 7.56 (s, 1H), 4.94 (brs, 1H), 4.33 (s, 2H), 3.87-3.77 (m, 3H), 3.52-3.46 (m, 1H), 3.34 (t, J = 11.3 Hz, 1H), 3.05 (t, J = 10.3 Hz, 1H), 2.65 (s, 2H), 2.61 (s, 3H), 2.02-1.88 (m, 1H), 1.54-1.43 (m, 1H), 1.16 (s, 6H). |
| 104 | 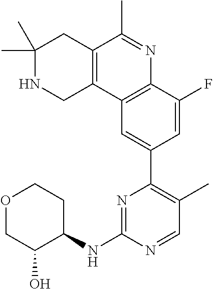 [SMILES: Cc1cnc(N[C@@H]2CCOC[C@H]2O)nc1-c1cc(F)c2nc(C)c3c(c2c1)CNC(C)(C)C3] | LC-MS (ESI): m/z 452.2 [M + H]⁺; ¹H NMR (500 MHz, DMSO-d₆) δ 8.26 (s, 1H), 7.93 (s, 1H), 7.71 (d, J = 11.6 Hz, 1H), 7.00 (d, J = 7.0 Hz, 1H), 4.99 (d, J = 4.6 Hz, 1H), 4.28 (s, 2H), 3.84-3.76 (m, 3H), 3.51-3.44 (m, 1H), 3.39- 3.35 (m, 1H), 3.05 (t, J = 10.3 Hz, 1H), 2.61 (s, 2H), 2.60 (s, 3H), 2.22 (s, 3H), 2.02-1.96 (m, 1H), 1.52-1.43 (m, 1H), 1.14 (s, 6H). |
| 105 | 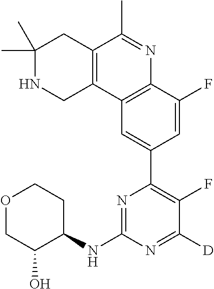 [SMILES: [2H]c1nc(N[C@@H]2CCOC[C@H]2O)nc(-c2cc(F)c3nc(C)c4c(c3c2)CNC(C)(C)C4)c1F] | LC-MS (ESI): m/z 457.2 [M + H]⁺; ¹H NMR (500 MHz, DMSO-d₆) δ 8.37 (s, 1H), 8.01 (d, J = 11.9 Hz, 1H), 7.36 (d, J = 7.5 Hz, 1H), 4.31 (s, 2H), 3.86-3.79 (m, 3H), 3.56-3.52 (m, 1H), 3.40-3.36 (m, 1H), 3.08 (t, J = 10.2 Hz, 1H), 2.63 (s, 2H), 2.61 (s, 3H), 2.06-1.97 (m, 1H), 1.53-1.44 (m, 1H), 1.15 (s, 6H). |
| 106a | 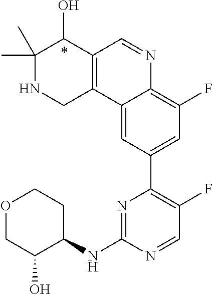 [SMILES: CC1(C)NCc2c(cnc3c(F)cc(-c4nc(N[C@@H]5CCOC[C@H]5O)ncc4F)cc23)C1O] Isomer 1 (earlier eluting isomer) | LC-MS (ESI): m/z 458.2 [M + H]⁺; ¹H NMR (500 MHz, DMSO-d₆) δ 9.04 (s, 1H), 8.54 (d, J = 3.1 Hz, 1H), 8.41 (s, 1H), 8.22-8.03 (m, 1H), 7.35 (d, J = 8.2 Hz, 1H), 4.96 (d, J = 4.7 Hz, 1H), 4.79-4.12 (m, 2H), 3.91-3.78 (m, 3H), 3.57-3.47 (m, 1H), 3.46-3.40 (m, 1H), 3.29- 3.20 (m, 1H), 3.14-3.04 (m, 1H), 2.03-1.98 (m, 1H), 1.52-1.47 (m, 1H), 1.32-1.14 (m, 6H). de: 100%; Retention time: 2.277 min; Instrument: Agilent- 1260, Column: CHIRALPAK AD-3, 4.6 * 50 mm, 3.0 um, Mobile phase: A for Hex (0.1% DEA) and B for IPA, Gradient: B 50%, Flow rate: 1 mL/min, High pressure: 110 bar, Column temperature: 25° C., Wavelength: 254 nm. |
| 106b | 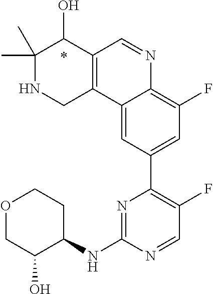 [SMILES: CC1(C)NCc2c(cnc3c(F)cc(-c4nc(N[C@@H]5CCOC[C@H]5O)ncc4F)cc23)C1O] Isomer 2 (later eluting isomer) | LC-MS (ESI): m/z 458.2 [M + H]⁺; ¹H NMR (500 MHz, DMSO-d₆) δ 9.04 (s, 1H), 8.54 (d, J = 3.1 Hz, 1H), 8.41 (s, 1H), 8.17-8.08 (m, 1H), 7.35 (d, J = 7.8 Hz, 1H), 4.95 (d, J = 5.5 Hz, 1H), 4.50 (s, 2H), 3.92-3.78 (m, 3H), 3.58- 3.48 (m, 1H), 3.42-3.36 (m, 1H), 3.33-3.27 (m, 1H), 3.09 (t, J = 9.9 Hz, 1H), 2.04-1.99 (m, 1H), 1.53-1.46 (m, 1H), 1.27-1.21 (m, 6H). de: 99.6%; Retention time: 3.012 min; Instrument: Agilent- 1260, Column: CHIRALPAK AD-3, 4.6 * 50 mm, 3.0 um, Mobile phase: A for Hex (0.1% DEA) and B for IPA, Gradient: B 50%, Flow rate: 1 mL/min, High pressure: 110 bar, Column temperature: 25° C., Wavelength: 254 nm. |
| 107 | 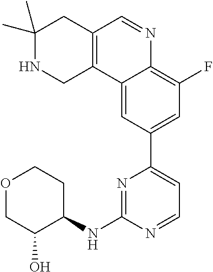 [SMILES: CC1(C)Cc2cnc3c(F)cc(-c4ccnc(N[C@@H]5CCOC[C@H]5O)n4)cc3c2CN1] | LC-MS (ESI): m/z 424.2 [M + H]⁺; ¹H NMR (500 MHz, DMSO-d₆) δ 8.69 (s, 1H), 8.48 (d, J = 1.5 Hz, 1H), 8.43 (d, J = 5.2 Hz, 1H), 8.21 (dd, J = 12.1, 1.8 Hz, 1H), 7.43 (d, J = 5.2 Hz, 1H), 7.24 (d, J = 7.6 Hz, 1H), 4.99 (brs, 1H), 4.41 (s, 2H), 3.98-3.81 (m, 3H), 3.58-3.52 (m, 2H), 3.14-3.06 (m, 1H), 2.76 (s, 2H), 2.19-1.84 (m, 1H), 1.62-1.40 (m, 1H), 1.15 (s, 6H). |
| 108a | 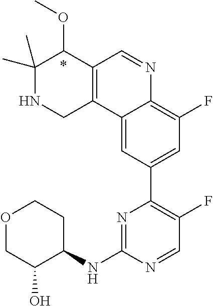 [SMILES: COC1c2cnc3c(F)cc(-c4nc(N[C@@H]5CCOC[C@H]5O)ncc4F)cc3c2CNC1(C)C] Isomer 1 (earlier eluting isomer) | LC-MS (ESI): m/z 472.2 [M + H]⁺; de: 100%; Retention time: 1.516 min; Instrument: Agilent- 1260, Column: CHIRALPAK IH-3, 4.6 * 50 mm, 3.0 um, Mobile phase: A for Hex (0.1% DEA) and B for EtOH, Gradient: B 20%, Flow rate: 1.67 mL/min, High pressure: 110 bar, Column temperature: 25° C., Wavelength: 254 nm. |
| 108b | 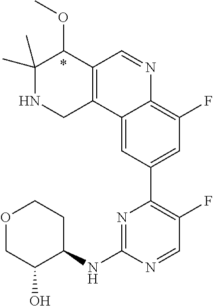 [SMILES: COC1c2cnc3c(F)cc(-c4nc(N[C@@H]5CCOC[C@H]5O)ncc4F)cc3c2CNC1(C)C] Isomer 2 (later eluting isomer) | LC-MS (ESI): m/z 472.2 [M + H]⁺; ¹H NMR (500 MHz, DMSO-d₆) δ 8.95 (s, 1H), 8.52 (d, J = 3.7 Hz, 1H), 8.41 (s, 1H), 8.10 (d, J = 12.4 Hz, 1H), 7.33 (d, J = 6.3 Hz, 1H), 4.94 (s, 1H), 4.44- 4.37 (m, 1H), 4.32-4.23 (m, 1H), 4.10 (s, 1H), 3.87-3.79 (m, 3H), 3.57 (s, 3H), 3.55-3.48 (m, 1H), 3.41-3.39 (m, 1H), 3.13-3.04 (m, 1H), 2.05-1.96 (m, 1H), 1.52-1.45 (m, 1H), 1.09 (s, 3H), 1.07 (s, 3H). de: 100%; Retention time: 2.177 min; Instrument: Agilent- 1260, Column: CHIRALPAK IH-3, 4.6*50 mm, 3.0 um, Mobile phase: A for Hex (0.1% DEA) and B for EtOH, Gradient: B 20%, Flow rate: 1.67 mL/min, High pressure: 110 bar, Column temperature: 25° C., Wavelength: 254 nm. |
| 109 | 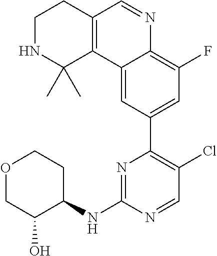 [SMILES: CC1(C)NCCc2cnc3c(F)cc(-c4nc(N[C@@H]5CCOC[C@H]5O)ncc4Cl)cc3c21] | LC-MS (ESI): m/z 458.1 [M + H]⁺; ¹H NMR (500 MHz, DMSO-d₆) δ 8.75 (s, 1H), 8.65-8.43 (m, 2H), 8.00-7.70 (m, 1H), 7.57 (d, J = 7.9 Hz, 1H), 4.95 (brs, 1H), 3.93-3.78 (m, 4H), 3.37- 3.26 (m, 1H), 3.13-3.06 (m, 2H), 3.06-2.99 (m, 1H), 2.98-2.92 (m, 2H), 2.08-1.88 (m, 1H), 1.72 (s, 6H), 1.58-1.44 (m, 1H). |
| 110 | 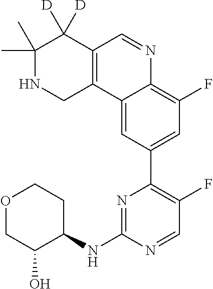 [SMILES: [2H]C1([2H])c2cnc3c(F)cc(-c4nc(N[C@@H]5CCOC[C@H]5O)ncc4F)cc3c2CNC1(C)C] | LC-MS (ESI): m/z 444.2 [M + H]⁺; ¹H NMR (500 MHz, DMSO-d₆) δ 8.73 (s, 1H), 8.51 (d, J = 3.7 Hz, 1H), 8.39 (s, 1H), 8.05 (d, J = 11.9 Hz, 1H), 7.31 (d, J = 7.5 Hz, 1H), 4.94 (brs, 1H), 4.35 (s, 2H), 3.89-3.76 (m, 3H), 3.55-3.52 (m, 1H), 3.44-3.38 (m, 1H), 3.08 (t, J = 10.3 Hz, 1H), 2.11-1.94 (m, 1H), 1.59-1.45 (m, 1H), 1.14 (s, 6H). |
| 111a | 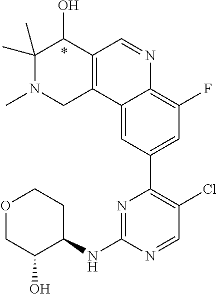 [SMILES: CN1Cc2c(cnc3c(F)cc(-c4nc(N[C@@H]5CCOC[C@H]5O)ncc4Cl)cc23)C(O)C1(C)C] Isomer 1 (earlier eluting isomer) | LC-MS (ESI): m/z 488.2 [M + H]⁺; ¹H NMR (500 MHz, DMSO-d₆) δ 9.06 (s, 1H), 8.49 (s, 1H), 8.36-8.08 (m, 1H), 8.03-7.77 (m, 1H), 7.68-7.45 (m, 1H), 5.82 (d, J = 6.9 Hz, 1H), 4.95 (d, J = 5.4 Hz, 1H), 4.45 (d, J = 6.9 Hz, 1H), 4.19 (d, J = 17.8 Hz, 1H), 4.02-3.73 (m, 4H), 3.55-3.47 (m, 1H), 3.40-3.36 (m, 1H), 3.06 (t, J = 10.5 Hz, 1H), 2.44 (s, 3H), 2.08- 1.86 (m, 1H), 1.62-1.41 (m, 1H), 1.21 (s, 3H), 0.83 (s, 3H). de: 100%; Retention time: 0.945 min; Instrument: Agilent- 1260, Column: CHIRAL Cellulose-SB, 4.6 * 50 mm, 3.0 um, Mobile phase: A for MtBE: Hex = 1:1 (0.1% DEA) and B for MeOH, Gradient: B 15%, Flow rate: 1.67 mL/min, High pressure: 110 bar; Column temperature: 25° C.; Wavelength: 254 nm. |
| 111b | 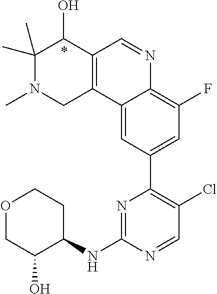 [SMILES: CN1Cc2c(cnc3c(F)cc(-c4nc(N[C@@H]5CCOC[C@H]5O)ncc4Cl)cc23)C(O)C1(C)C] Isomer 2 (later eluting isomer) | LC-MS (ESI): m/z 488.2 [M + H]⁺; ¹H NMR (500 MHz, DMSO-d₆) δ 9.05 (s, 1H), 8.48 (s, 1H), 8.33-8.04 (m, 1H), 8.01-7.77 (m, 1H), 7.71-7.48 (m, 1H), 5.81 (d, J = 7.7 Hz, 1H), 4.94 (d, J = 5.6 Hz, 1H), 4.43 (d, J = 7.3 Hz, 1H), 4.18 (d, J = 17.8 Hz, 1H), 3.97-3.75 (m, 4H), 3.55-3.48 (m, 1H), 3.39-3.35 (m, 1H), 3.06 (t, J = 10.5 Hz, 1H), 2.42 (s, 3H), 2.05- 1.83 (m, 1H), 1.59-1.42 (m, 1H), 1.20 (s, 3H), 0.81 (s, 3H). de: 100%; Retention time: 1.343 min; Instrument: Agilent- 1260, Column: CHIRAL Cellulose-SB, 4.6 * 50 mm, 3.0 um, Mobile phase: A for MtBE: Hex = 1:1 (0.1% DEA) and B for MeOH; Gradient: B 15%; Flow rate: 1.67 mL/min, High pressure: 110 bar; Column temperature: 25° C.; Wavelength: 254 nm. |

### Biological Example 1. Measurement of Kinase Inhibitory Activity

CDK2/CyclinE1 kinase inhibitory activity (IC50): 5 μl of various dilutions of test compounds in 1×kinase buffer (50 mM HEPES pH 7.5, 10 mM MgCl₂, 2 mM DTT and 0.0100 Brij-35) were mixed with 10 μL of CDK2/CyclinE1 (Carna, 04-165 #, final concentration 3 nM in 1×Kinase buffer) in 384 plates and incubated at room temperature for 10 min. To initiate each reaction, 10 μL of peptide solution containing fluorescently-labeled-peptide 18(5-FAM-QSPKKG-CONH2) (GL, 114202 #, final concentration 3000 nM) and ATP (final concentration 77 μM) in 1×Kinase buffer was added to each of the wells containing test compound and CDK2/CyclinE1 mixture. The reaction was then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 18 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK1/CyclinB kinase inhibitory activity (IC50): 5 μl of various dilutions of test compound in 1× kinase buffer (50 mM HEPES pH 7.5, 10 mM MgCl₂, 2 mM DTT and 0.01% Brij-35) was mixed with 10 μL of CDK1/CyclinB (Millipore, 14-450M #, final concentration 3 nM in 1× Kinase buffer) in 384 plates and incubated at room temperature for 10 min. To initiate each reaction, 10 μL of peptide solution containing fluorescently-labeled-peptide 18(5-FAM-QSPKKG-CONH2) (GL, 114202 #, final concentration 3000 nM) and ATP (final concentration 20 μM) in 1× Kinase buffer was added to each of the wells containing test compound and CDK1/CyclinB mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 18 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK4/CyclinD1 kinase inhibitory activity (IC50): 5 μl of various dilutions of test compound in 1× kinase buffer (20 mM HEPES, pH 7.5, 10 mM MgCl₂, 2 mM DTT and 0.01% Triton X-100) was mixed with 10 μL of either CDK4/Cyclin D1 (ProQinase, 0142-0143-1 #, final concentration 20 nM in 1× Kinase buffer) or in 384 plates and incubated at room temperature for 10 min. To initiate each reaction, 10 μL of peptide solution containing fluorescently-labeled-peptide 8(5-FAM-IPTSPITTTYFFFKKK-COOH, GL, 112396 #, final concentration 3000 nM) and ATP (final concentration 672 uM for CDK4/CyclinD1) in 1×Kinase buffer was added to each of the wells containing test compound and CDK4/CyclinD1 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 8 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK6/CyclinD3 kinase inhibitory activity (IC50): 5 μl of various dilutions of test compound in 1× kinase buffer (50 mM HEPES pH 7.5, 10 mM MgCl₂, 2 mM DTT and 0.01% Brij-35) was mixed with 10 μL of CDK6/CyclinD3 (Carna, 04-107 #, final concentration 15 nM in 1× Kinase buffer) in 384 plates and incubated at room temperature for 10 min. To initiate each reaction, 10 μL of peptide solution containing fluorescently-labeled -peptide 8(5-FAM-IPTSPITTTYFFFKKK-COOH, GL, 112396 #, final concentration 3000 nM) and ATP (final concentration 800 μM for CDK6/CyclinD3 in 1× Kinase buffer was added to each of the wells containing test compound and CDK6/CyclinD3. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide 8 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK7/CyclinH/MAT1 kinase inhibitory activity (IC50): 5 μl of various dilutions of test compound in 1× kinase buffer (20 mM HEPES, pH 7.5, 10 mM MgCl₂, 2 mM DTT and 0.01% Triton X-100) was mixed with 10 μL of CDK7/CyclinH/MAT1 (Millipore, 14-476M #, final concentration 12.5 nM in 1× Kinase buffer) in 384 plates and incubated at room temperature for 10 min. To initiate each reaction, 10 μL of peptide solution containing fluorescently-labeled-peptide CTD3 (5-FAM-ACSYSPTSPSYSPTSPSYSPTSPSKK, GL, SY346885 #, final concentration 3000 nM) and ATP (final concentration 70 μM) in 1× Kinase buffer was added to each of the wells containing test compound and CDK7/CyclinH/MAT1 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphoiylated (product) and the unphosphorylated (substrate) fluorescently-labeled peptide CTD3 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

CDK9/CyclinT1 kinase inhibitory activity (IC50): 5 μl of various dilutions of test compound in 1× kinase buffer (20 mM HEPES, pH 7.5, 10 mM MgCl₂, 2 mM DTT and 0.01% Triton X-100) was mixed with 10 μL of CDK9/CyclinT1 (Millipore, 14-685M #, final concentration 12.5 nM in 1× Kinase buffer) in 384 plates and incubated at room temperature for 10 min. To initiate each reaction, 10 μL of peptide solution containing fluorescently-labeled -peptide CTD3 (5-FAM-ACSYSPTSPSYSPTSPSYSPTSPSKI, GL, SY346885 #, final concentration 3000 nM) and ATP (final concentration 10M) in 1× Kinase buffer was added to each of the wells containing test compound and CDK9/CyclinT1 mixture. The reaction is then allowed to proceed at 28° C. for 30 min and terminated by the addition of 25 μL stop buffer (100 mM HEPES pH 7.5, 50 mM EDTA, 0.2% Coating Reagent #3 (Perkin Elmer, 760050 #) and 0.015% Brij-35).

Following the kinase reaction, Caliper EZ reader II (Downstream voltages: −500V, Upstream voltages: −2250V, Base pressure −0.5 PSI, Screen pressure −1.2 PSI) was used to separate the phosphorylated (product) and the unphosphorylated (substrate) fluorescently-labeled peptide CTD3 based on their different mobility. Both substrate and product were measured and the ratio of these values were used to generate % conversion by Caliper EZ reader II. These conversion values were then transformed into % inhibition of kinase activity using the formula: % Inhibition=[(MA−X)/(MA−MI)]×100% where MA=conversion value of DMSO only controls, MI=conversion value of no enzyme controls and X=conversion value at any given compound dose. IC50 values were then calculated by plotting dose-response curves and then using the XLfit application in Excel software.

Biological activity data for representative compounds of the present disclosure are provided in Table 2 below. Exemplary results are presented as calculated IC₅₀ values. In Table 2, “A” represents a calculated IC₅₀ value of less than 10 nM; “B” represents a calculated IC₅₀ value of greater than or equal to 10 nM and less than 100 nM; “C” represents a calculated IC₅₀ value of greater than or equal to 100 nM and less than 1 μM; and “D” represents a calculated IC₅₀ value of 1 μM or greater.

TABLE 2
Selected in vitro data on different CDKs

| | CDK1/ | CDK2/ | CDK4/ | CDK6/ | CDK7/ | CDK9/ |
|---|---|---|---|---|---|---|
| Exam- | Cyclin | Cyclin | Cyclin | Cyclin | Cyclin | Cyclin |
| ple | B1 | E1 | D1 | D3 | H/MAT1 | T1 |
| num- | IC50 | IC50 | IC50 | IC50 | IC50 | IC50 |
| ber | (nM) | (nM) | (nM) | (nM) | (nM) | (nM) |
| 1 | D | D | A | C | D | D |
| 2 | | | B | C | | |
| 3 | | | B | C | | |
| 4 | | | C | C | | |
| 5 | D | C | A | C | D | C |
| 6 | | | B | C | | |
| 7 | C | C | A | B | D | C |
| 8 | B | A | A | B | B | B |
| 9 | C | C | A | C | C | C |
| 10 | | | C | D | | |
| 11 | | | B | C | | |
| 12 | D | D | B | C | D | C |
| 13 | | | B | C | | |
| 14 | B | B | A | C | C | C |
| 15a | C | C | A | B | D | C |
| 15b | C | C | A | B | C | C |
| 16 | | | A | C | | |
| 17 | C | C | A | B | D | C |
| 18 | C | C | A | C | D | D |
| 19 | B | B | A | B | B | B |
| 20a | | | C | D | | |
| 20b | C | C | A | C | C | C |
| 21 | C | C | A | C | D | C |
| 22 | C | C | A | B | C | C |
| 23 | C | B | A | B | B | C |
| 24 | C | B | A | B | C | B |
| 25 | B | B | A | B | B | B |
| 26 | C | C | B | D | D | C |
| 27 | | | A | B | | |
| 28 | | | B | C | | |
| 29 | C | C | B | C | D | C |
| 30 | C | C | A | B | D | B |
| 31a | | | B | D | | |
| 31b | | | A | C | | |
| 32a | C | B | A | C | D | C |
| 32b | | | B | C | | |
| 33 | | | A | C | | |
| 34a | C | B | A | B | C | B |
| 34b | D | C | A | C | D | C |
| 35a | C | C | A | B | D | C |
| 35b | D | C | A | C | D | D |
| 36 | D | C | B | C | D | C |
| 37 | D | D | A | C | D | D |
| 38a | | | A | B | | |
| 38b | | | A | B | | |
| 39a | | | A | C | | |
| 39b | | | A | C | | |
| 40a | | C | A | C | C | C |
| 40b | | | A | C | | |
| 41 | D | C | A | B | D | D |
| 42 | | | A | C | | |
| 43 | | | A | C | | |
| 44 | | | A | B | | |
| 45a | C | C | A | C | D | C |
| 45b | D | D | A | C | D | D |
| 46 | | | A | C | | |
| 47 | | | B | C | | |
| 48a | | | A | C | | |
| 48b | | | B | C | | |
| 49 | D | C | A | C | D | C |
| 50 | | | B | D | | |
| 51 | | | A | C | | |
| 52 | D | D | A | B | D | D |
| 53 | | | A | C | | |
| 54a | | | A | C | | |
| 54b | | | A | C | | |
| 55 | | | A | D | | |
| 56 | B | B | A | C | B | B |
| 57a | C | C | A | C | D | C |
| 57b | | | B | D | | |
| 58 | D | D | A | C | D | D |
| 59a | | | B | C | | |
| 59b | | | B | C | | |
| 60 | D | D | A | C | D | C |
| 61 | | | B | C | | |
| 62 | | | A | B | | |
| 63a | | | B | C | | |
| 63b | | | B | C | | |
| 64a | | | B | C | | |
| 64b | | | B | C | | |
| 65 | C | C | A | C | C | C |
| 66 | | | B | C | | |
| 67 | D | D | B | D | D | C |
| 68 | C | C | A | B | C | C |
| 69 | | | A | C | | |
| 70 | | | A | C | | |
| 71 | | | A | C | | |
| 72 | | | A | C | | |
| 73a | | | B | C | | |
| 73b | C | C | A | B | D | C |
| 74a | | | A | C | | |
| 74b | | | A | C | | |
| 75 | | | B | C | | |
| 76 | | | B | C | | |
| 77a | | | A | C | | |
| 77b | | | A | C | | |
| 78 | | | A | C | | |
| 79a | | | A | C | | |
| 79b | | | B | C | | |
| 80 | | | B | D | | |
| 81a | C | C | A | B | C | B |
| 81b | C | C | A | B | C | C |
| 82a | | | B | C | | |
| 82b | | | B | C | | |
| 83 | C | C | A | C | C | C |
| 84a | | | A | B | | |
| 84b | | | A | B | | |
| 85a | | | A | B | | |
| 85b | | | A | B | | |
| 87 | | | B | C | | |
| 88a | C | C | A | B | C | C |
| 88b | | | A | B | | |
| 89 | C | C | A | B | D | B |
| 90a | | | B | C | | |
| 90b | | | A | C | | |
| 91a | | | B | C | | |
| 91b | D | C | A | C | D | D |
| 92 | | | A | C | | |
| 93a | | | B | C | | |
| 93b | | | A | C | | |
| 94 | C | C | A | B | B | C |
| 95 | | | A | B | | |
| 96 | | | B | D | | |
| 97 | | | B | C | | |
| 98 | D | C | A | C | D | C |
| 99 | | | B | D | | |
| 100 | C | C | A | B | C | B |
| 101 | | | A | C | | |
| 102 | D | D | A | C | D | C |
| 103 | | | A | C | | |
| 104 | | | B | D | | |
| 105 | | | A | C | | |
| 106a | | | A | C | | |
| 106b | C | C | A | B | C | C |
| 107 | C | C | A | C | C | C |
| 108a | | | B | D | | |
| 108b | | | A | C | | |
| 109 | | | A | C | | |
| 110 | | | A | C | | |
| 111a | | | B | C | | |
| 111b | | | C | D | | |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 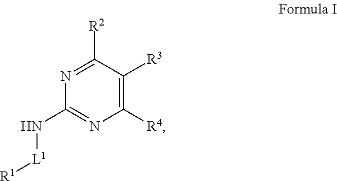
wherein:
L¹ is an optionally substituted phenylene, optionally substituted 5- or 6-membered heteroarylene, optionally substituted 4-8-membered heterocyclylene, or optionally substituted C₃₋₈ carbocyclylene;
R¹ is hydrogen, OH, NH₂, NHCH₃, or N(CH₃)₂;
X is N or CR¹⁰;
R² is an optionally substituted ring system selected from a 10 membered heteroaryl, 9-12 membered bicyclic heterocyclic ring, or 12-16 membered heteroaryl or heterocyclic ring structure having three or more rings, wherein the ring system includes at least one phenyl or heteroaryl ring, and wherein R² connects to the remainder of Formula I through a ring atom of a phenyl or heteroaryl portion of the ring system, and provided that said phenyl or heteroaryl portion is not fused to a 5-membered heteroaryl;
R³ is hydrogen, deuterium, halogen (e.g., F, Cl), CN, OR¹¹, NR¹²R¹³, C(O)NR¹²R¹³, COOR^{A}, COR^{B}, optionally substituted C₁₋₆ alkyl, optionally substituted C₂₋₄ alkenyl, optionally substituted C₂₋₄ alkynyl, optionally substituted C₁₋₄ heteroalkyl, optionally substituted C₃₋₈ carbocyclyl, optionally substituted 4-10 membered heterocyclyl, or optionally substituted 5-10 membered heteroaryl;
R⁴ is hydrogen, deuterium, halogen (e.g., F), optionally substituted C₁₋₆ alkyl, or NR¹²R¹³;
wherein:
R¹⁰ is hydrogen, halogen (e.g., F), CN, —OH, an optionally substituted C₁₋₄ alkyl, optionally substituted C₁₋₄ heteroalkyl, optionally substituted C₃₋₈ carbocyclyl, or optionally substituted 4-10 membered heterocyclyl;
R¹¹ is hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or an oxygen protecting group;
each of R¹² and R¹³, at each occurrence, is independently hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or a nitrogen protecting group; or R¹² and R¹³ can be joined to form an optionally substituted 4-10 membered heterocyclyl or 5- or 6-membered heteroaryl;
R^{A} is hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or an oxygen protecting group; and
R^{B} is hydrogen, an optionally substituted C₁₋₆ alkyl, optionally substituted C₃₋₈ carbocyclyl, optionally substituted phenyl, optionally substituted 4-10 membered heterocyclyl, or optionally substituted heteroaryl (e.g., 5- or 6-membered heteroaryl).

2. The compound of claim 1, or a pharmaceutically acceptable salt thereof, characterized as having a structure according to Formula A: 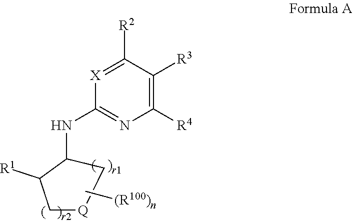
wherein:
Q is
(1) O;
(2) NR¹⁴, wherein R¹⁴ is hydrogen, G^{A}, SO₂G^{A}, SO₂NG^{B}G^{C}, S(O)(NH)G^{A}, COG^{A}, COOG^{A}, or C(O)NG^{B}G^{C};
(3) CR¹⁵R¹⁶, wherein R¹⁵ and R¹⁶ are joined with the carbon atom they are attached to form an optionally substituted 4-6 membered heterocyclic ring having 1 or 2 ring heteroatoms independently selected from 0 and N; or
(4) absent;
r1 is 1, 2, or 3; and
r2 is 0, 1, or 2;
n is 0, 1, 2, 3, or 4, as valency permits; and
(i) R¹⁰⁰ at each occurrence is independently selected from halogen (e.g., F or Cl), CN, OH, COOH, G^{A}, OG^{A}, NG^{B}G^{C}, NG^{B}G^{C}SO₂G^{A}, NG^{B}G^{C}SO₂NG^{B}G^{C}, NG^{B}G^{C}S(O)(NH)G^{A}, NG^{B}G^{C}COG^{A}, NG^{B}G^{C}COOG^{A}, NG^{B}G^{C}C(O)NG^{B}G^{C}, SO₂G^{A}, SO₂NG^{B}G^{C}, S(O)(NH)G^{A}, COG^{A}, COOG^{A}, or C(O)NG^{B}G^{C}; or
(ii) two instances of R¹⁰⁰ are joined together with the intervening atom(s) to form an optionally substituted ring, such as an optionally substituted 3-6 membered ring, and any remaining R¹⁰⁰ at each occurrence is as defined in (i);
wherein:
G^{A} at each occurrence is independently an optionally substituted C₁₋₆ alkyl, optionally substituted C₃₋₈ carbocyclyl, optionally substituted phenyl, optionally substituted heteroaryl (e.g., 5- or 6-membered heteroaryl), or optionally substituted 4-10 membered heterocyclyl; and
each of G^{B} and G^{C}, at each occurrence, is independently hydrogen, an optionally substituted C₁₋₆ alkyl, optionally substituted C₃₋₈ carbocyclyl, optionally substituted phenyl, optionally substituted heteroaryl (e.g., 5- or 6-membered heteroaryl), optionally substituted 4-10 membered heterocyclyl; or a nitrogen protecting group; or G^{B} and G^{C} can be joined to form an optionally substituted 4-10 membered heterocyclyl or 5- or 6-membered heteroaryl.

3. The compound of claim 2, or a pharmaceutically acceptable salt thereof, wherein n is 0.

4. The compound of claim 2, or a pharmaceutically acceptable salt thereof, wherein n is 1 or 2, and R¹⁰⁰ at each occurrence is independently selected from F, Cl, CN, OH, C₁₋₄ alkyl optionally substituted with F, C₁₋₄ alkoxy optionally substituted with F, and C₁₋₄ heteroalkyl optionally substituted with F.

5. The compound of claim 2, or a pharmaceutically acceptable salt thereof, wherein n is 1, and R¹⁰⁰ is F, Cl, CN, OH, methyl, fluorine-substituted methyl such as CF₃, methoxy, or fluorine-substituted methoxy.

6. The compound of claim 2, or a pharmaceutically acceptable salt thereof, characterized as having Formula A-1 or A-2: 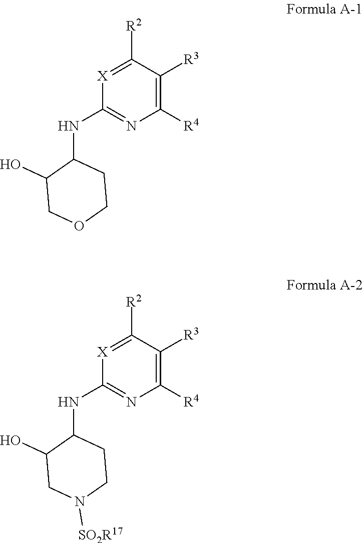
wherein:
R¹⁷ is
(1) C₁₋₄ alkyl optionally substituted with 1-3 substituents independently selected from deuterium, F, and OH, such as CHF₂, CF₃, etc.;
(2) phenyl, pyridyl, or pyrimidyl, each of which is optionally substituted with 1-3 substituents independently selected from deuterium, halogen, CN, OH, C₁₋₃ alkyl optionally substituted with F, or CI-3 alkoxy optionally substituted with F; or
(3) 5-membered heteroaryl optionally substituted with 1-3 substituents independently selected from deuterium, halogen, CN, OH, CI-3 alkyl optionally substituted with F, or C₁₋₃ alkoxy optionally substituted with F.

7. The compound of claim 6, or a pharmaceutically acceptable salt thereof, wherein R¹⁷ is selected from:

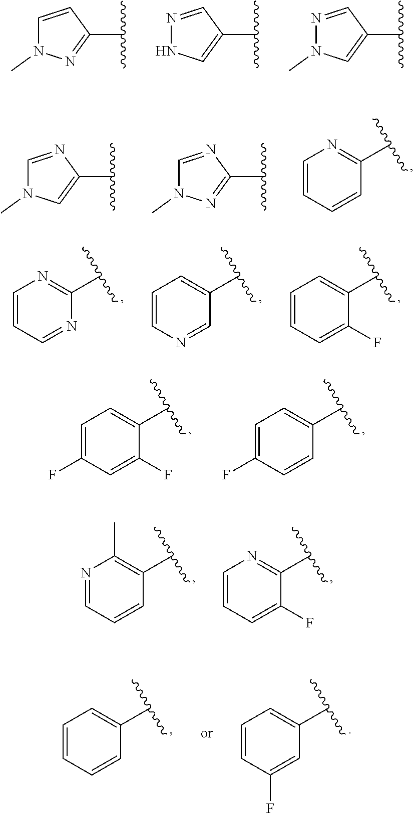

8. The compound of claim 1, or a pharmaceutically acceptable salt thereof, wherein L¹-R¹ in Formula I is selected from: 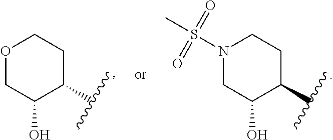 [SMILES: *[C@H]1CCOC[C@H]1O ; *[C@@H]1CCN(S(C)(=O)=O)C[C@H]1O]
or L¹-R¹ in Formula I is selected from:

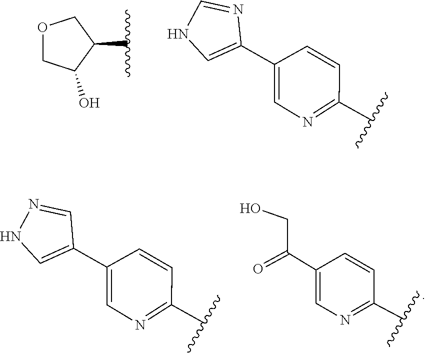 [SMILES: *[C@@H]1COC[C@H]1O ; *c1ccc(-c2c[nH]cn2)cn1 ; *c1ccc(-c2cn[nH]c2)cn1 ; *c1ccc(C(=O)CO)cn1]

9. The compound of any one of claims 1-8, or a pharmaceutically acceptable salt thereof, wherein X is N.

10. The compound of any one of claims 1-9, or a pharmaceutically acceptable salt thereof, wherein R² is a 10-membered bicyclic heteroaryl having 1-4 ring nitrogen atoms, such as quinolinyl, naphthyridinyl, etc., which is optionally substituted with one or more (e.g., 1, 2, or 3) substituents independently selected from halo (e.g., F), CN, G¹, OH, COOH, C(O)-G¹, O-G¹, C(O)—O-G¹, NH₂, NH(G¹), N(G¹)(G¹), C(O)—NH₂, C(O)—NH(G¹), C(O)—N(G¹)(G¹), G², O-G², NH(G²), N(G¹)(G²), C(O)—NH(G²), and C(O)—N(G¹)(G²),
wherein G¹ at each occurrence is independently a C₁₋₄ alkyl optionally substituted with 1-3 substituents independently selected from F, CN, OH, and C₁₋₄ heteroalkyl, or a C₃₋₆ cycloalkyl optionally substituted with 1-3 substituents independently selected from F, CN, OH, and C₁₋₄ heteroalkyl;
wherein G² at each occurrence is independently a 4-6 membered heterocyclyl having 1-2 ring heteroatoms independently selected from N, O, and S, phenyl or 5- or 6-membered heteroaryl, each of which is optionally substituted with 1-3 substituents independently selected from oxo (as applicable), halo (e.g., F), CN, G¹, OH, O-G¹, NH₂, NH(G¹), and N(G¹)(G¹); and
wherein two optional substituents of the 10-membered bicyclic heteroaryl group, together with the intervening atom(s), can optionally be joined to form a fused ring structure.

11. The compound of any one of claims 1-9, or a pharmaceutically acceptable salt thereof, wherein R² has a structure according to M-1: 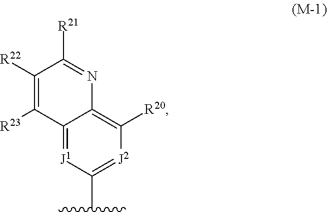
wherein:
J¹ and J² are independently N or CR³⁰, wherein R³⁰ at each occurrence is independently hydrogen, halogen, C₁₋₄ alkyl optionally substituted with F, or C₁₋₄ heteroalkyl optionally substituted with F;
R²⁰ is hydrogen, halogen (e.g., F or Cl), CN, C₁₋₄ alkyl optionally substituted with F, C₁₋₄ heteroalkyl optionally substituted with F, or an optionally substituted 3-6 membered ring structure (e.g., cyclopropyl, cyclobutyl, 4-6 membered heterocyclic ring, or 5 membered heteroaryl);
R²¹, R²², and R²³ are each independently: (1) hydrogen or deuterium; (2) halogen (e.g., F or Cl), (3) CN, (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) a 3-6 membered ring structure (e.g., cyclopropyl, cyclobutyl, 4-6 membered heterocyclic ring, or 5 membered heteroaryl) optionally substituted 1-3 substituents each independently oxo, halo (e.g., F), OH, NH₂, C₁₋₄ alkyl optionally substituted with F, or C₁₋₄ heteroalkyl optionally substituted with F, or R²¹ and R²², together with the intervening atoms, are joined to form a 5-8 membered ring (e.g., 5-7 membered carbocyclic or heterocyclic ring), which is optionally substituted 1-3 substituents each independently oxo, deuterium, halo (e.g., F), OH, NH₂, C₁₋₄ alkyl optionally substituted with F, or C₁₋₄ heteroalkyl optionally substituted with F, and R²³ is defined above;
or R²² and R²³, together with the intervening atoms, are joined to form a 5-8 membered ring (e.g., 5-7 membered carbocyclic, aryl, heteroaryl, or heterocyclic ring), which is optionally substituted 1-3 substituents each independently oxo, deuterium, halo (e.g., F), OH, NH₂, C₁₋₄ alkyl optionally substituted with F, or C₁₋₄ heteroalkyl optionally substituted with F, and R²¹ is defined above;
preferably, at least one of R²¹, R²², and R²³ contains a hydrogen bond donor, more preferably, only one of R²¹, R²², and R²³ contains a hydrogen bond donor, or the ring structure formed by R²¹ and R²², or R²² and R²³, contains a hydrogen bond donor.

12. The compound of claim 11, or a pharmaceutically acceptable salt thereof, wherein J^{P} is CR³⁰, and R³⁰ is hydrogen.

13. The compound of claim 11, or a pharmaceutically acceptable salt thereof, wherein J¹ is N.

14. The compound of any one of claims 11-13, or a pharmaceutically acceptable salt thereof, wherein J² is CR³⁰, and R³⁰ is hydrogen.

15. The compound of any one of claims 11-13, or a pharmaceutically acceptable salt thereof, wherein J² is N.

16. The compound of claim 11, or a pharmaceutically acceptable salt thereof, wherein R² has a structure according to M-1-1: 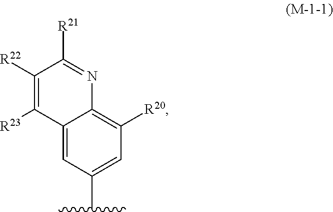
wherein:
R²¹, R²², and R²³ are each independently: (1) hydrogen or deuterium; (2) halogen (e.g., F or Cl), (3) CN, (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) a 3-6 membered ring structure (e.g., cyclopropyl, cyclobutyl, 4-6 membered heterocyclic ring, or 5 membered heteroaryl) optionally substituted 1-3 substituents each independently oxo, halo (e.g., F), OH, NH₂, C₁₋₄ alkyl optionally substituted with F, or C₁₋₄ heteroalkyl optionally substituted with F.

17. The compound of claim 11 or 16, or a pharmaceutically acceptable salt thereof, wherein the compound is a compound of Formula I-1:

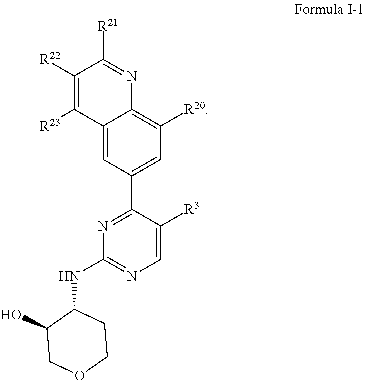

18. The compound of any one of claims 11-17, or a pharmaceutically acceptable salt thereof, wherein R²⁰ is hydrogen, halogen, or C₁₋₄ alkyl.

19. The compound of any one of claims 11-17, or a pharmaceutically acceptable salt thereof, wherein R²⁰ is hydrogen, F, Cl, methyl, or ethyl, preferably, R²⁰ is F.

20. The compound of any one of claims 11-19, or a pharmaceutically acceptable salt thereof, wherein R²¹ is hydrogen, deuterium, halogen (e.g., F, or Cl), C₁₋₄ alkyl (e.g., methyl or ethyl), or C₁₋₄ heteroalkyl having 1 or 2 heteroatoms independently oxygen or nitrogen, preferably, R²¹ is hydrogen, deuterium, F, or methyl.

21. The compound of any one of claims 11-19, or a pharmaceutically acceptable salt thereof, wherein R²¹ is C₁₋₄ alkyl substituted with OH, NH₂, or NH(C₁₋₄ alkyl).

22. The compound of any one of claims 11-19, or a pharmaceutically acceptable salt thereof, wherein R²¹ is

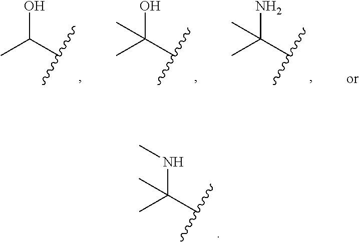

23. The compound of any one of claims 11-19, or a pharmaceutically acceptable salt thereof, wherein R²¹ is a 4-6 membered heterocyclic ring having 1 or 2 ring heteroatoms, e.g., azetidinyl, pyrrolidinyl, piperidinyl, piperazinyl, etc., wherein the 4-6 membered heterocyclic ring is optionally substituted with 1-3 substituents each independently oxo, F, OH, NH₂, C₁₋₄ alkyl optionally substituted with F, C₁₋₄ alkoxy optionally substituted with F, or NH(C₁₋₄ alkyl).

24. The compound of any one of claims 11-19, or a pharmaceutically acceptable salt thereof, wherein R²¹ is azetidinyl or pyrrolidinyl, such as

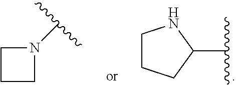 [SMILES: *N1CCC1 ; *C1CCCN1]

25. The compound of any one of claims 11-19, or a pharmaceutically acceptable salt thereof, wherein R²¹ is a 5 membered heteroaryl ring having 2-4 ring heteroatoms, e.g., imidazolyl, pyrazolyl, etc., wherein the 5 membered heteroaryl ring is optionally substituted with 1-3 substituents each independently F, OH, NH₂, C₁₋₄ alkyl optionally substituted with F, C₁₋₄ alkoxy optionally substituted with F, or NH(C₁₋₄ alkyl).

26. The compound of any one of claims 11-19, or a pharmaceutically acceptable salt thereof, wherein R²¹ is an imidazolyl or pyrazolyl, optionally substituted with one or two C₁₋₄ alkyl such as methyl, such as

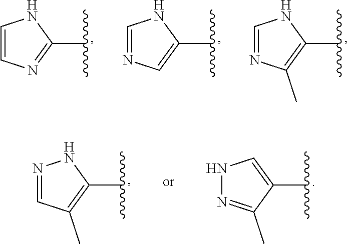

27. The compound of any one of claims 11-26, or a pharmaceutically acceptable salt thereof, wherein R²² is hydrogen, halogen (e.g., F, or Cl), C₁₋₄ alkyl (e.g., methyl or ethyl), or C₁₋₄ heteroalkyl having 1 or 2 heteroatoms independently oxygen or nitrogen.

28. The compound of any one of claims 11-26, or a pharmaceutically acceptable salt thereof, wherein R²² is C₁₋₄ alkyl substituted with OH, NH₂, or NH(C₁₋₄ alkyl), or R²² is C₃₋₆ cycloalkyl substituted with OH, NH₂, or NH(C₁₋₄ alkyl).

29. The compound ofany one of claims 11-26, or a pharmaceutically acceptable salt thereof, wherein R²² is 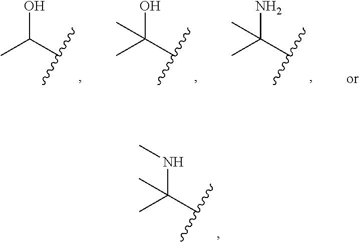
or R²² is 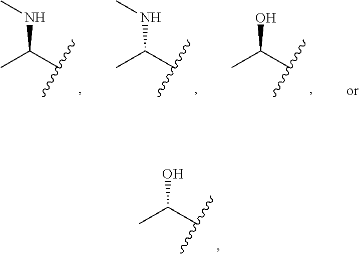
or R²² is 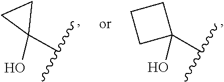 [SMILES: *C1(O)CC1 ; *C1(O)CCC1]
or R²² is 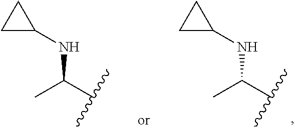
or R²² is

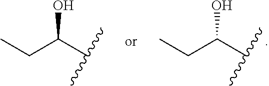

30. The compound of any one of claims 11-26, or a pharmaceutically acceptable salt thereof, wherein R²² is a 4-6 membered heterocyclic ring having 1 or 2 ring heteroatoms, e.g., azetidinyl, pyrrolidinyl, piperidinyl, etc., wherein the 4-6 membered heterocyclic ring is optionally substituted with 1-3 substituents each independently oxo, F, OH, NH₂, C₁₋₄ alkyl optionally substituted with F, C₁₋₄ alkoxy optionally substituted with F, or NH(C₁₋₄ alkyl).

31. The compound of any one of claims 11-26, or a pharmaceutically acceptable salt thereof, wherein R²² is azetidinyl or pyrrolidinyl, such as 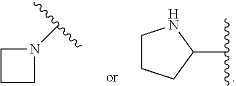 [SMILES: *N1CCC1 ; *C1CCCN1]
or R²² is

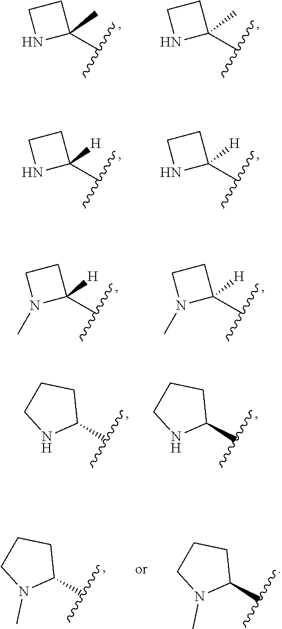

32. The compound of any one of claims 11-26, or a pharmaceutically acceptable salt thereof, wherein R²² is a 5 membered heteroaryl ring having 2-4 ring heteroatoms, e.g., imidazolyl, pyrazolyl, etc., wherein the 5 membered heteroaryl ring is optionally substituted with 1-3 substituents each independently F, OH, NH₂, C₁₋₄ alkyl optionally substituted with F, C₁₋₄ alkoxy optionally substituted with F, or NH(C₁₋₄ alkyl), preferably, R²² is an imidazolyl or pyrazolyl, optionally substituted with one or two C₁₋₄ alkyl such as methyl, such as

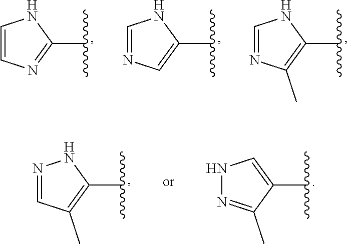

33. The compound of any one of claims 11-19, or a pharmaceutically acceptable salt thereof, wherein R²¹ and R²², together with the intervening atoms, are joined together to form a 5 or 6 membered ring, preferably, a 5 or 6 membered carbocyclic ring, which is substituted with 1-3 substituents each independently F, OH, NH₂, C₁₋₄ alkyl optionally substituted with F, C₁₋₄ alkoxy optionally substituted with F, or NH(C₁₋₄ alkyl), preferably, one of the substitutent(s) comprises a hydrogen bond donor, such as OH, NH₂, or NH(C₁₋₄ alkyl).

34. The compound of any one of claims 11-33, or a pharmaceutically acceptable salt thereof, wherein R²³ is hydrogen, deuterium, halogen (e.g., F, or Cl), C₁₋₄ alkyl (e.g., methyl or ethyl), or C₁₋₄ heteroalkyl having 1 or 2 heteroatoms independently oxygen or nitrogen, such as N(CH₃)₂, preferably, R²³ is hydrogen, deuterium, F, Cl, or methyl.

35. The compound of any one of claims 11-33, or a pharmaceutically acceptable salt thereof, wherein R²³ is C₁₋₄ alkyl substituted with OH, NH₂, or NH(C₁₋₄ alkyl).

36. The compound of any one of claims 11-33, or a pharmaceutically acceptable salt thereof, wherein R²³ is

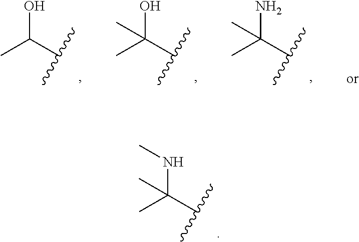

37. The compound of any one of claims 11-33, or a pharmaceutically acceptable salt thereof, wherein R²³ is a 4-6 membered heterocyclic ring having 1 or 2 ring heteroatoms, e.g., azetidinyl, pyrrolidinyl, piperidinyl, etc., wherein the 4-6 membered heterocyclic ring is optionally substituted with 1-3 substituents each independently oxo, F, OH, NH₂, C₁₋₄ alkyl optionally substituted with F, C₁₋₄ alkoxy optionally substituted with F, or NH(C₁₋₄ alkyl).

38. The compound of any one of claims 11-33, or a pharmaceutically acceptable salt thereof, wherein R²³ is azetidinyl or pyrrolidinyl, such as

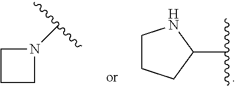 [SMILES: *N1CCC1 ; *C1CCCN1]

39. The compound of any one of claims 11-33, or a pharmaceutically acceptable salt thereof, wherein R²³ is a 5 membered heteroaryl ring having 2-4 ring heteroatoms, e.g., imidazolyl, pyrazolyl, etc., wherein the 5 membered heteroaryl ring is optionally substituted with 1-3 substituents each independently F, OH, NH₂, C₁₋₄ alkyl optionally substituted with F, C₁₋₄ alkoxy optionally substituted with F, or NH(C₁₋₄ alkyl), preferably, R²³ is an imidazolyl or pyrazolyl, optionally substituted with one or two C₁₋₄ alkyl such as methyl, such as

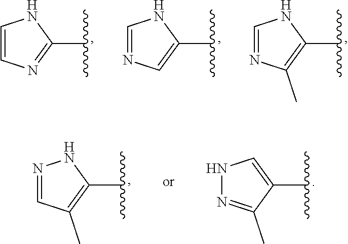

40. The compound of any one of claims 11-26, or a pharmaceutically acceptable salt thereof, wherein R²² and R²³, together with the intervening atoms, are joined together to form a 5 or 6 membered ring, preferably, a 5 or 6 membered carbocyclic ring, which is substituted with 1-3 substituents each independently F, OH, NH₂, C₁₋₄ alkyl optionally substituted with F, C₁₋₄ alkoxy optionally substituted with F, or NH(C₁₋₄ alkyl), preferably, one of the substitutent(s) comprises a hydrogen bond donor, such as OH, NH₂, or NH(C₁₋₄ alkyl).

41. The compound of any one of claims 1-9, or a pharmaceutically acceptable salt thereof, wherein R² has a structure according to M-2 or M-5: 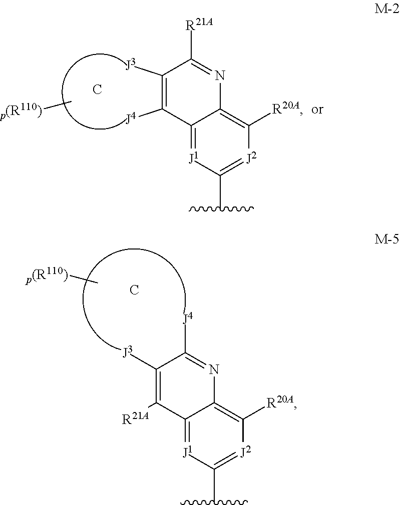
wherein:
J¹ and J² are independently N or CR³⁰, wherein R³⁰ at each occurrence is independently hydrogen, halogen, C₁₋₄ alkyl optionally substituted with F, or C₁₋₄ heteroalkyl optionally substituted with F;
Ring C is a heterocyclic ring or heteroaryl ring, which is optionally substituted with 1-5 instances of R¹⁰, i.e., p is 0-5, as valency permits, wherein
R¹¹⁰ at each occurrence is independently (1) oxo, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) two instances of R¹¹⁰, together with the intervening atom(s), are joined to form a 3-6 membered ring structure (e.g., cyclopropyl, cyclobutyl, 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F, J³ and J⁴ are independently N, NR³¹, CR³²R³³, or CR³⁴, wherein
R³¹ at each occurrence is independently hydrogen, C₁₋₄ alkyl optionally substituted with F, or an optionally substituted 3-6 membered ring structure (e.g., cyclopropyl, cyclobutyl, 4-6 membered heterocyclic ring, or 5 membered heteroaryl),
each of R³² and R³³ at each occurrence is independently (1) hydrogen or deuterium, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) an optionally substituted 3-6 membered ring structure (e.g., cyclopropyl, cyclobutyl, 4-6 membered heterocyclic ring, or 5 membered heteroaryl); or (7) R³² and R³³, together with the carbon atom they are both bond to, are joined to form a carbonyl (CO) or a 3-6 membered ring structure (e.g., cyclopropyl, cyclobutyl, or 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F; or
two instances of R³², or one instance of R³² and one instance of R¹⁰⁰, together with the intervening atom(s), are joined to form a 3-6 membered ring structure (e.g., cyclopropyl, cyclobutyl, 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F;
R³⁴ at each occurrence is independently (1) hydrogen, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) an optionally substituted 3-6 membered ring structure (e.g., cyclopropyl, cyclobutyl, 4-6 membered heterocyclic ring, or 5 membered heteroaryl); and
R^{20A} and R^{21A} are each independently hydrogen, deuterium, halogen (e.g., F or Cl), CN, C₁₋₄ alkyl optionally substituted with F, C₁₋₄ heteroalkyl optionally substituted with F, or an optionally substituted 3-6 membered ring structure (e.g., cyclopropyl, cyclobutyl, 4-6 membered heterocyclic ring, or 5 membered heteroaryl).

42. The compound of claim 41, or a pharmaceutically acceptable salt thereof, wherein Ring C is a heterocyclic ring having 1 or 2 ring heteroatoms, such as a 5 or 6 membered heterocyclic ring having 1 ring nitrogen atom; or Ring C is a 5-membered heteroaryl ring having 1 or 2 ring heteroatoms.

43. The compound of claim 41 or 42, or a pharmaceutically acceptable salt thereof, wherein J¹ is CR³⁰, and R³⁰ is hydrogen.

44. The compound of claim 41 or 42, or a pharmaceutically acceptable salt thereof, wherein J¹ is N.

45. The compound of any one of claims 41-44, or a pharmaceutically acceptable salt thereof, wherein J² is CR³⁰, and R³⁰ is hydrogen.

46. The compound of any one of claims 41-44, or a pharmaceutically acceptable salt thereof, wherein J² is N.

47. The compound of claim 41, or a pharmaceutically acceptable salt thereof, wherein the compound is a compound of Formula I-2:

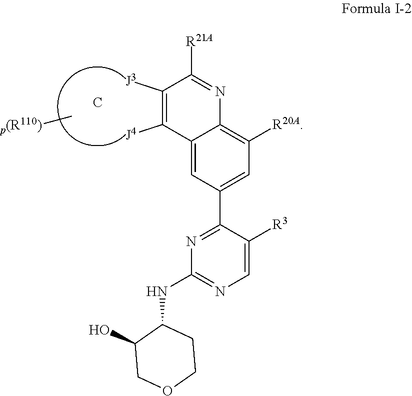

48. The compound of any one of claims 41-47, or a pharmaceutically acceptable salt thereof, wherein R^{20A} is hydrogen, halogen, or C₁₋₄ alkyl.

49. The compound of any one of claims 41-47, or a pharmaceutically acceptable salt thereof, wherein R^{20A} is hydrogen, F, Cl, methyl, or ethyl, preferably, R^{20A} is F.

50. The compound of any one of claims 41-49, or a pharmaceutically acceptable salt thereof, wherein R^{21A} is hydrogen, deuterium, halogen, or C₁₋₄ alkyl.

51. The compound of any one of claims 41-49, or a pharmaceutically acceptable salt thereof, wherein R^{21A} is hydrogen, deuterium, F, Cl, methyl, or ethyl, preferably, R^{21A} is hydrogen, deuterium, or methyl.

52. The compound of any one of claims 41-51, or a pharmaceutically acceptable salt thereof, wherein p is 0, or p is 1 and R¹¹⁰ is OH, NH₂, NH(C₁₋₄ alkyl), or N(C₁₋₄ alkyl)(C₁₋₄ alkyl), or C₁₋₄ alkyl.

53. The compound of any one of claims 41-51, or a pharmaceutically acceptable salt thereof, wherein p is 2, wherein R¹¹⁰ at each occurrence is independently C₁₋₄ alkyl (e.g., methyl, ethyl); or two R¹¹⁰ are joined together with the intervening atom(s) to form a 3-6 membered ring, such as a cyclopropyl ring.

54. The compound of any one of claims 41-53, or a pharmaceutically acceptable salt thereof, wherein J³ is NR³¹, and R³¹ is hydrogen or C₁₋₄ alkyl (e.g., methyl or ethyl).

55. The compound of any one of claims 41-53, or a pharmaceutically acceptable salt thereof, wherein J³ is N.

56. The compound of any one of claims 41-53, or a pharmaceutically acceptable salt thereof, wherein J³ is CR³⁴, and R³⁴ is hydrogen or C₁₋₄ alkyl (e.g., methyl or ethyl).

57. The compound of any one of claims 41-53, or a pharmaceutically acceptable salt thereof, wherein J³ is CR³²R³³, and R³² is hydrogen, deuterium, F, or C₁₋₄ alkyl (e.g., methyl or ethyl) and R³³ is hydrogen, deuterium, F, or C₁₋₄ alkyl (e.g., methyl or ethyl), e.g., J³ is CH₂, CD₂, CF₂, CH(CH₃), or C(CH₃)₂;

58. The compound of any one of claims 41-53, or a pharmaceutically acceptable salt thereof, wherein J³ is CR³²R³³, and R³² and R³³ together with the carbon atom they are both bonded to form a 3-4 membered ring, such as a cyclopropyl.

59. The compound of any one of claims 41-58, or a pharmaceutically acceptable salt thereof, wherein J⁴ is NR³¹, and R³¹ is hydrogen or C₁₋₄ alkyl (e.g., methyl or ethyl).

60. The compound of any one of claims 41-58, or a pharmaceutically acceptable salt thereof, wherein J⁴ is N.

61. The compound of any one of claims 41-58, or a pharmaceutically acceptable salt thereof, wherein J⁴ is CR³⁴, and R³⁴ is hydrogen or C₁₋₄ alkyl (e.g., methyl or ethyl).

62. The compound of any one of claims 41-58, or a pharmaceutically acceptable salt thereof, wherein J⁴ is CR³²R³³, and R³² is hydrogen, deuterium, F, or C₁₋₄ alkyl (e.g., methyl or ethyl) and R³³ is hydrogen, deuterium, F, or C₁₋₄ alkyl (e.g., methyl or ethyl), e.g., J⁴ is CH₂, CD₂, CF₂, CH(CH₃), or C(CH₃)₂.

63. The compound of any one of claims 41-58, or a pharmaceutically acceptable salt thereof, wherein J⁴ is CR³²R³³, and R³² and R³³ together with the carbon atom they are both bonded to form a 3-4 membered ring, such as a cyclopropyl; or J⁴ is C(O).

64. The compound of any one of claims 41-63, or a pharmaceutically acceptable salt thereof, wherein as applicable, R² is characterized as having a structure according to formula F-1, F-2, F-3, F-4, F-5, F-6, F-7, F-8, F-9, F-10, F-11, F-12, F-13, or F-14: 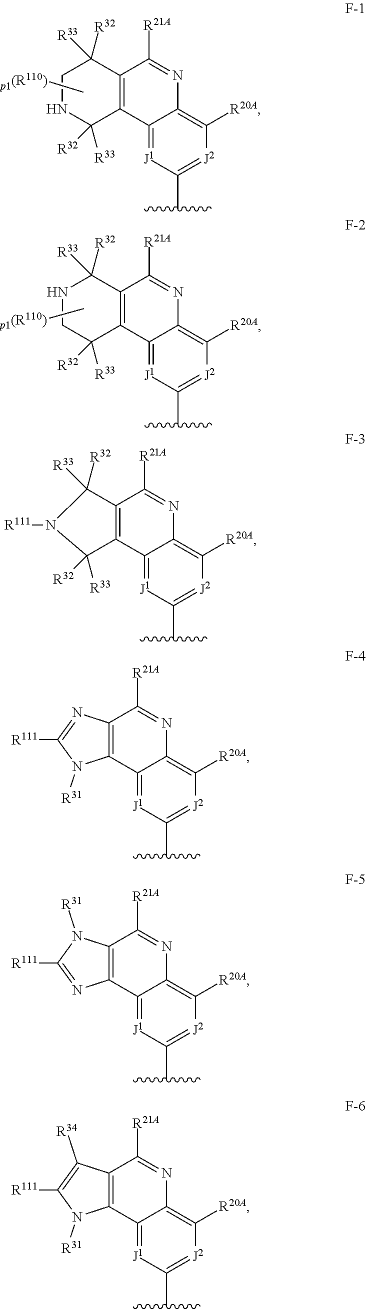 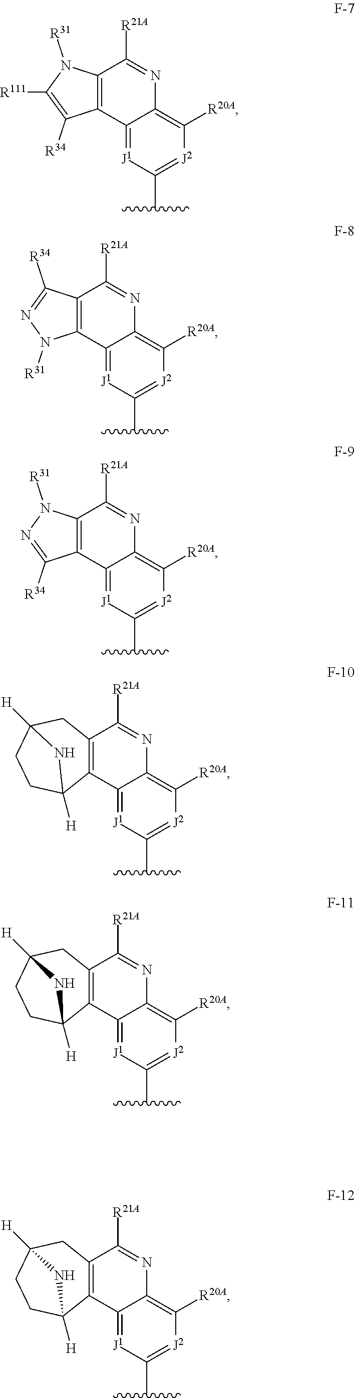 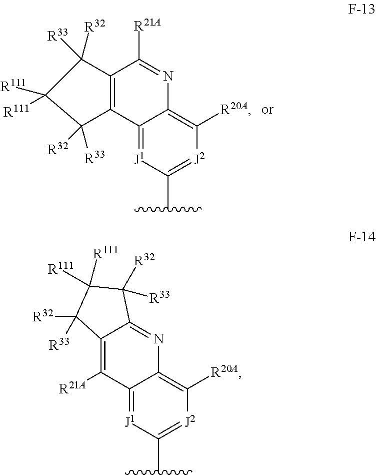
wherein:
p1 is 0, 1, 2, or 3, as valency permits; and
R¹¹¹ is hydrogen or R¹¹⁰, and
the variables J¹, J², R^{20A}, R^{21A}, R³¹, R³², R³³, R³⁴, and R¹¹⁰ are as defined in any of claims 39-60, as applicable.

65. The compound of claim 64, or a pharmaceutically acceptable salt thereof, wherein as applicable, R² is characterized as having a structure according to formula F-1-1, F-2-1, F-3-1, F-4-1, F-5-1, F-6-1, F-7-1, F-8-1, F-9-1, F-10-1, F-11-1, F-12-1, F-13-1, or F-14-1:

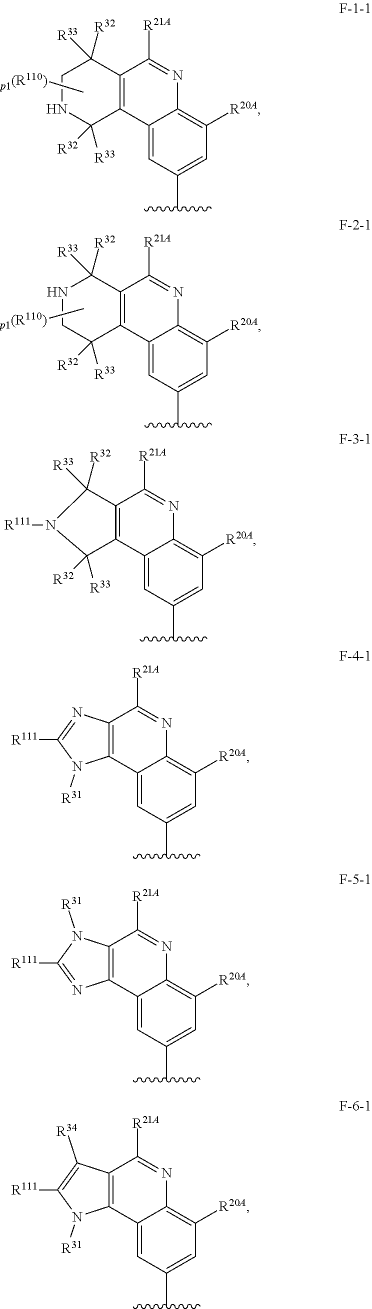

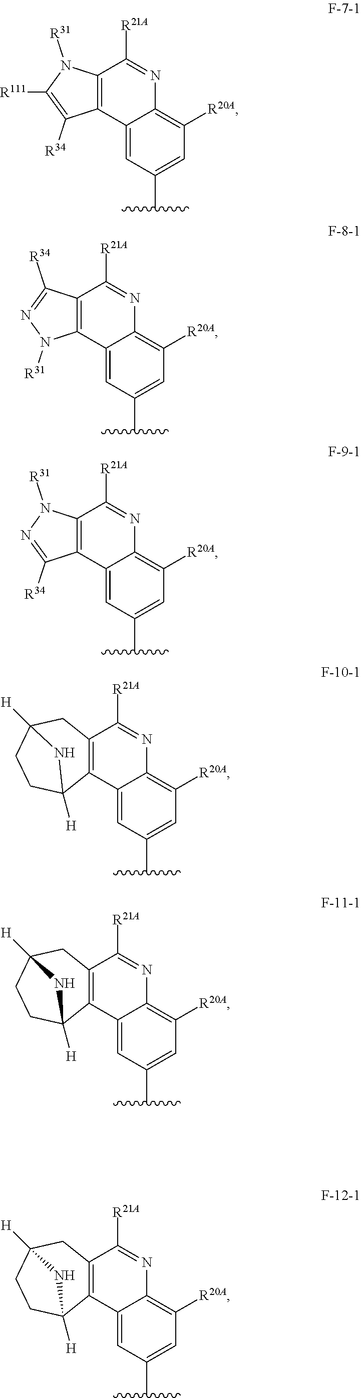

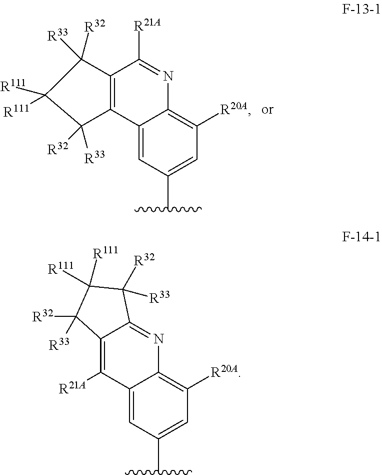

66. The compound of claim 64 or 65, or a pharmaceutically acceptable salt thereof, wherein in F-1 or F-2 (or sub-formula thereof), p1 is 0, or p1 is 1 and R¹¹⁰ is C₁₋₄ alkyl.

67. The compound of claim 64 or 65, or a pharmaceutically acceptable salt thereof, wherein in F-1 or F-2 (or sub-formula thereof), p is 2, wherein R¹¹⁰ at each occurrence is independently C₁₋₄ alkyl (e.g., methyl, ethyl); or two R¹¹⁰ are joined together with the intervening atom(s) to form a 3-6 membered ring, such as a cyclopropyl ring.

68. The compound of claim 64 or 65, or a pharmaceutically acceptable salt thereof, wherein in F-3, F-4, F-5, F-6, or F-7 (or sub-formula thereof), R¹¹¹ is hydrogen or C₁₋₄ alkyl.

69. The compound of claim 64 or 65, or a pharmaceutically acceptable salt thereof, wherein in F-4, F-5, F-6, or F-7 (or sub-formula thereof), R¹¹¹ is NH₂ or NH(C₁₋₄ alkyl).

70. The compound of any one of claims 1-9, or a pharmaceutically acceptable salt thereof, wherein R² has a structure according to M-3 or M-4: 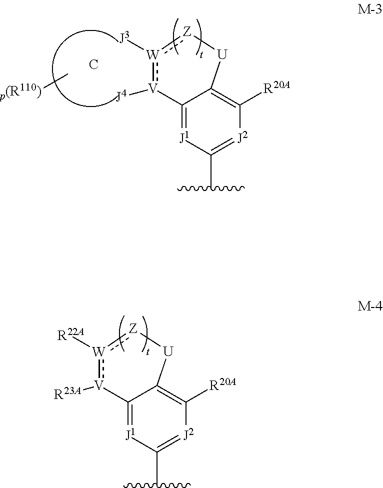
wherein:
J¹ and J² are independently N or CR³⁰, wherein R³⁰ at each occurrence is independently hydrogen, halogen, C₁₋₄ alkyl optionally substituted with F, or C₁₋₄ heteroalkyl optionally substituted with F;
Ring C is a heterocyclic ring or heteroaryl ring, which is optionally substituted with 1-5 instances of R¹⁰, i.e., p is 0-5, as valency permits, wherein
R¹¹⁰ at each occurrence is independently (1) oxo, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) two instances of R¹⁰ together with the intervening atom(s), are joined to form a 3-6 membered ring structure (e.g., cyclopropyl, cyclobutyl, 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F, U is O or C(═O);
W and V are independently C, N, or CR³⁴;
t is 0, 1, 2, or 3, each Z is independently O, N, NR³¹, CR³²R³³, or CR³⁴;
wherein the bond between W and V, W and Z, or two consecutive Z, is a single bond or a double bond, as valency permits,
J³ and J⁴ are independently N, NR³¹, CR³²R¹³, or CR³⁴, wherein
R³¹ at each occurrence is independently hydrogen, C₁₋₄ alkyl optionally substituted with F, or an optionally substituted 3-6 membered ring structure (e.g., cyclopropyl, cyclobutyl, 4-6 membered heterocyclic ring, or 5 membered heteroaryl),
each of R³² and R³³ at each occurrence is independently (1) hydrogen, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) an optionally substituted 3-6 membered ring structure (e.g., cyclopropyl, cyclobutyl, 4-6 membered heterocyclic ring, or 5 membered heteroaryl); or (7) R³² and R³³, together with the carbon atom they are both bond to, are joined to form a carbonyl (CO) or a 3-6 membered ring structure (e.g., cyclopropyl, cyclobutyl, or 4-6 membered heterocyclic ring) which is optionally substituted 1-3 substituents each independently oxo, halo (e.g., F), OH, NH₂, C₁₋₄ alkyl optionally substituted with F, or C₁₋₄ heteroalkyl optionally substituted with F;
R³⁴ at each occurrence is independently (1) hydrogen, (2) halogen (e.g., F or Cl), (3) OH, NH₂, NH(C₁₋₄ alkyl), or N(C₁₋₄ alkyl)(C₁₋₄ alkyl), (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) an optionally substituted 3-6 membered ring structure (e.g., cyclopropyl, cyclobutyl, 4-6 membered heterocyclic ring, or 5 membered heteroaryl);
or J³ and one instance of R¹¹⁰ or J⁴ and one instance of R¹¹⁰ are joined together with the intervening atom(s) to form a ring structure;
R^{20A} is hydrogen, halogen (e.g., F or Cl), CN, C₁₋₄ alkyl optionally substituted with F, C₁₋₄ heteroalkyl optionally substituted with F, or an optionally substituted 3-6 membered ring structure (e.g., cyclopropyl, cyclobutyl, 4-6 membered heterocyclic ring, or 5 membered heteroaryl); and
R^{22A} and R^{23A} are each independently: (1) hydrogen; (2) halogen (e.g., F or Cl), (3) CN, (4) C₁₋₄ alkyl optionally substituted with 1-3 substituents each independently F, OH, NH₂, NH(C₁₋₄ alkyl), N(C₁₋₄ alkyl)(C₁₋₄ alkyl), NH(C₃₋₆ cycloalkyl), N(C₁₋₄ alkyl)(C₃₋₆ cycloalkyl), or N(C₃₋₆ cycloalkyl)(C₃₋₆ cycloalkyl), (5) C₁₋₄ heteroalkyl optionally substituted with F, or (6) a 3-6 membered ring structure (e.g., cyclopropyl, cyclobutyl, 4-6 membered heterocyclic ring, or 5 membered heteroaryl) optionally substituted 1-3 substituents each independently oxo, halo (e.g., F), OH, NH₂, C₁₋₄ alkyl optionally substituted with F, C₁₋₄ heteroalkyl optionally substituted with F.

71. The compound of claim 70, or a pharmaceutically acceptable salt thereof, wherein Ring C is a heterocyclic ring having 1 or 2 ring heteroatoms, such as a 5 or 6 membered heterocyclic ring having 1 or 2 ring nitrogen atoms; or Ring C is a 5-membered heteroaryl ring having 1 or 2 ring heteroatoms.

72. The compound of claim 70 or 71, or a pharmaceutically acceptable salt thereof, wherein J^{P} is CR³⁰, and R³⁰ is hydrogen.

73. The compound of claim 70 or 71, or a pharmaceutically acceptable salt thereof, wherein J¹ is N.

74. The compound of any one of claims 70-73, or a pharmaceutically acceptable salt thereof, wherein J² is CR³⁰, and R³⁰ is hydrogen.

75. The compound of any one of claims 70-73, or a pharmaceutically acceptable salt thereof, wherein J² is N.

76. The compound of claim 70, or a pharmaceutically acceptable salt thereof, wherein the compound is a compound of Formula I-3 or I-4:

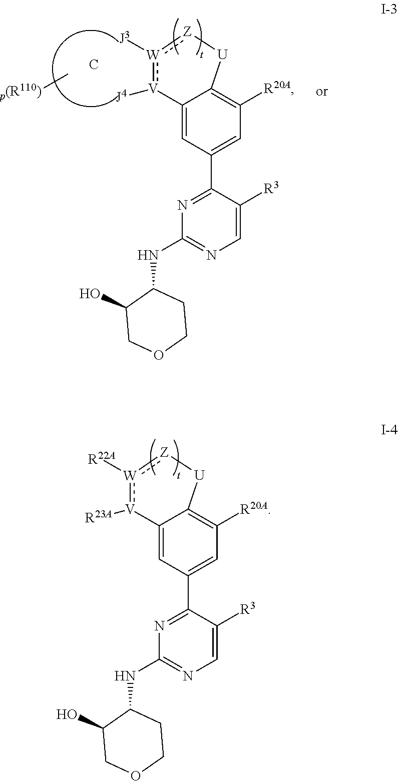

77. The compound of any one of claims 70-76, or a pharmaceutically acceptable salt thereof, wherein R^{21A} is hydrogen, halogen, or C₁₋₄ alkyl.

78. The compound of any one of claims 70-76, or a pharmaceutically acceptable salt thereof, wherein R^{20A} is hydrogen, F, Cl, methyl, or ethyl.

79. The compound of any one of claims 70-78, or a pharmaceutically acceptable salt thereof, wherein p is 0, or p is 1 and R¹¹⁰ is OH, NH₂, NH(C₁₋₄ alkyl), or N(C₁₋₄ alkyl)(C₁₋₄ alkyl), or C₁₋₄ alkyl.

80. The compound of any one of claims 70-78, or a pharmaceutically acceptable salt thereof, wherein p is 2, wherein R¹¹⁰ at each occurrence is independently C₁₋₄ alkyl (e.g., methyl, ethyl); or two R¹¹⁰ are joined together with the intervening atom(s) to form a 3-6 membered ring, such as a cyclopropyl ring.

81. The compound of any one of claims 70-80, or a pharmaceutically acceptable salt thereof, wherein J³ is NR³¹, and R³¹ is hydrogen or C₁₋₄ alkyl (e.g., methyl or ethyl).

82. The compound of any one of claims 70-80, or a pharmaceutically acceptable salt thereof, wherein J³ is N.

83. The compound of any one of claims 70-80, or a pharmaceutically acceptable salt thereof, wherein J³ is CR³⁴, and R³⁴ is hydrogen or C₁₋₄ alkyl (e.g., methyl or ethyl).

84. The compound of any one of claims 70-80, or a pharmaceutically acceptable salt thereof, wherein J³ is CR³²R³³, and R³² is hydrogen or C₁₋₄ alkyl (e.g., methyl or ethyl) and R³³ is hydrogen or C₁₋₄ alkyl (e.g., methyl or ethyl), e.g., J³ is CH₂, CH(CH₃), or C(CH₃)₂;

85. The compound of any one of claims 70-80, or a pharmaceutically acceptable salt thereof, wherein J³ is CR³²R³³, and R³² and R³³ together with the carbon atom they are both bonded to form a 3-4 membered ring, such as a cyclopropyl.

86. The compound of any one of claims 70-85, or a pharmaceutically acceptable salt thereof, wherein J⁴ is NR³¹, and R³¹ is hydrogen or C₁₋₄ alkyl (e.g., methyl or ethyl).

87. The compound of any one of claims 70-85, or a pharmaceutically acceptable salt thereof, wherein J⁴ is N.

88. The compound of any one of claims 70-85, or a pharmaceutically acceptable salt thereof, wherein J⁴ is CR³⁴, and R³⁴ is hydrogen or C₁₋₄ alkyl (e.g., methyl or ethyl).

89. The compound of any one of claims 70-85, or a pharmaceutically acceptable salt thereof, wherein J⁴ is CR³²R³³, and R³² is hydrogen or C₁₋₄ alkyl (e.g., methyl or ethyl) and R³³ is hydrogen or C₁₋₄ alkyl (e.g., methyl or ethyl), e.g., J⁴ is CH₂, CH(CH₃), or C(CH₃)₂.

90. The compound of any one of claims 70-85, or a pharmaceutically acceptable salt thereof, wherein J⁴ is CR³²R³³, and R³² and R³³ together with the carbon atom they are both bonded to form a 3-4 membered ring, such as a cyclopropyl.

91. The compound of any one of claims 70-90, or a pharmaceutically acceptable salt thereof, wherein U is O.

92. The compound of any one of claims 70-90, or a pharmaceutically acceptable salt thereof, wherein U is C(═O).

93. The compound of any one of claims 70-92, or a pharmaceutically acceptable salt thereof, wherein t is 1 or 2.

94. The compound of any one of claims 70-93, or a pharmaceutically acceptable salt thereof, wherein Z at each occurrence is independently CR³²R³³, and R³² is hydrogen or C₁₋₄ alkyl (e.g., methyl or ethyl) and R³³ is hydrogen or C₁₋₄ alkyl (e.g., methyl or ethyl), e.g., Z is CH₂, CH(CH₃), or C(CH₃)₂.

95. The compound of any one of claims 70-93, or a pharmaceutically acceptable salt thereof, wherein one instance of Z is CR³²R³³, wherein R³² and R³³ together with the carbon atom they are both bonded to form a 3-4 membered ring, such as a cyclopropyl; and any remaining instances of Z is independently CR³²R³³, wherein R³² is hydrogen or C₁₋₄ alkyl (e.g., methyl or ethyl) and R³³ is hydrogen or C₁₋₄ alkyl (e.g., methyl or ethyl), e.g., the remaining instances of Z is independently CH₂, CH(CH₃), or C(CH₃)₂.

96. The compound of any one of claims 70-93, or a pharmaceutically acceptable salt thereof, wherein W is C, and the Z directly connecting to W is CR³⁴, wherein R³⁴ is hydrogen or C₁₋₄ alkyl (e.g., methyl or ethyl), and the bond between Z and W is a double bond.

97. The compound of any one of claims 70-95, or a pharmaceutically acceptable salt thereof, wherein W is C or CH.

98. The compound of any one of claims 70-97, or a pharmaceutically acceptable salt thereof, wherein V is C or N, or V is Cu.

99. The compound of any one of claims 70-90, or a pharmaceutically acceptable salt thereof, wherein R² has a structure according to M-3-A, M-4-A, M-3-B, M-4-B, M-3-C, M-4-C, M-3-D, or M-4-D:

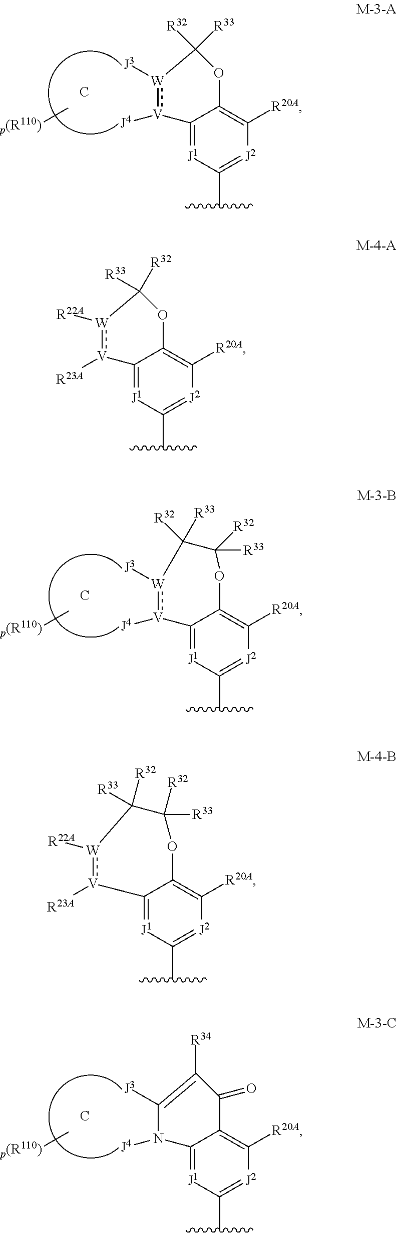

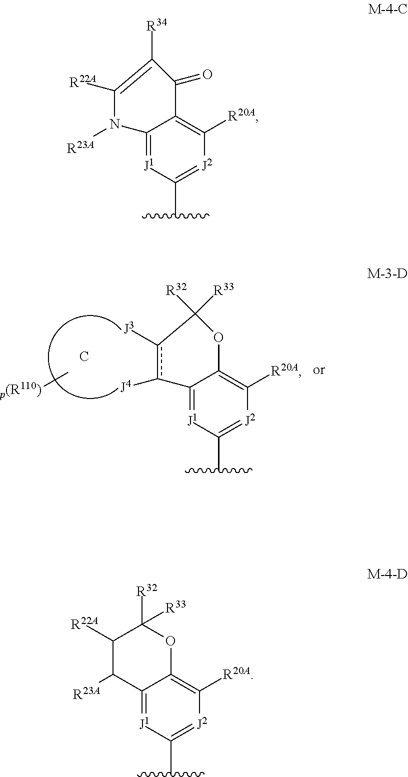

100. The compound of claim 99, or a pharmaceutically acceptable salt thereof, wherein R² has a structure according to M-3-A-1, M-3-A-2, M-3-A-3, M-4-A-1, M-3-B-1, M-3-B-2, or M-3-C-1: 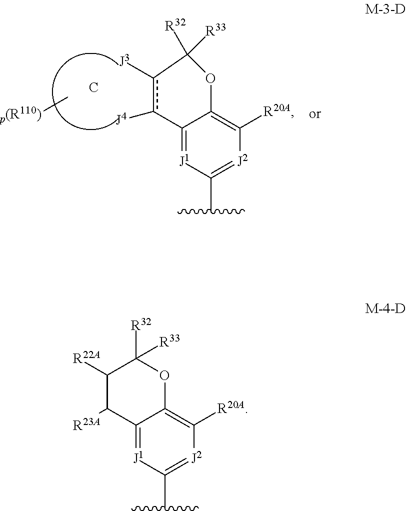
wherein:
R¹¹ is hydrogen or R¹¹⁰.

101. The compound of any one of claims 70-100, or a pharmaceutically acceptable salt thereof, wherein R^{22A} is hydrogen, C₁₋₄ alkyl (e.g., methyl or ethyl), or C₁₋₄ heteroalkyl having 1 or 2 heteroatoms independently oxygen or nitrogen.

102. The compound of any one of claims 70-100, or a pharmaceutically acceptable salt thereof, wherein R^{22A} is C₁₋₄ alkyl substituted with OH, NH₂, or NH(C₁₋₄ alkyl).

103. The compound of any one of claims 70-102, or a pharmaceutically acceptable salt thereof, wherein R^{23A} is hydrogen, C₁₋₄ alkyl (e.g., methyl or ethyl), or C₁₋₄ heteroalkyl having 1 or 2 heteroatoms independently oxygen or nitrogen.

104. The compound of any one of claims 70-102, or a pharmaceutically acceptable salt thereof, wherein R^{23A} is C₁₋₄ alkyl substituted with OH, NH₂, or NH(C₁₋₄ alkyl).

105. The compound of any one of claims 1-9, or a pharmaceutically acceptable salt thereof, wherein R² is selected from: 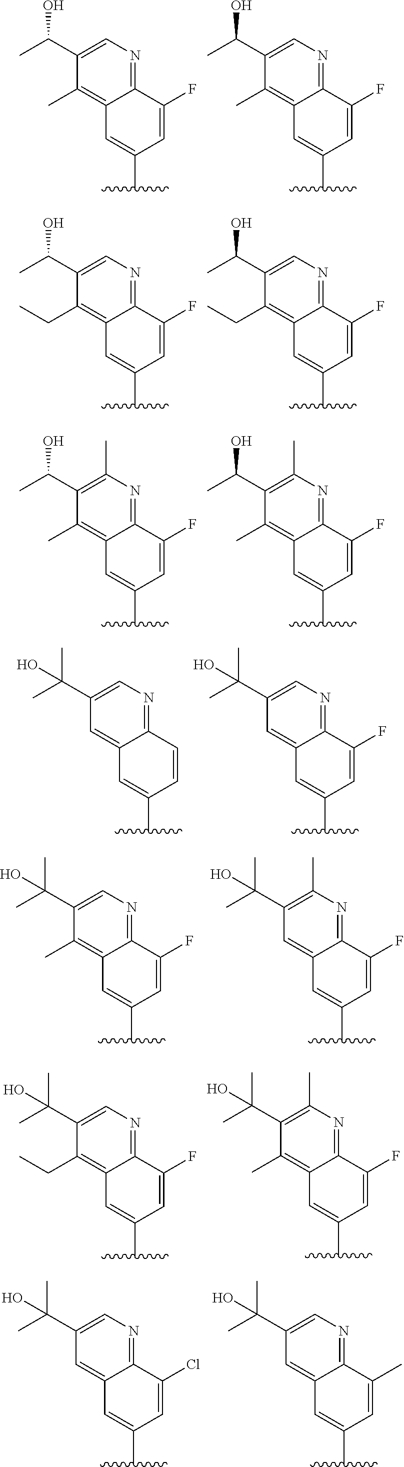 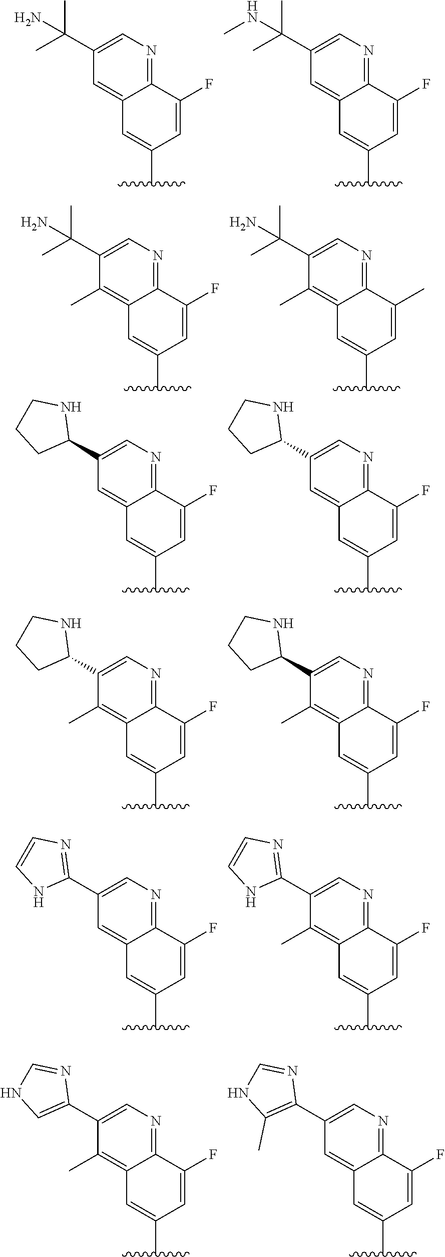 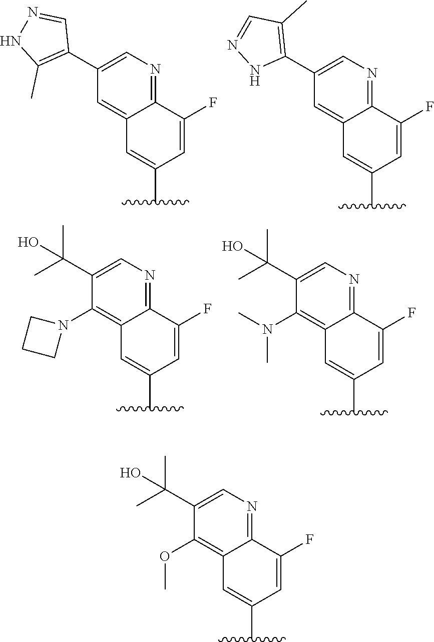
or R² is selected from: 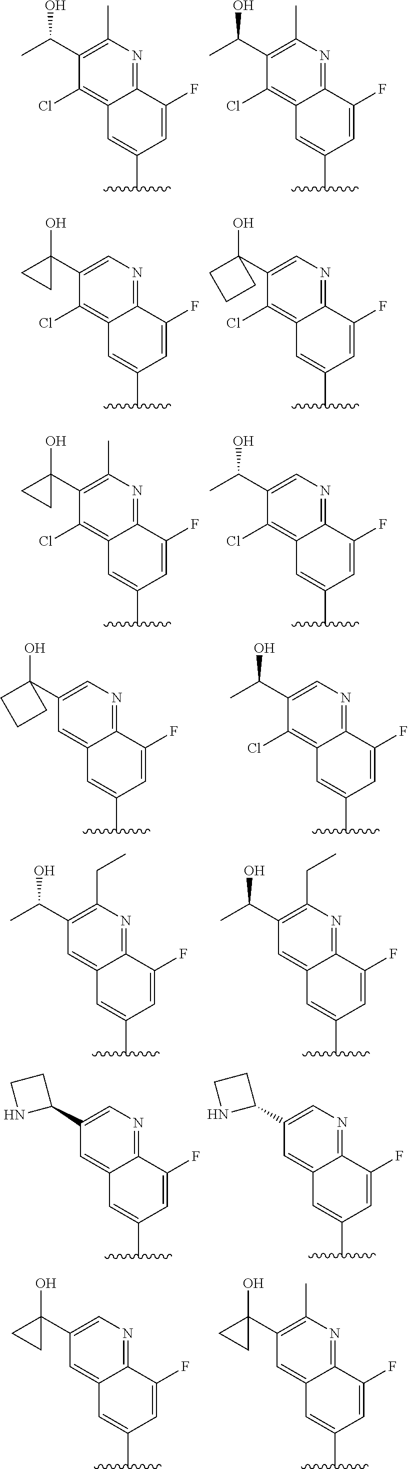 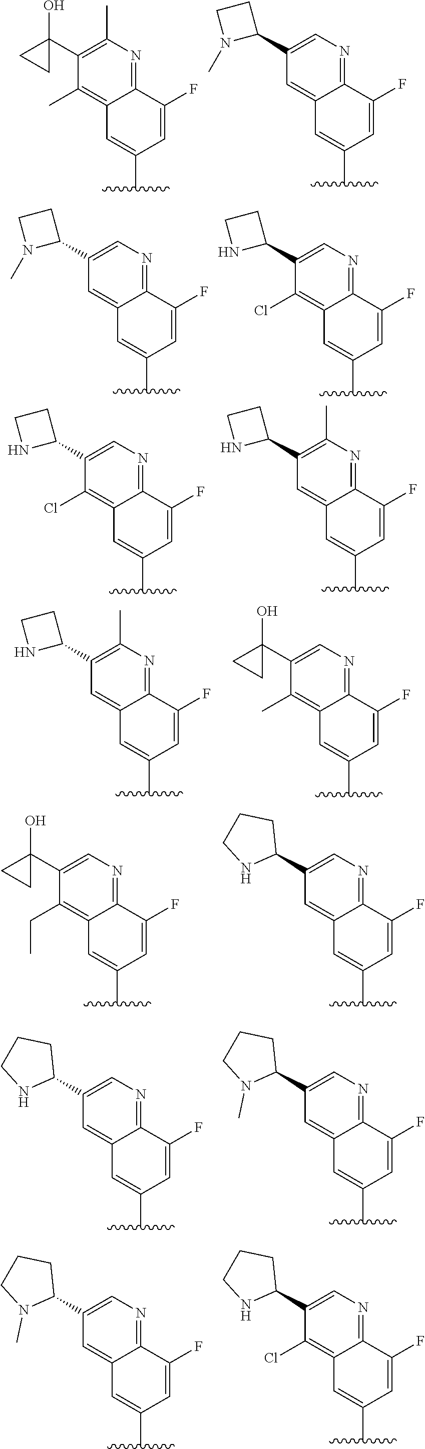 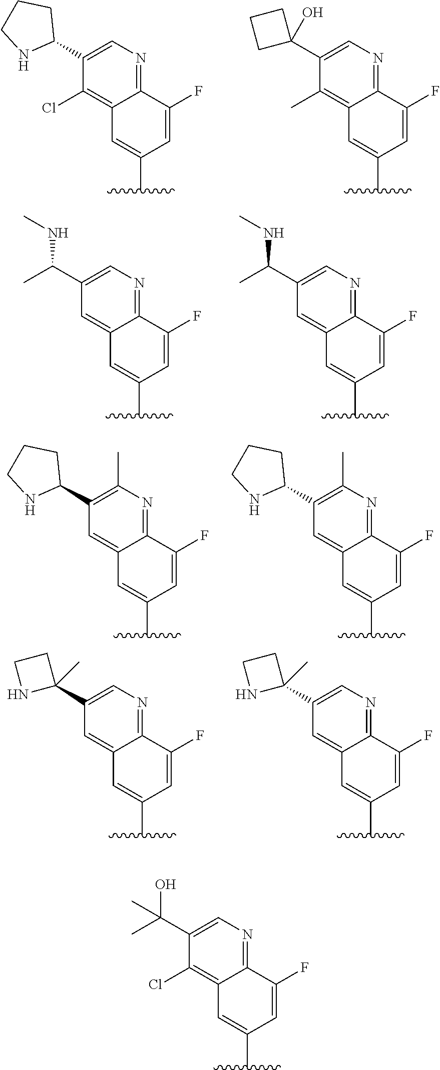
or R² is selected from:

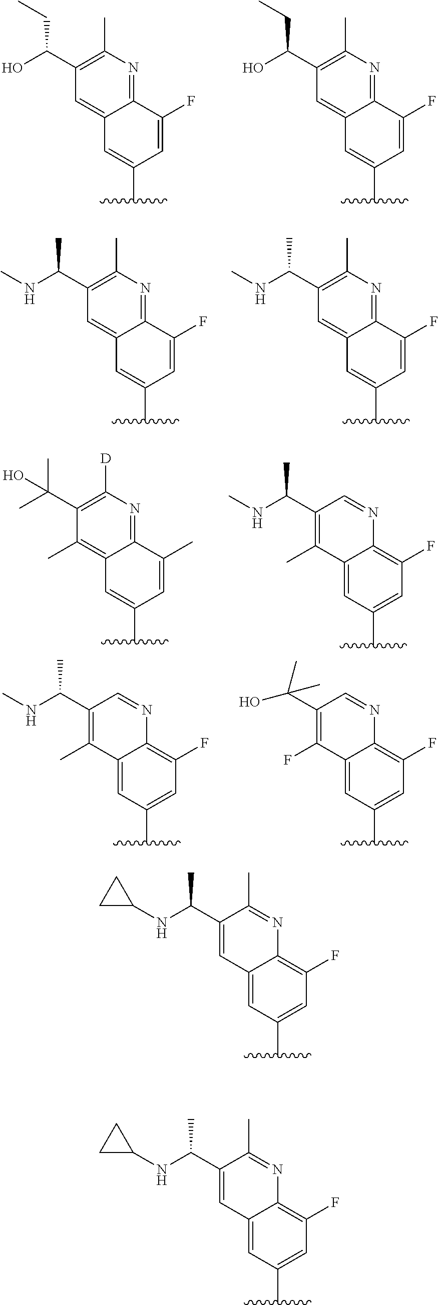

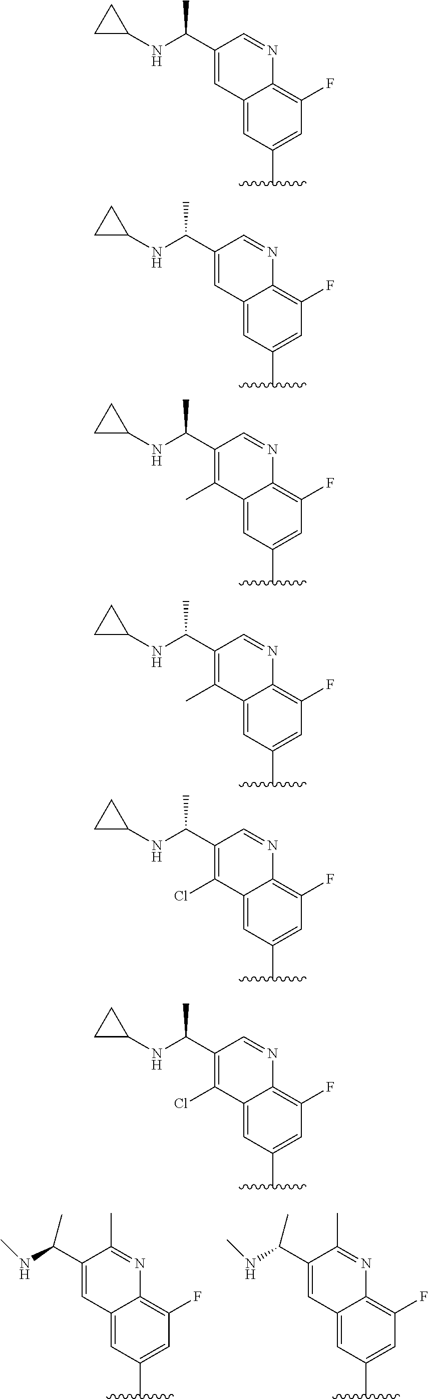

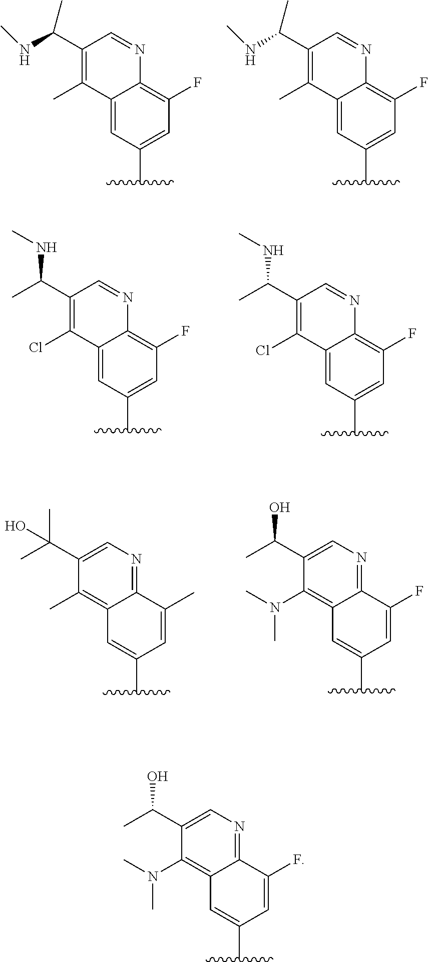

106. The compound of any one of claims 1-9, or a pharmaceutically acceptable salt thereof, wherein R² is selected from: 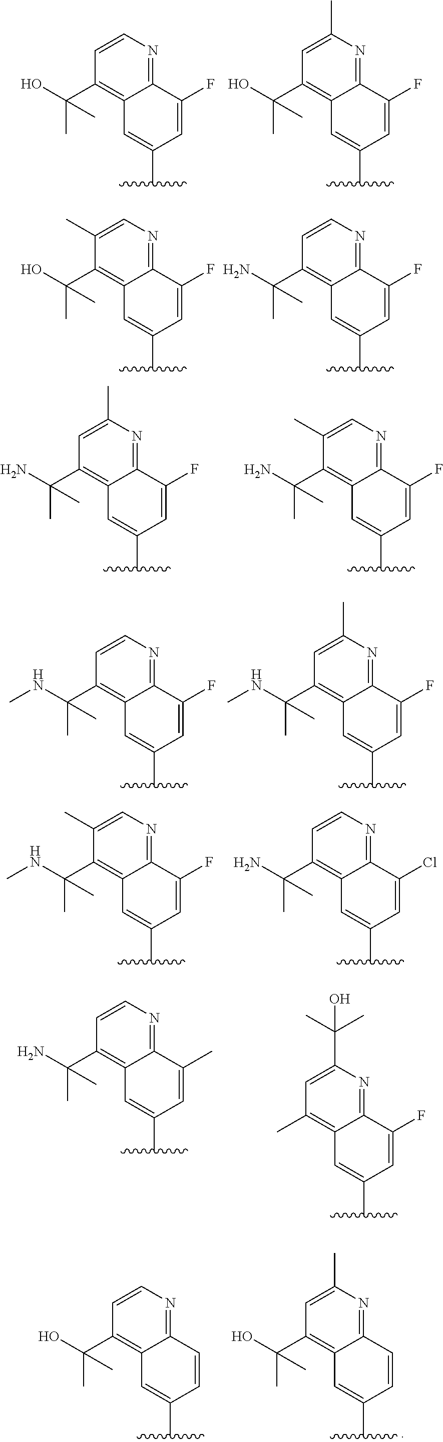
or R² is selected from

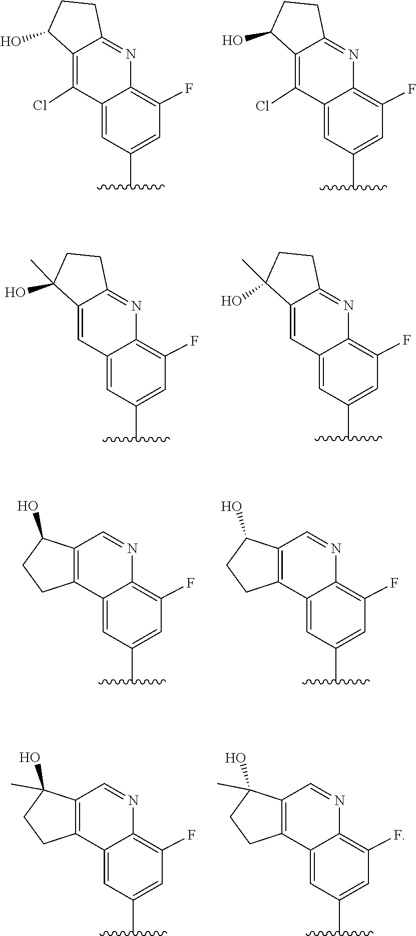

107. The compound of any one of claims 1-9, or a pharmaceutically acceptable salt thereof, wherein R² is selected from: 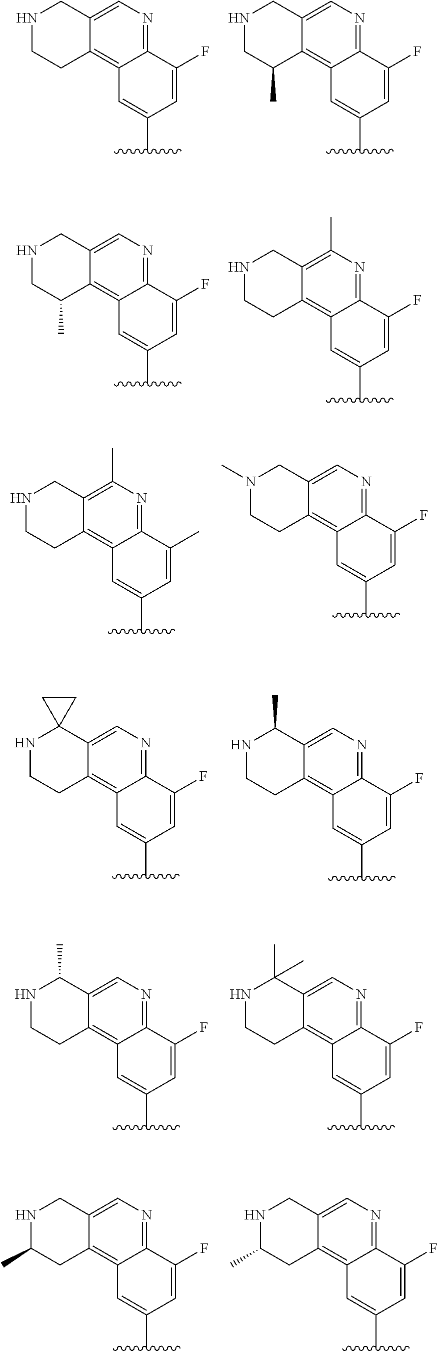 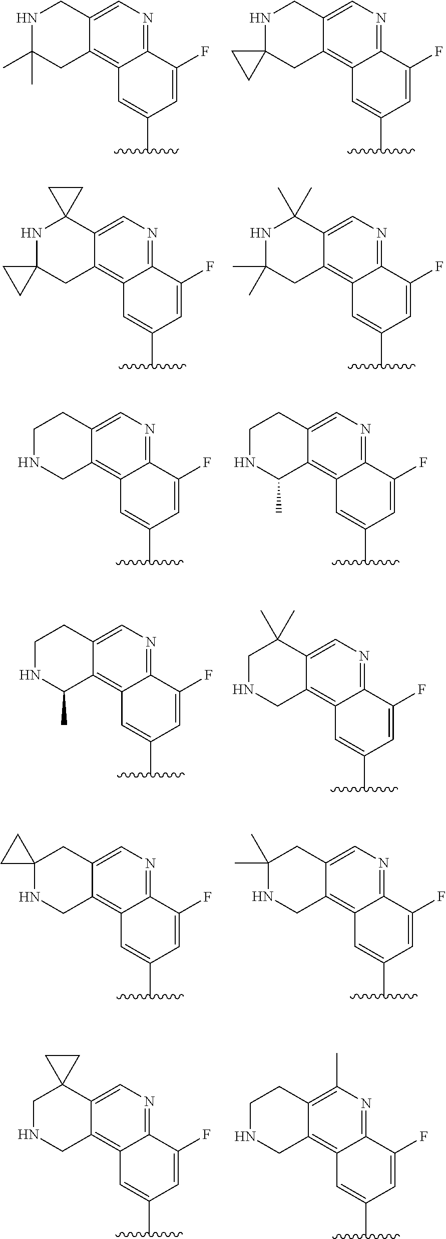 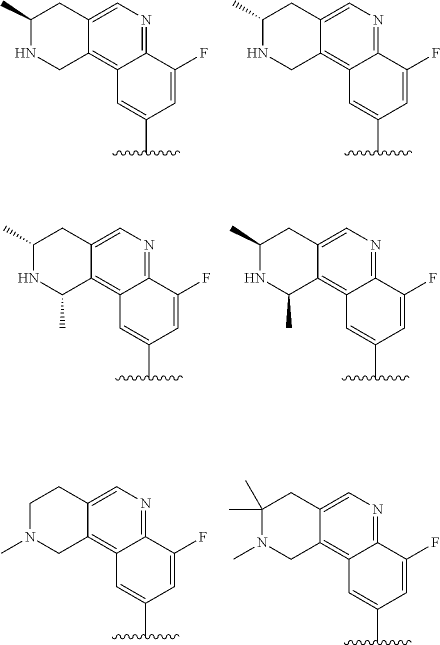
or R² is selected from 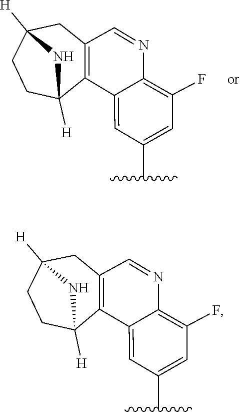 [SMILES: *c1cc(F)c2ncc3c(c2c1)[C@@H]1CC[C@H](C3)N1 ; *c1cc(F)c2ncc3c(c2c1)[C@H]1CC[C@@H](C3)N1]
or R² is selected from:

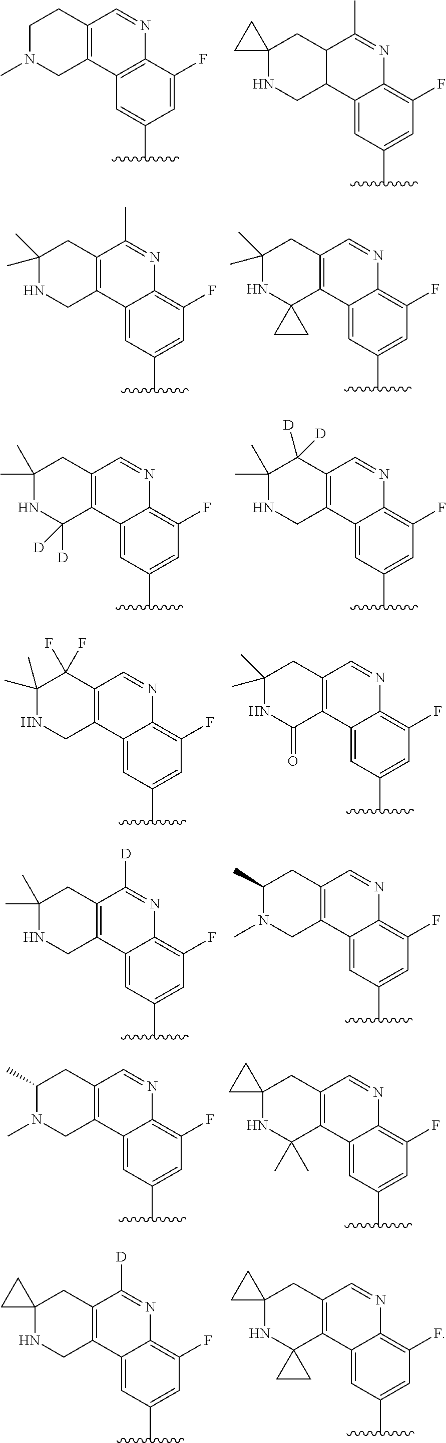

108. The compound of any one of claims 1-9, or a pharmaceutically acceptable salt thereof, wherein R² is selected from:

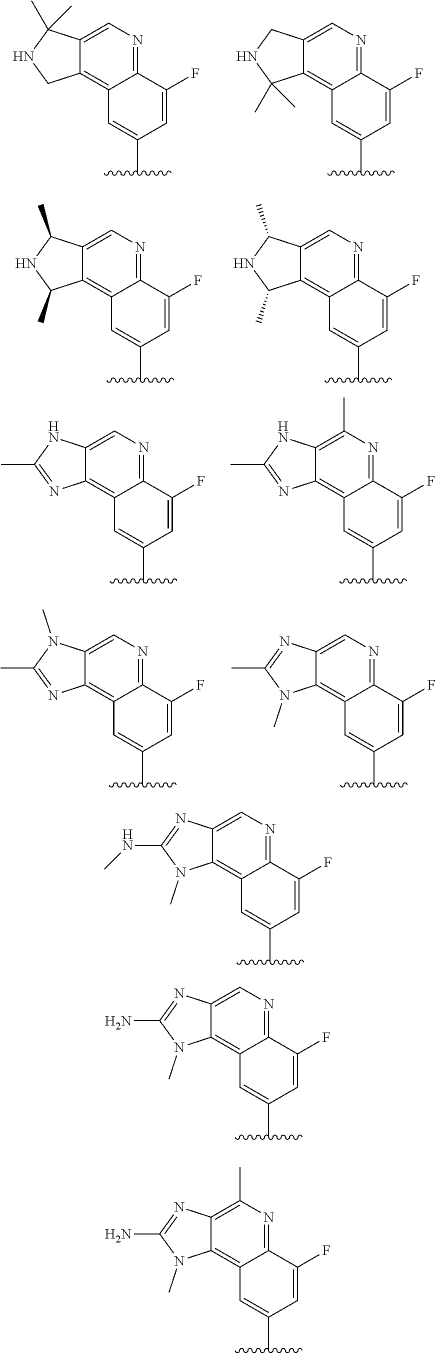

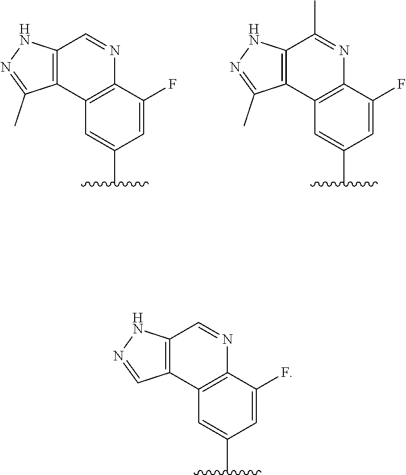 [SMILES: *c1cc(F)c2ncc3[nH]nc(C)c3c2c1 ; *c1cc(F)c2nc(C)c3[nH]nc(C)c3c2c1 ; *c1cc(F)c2ncc3[nH]ncc3c2c1]

109. The compound of any one of claims 1-9, or a pharmaceutically acceptable salt thereof, wherein R² is selected from:

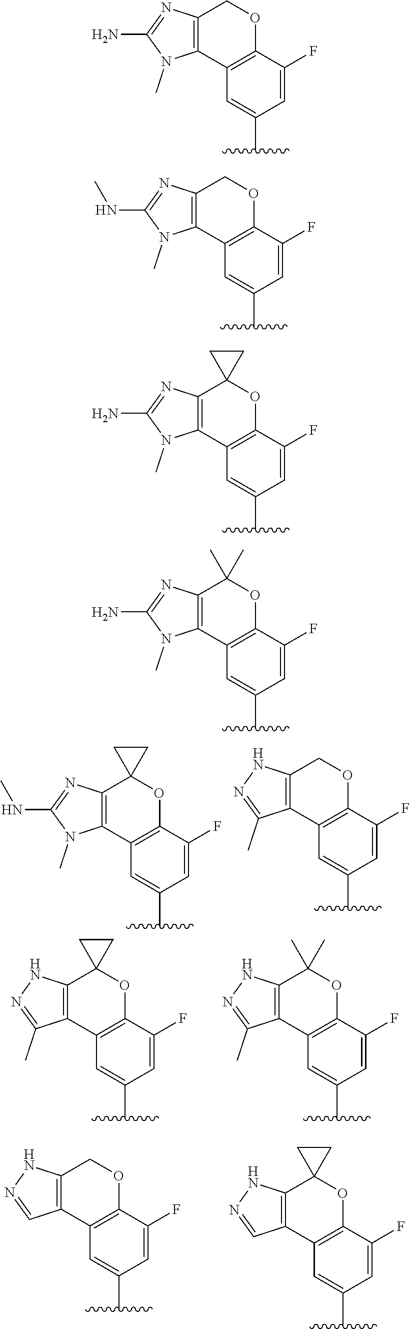

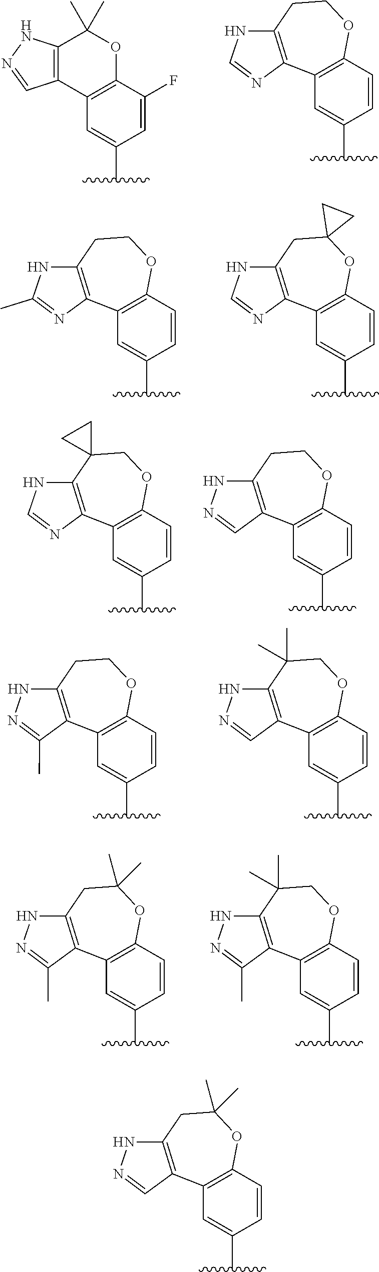

110. The compound of any one of claims 1-9, or a pharmaceutically acceptable salt thereof, wherein R² is selected from: 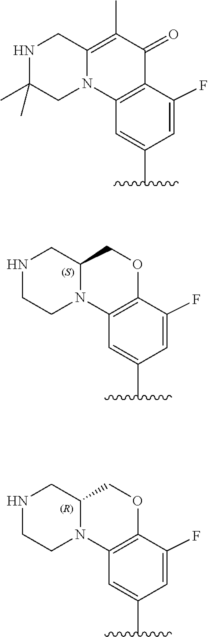
or R² is selected from

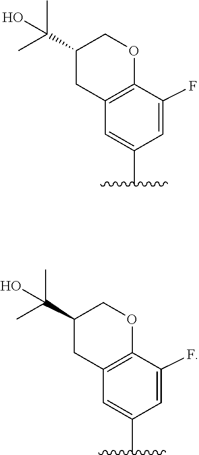 [SMILES: *c1cc(F)c2c(c1)C[C@H](C(C)(C)O)CO2 ; *c1cc(F)c2c(c1)C[C@@H](C(C)(C)O)CO2]

111. The compound of any one of claims 1-110, or a pharmaceutically acceptable salt thereof wherein R³ is hydrogen, F, Cl, Br, C₁₋₄ alkyl optionally substituted with F and/or deuterium (e.g., methyl, CD₃, ethyl, CHF₂, CF₂CH₃, CF₂CF₃, CH₂CH₂F, CH₂CF₂H, or CF₃), or CN.

112. The compound of any one of claims 1-110, or a pharmaceutically acceptable salt thereof wherein R³ is a C₃₋₆ cycloalkyl, 4-6 membered heterocyclyl having 1-2 ring heteroatoms independently selected from N, O, and S, or 5-6 membered heteroaryl having 1-4 ring heteroatoms independently selected from N, O, and S, each of which is optionally substituted with 1-3 substituents independently selected from oxo (as applicable), deuterium, F, CN, G¹, OH, O-G¹, NH₂, NH(G¹), N(G¹)(G¹), C(O)—NH₂, C(O)—NH(G¹), and C(O)—N(G¹)(G¹), wherein G¹ at each occurrence is independently a C₁₋₄ alkyl optionally substituted with 1-3 substituents independently selected from deuterium, F, CN, OH, and C₁₋₄ heteroalkyl, or a C₃₋₆ cycloalkyl optionally substituted with 1-3 substituents independently selected from deuterium, F, CN, OH, and C₁₋₄ heteroalkyl.

113. The compound of any one of claims 1-110, or a pharmaceutically acceptable salt thereof wherein R³ is selected from:

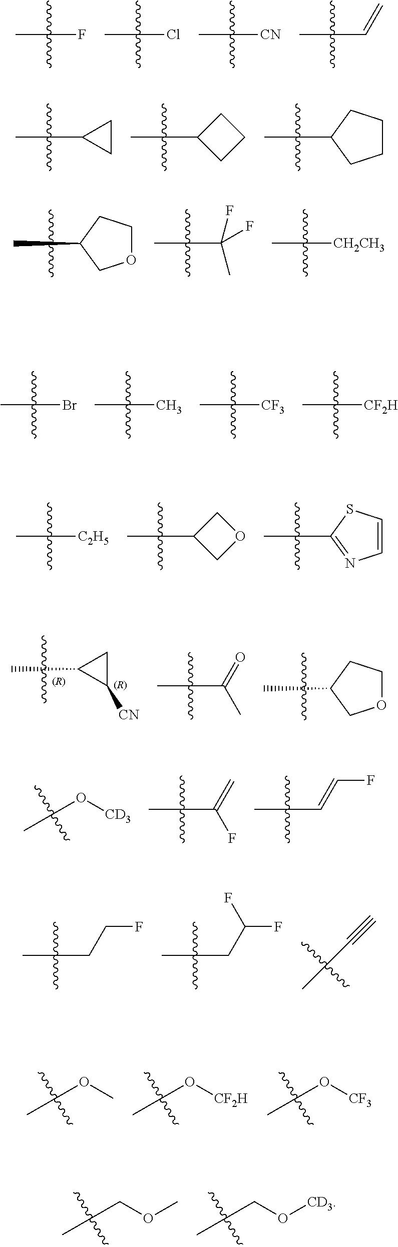

114. The compound of any one of claims 1-113, or a pharmaceutically acceptable salt thereof wherein R⁴ is hydrogen or deuterium.

115. A compound selected from Examples 1-111 or the compounds shown in Table 1 herein, a stereoisomer thereof, a deuterated analog thereof, or a pharmaceutically acceptable salt thereof.

116. A pharmaceutical composition comprising the compound of any one of claims 1-115, or a pharmaceutically acceptable salt thereof, and a pharmaceutically acceptable excipient.

117. A method of treating cancer in a subject in need thereof, the method comprising administering to the subject a therapeutically effective amount of the compound of any one of claims 1-115, or a pharmaceutically acceptable salt thereof, or the pharmaceutical composition of claim 116.

118. The method of claim 117, wherein the cancer is selected from breast cancer, ovarian cancer, bladder cancer, uterine cancer, prostate cancer, lung cancer (including NSCLC, SCLC, squamous cell carcinoma or adenocarcinoma), esophageal cancer, head and neck cancer, colorectal cancer, kidney cancer (including RCC), liver cancer (including HCC), pancreatic cancer, stomach (i.e., gastric) cancer and thyroid cancer.

119. The method of claim 117, wherein the cancer is selected from breast cancer, ovarian cancer, bladder cancer, uterine cancer, prostate cancer, lung cancer, esophageal cancer, liver cancer, pancreatic cancer and stomach cancer.

120. The method of claim 117, wherein the cancer is breast cancer selected from ER-positive/HR-positive, HER2-negative breast cancer; ER-positive/HR-positive, HER2-positive breast cancer; triple negative breast cancer (TNBC); and inflammatory breast cancer.

121. The method of claim 117, wherein the cancer is breast cancer selected from endocrine resistant breast cancer, trastuzumab resistant breast cancer, or breast cancer demonstrating primary or acquired resistance to CDK4/CDK6 inhibition.

122. The method of claim 117, wherein the cancer is advanced or metastatic breast cancer.

123. The method of claim 117, wherein the cancer is ovarian cancer.

124. The method of any one of claims 117-123, wherein the cancer is mediated by CDKs, such as CDK4.
